(19) United States
(12) Patent Application Publication
(10) Pub. No.: US 2025/0120695 A1
(43) Pub. Date: Apr. 17, 2025

(54) METHOD OF OPERATING A SURGICAL STAPLING INSTRUMENT

(71) Applicant: Cilag GmbH International, Zug (CH)

(72) Inventors: Frederick E. Shelton, IV, Hillsboro, OH (US); Shane R. Adams, Lebanon, OH (US); Taylor W. Aronhalt, Loveland, OH (US); Nicholas J. Ross, Franklin, OH (US); Adam D. Hensel, Gahanna, OH (US); Kevin M. Fiebig, Cincinnati, OH (US); Matthew D. Cowperthwait, Cincinnati, OH (US); Eric B. LaFay, Madeira, OH (US)

(21) Appl. No.: 18/379,759

(22) Filed: Oct. 13, 2023

(51) Int. Cl. A61B 17/072; A61B 17/00; A61B 90/00

(52) U.S. Cl. CPC A61B 17/072; A61B 2017/00022; A61B 2017/00084; A61B 2017/00398; A61B 2017/07271; A61B 2017/07285; A61B 2090/064

(57) ABSTRACT

A method for sequential firings of staple cartridges is disclosed. The sequential firings including a first firing that deploys first staples into a first tissue portion from a first staple cartridge and a second firing that deploys second staples into a second tissue portion from a second staple cartridge. The method comprises monitoring a parameter indicative of a tissue response associated with the first firing, assessing the tissue response based on the parameter, and adjusting an operational parameter associated with the second firing based on the tissue response during the first firing.

### BACKGROUND

The present disclosure relates to surgical instruments and, in various arrangements, to surgical stapling and cutting instruments and staple cartridges for use therewith that are to staple and cut tissue.

### SUMMARY

In accordance with the present disclosure, a method for sequential firings of staple cartridges is disclosed. The sequential firings including a first firing that deploys first staples into a first tissue portion from a first staple cartridge and a second firing that deploys second staples into a second tissue portion from a second staple cartridge. The method comprises monitoring a parameter indicative of a tissue response associated with the first firing, assessing the tissue response based on the parameter, and adjusting an operational parameter associated with the second firing based on the tissue response during the first firing.

In accordance with the present disclosure, a method executable by a control circuit of a surgical system including an end effector and a motor powered by a power source is disclosed. The method comprises setting a power source lower threshold, transitioning the motor to an on state for a first period, the on state in which the motor drives a motion at the end effector to perform a tissue treatment event, detecting a dropped voltage potential of the power source at the end of the first period, conducting a first comparison between the dropped voltage potential and the power source lower threshold, and transitioning the motor to an off state for a second period based on the first comparison, the off state in which the motor ceases to the motion at the end effector.

### BRIEF DESCRIPTION OF THE DRAWINGS

Various features of the embodiments described herein, together with advantages thereof, may be understood in accordance with the following description taken in conjunction with the accompanying drawings as follows:

FIG. 1 illustrates an exploded view of an end effector and a shaft portion of a surgical stapling system, in accordance with the present disclosure.

FIG. 2 is a diagram illustrating components of a staple cartridge, in accordance with the present disclosure.

FIG. 3 is a graph depicting positional data on the X-axis and force data on the Y-axis, in accordance with the present disclosure.

FIG. 3A is a graph depicting positional data on the X-axis and force data on the Y-axis, in accordance with the present disclosure.

FIG. 3B is a graph depicting positional data on the X-axis and force data on the Y-axis, in accordance with the present disclosure.

FIG. 4 is a diagram illustrating various components of a surgical stapling system, in accordance with the present disclosure.

FIG. 5 is a flow diagram of a method for non-visually detecting staple malformation, in accordance with the present disclosure.

FIG. 6 is a graph depicting positional data on the X-axis and force data associated with a single staple driver on the Y-axis, in accordance with the present disclosure.

FIG. 7 is a graph depicting positional data on the X-axis and combined force data associated with groups of staple drivers on the Y-axis, in accordance with the present disclosure.

FIG. 8 a flow diagram of a method for non-visually detecting staple malformation, in accordance with the present disclosure.

FIG. 9 a flow diagram of a method for predicatively and autonomously implementing a future pause during a firing stroke based on a tissue response to a previous pause, in accordance with the present disclosure.

FIG. 10 is a flow diagram illustrating one embodiment of implementing a threshold-based pause of the tissue treatment event.

FIG. 11 is a graph illustrating a firing force data associated with a surgical stapling system, in accordance with the present disclosure.

FIG. 12 is a graph illustrating a firing force data associated with a surgical stapling system, in accordance with the present disclosure.

FIG. 13 illustrates a table stored in a memory circuit of a surgical stapling system, in accordance with the present disclosure.

FIG. 14 is a graph illustrating two non-threshold based pauses of a tissue treatment event by the surgical stapling system, in accordance with at the present disclosure.

FIG. 15 is flow diagram illustrating a logic configuration of a method for adjusting operational parameters of a surgical stapling system, in accordance with the present disclosure.

FIG. 16 is a graph illustrating an adaptive behavior of a surgical stapling system during a surgical procedure that involves a sequential firing of multiple staple cartridges, in accordance with the present disclosure.

FIG. 17 is a flow diagram illustrating a logic configuration of a method for adjusting a second firing based on a change in the tissue response in predetermined segments of a first firing, in accordance with the present disclosure.

FIG. 18 illustrates a logic configuration of a method that utilizes learned triggers for motor control adaptation, learned triggers for motor control adaptation, in accordance with the present disclosure.

FIG. 19 is a graph illustrating various parameters associated with a staple cartridge firing by the surgical system, which are indicative of the tissue response, in accordance with the present disclosure.

FIG. 20 is a block diagram of a surgical system comprising an end effector, a motor-driven drive assembly, and a control circuit configured to control actuation of the motor-driven drive assembly, in accordance with the present disclosure;

FIG. 21 is a partial perspective view of a surgical stapling assembly and tissue stapled and cut by the surgical stapling assembly, in accordance with the present disclosure;

FIG. 22 is a partial perspective view of the surgical stapling end effector of FIG. 21, wherein the surgical stapling assembly comprises a shaft, a first jaw, and a second jaw movable relative to the first jaw to clamp tissue therebetween;

FIG. 23 is a perspective view of a drive assembly for use with a surgical stapling assembly, wherein the drive assembly comprises a motor, a drive train, and an output, in accordance with the present disclosure;

FIG. 24 is an elevational view of the drive train of FIG. 23;

FIG. 25 is a perspective view of a gear stage for use with a gear train of a surgical instrument drive assembly, wherein the gear stage comprises a carrier and a plurality of planet gears rotatably mounted to the carrier, in accordance with the present disclosure;

FIG. 26 is a perspective view of a gear stage for use with a gear train of a surgical instrument drive assembly, wherein the gear stage comprises a carrier and a plurality of planet gears rotatably mounted to the carrier, in accordance with the present disclosure;

FIG. 27 is a perspective view of a gear stage for use with a gear train of a surgical instrument drive assembly, wherein the gear stage comprises a carrier and a plurality of planet gears rotatably mounted to the carrier, in accordance with the present disclosure;

FIG. 28 is a perspective view of a gear stage for use with a gear train of a surgical instrument drive assembly, wherein the gear stage comprises a carrier and a plurality of planet gears rotatably mounted to the carrier, and wherein the carrier comprises carrier posts positioned between each planet gear to increase the inertial properties of the gear stage, in accordance with the present disclosure;

FIG. 29 is a perspective view of a carrier for use with a gear stage, wherein the carrier comprises mounting posts for rotatably supporting planet gears thereon and a pinion configured to drive a subsequent gear stage, in accordance with the present disclosure;

FIG. 30 is a perspective view of a carrier for use with a gear stage, wherein the carrier comprises mounting posts for rotatably supporting planet gears thereon and a pinion configured to drive a subsequent gear stage, in accordance with the present disclosure;

FIG. 31 is a perspective view of a carrier for use with a gear stage, wherein the carrier comprises mounting posts for rotatably supporting planet gears thereon and a pinion configured to drive a subsequent gear stage, and wherein the carrier further comprises secondary posts extending therefrom to increase the inertia of the carrier, in accordance with the present disclosure;

FIG. 32 is a perspective view of a carrier for use with a gear stage, wherein the carrier comprises mounting posts for rotatably supporting planet gears thereon and a pinion configured to drive a subsequent gear stage, and wherein the carrier further comprises secondary posts extending therefrom to increase the inertia of the carrier, in accordance with the present disclosure;

FIG. 33 is an elevational view of a variable flywheel for use with a drive assembly of a surgical instrument, wherein the variable flywheel comprises movable masses for varying the storable kinetic energy of a drive assembly, and wherein the masses are illustrated in a collapsed position, in accordance with the present disclosure;

FIG. 34 is an elevational view of the variable flywheel of FIG. 33, wherein the masses are illustrated in an expanded position;

FIG. 35 is an cross-sectional view of a motor for use with a drive assembly of a surgical instrument, in accordance with the present disclosure;

FIG. 36 is an cross-sectional view of a motor for use with a drive assembly of a surgical instrument, in accordance with the present disclosure;

FIG. 37 is a logic flow diagram configured to be executed by a control circuit, wherein the control circuit is configured to adjust a sensitivity of a parameter threshold indicative of a motor stall condition, in accordance with the present disclosure; and

FIG. 38 is a graph of an example drive stroke of a surgical instrument employing the logic flow diagram of FIG. 37, in accordance with the present disclosure.

FIG. 39 is a power recovery circuit for use with a surgical instrument, in accordance with the present disclosure;

FIG. 40 is a power recovery circuit for use with a surgical instrument, in accordance with the present disclosure;

FIG. 41 illustrates a perspective view of a surgical stapling system, in accordance with the present disclosure;

FIG. 42 illustrates an exploded view of the surgical stapling system of FIG. 41, in accordance with the present disclosure;

FIG. 43 illustrates a block diagram of a surgical stapling system, in accordance with the present disclosure;

FIG. 44 illustrates a block diagram of a surgical stapling system, in accordance with the present disclosure;

FIG. 45 illustrates graphs for exemplary firing strokes of a firing driver, in accordance with of the present disclosure;

FIG. 46 illustrates a circuit that includes capacitors in-line with field effect transistors, in accordance with the present disclosure;

FIG. 47 illustrates a switching capacitor circuit and graphs associated therewith, in accordance with the present disclosure;

FIGS. 48A-48D illustrate voltage drops of power sources with different battery materials over time;

FIG. 49 illustrates graphs for an exemplary firing stroke of a firing driver, in accordance with the present disclosure;

FIG. 50 illustrates a method for controlling a surgical system, in accordance with the present disclosure;

FIG. 51 illustrates a battery, in accordance with the present disclosure;

FIG. 52 illustrates graphs for an exemplary firing stroke of a firing driver using the battery of FIG. 51, in accordance with the present disclosure;

FIG. 53 illustrates the load curve profile for a 15 W power supply;

FIG. 54 illustrates a circuit, in accordance with the present disclosure;

FIG. 55 illustrates a graph that illustrates button position of a surgical system, a switch position, and firing status of a motor over time, in accordance with the present disclosure;

FIG. 56 illustrates a method for controlling a surgical system, in accordance with the present disclosure;

FIG. 57 illustrates a method for controlling a surgical system, in accordance with the present disclosure;

FIG. 58 illustrates a printed circuit board including a heat sink, in accordance with the present disclosure.

FIG. 59 is an example of a control circuit, in accordance with the present disclosure.

FIG. 60 is an example of a flux ring, in accordance with the present disclosure.

FIG. 61 is an illustration of the flux ring disposed around a motor, in accordance with the present disclosure.

FIG. 62 illustrates a vapor chamber to remove heat from a motor, in accordance with the present disclosure.

FIG. 63 is a graph 4100 illustrating characteristics of typical MOSFET devices.

FIG. 64 illustrates a graph of characteristic Drain to Source Voltage (V_{DS}) vs Drain Current (ID) curves for a MOSFET device operating at different values of Gate to Source Voltage (V_{GS}).

FIG. 65 is a Torque Load vs Speed graph illustrating an example of characteristics of operating a motorized surgical instrument, in accordance with the present disclosure.

FIG. 66 is an example of a portion of a powered surgical stapler to control a motor of a powered surgical instrument, in accordance with the present disclosure.

FIG. 67 is a graph illustrating an example of a stored profile of voltage during a firing stroke, in accordance with the present disclosure.

FIG. 68 is an example of a circuit to control the motor of a powered surgical stapler, in accordance with the present disclosure.

FIG. 69 is an example of a circuit to control the motor of a powered surgical stapler, in accordance with the present disclosure.

FIG. 70 is a circuit, in accordance with the present disclosure.

FIG. 71 is a circuit, in accordance with the present disclosure.

FIG. 72 illustrates an example of a DC/DC converter type buck-boost converter circuit, in accordance with the present disclosure.

FIG. 73 illustrates example operational modes of an example DC/DC converter type buck-boost converter circuit such as the unloaded buck-boost converter circuit shown in FIG. 72, in accordance with the present disclosure.

FIG. 74 illustrates an example of a DC/DC converter type buck-boost converter circuit in accordance with the present disclosure.

FIG. 75 illustrates example power loss and efficiency at different load currents for a buck-boost DC/DC converter type buck-boost converter circuit such as the loaded buck-boost converter circuit shown in FIG. 74, in accordance with the present disclosure.

FIG. 76 illustrates a basic topology of a buck converter circuit that may be employed in accordance with the present disclosure.

FIG. 77 illustrates the output current of the buck converter circuit IOUT shown in FIG. 76 in accordance with the present disclosure.

FIG. 78 illustrates the current I_{Q1} through the first transistor in FIG. 76, in accordance with the present disclosure.

FIG. 79 illustrates the current I_{Q2} through the second transistor in FIG. 76, in accordance with the present disclosure.

FIG. 80 is a graph of efficiency (%) as a function of output current (A) over a range of output currents of the buck converter circuit shown in FIG. 76 at discrete voltage values, in accordance with the present disclosure.

FIG. 81 is a graph of efficiency (%) as a function of load current (A) over a range of load currents for a LDO regulator, such as the LDO regulator (FIGS. 70 and 71) and a DC/DC converter type buck-boost converter circuits (FIGS. 72 and 74), in accordance with the present disclosure.

FIG. 82 illustrates a surgical system, in accordance with the present disclosure.

FIG. 83 illustrates an example implementation of a voltage boost converter circuit, in accordance with the present disclosure.

FIG. 84 is a graph illustrating efficiency (%) as a function of output current (A) for a DC/DC boost regulator, in accordance with the present disclosure.

FIG. 85 is a variable potentiometer, in accordance with the present disclosure.

FIG. 86 is a graph of drain current (ID) as a function of drain-to-source voltage (V_{DS}) for switching a field effect transistor, in accordance with the present disclosure.

FIG. 87 is a graphical depiction of leakage current losses, in accordance with the present disclosure.

FIG. 88 is a graph of a battery discharge curve, in accordance with the present disclosure.

FIG. 89 is a super capacitor charging circuit, in accordance with the present disclosure.

FIG. 90 illustrates a motor drive circuit with boost circuit connections, in accordance with the present disclosure.

FIG. 91 is a vibration circuit to harvest energy from vibrations of the surgical stapling instrument (FIGS. 1-4), in accordance with the present disclosure.

FIG. 92 is a circuit to collect power from the movement of a drive bar of the surgical stapling instrument, in accordance with the present disclosure.

FIG. 93 is a circuit to collect power from the movement of a drive bar of the surgical stapling instrument, in accordance with the present disclosure.

FIG. 94 illustrates a graph of additional energy applied to the system from the circuit, in accordance with the present disclosure.

FIG. 95 is a distal perspective view of a surgical instrument, in accordance with the present disclosure.

FIG. 96 is a partial perspective view of the surgical instrument of FIG. 95 showing a flex circuit, in phantom, extending through an articulation joint assembly, in accordance with the present disclosure.

FIG. 97 is a partial cross section view of the surgical instrument of FIG. 95 showing the flex circuit of FIG. 96.

FIG. 98 illustrates the surgical instrument of FIG. 95 in a first fully articulated position.

FIG. 99 illustrates the surgical instrument of FIG. 95 in an unarticulated position.

FIG. 100 illustrates the surgical instrument of FIG. 95 in a second fully articulated position.

FIG. 101 illustrates a perspective view of a surgical instrument, in accordance with the present disclosure;

FIG. 102 illustrates an articulation joint assembly, in accordance with the present disclosure;

FIG. 103 illustrates articulation axes and articulation planes of the articulation joint assembly of FIG. 102;

FIG. 104 illustrates a perspective view of a wiring harness in a non-articulated state, in accordance with the present disclosure;

FIG. 105 illustrates an axial view of the of the wiring harness of FIG. 104;

FIG. 106 illustrates bending planes of the wiring harness of FIG. 104;

FIG. 107 illustrates a perspective view of a wiring harness in an articulated state, in accordance with the present disclosure;

FIG. 108 illustrates a partial plan view of the wiring harness perspective view of the wiring harness of FIG. 107;

FIG. 109A illustrates conductive elements of a wiring harness, in accordance with the present disclosure;

FIG. 109B illustrates an exploded view of conductive elements of FIG. 109A;

FIG. 110 illustrates a perspective view of a retainer, in accordance with the present disclosure;

FIG. 111 illustrates a perspective view of a retainer, in accordance with the present disclosure;

FIG. 112 illustrates scissoring portions of a wiring assembly, in accordance with the present disclosure; and

FIG. 113 illustrates linkages of the scissoring portions of FIG. 112.

FIG. 114 illustrates a perspective view of an articulation joint, in accordance with the present disclosure.

FIG. 115 is a partial perspective view of a surgical instrument including an end effector with a channel antenna for wireless transmission of power and/or data, in accordance with the present disclosure.

FIG. 116 is a partial cross-sectional view of the surgical instrument of FIG. 115 in an assembled configuration with a staple cartridge including a cartridge antenna, in accordance with the present disclosure.

FIG. 117 is a partial cross-sectional view of the surgical instrument of FIG. 115, in accordance with the present disclosure.

FIG. 118 is a cross-sectional view of the end effector of FIG. 116, in accordance with the present disclosure.

FIG. 119 is a partial cross-sectional view of a longitudinal channel including an antenna and an insulative layer, in accordance with the present disclosure.

FIG. 120 is partial cross-sectional view of a longitudinal channel including an antenna and a ferrite layer, in accordance with the present disclosure.

FIG. 121 is partial cross-sectional view of a longitudinal channel and a staple cartridge in an assembled configuration, in accordance with the present disclosure.

FIG. 122 is a partial perspective view of a staple cartridge and a longitudinal channel, in accordance with the present disclosure.

FIG. 123 is a partial cross-sectional view of a surgical instrument including a staple cartridge assembled with a longitudinal channel, in accordance with the present disclosure.

FIG. 124 is a block diagram of a surgical system comprising an end effector, a motor-driven drive assembly, and a control circuit configured to control actuation of the motor-driven drive assembly, wherein the control circuit is electrically coupled with electronic circuits of a replaceable staple cartridge of the end effector, in accordance with the present disclosure;

FIG. 125 is a partial perspective view of a surgical stapling assembly and tissue stapled and cut by the surgical stapling assembly, in accordance with the present disclosure;

FIG. 126 is a partial perspective view of the surgical stapling end effector of FIG. 125, wherein the surgical stapling assembly comprises a shaft, a first jaw, and a second jaw movable relative to the first jaw to clamp tissue therebetween;

FIG. 127 is a perspective view of a surgical stapling end effector comprising a shaft, a first jaw, a second jaw movable relative to the first jaw, and a replaceable staple cartridge, in accordance with the present disclosure;

FIG. 128 is a perspective view of the surgical stapling end effector of FIG. 127, wherein the replaceable staple cartridge is illustrated in an uninstalled position, and wherein the replaceable staple cartridge comprises a plurality of onboard electronic systems, and wherein the plurality of onboard electronic systems comprises a modular electronics package and wireless transmission coils;

FIG. 129 is a perspective view of the first jaw and the second jaw of FIG. 127, wherein the first jaw comprises a cartridge channel comprising corresponding wireless transmission coils configured to transmit power and/or data to and/or from the replaceable staple cartridge upon installation of the replaceable staple cartridge into the cartridge channel;

FIG. 130 is a perspective view of the first jaw of FIG. 127 illustrating the wireless transmission coils of the first jaw and the replaceable staple cartridge and the modular electronics package;

FIG. 131 is a cross-sectional view of the surgical stapling end effector of FIG. 127;

FIG. 132 is a perspective view of a portion of a surgical stapling end effector comprising a first jaw, a second jaw, and a replaceable staple cartridge positionable within the first jaw (illustrated in a partially installed position), wherein the replaceable staple cartridge comprises an electrical connector at the proximal end couplable with a corresponding electrical connector positioned within the first jaw in accordance with the present disclosure;

FIG. 133 is a perspective view of the surgical stapling end effector of FIG. 132;

FIG. 134 is a perspective view of a replaceable staple cartridge comprising onboard electronics and an electrical connector extending from a proximal end of the replaceable staple cartridge in accordance with the present disclosure;

FIG. 135 is a perspective view of a surgical stapling end effector comprising a cartridge channel jaw, a replaceable staple cartridge configured to be installed in the cartridge channel jaw, and an electronic system comprising an electrical connector positioned at a proximal end of the replaceable staple cartridge and a corresponding electrical connector positioned within the first jaw configured to be electrically coupled to the electrical connector of the replaceable staple cartridge in accordance with the present disclosure;

FIG. 136 is a perspective view of a portion of the surgical stapling end effector of FIG. 135;

FIG. 137 is a perspective view of a portion of a surgical stapling end effector comprising a replaceable staple cartridge configured to be installed in a jaw of the surgical stapling end effector and an electronics interface comprising wireless transmission coils to communicate power and/or data to and/or from the replaceable staple cartridge in accordance with the present disclosure;

FIG. 138 is a perspective view of an electrical connector in accordance with the present disclosure;

FIG. 139 is a perspective view of the electrical connector of FIG. 15;

FIG. 140 is a perspective view of an electrical connector in accordance with the present disclosure;

FIG. 141 is a perspective view of the electrical connector of FIG. 140;

FIG. 142 is an elevational view of the electrical connectors in a connected configuration of FIGS. 138 and 140;

FIG. 143 is an elevational view of an electrical connector system of a surgical stapling end effector in accordance with the present disclosure;

FIG. 144 is a perspective view of a replaceable staple cartridge comprising onboard electronics systems including a modular electronics package, a wireless transmission coil interface, and an electrical circuit indicative of one or more properties of the replaceable staple cartridge detectable by a surgical instrument in accordance with the present disclosure;

FIG. 145 is a partial perspective view of replaceable staple cartridge of FIG. 144;

FIG. 146 is a logic flow diagram configured to be executed by a control circuit, wherein the control circuit is configured to determine a state of the tissue clamped between a first jaw and a second jaw based on a comparison of end effector parameter readings of various sensors in accordance with the present disclosure;

FIG. 147 is an elevational view of a surgical stapling end effector comprising a plurality of sensors, wherein the surgical stapling end effector is in an unclamped configuration in accordance with the present disclosure;

FIG. 148 is an elevational view of the surgical stapling end effector of FIG. 147, wherein the surgical stapling end effector is in a clamped configuration;

FIG. 149 is an elevational view of the surgical stapling end effector of FIG. 147, wherein the surgical stapling end effector is clamped onto a foreign object;

FIG. 150 is a graph of multiple firing strokes of a stapling end effector including multiple discrete sensor zones;

FIG. 151 is a graph of multiple firing strokes of a stapling end effector where one of the firing strokes is pulsed to alleviate firing load; and

FIG. 152 is a graph of multiple firing strokes of a stapling end effector where one of the firing strokes is pulsed to alleviate firing load.

Corresponding reference characters indicate corresponding parts throughout the several views.

### DESCRIPTION

Applicant of the present application owns the following U.S. Patent Applications that were filed on even date herewith and which are each herein incorporated by reference in their respective entireties:
U.S. Patent Application, titled SURGICAL STAPLING SYSTEMS WITH ADAPTIVE STAPLE FIRING ALGORITHMS; Attorney Docket No. END9484USNP3/220491-3;
U.S. Patent Application, titled LEARNED TRIGGERS FOR ADAPTIVE CONTROL OF SURGICAL STAPLING SYSTEMS; Attorney Docket No. END9484USNP4/220491-4;
U.S. Patent Application, titled CONTROL CIRCUIT FOR ACTUATING MOTORIZED FUNCTION OF SURGICAL STAPLING INSTRUMENT UTILIZING INERTIAL DRIVE TRAIN PROPERTIES; Attorney Docket No. END9484USNP5/220491-5;
U.S. Patent Application, titled PROPORTIONATE BALANCING OF THE FUNCTION IMPACT MAGNITUDE OF BATTERY OUTPUT TO PEAK MOTOR CURRENT; Attorney Docket No. END9484USNP6/220491-6;
U.S. Patent Application, titled MOTOR OPTIMIZATION BY MINIMIZATION OF PARASITIC LOSSES AND TUNING MOTOR DRIVE CONFIGURATION; Attorney Docket No. END9484USNP7/220491-7;
U.S. Patent Application, titled APPARATUS AND METHOD TO REDUCE PARASITIC LOSSES OF THE ELECTRICAL SYSTEM OF A SURGICAL INSTRUMENT; Attorney Docket No. END9484USNP8/220491-8;
U.S. Patent Application, titled SURGICAL TOOL WITH RELAXED FLEX CIRCUIT ARTICULATION; Attorney Docket No. END9484USNP9/220491-9;
U.S. Patent Application, titled WIRING HARNESS FOR SMART STAPLER WITH MULTI AXIS ARTICULATION; Attorney Docket No. END9484USNP10/220491-10;
U.S. Patent Application, titled SURGICAL SYSTEM WITH WIRELESS ARRAY FOR POWER AND DATA TRANSFER; Attorney Docket No. END9484USNP11/220491-11; and
U.S. Patent Application, titled SURGICAL STAPLE CARTRIDGE COMPRISING REPLACEABLE ELECTRONICS PACKAGE; Attorney Docket No. END9484USNP12/220491-12.

Applicant of the present application owns the following U.S. Patent Applications that were filed on even date herewith and which are each herein incorporated by reference in their respective entireties:
U.S. Patent Application, titled METHOD OF ASSEMBLING A STAPLE CARTRIDGE; Attorney Docket No. END9484USNP13/220491-13M;
U.S. Patent Application, titled CONTROL SURFACES ON A STAPLE DRIVER OF A SURGICAL STAPLE CARTRIDGE; Attorney Docket No. END9484USNP14/220491-14;
U.S. Patent Application, titled INTEGRAL CARTRIDGE STIFFENING FEATURES TO REDUCE CARTRIDGE DEFLECTION; Attorney Docket No. END9484USNP15/220491-15;
U.S. Patent Application, titled STAPLE CARTRIDGE COMPRISING WALL STRUCTURES TO REDUCE CARTRIDGE DEFLECTION; Attorney Docket No. END9484USNP16/220491-16;
U.S. Patent Application, titled PAN-LESS STAPLE CARTRIDGE ASSEMBLY COMPRISING RETENTION FEATURES FOR HOLDING STAPLE DRIVERS AND SLED; Attorney Docket No. END9484USNP17/220491-17;
U.S. Patent Application, titled STAPLE CARTRIDGE COMPRISING A SLED HAVING A DRIVER LIFT CAM; Attorney Docket No. END9484USNP18/220491-18;
U.S. Patent Application, titled SURGICAL STAPLE CARTRIDGES WITH SLEDS CONFIGURED TO BE COUPLED TO A FIRING DRIVER OF A COMPATIBLE SURGICAL STAPLER; Attorney Docket No. END9484USNP19/220491-19;
U.S. Patent Application, titled STAPLE CARTRIDGE COMPRISING A COMPOSITE SLED; Attorney Docket No. END9484USNP20/220491-20;
U.S. Patent Application, titled SURGICAL INSTRUMENTS WITH JAW AND FIRING ACTUATOR LOCKOUT ARRANGEMENTS LOCATED PROXIMAL TO A JAW PIVOT LOCATION; Attorney Docket No. END9484USNP21/220491-21;
U.S. Patent Application, titled SURGICAL INSTRUMENTS WITH LATERALLY ENGAGEABLE LOCKING ARRANGEMENTS FOR LOCKING A FIRING ACTUATOR; Attorney Docket No. END9484USNP22/220491-22;
U.S. Patent Application, titled DUAL INDEPENDENT KEYED LOCKING MEMBERS ACTING ON THE SAME DRIVE MEMBER; Attorney Docket No. END9484USNP23/220491-23;
U.S. Patent Application, titled ADJUNCTS FOR USE WITH SURGICAL STAPLING INSTRUMENTS; Attorney Docket No. END9484USNP24/220491-24;
U.S. Patent Application, titled ADJUNCTS FOR USE WITH SURGICAL STAPLING INSTRUMENTS; Attorney Docket No. END9484USNP25/220491-25;
U.S. Patent Application, titled JAW CONTROL SURFACES ON A SURGICAL INSTRUMENT JAW; Attorney Docket No. END9484USNP26/220491-26;
U.S. Patent Application, titled ZONED ALGORITHM ADAPTIVE CHANGES BASED ON CORRELATION OF COOPERATIVE COMPRESSION CONTRIBUTIONS OF TISSUE; Attorney Docket No. END9484USNP27/220491-27;
U.S. Patent Application, titled STAPLE CARTRIDGES COMPRISING TRACE RETENTION FEATURES; Attorney Docket No. END9484USNP29/220491-29; and
U.S. Patent Application, titled STAPLE CARTRIDGES COMPRISING STAPLE RETENTION FEATURES; Attorney Docket No. END9484USNP30/220491-30.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the described and illustrated embodiments are non-limiting examples, and thus it can be appreciated that the specific structural and functional details disclosed herein may be representative and illustrative. Variations and changes may be made without departing from the scope of the claims.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working frame through which the end effector and elongate shaft of a surgical instrument can be advanced.

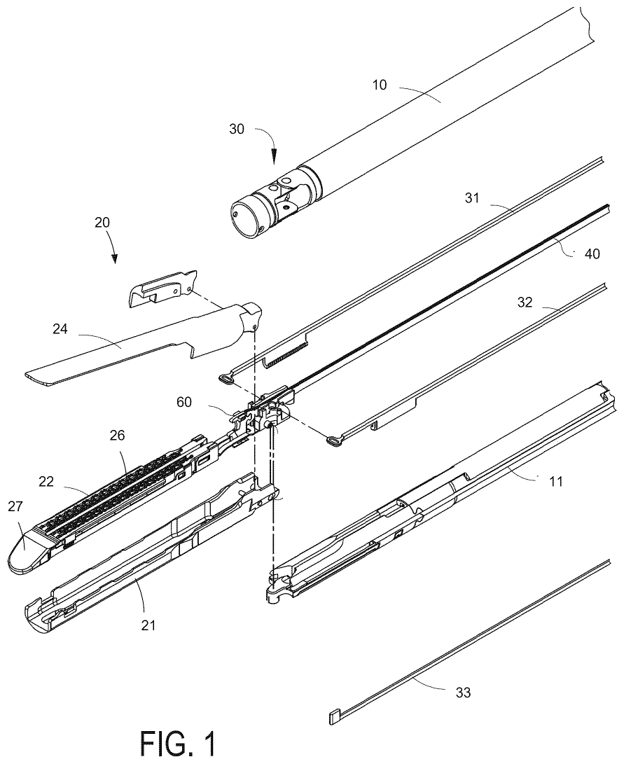

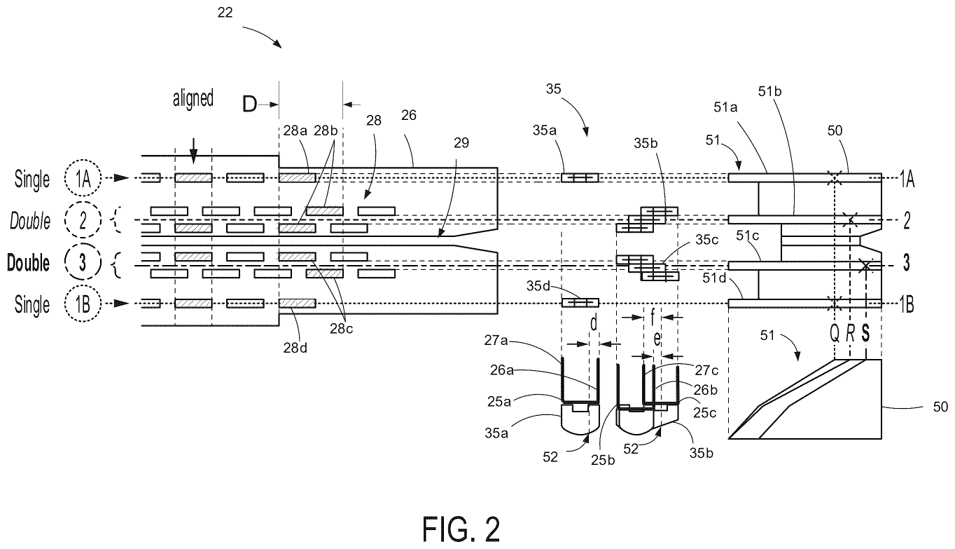

Referring to FIGS. 1-4, a surgical stapling system 5 includes a shaft 10 and an end effector 20 extending from the shaft 10. The end effector 20 includes a first jaw 19 and a second jaw 23. The first jaw 19 defines a channel 21 and a staple cartridge 22 removably positionable in the channel 21. Other embodiments are envisioned in which a staple cartridge is not removable, or at least readily replaceable, from the first jaw 19. The second jaw 23 includes an anvil 24 configured to deform staples 25 (See FIG. 2) ejected from the staple cartridge 22. The second jaw 23 is pivotable relative to the first jaw 19 about a closure axis to transition the end effector 20 between an open configuration and a closed configuration. Other embodiments are envisioned in which the first jaw 19 is pivotable relative to the second jaw 23.

The surgical stapling system 5 further includes an articulation joint 30 configured to permit the end effector 20 to be rotated, or articulated, relative to the shaft 10. The end effector 20 is rotatable about an articulation axis extending through the articulation joint 30. Other embodiments are envisioned which do not include an articulation joint. In the illustrated example, cooperating articulation rods 31, 32 are configured to articulate the end effector 20 relative to the shaft 10 about an articulation joint 30. The surgical stapling system 5 further includes an articulation lock bar 33 configured to selectively prevent the articulation of the end effector 20.

The staple cartridge 22 includes a cartridge body 27 with a proximal end, a distal end, and a deck 26 extending between the proximal end and the distal end. In use, the staple cartridge 22 is positioned on a first side of the tissue to be stapled and the anvil 24 is positioned on a second side of the tissue. In accordance with the present disclosure, the anvil 24 may be moved toward the staple cartridge 22 to compress and clamp the tissue against the deck 26. Further, in accordance with the present disclosure, the staple cartridge 22 may be moved relative to the anvil 24 or, alternatively, both the staple cartridge 22 and the anvil 24 may be moved to compress and clamp the tissue.

A drive shaft 40 is movable distally to motivate a firing beam 60 to transition the end effector 20 toward the closed configuration, thereby compressing the tissue. In the illustrated example, the firing beam 60 is in the form of an I-beam that includes a first cam and a second cam configured to engage the first jaw 19 and second jaw 23, respectively. As the firing beam 60 is advanced distally, the first cam and the second cam can control the distance, or tissue gap, between the deck of the staple cartridge 22 and the anvil 24. In the illustrated example, the firing beam 60 motivates a sled 50 to deploy the staples 25 from the staple cartridge 22. In accordance with the present disclosure, a separate closure mechanism, e.g., a closure tube, can be employed to transition the end effector 20 toward the closed configuration. Also in accordance with the present disclosure, the firing beam 60 may or may not include the first and second cams. Further, in accordance with the present disclosure, the firing beam 60 may be in the form of an E-beam with first, second, and third cams. In accordance with the present disclosure, the firing beam 60 and the closure tube may cooperatively effect closure of the end effector 20. Also, in accordance with the present disclosure, the firing beam 60 may only effect deployment of the staples 25.

In accordance with the present disclosure, as illustrated in FIG. 1, the firing beam 60 may include a knife configured to incise the tissue captured intermediate the staple cartridge 22 and the anvil 24. It is desirable for the knife to be positioned at least partially proximal to the ramped surfaces such that the staples are ejected ahead of the knife. More details about alternative embodiments of surgical stapling systems, suitable for use with the present disclosure, are disclosed in U.S. patent application Ser. No. 15/385,887 entitled METHOD FOR ATTACHING A SHAFT ASSEMBLY TO A SURGICAL INSTRUMENT AND, ALTERNATIVELY, TO A SURGICAL ROBOT, and U.S. patent application Ser. No. 16/209,416, entitled METHOD OF HUB COMMUNICATION, PROCESSING, DISPLAY, AND CLOUD ANALYTICS, which are hereby incorporated by reference herein in their entireties.

The staples 25 removably stored in the cartridge body 27 can be deployed into the tissue. The cartridge body 27 includes staple cavities 28 defined therein wherein staples 25 are removably stored in the staple cavities 28. The staple cavities 28 are arranged in longitudinal rows. In the illustrated example, three rows of staple cavities 28 are positioned on a first side of a longitudinal slot 29 and three rows of staple cavities 28 are positioned on a second side of the longitudinal slot. Other arrangements of staple cavities 28 and staples 25 are possible.

The staples 25 are supported by staple drivers 35 in the cartridge body 27. The staple drivers 35 are movable between a first, or unfired position, and a second, or fired, position to eject the staples 25 from the staple cavities 28. The staple drivers 35 are movable between their unfired positions and their fired positions by a sled 50 that includes ramped surfaces 51 configured to slide under the staple drivers 35 and lift the staple drivers 35, and the staples 25 supported thereon, toward the anvil 24. In the illustrated example, the distal movement of the drive shaft 40 causes the sled 50 to move distally within the staple cartridge 22 to deploy the staples 25.

Referring primarily to FIG. 2, the sled 50 includes a first ramped surface 51a, a second ramped surface 51b, a third ramped surface 51c, and a fourth ramped surface 51d configured to engage a first staple drive 35a, a second staple driver 35b, a third staple driver 35c, and a fourth staple driver 35d, respectively, along a staple-forming distance (D) to deploy staples 25 from corresponding staple cavities 28a, 28b, 28c, 28d for forming against corresponding forming pockets in the anvil 24. In the illustrated example, the staple drivers 35b, 35c are double drivers, while the staple drivers 35a, 35d are single drivers. Double drivers support two staples in two separate staple cavities, while single drivers support a single staple in a single staple cavity.

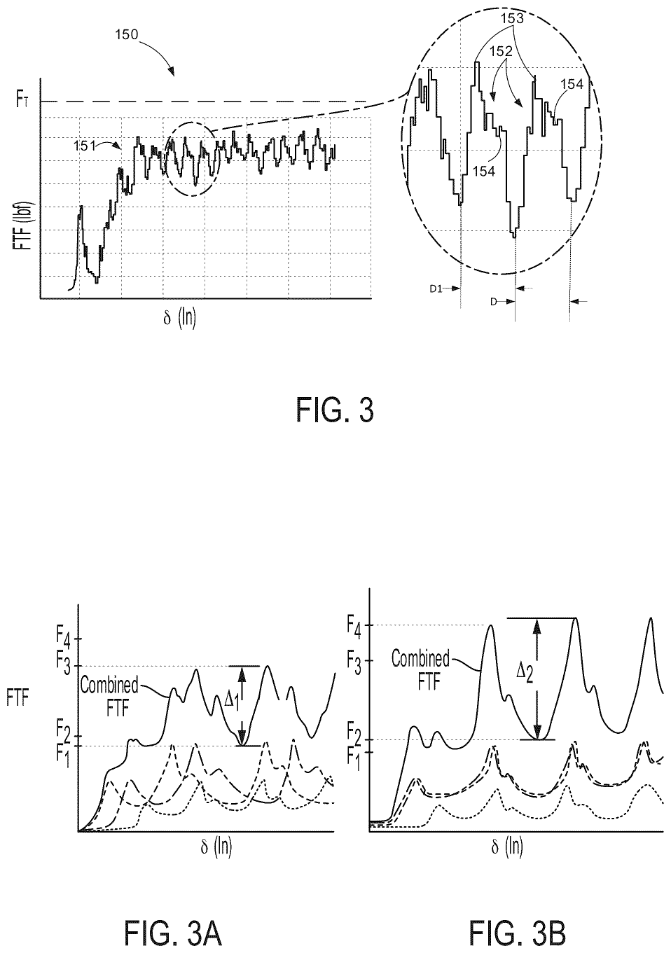

FIG. 3 illustrates one exemplification of a staple-forming distance (D). In accordance with the present disclosure, the staple-forming distance (D) can be characterized as a distance spanning a number of the staple cavities 28 housing staple drivers 35 that are simultaneously engaged by the sled 50. Further, in accordance with the present disclosure, the staple-forming distance (D) can be characterized as a distance travelled by the sled 50 while simultaneously engaging a group of staple drivers 35 to transition the staple drivers 35 between their unfired positions and their fired positions, thereby causing the staples 25 positioned on the staple drivers 35 to be formed against the anvil 24.

The sled 50 and the staple drivers 35a-d are configured to stagger staple formation of staples 25 in staple cavities 28a-d. The ramped surfaces 51 are shaped and arranged to facilitate an offset firing of the staples 25 in the staple cavities 28a-d to reduce the forces experienced by the sled 50 as the sled 50 is moved along the staple-forming distance (D). In other words, the ramped surfaces 51 can be tailored to stagger staple-formation progress in a manner that maintains the firing forces experienced by the sled 50 at, or below, a predetermined threshold (F_{T}) (FIG. 3).

In the example illustrated in FIG. 2, the ramped surfaces 51a, 51d are identical, or at least substantially the same, which causes the single staple drivers 35a, 35b to be simultaneously lifted by the sled 50 at the same pace. The ramped surfaces 51b, 51c are different from each other, and different from the ramped surfaces 51a, 51b, which causes the double staple drivers to be lifted by the sled 50 at a different pace from each other, and at a different pace from the staple drivers 35a, 35d. In other words, the peak forces associated with the double drivers 35b, 35c are out of phase with each other, and are also out of phase with peak forces associated with the single drivers 35a, 35d.

With continued reference to FIG. 2, the ramped surfaces 51 cause a peak force associated with simultaneously forming the staples supported by the single staple drivers 35a, 35d to occur, at Q, ahead of a peak force associated with forming staples supported by the staple drivers 35b (at R), 35c (at S). Q, R, S represent positions on the ramped surfaces 51 of the sled 50 corresponding to the staggered peak forces associated with staple drivers 35a, 35d, staple driver 35b, and staple driver 35c, respectively. Staggering the staple-formation progress of staples in staple cavities 28a-d positioned along the staple forming distance (D) reduces the forces experienced by the sled 50 as the sled 50 simultaneously lifts the staple drivers 35a-d through various stages of staple formation including, for example, a staple buckling stage and/or a staple final-crunching stage. Peak forces associated with other groups of staple drivers 35 can be similarly staggered.

Other factors can influence staple-formation progress and peak forces such as, for example, geometries of cam surfaces of the staple drivers, and contact locations between the ramped surfaces 51 and the staple drivers 35. In the illustrated example, contact locations 52, 53 define locations on the cam surfaces of the staple drivers 35a-d where the ramped surfaces 51 of the sled 50 first engage and lift the staple drivers 35a-d to deploy the staples 25. The staggered staple-formation progress is aided by geometries of cam surfaces of the staple drivers 35a-d and/or contact locations 52, 53 between the cam surfaces of the staple drivers 35a-d and corresponding ramped surfaces 51a-d.

In the illustrated example, the contact location 52 of the staple driver 35a is a distance (d) from a proximal staple leg 26a of a staple 25a supported by the staple driver 35a. The contact location 52 is closer to the proximal staple leg 26a than a distal staple leg 27a of the staple 25a. Furthermore, the contact location 53 of the staple driver 35b is a distance (e) from a proximal leg 26b of a distal staple 25b supported by the staple driver 35b, and a distance (f) from a distal staple leg 27c of a proximal staple 25c supported by the staple driver 35b. The distances d, e, f are different from one another, and are tailored to support the staggered staple-formation progress along the staple-forming distance (D). In the illustrated example, the distance (f) is greater than the distance (d), and the distance (d) is greater than the distance (e). Similar contact locations and/or contact surfaces geometries are implemented in the staple drivers 35c, 35d, and other staple drivers 35 along the firing stroke.

FIG. 3 is a graph 150 of forces exerted against the sled 50 at various positions along a firing stroke that yields a deployment of the staples from the staple cartridge 22. The graph 150 depicts displacement/positional data of the sled 50 on the X-axis and load/force data exerted against the sled 50 on the Y-axis. Curved line 151 demonstrates the collective force excreted by consecutive groups of staple drivers 35 against the sled 50 at different positions along the firing stroke. Each group (e.g., 35a-d) of staple drivers spans a staple-forming distance (e.g., D), and is represented on the graph 150 by a force profile 152, along each staple-forming distance, which includes a lowest peak 153 and a highest peak 154. The firing stroke encompasses multiple staple-forming distances, the number of which depends on the number of groups of staple drivers 35 in the staple cartridge 22.

Since the forces exerted by the staple drivers 35a-d against the sled 50 vary depending on the staple formation stages of the staples 25 in the staple cavities 28a-d along the staple forming distance (D), staggering the transition of such staples through the different stages maintains the overall firing force required to move the sled 50 along the staple-forming distances (e.g. D, D1) at, or below, a predetermined threshold (F_{T}), as illustrated in FIG. 3. In addition, staggering the staple formation progress also ensures a smoother force curve. FIGS. 3A and 3B provide a comparison between the staggered approach (FIG. 3A) and the non-staggered approach (FIG. 3B). The differences (41, 42) between similarly situated peak and valley forces indicate that the staggered approach yields a smoother force curve than the non-staggered approach.

Various methods, devices, and systems provided by the present disclosure yield useful clinical outcomes extrapolated from the positional data and the force data of the graph 150. As discussed in greater detail below, the shape of the force profiles 152, location of the highest peaks 153, and/or other characteristics of the curved line 151 can be considered along with various characteristics of the staple cartridge 22 to yield useful clinical outcomes such as, for example, detecting staple malformation and/or controlling various operational parameters associated with the surgical stapling system 5.

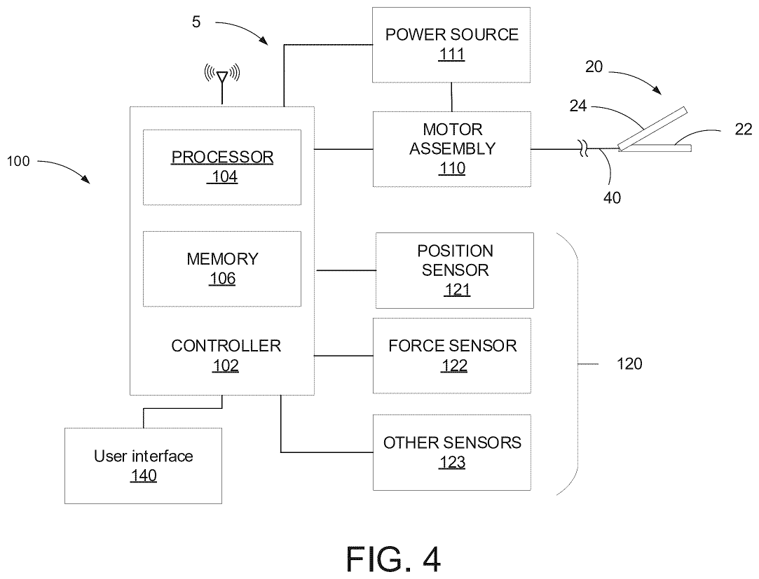

FIG. 4 is a block diagram illustrating one exemplification of the surgical stapling system 5. Various components of the surgical stapling system 5 communicate with a control circuit 100. Such components may receive signals from and/or transmit signals to the control circuit 100. Such signals include command signals, status signals, sensor signals, and/or any other suitable signals. The control circuit 100 can be configured to implement various methods described herein with the aid of various components of the surgical stapling system in communication with the control circuit 100. In the illustrated example, the control circuit 100 includes a controller 102 comprising one or more processors 104 (e.g., microprocessor, microcontroller) communicatively connected to at least one memory circuit 106. The memory circuit 106 stores machine executable instructions that when executed by the processor 104, cause the processor 104 to execute machine instructions to implement various processes described herein. The processor 104 may be any one of a number of single or multi-core processors known in the art. The memory circuit 106 may comprise volatile and non-volatile storage media. The processor 104 may include an instruction processing unit and an arithmetic unit. The instruction processing unit may be configured to receive instructions from the memory circuit 106. In accordance with the present disclosure, the control circuit 100 may include a combinational logic circuit and/or a sequential logic circuit.

In accordance with the present disclosure, the control circuit 100 may be configured to communicate with a motor assembly 110 that includes a motor 113 and a motor controller, for example. The motor assembly 110 may generate rotational motion to effect a translating motion of the drive shaft 40. The control circuit 160 may generate a motor set point signal. The motor set point signal may be provided to a motor controller. The motor controller may comprise one or more circuits configured to provide a motor drive signal to a motor to drive the motor 113 as described herein. In some examples, the motor 113 may be a brushed DC electric motor. For example, the velocity of the motor 113 may be proportional to the motor drive signal. Further, in accordance with the present disclosure, the motor 113 may be a brushless DC electric motor and the motor drive signal may comprise a PWM signal provided to one or more stator windings of the motor. Also, in accordance with the present disclosure, the motor controller may be omitted, and the control circuit 100 may generate the motor drive signal directly. The position, movement, displacement, and/or translation of the drive shaft 40, the firing beam 60 and/or the sled 50 (collectively referred to herein as the “firing assembly”) can be measured/monitored by the control circuit 100 based on input from one or more sensors 120.

The motor assembly 110 may be powered by a power source 111 that in one form may comprise a removable power pack. The power pack may include a housing configured to support a plurality of batteries that may each include, for example, a Lithium Ion (“LI”) or other suitable battery, and may be connected in series, for example. The power source 111 may be replaceable and/or rechargeable. Other power sources are contemplated by the present disclosure.

The sensors 120 may include a position sensor 121 configured to sense a position, movement, displacement, and/or translation of one or more components of the firing assembly such as, for example, the drive shaft 40, the firing beam 60 and/or the sled 50. The sensor 121 may include any type of sensor that is capable of generating position data that indicate a position of the firing assembly. In some examples, the sensor 121 may include an encoder configured to provide a series of pulses to the control circuit 100 as the firing assembly translates distally and proximally. The control circuit 100 may track the pulses to determine the position, movement, displacement, and/or translation of the components of the firing assembly. Other suitable position sensors may be used, including, for example, a proximity sensor. Other types of position sensors may provide other signals indicating motion of the firing assembly. Where the motor 113 is a stepper motor, the control circuit 100 may track the position of components of the firing assembly by aggregating the number and direction of steps that the motor 113 has been instructed to execute. The sensors 120 may be located in the end effector 20 or at any other portion of the surgical stapling system 5.

Various sensors 120 may be adapted to measure various other parameters such as gap distance versus time, tissue compression versus time, and anvil strain versus time. The sensors 120 may comprise a magnetic sensor, a magnetic field sensor, a strain gauge, a pressure sensor, a force sensor, an inductive sensor such as an eddy current sensor, a resistive sensor, a capacitive sensor, an optical sensor, and/or any other suitable sensor for measuring one or more parameters of the end effector 20. The one or more sensors 120 may be sampled in real time during a clamping operation by the processor 104 of the control circuit 100. The control circuit 100 receives real-time sample measurements to provide and analyze time-based information and assess, in real time, a measured parameter such as, for example, force and/or position parameters.

The one or more sensors 120 may comprise a strain gauge, such as a micro-strain gauge, configured to measure the magnitude of the strain in the anvil 24 during a clamped condition. The strain gauge provides an electrical signal whose amplitude varies with the magnitude of the strain. The sensors 120 may comprise a pressure sensor configured to detect a pressure generated by the presence of compressed tissue between the anvil 24 and the staple cartridge 22. The sensors 120 may be configured to detect impedance of a tissue section located between the anvil 24 and the staple cartridge 22 that is indicative of the thickness and/or fullness of tissue located therebetween.

The sensors 120 may include a force sensor 122 configured to measure forces associated with firing and/or closure conditions. For example, sensor 122 can be at an interaction point between a closure tube and the anvil 24 to detect the closure forces applied by a closure tube to the anvil 24. The forces exerted on the anvil 24 can be representative of the tissue compression experienced by the tissue section captured between the anvil 24 and the staple cartridge 22. The sensor 122 can be positioned at various interaction points along the closure drive system to detect the closure forces applied to the anvil 24.

Similarly, a force sensor 122 can be at an interaction point between components of the firing assembly to detect the firing forces applied by the firing assembly to advance the firing beam 60 and the sled 50 to deploy the staples into tissue and cut the tissue. The measured forces represent a firing load experienced by the firing assembly. Alternatively, or additionally, a current sensor can be employed to measure the current drawn by the motor of the motor assembly 110. The force required to advance the firing assembly corresponds to the current drawn by the motor 113. The measured force can be converted to a digital signal and provided to the control circuit 100.

Further to the above, the surgical stapling system 5 includes a user interface 140 having an input device (e.g., a capacitive touchscreen or a keyboard) for receiving inputs from a user and an output device (e.g., a display screen) for providing outputs to a user. Outputs can include data from a query input by the user, suggestions for products or mixes of products to use in a given procedure, and/or instructions for actions to be carried out before, during, or after surgical procedures. The user interface 140 can be in communication with the control circuit 100, as illustrated in FIG. 4.

Various algorithms, instruments, and systems are provided for non-visually detecting staple malformation. Staple malformation occurs when staples 25 deployed into the tissue grasped by the end effector 20 are not properly formed between the anvil 24 and the staple drivers 35 of the staple cartridge 22. Examples of malformed staples include staples where at least one of the staple legs is over-formed or under-formed thereby yielding a formed-staple shape that deviates from a standard B-shape. Staple malformation can lead to clinical complication including, for example, excessive bleeding and/or improper tissue healing. A clinician may not be able to visually detect an occurrence of staple malformation. Without detection, choices that caused a staple malformation can be repeated, thereby leading to additional complications.

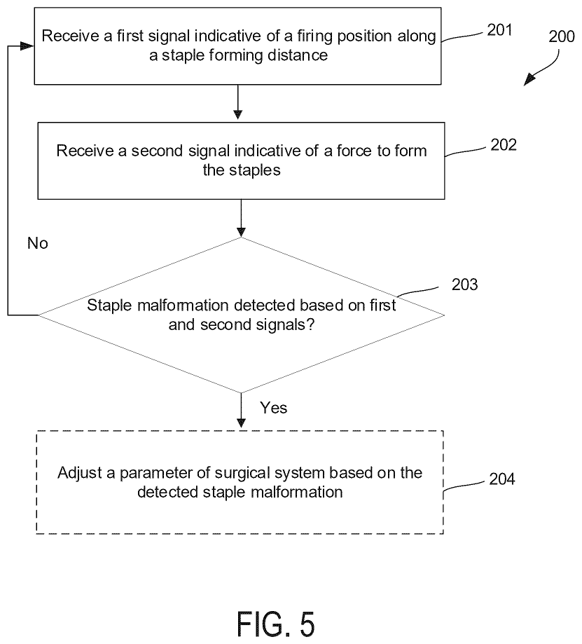

FIG. 5 is a method 200 for detecting staple malformation. With reference to FIG. 5 together with FIG. 4, in accordance with the present disclosure, the control circuit 100 may execute the method 200 to detect staple malformation between the anvil 24 and the staple drivers 35. Program instructions in accordance with the method 200, or portions of the method 200, are stored in a memory circuit such as, for example, the memory circuit 106. The processor 104 may execute the program instructions to achieve a clinical outcome associated with staple malformation. In accordance with the present disclosure, portions of the method 200 can be executed by the processor 104 independently from other portions of the method 200, or by another processer that may be in communication with the processor 104, for example.

According to the method 200, the control circuit 100 receives 201 a first signal from the position sensor 121. The first signal is indicative of a firing position along a staple-forming distance (e.g., D, D1). The control circuit 100 receives 202 a second signal from the force sensor 122. The second signal is indicative of a force to form staples 25 residing in staple cavities 28 positioned along the staple-forming distance. Further to the method 200, the control circuit 100 detects 203 malformation of the staples 25 based on the first signal and based on the second signal, as described in greater detail below. Optionally, according to the method 200, the control circuit 100 can perform a clinical action in response to detecting 203 the staple malformation. In the illustrated example, according to the clinical action, the control circuit 100 adjusts 204 a parameter of the surgical stapling system 5 based on the detection 203 of the staple malformation. The parameter can be, for example, a cartridge selection for the next staple cartridge reload to address detected 203 staple malformation.

In one exemplification, adjusting 204 a parameter of the surgical stapling system 5 can include recommending a different type of cartridge for the next staple cartridge reload to address staple formation. The control circuit 100 can be configured to receive an input indicative of a type of staple cartridge 22 that yielded the detected 203 staple malformation. The input can be in the form of a user input through the user interface 140, for example. Additionally, or alternatively, the input can be received from an identification chip on the staple cartridge 22 that is configured to communicate a staple cartridge identifier to the control circuit 100. In response to detecting 203 a staple malformation, the processor 104 may query a database 124 that stores various staple cartridge types for a staple cartridge reload different from the identified staple cartridge that yielded the staple malformation. The control circuit 100 may recommend a staple cartridge reload based on the results of the query through the user interface 140, for example. As described in greater detail below, the selection can be based on the received 201 first signal and based on the received 202 second signal. In accordance with the present disclosure, the control circuit 100 may issue an alert through the user interface 140 in response to detecting 203 staple malformation. The control circuit 100 also may prompt for a user input through the user interface 140 in response to detecting 203 the staple malformation, for example.

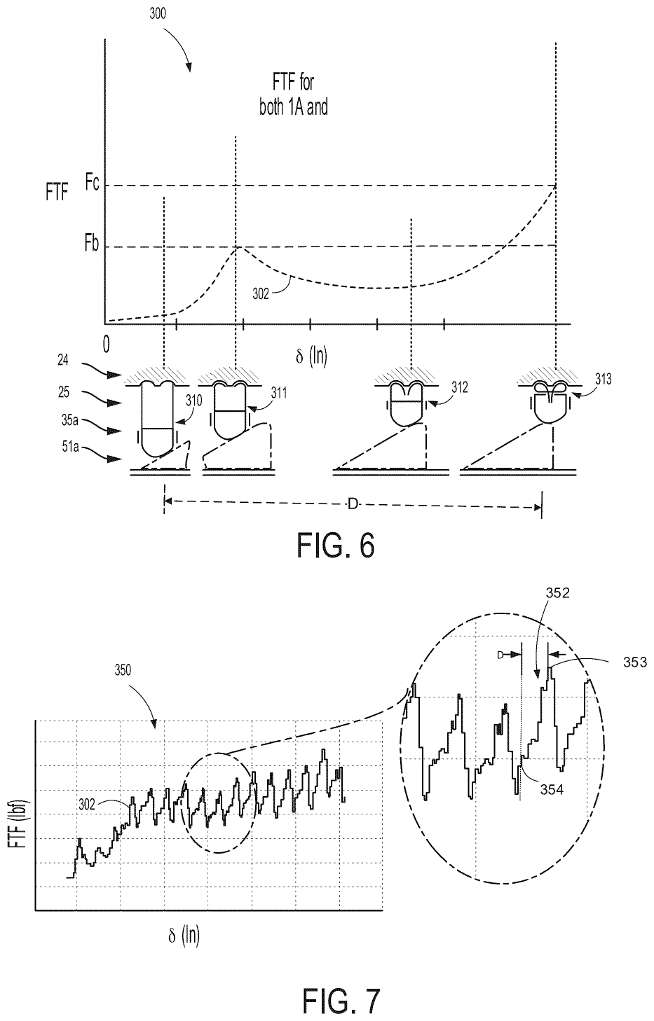

FIGS. 6 and 7 are graphs 300, 320, respectively, that illustrate certain characteristics of force data and positional data indicative of staple malformation. Graphs 300, 320 depict displacement/positional data of the sled 50 on the X-axis and load/force data exerted against the sled 50 on the Y-axis. Graph 300 depicts a force curve 302 that illustrates the force exerted against the ramped surface 51a of the sled 50 by a single staple driver 35a (excluding forces associated with staple drivers 35b-d) along a staple-forming distance (D), as the staple 25 transitions through the staple forming stages, including a first contact stage 310, a buckling stage 311, a forming stage 312, and an over-crunching stage 313. While the graph 300 focuses on the force associated with a single driver 35a, the graph 320 of FIG. 7 focuses on combined forces exerted against the sled 50 by groups of staple drivers (e.g. staple drivers 35a-d) along multiple staple forming distances (e.g. D) along the firing stroke.

In the example illustrated in FIG. 6, over crunching 313 is associated with over-forming the staple 25 such that the staple legs are pushed below the base of the staple leg. Over crunching 313 can be caused by a staple cartridge reload that is too thin. The staple forming forces over form, or over crunch, the staple, which alters the standard B-shape of the formed staples. Staple cartridge reloads comprise staples with cross-sectional dimensions (e.g. thin, medium, thick) that are designed for various different applications (e.g. different tissue types).

As illustrated in FIG. 6, over crunching 313 yields an unexpected peak force Fc toward the end of the staple-forming distance (D), which is greater than a peak force Fb associated with the staple buckling stage 311. In contrast, a normal staple formation is characterized by a higher buckling force than a crunching force, wherein the higher buckling force precedes the lower crunching force along the staple forming distance. In accordance with the present disclosure, a normal staple formation can be associated with a predetermined delta, or difference, between a peak buckling force, at the buckling stage 311, and a peak crunching force, at the forming stage 312, for example. In contrast, a staple malformation can be detected where the measured delta deviates from the predetermined delta. To detect force discrepancies characteristic of staple malformation at the individual staple driver level, a force sensor 122 can be placed at a point of engagement between the cam surface of a staple driver 35 and a ramped surface of the sled 50, for example.

In accordance with the present disclosure, the staple cartridge reload may be too thick (i.e. includes staples with relatively large cross-sectional dimensions for a selected application), and a staple formation may not be properly completed. Consequently, the buckling forces may be lower than a predefined threshold, and the final forming, or crunching, forces may also deviate from a predefined range, which indicates that the final forming, or crunching, may not have occurred.

The force discrepancies at the individual staple driver level translate into force discrepancies at the combined forces level associated with all the staple drivers 35, as illustrated in FIG. 7. A comparison between graph 150 of FIG. 3, which represents normal staple formation, and graph 320 of FIG. 7, which represents staple malformation, outlines the force discrepancies associated with staple malformation at the combined forces level. In accordance with the present disclosure, staple malformation may be detected 203 based on the shape of the force profile along a predefined staple forming distance (D), for example. FIG. 7 illustrates a generally ascending force profile 322 along the staple forming distance (D), while FIG. 3 illustrates a generally descending force profile 152 that is characteristic of normal staple malformation. Accordingly, the control circuit 100 can be configured to determine a force profile along a predefined staple forming distance based on the received 201 first signal and the received 202 second signal, and detect 203 staple malformation based on the determined force profile.

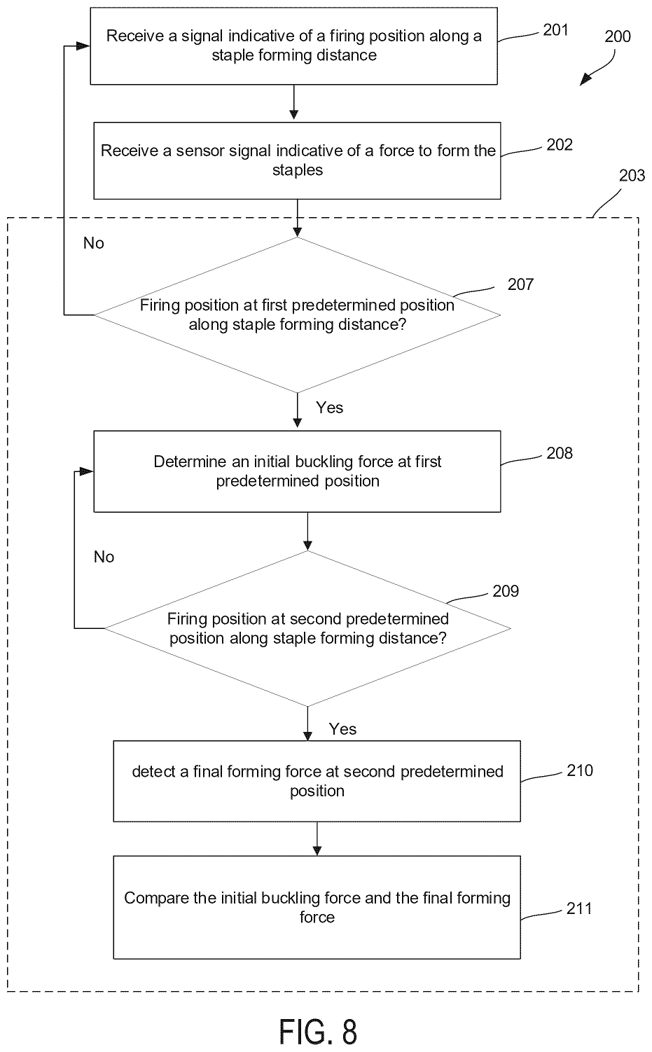

FIG. 8 is a flow diagram of one embodiment of a method 350 for detecting staple malformation, wherein detecting 353 staple malformation is based on detecting an initial staple-buckling force and a final staple-forming force at predefined firing positions along a staple forming distance (D). With reference now to FIG. 8 together with FIG. 5, in the illustrated embodiment, according to the method 350, the control circuit 100 monitors firing position of the sled 50 based on the received 351 the first signal, and corresponding forces exerted against the sled 50 based on the received 352 second signal. The control circuit 100 then determines 357 whether the firing position is at a first predetermined position along a staple forming distance (D). If the firing position is not at a first predetermined position along a staple forming distance (D), the method 350 continues along the No path. If the firing position is at a first predetermined position along a staple forming distance (D), the method 350 continues along the Yes path, and control circuit 100 determines 358 an initial buckling force based on force sensor 122 measurements. Additionally, the method 350 determines 359 whether the firing position is at a second predetermined position along the staple forming distance (D). If the firing position is not at a second predetermined position along the staple forming distance (D), the method 350 continues along the No path. If the firing position is a second predetermined position along a staple forming distance (D), the method 350 continues along the Yes path, and the control circuit 100 determines 360 a final forming force.

According to the method 350, the control circuit 100 then detects staple malformation based on the initial buckling force and the final staple forming force. For example, according to the method 350, the control circuit compares 361 the initial buckling force and the final forming force to each other, or to predetermined thresholds, and may detect staple malformation based on the comparison. In accordance with the present disclosure, the staple malformation may be detected 353 based on a result of a mathematical relation between the determined values of the initial buckling force and the final forming force. Further, in accordance with the present disclosure, staple malformation may be detected 353 if the initial buckling force is less than the final forming force. Also, in accordance with the present disclosure, staple malformation may be detected 353 based on a ratio between the final forming force and the initial buckling. Further, in accordance with the present disclosure, staple malformation may be detected 353 if the initial buckling force is outside a predefined threshold range, and/or if a ratio between the final forming force and the initial buckling force is outside a predefined threshold.

It will be appreciated that the first and second predetermined positions along the staple forming distance (D) depend on various characteristics of the staple cartridge 22 such as, for example, the shape of the cam surfaces of the staple drivers, the shape of the ramped surfaces of the sled, and/or the spacing of the staple cavities. Accordingly, the control circuit 100 can be configured to identify the first and second predetermined positions based on a cartridge identifier that can be received by the control circuit 100 through the user interface 140, or via a communication from an identification chip of the staple cartridge 22, for example. In accordance with the present disclosure, the first and second predetermined positions can be identified by the processor 104 in a look-up table or a database 124 based on the received cartridge identifier.

After identifying the first and second predetermined positions, the control circuit 100 can receive position data from the position sensor 121 to determine the position of the sled 50 and detect the sled 350 reaching the first determined position and the second predetermined position. The control circuit 100 can receive force measurement data from the force sensor 122 to determine the initial buckling force at the first predetermined position, and the final forming, or crunching, force at the second predetermined position. Accordingly, the initial buckling force and the final forming, or crunching, force can be determined by the control circuit 100 based on input from both the position sensor 121 and the force sensor 122.

As previously described with reference to FIGS. 4 and 5, the initial buckling and final forming, or crunching, force values can inform a selection of the next cartridge reload. In accordance with the present disclosure, the control circuit 100 can be configured to adjust 204 a parameter of the surgical stapling system 5 by recommending a thinner or thicker staple cartridge reload based on the determined values of the initial buckling and final forming, or crunching, forces.

With reference to FIGS. 1 and 4, the present disclosure provides various methods, devices, and systems for lowering force-to-fire (FTF), which is a force needed to advance the drive shaft 40 during a tissue treatment event (e.g., firing stroke) by a surgical stapling system 5. In accordance with the present disclosure, the motor assembly 110 may be operable in the tissue treatment event to move the drive shaft 40 to motivate the firing beam 60 and the sled 50 to deploy staples 25 from a staple cartridge 22 into tissue between the anvil 24 and the staple cartridge 22, and, optionally, to cut the tissue with the knife of the firing beam 60, for example.

Various characteristics of the tissue grasped between the staple cartridge 22 and the anvil 24 can influence the FTF. For example, tissue thickness and/or tissue type can influence FTF. An inexperienced clinician may operate the end effector 25 to grasp an excessively thick tissue bite. The increased thickness and/or stiffness of the tissue can yield an increased resistance to the advancement of the drive shaft 40 by the motor assembly 110 during a tissue treatment event, which increases the FTF. The control circuit 100 may utilize a predetermined parameter threshold to maintain the FTF within a safe range. The control circuit 100 may pause the tissue treatment event if the parameter reaches, or exceeds, the predetermined parameter threshold. As discussed in greater detail below, the parameter is indicative of the tissue response to the tissue treatment event, and can be measured by one or more of the sensors 120.

In accordance with the present disclosure, pausing the tissue treatment event can give the tissue time to relax, and time for fluid within the tissue to egress to parts of the tissue not grasped by the end effector 20, which reduces the tissue resistance, and ultimately reduces the FTF when the tissue treatment event is resumed. In accordance with the present disclosure, however, where the tissue resistance may be more due to the tissue stiffness than the tissue fluid build-up, the tissue response during a pause of the tissue treatment even can be less prominent. Stiffness of tissue change based on the tissue type and patient specific issues. Understanding how the tissue responds to a pause in the tissue treatment event informs future pauses and/or generally informs changes to aspects of the tissue treatment event such as, for example, the motion of the drive shaft 40 and/or current supplied to the motor assembly 110, for example.

The present disclosure further provides methods, devices, and systems for predictively and autonomously implementing a future pause to the tissue treatment event based on the tissue response during the threshold-based pause. In accordance with the present disclosure, the future pauses may be based on the tissue response during a previous threshold-based pause interval, but are independent of the FTF threshold. In other words, the future pauses are not triggered by reaching, or exceeding, an FTF threshold. Rather, they are predictively and autonomously implemented based on the tissue response to one or more previous pauses. Further, in accordance with the present disclosure, a future pause may be implemented based on one or more previous non-threshold-based pauses, one or more previous threshold-based pauses or a combination of one or more threshold-based pauses, and one or more non-threshold-based pauses.

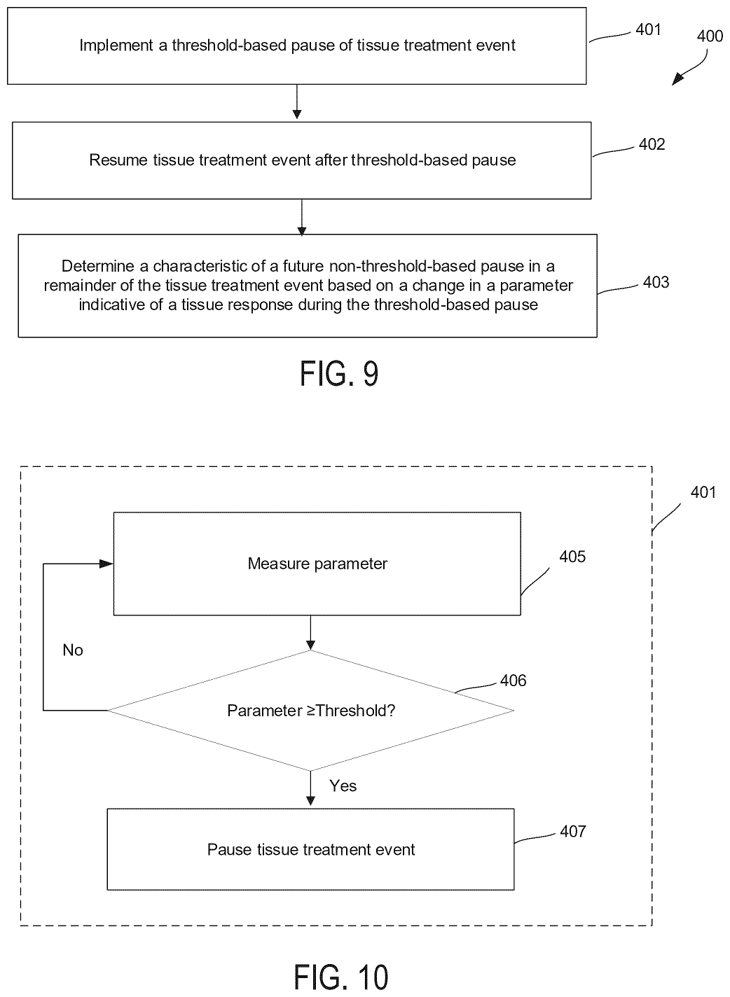

FIG. 9 is a flow diagram of a method 400 for predicatively and autonomously implementing a future non-threshold-based pause of a tissue treatment event being run by the motor assembly 110 based on a tissue response during a previous threshold-based pause of the tissue treatment event. With reference to FIG. 9 together with FIG. 4, according to the method 400, the control circuit 100 implements 401 a threshold-based pause of the tissue treatment event, resumes 402 the tissue treatment event after the threshold-based pause, and determines 403 a characteristic of a future pause in a remainder of the tissue treatment event based on a change in a parameter indicative of a tissue response during the previous pause.

In accordance with the present disclosure of the method 400, the previous pause may be a threshold-based pause and the future pause is a non-threshold based pause. Also, in accordance with the present disclosure of the method 400, the previous pause may be a threshold-based pause and the future pause is also a threshold-based pause. Further, in accordance with the present disclosure of the method 400, the previous pause is a non-threshold-based pause and the future pause is a threshold-based pause. Further, in accordance with the present disclosure of the method 400, the previous pause is a non-threshold-based pause and the future pause is a non-threshold-based pause.

In accordance with the present disclosure, the characteristic of the future pause determined 403 by the method 400 can be a starting point of the future pause. Further, in accordance with the present disclosure, the starting point can be a selected position along the firing stroke. Also, in accordance with the present disclosure, the starting point can be a selected time during the tissue treatment event. Further, in accordance with the present disclosure, the characteristic of the future pause determined 403 by the method 400 can be a duration (D) of the future pause. Also, in accordance with the present disclosure, the duration can be a time-based duration. Further, in accordance with the present disclosure, the duration (D) may be based on a tissue response. For example, a pause can be maintained until a parameter (e.g. force exerted against the drive shaft 40) associated with the tissue response reaches a predetermined value.

In accordance with the present disclosure, the characteristic of the future pause determined 403 by the method 400 can be a frequency of repetition of the future pause. Further, in accordance with the present disclosure, the characteristic of the future pause determined 403 by the control circuit 100 according to the method 400 can be any combination selected from a group that includes the starting point, the duration, and/or the frequency parameters. Other characteristics of the future pause can also be determined 403 by the control circuit 100.

In accordance with the present disclosure, the parameter can be indicative of the tissue resistance exerted against the drive shaft 40 during the threshold-based pause. Also, in accordance with the present disclosure, the parameter can be measured by one or more of the sensors 120. Further, in accordance with the present disclosure, the parameter can be a force exerted by the tissue against the drive shaft 40 during the threshold-based pause, which can be measured by a force sensor 122, for example.

FIG. 10 is a flow diagram illustrating one embodiment of implementing 401 a threshold-based pause of the tissue treatment event, in accordance with the method 400. With reference to FIG. 10 together with FIGS. 4 and 9, in the illustrated example, the control circuit 100 employs one or more sensors 120 to measure 405 the parameter. The one or more sensors 120 can transmit signals to the processor 104 indicative of the parameter. If 406 the parameter reaches, or exceeds, a predetermined threshold, the processor 104 pauses 407 the tissue treatment event.

In accordance with the present disclosure, the control circuit may pause 407 the tissue treatment event by disconnecting power from the power source 111 to the motor assembly 110. Alternatively, the control circuit may pause 407 the tissue treatment event by reducing power supplied to the motor assembly 110. The reduced power can be a power level sufficient to maintain the drive shaft 40 at a current position, but not cause an additional advancement of the drive shaft 40 beyond the current position. The control circuit 100 can be configured to receive positional data from a position sensor 121, for example, indicative of the position of the drive shaft 40, and gradually reduce the current supplied to the motor 113 of the motor assembly 110 until the positional data indicate that no additional change is detected in the position of the drive shaft 40, for example.

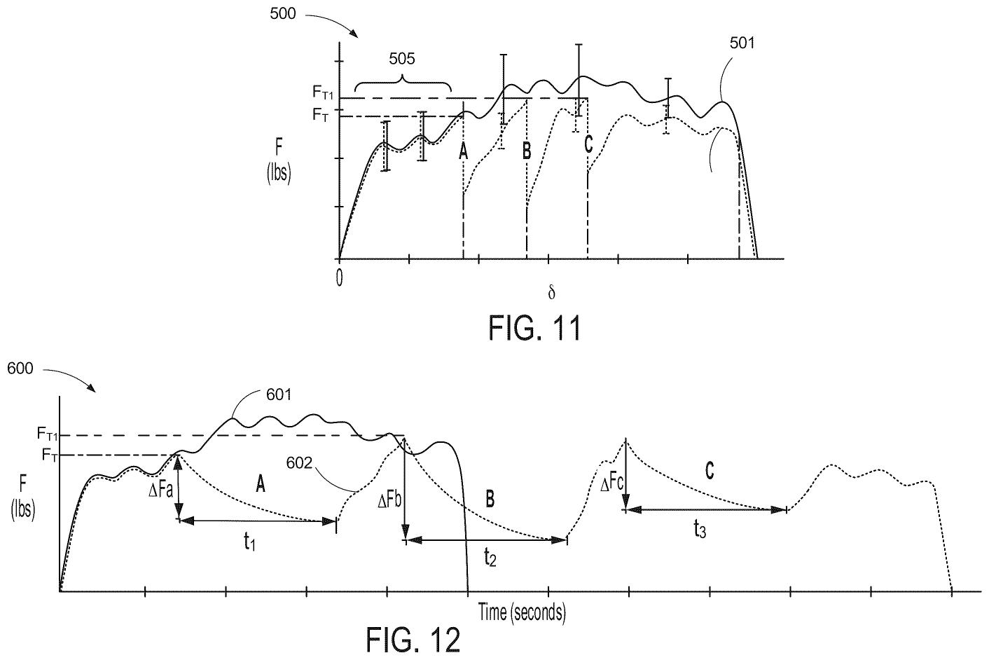

Referring to FIGS. 11, 12, graphs 500, 600 illustrate some of the benefits of implementing pauses during a tissue treatment event by the surgical stapling system 5. Graphs 500, 600 compare tissue responses during a tissue treatment event implemented with and without pausing, by comparing the behavior of the force exerted against the drive shaft 40 (FIGS. 1 and 4) with and without pausing. Graph 500 depicts force data on the Y-axis and position data on the X-axis, while the graph 600 depicts the force data on the Y-axis and time on the X-axis. Curve forces 501, 601 represent a tissue treatment event implemented by the surgical stapling system 5 at a constant speed and without pauses, while curves 502, 602 represent a comparable tissue treatment event implemented by the surgical stapling system 5 with three pauses. It will be appreciated that the election of three pause is for illustrative purposes, and that more or less than three pauses are contemplated by the present disclosure.

With reference now to FIGS. 1, 4, 11, and 12, the position data can be measured by one or more positions sensors 121, and indicate an advancement progress of one or more components of the firing assembly (e.g., drive shaft 40, sled, 50, firing beam 60) along a firing stroke that yields a deployment of the staple into tissue grasped by the end effector 20, and cutting of the tissue by the knife of the firing beam 60, for example. The force data can be measured by one or more force sensors 122 that can be position along one or more engagement points of the firing assembly, for example. Time can be measured by the processor 104, or can be measured separately by a separate clock/timer, for example.

During an initial part of the firing stroke, prior to pausing, the force curves 501, 601 are similar to the force curves 502, 602, respectively. In the illustrated examples, corresponding standard deviations determined during the initial part 505 of the firing stroke are identical, or virtually identical. In accordance with the present disclosure, the standard deviations may be calculated based on a force data set along a portion of the firing stroke. Further, in accordance with the present disclosure, the standard deviations may be calculated based on a force data set that includes force peak and valley portions. Tight standard deviations are indicative of a smooth FTF during a tissue treatment event.

In the illustrated example, a first pause (pause A) of the tissue treatment event is implemented by the control circuit 100 based on reaching, or exceeding, a predetermined force threshold (F_{T}). Tissue grasped by the end effector 20 is allowed to relax during the Pause A, which reduces the force exerted against the drive shaft 40, as evident from comparing the force curves 601, 602. In the illustrated example, pause A is implemented for a time period (t₁). The force (F) exerted against the drive shaft 40 by the tissue gradually decreases by an amount (ΔFa) during the time period (t₁). Upon completion of the pause A, the control circuit 100 resumes the tissue treatment event.

As previously discussed in the present disclosure, a change in one or more parameters during a pause can be informative of the tissue response during a tissue treatment event. In the illustrated example, the change in the force (F) exerted against the drive shaft 40 by the amount (ΔFa) can indicate characteristics of the tissue response that can be useful in implementing future pauses of the tissue treatment event.

In the illustrated example, the control circuit 100 can determine the amount of change in force (ΔFa), and utilize this value to determine one or more characteristics of one or more future pauses such as, for example, pause B and/or pause C. Additionally, or alternatively, the control circuit 100 can determine a rate of change of the force (F) during that time period (t₁), and utilize this value to determine one or more characteristics of one or more of future pauses such as, for example, pause B and/or pause C.

In accordance with the present disclosure, the control circuit 100 may determine one or more characteristics of one or more of future pauses such as, for example, pause B and/or pause C based on the shape of the force curve 601 during a previous pause such as, for example, pause A. For example, a slope of the curve force during pause A can inform decisions regarding future pauses B, C. Also, in accordance with the present disclosure, a steep slope can indicate a greater thickness and/or flexibility of the tissue grasped by the end effector 20. In contrast, a shallow slope can indicate a lesser thickness and/or flexibility of the tissue. Further, in accordance with the present disclosure, the slope value may be directly proportional to the thickness and/or flexibility of the grasped tissue.

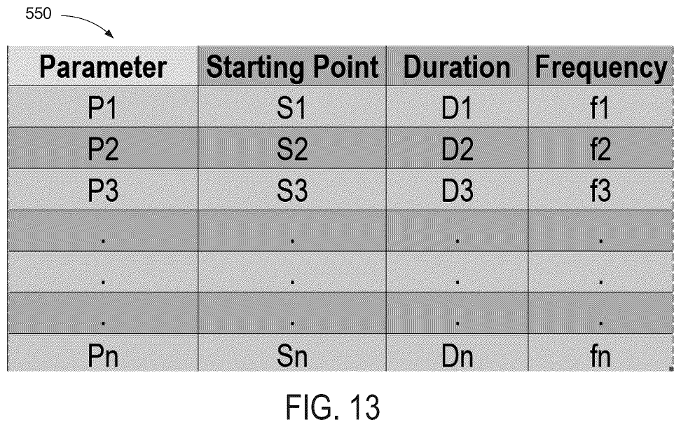

With reference to FIGS. 4 and 13, in accordance with the present disclosure, the processor 104 may query a table 550 stored in the database 124, or which can be stored in a memory circuit (e.g., the memory circuit 105) to determine one or more characteristics of a future pause (e.g., pause B, C) based on a change in a parameter indicative of the tissue response during a previous pause (e.g., Pause A). The table 550 or table may store values representative of the change in the parameter during the previous pause, and values representative of corresponding characteristics of future pauses. As described above, the characteristics of the future pause may include a starting point(S), a duration (D), and/or a frequency (f) of a future pause.

Referring back to FIGS. 11 and 12 together with FIG. 4, the control circuit 100 implements a second pause (pause B). In the illustrated example, a starting point(S) of the pause B is based on a force threshold (F_{T1}). Said another way, the pause B is triggered by the force (F) reaching, or exceeding, the predetermined force threshold (F_{T1}). In accordance with the present disclosure, a starting point(S) of the pause B can be determined based on a parameter associated with the pause A, for example.

In any event, certain characteristics of the pause B are determined based on the pause A. In the illustrated example, a duration (D) of the pause B is determined based on the amount of change in force (ΔFa) detected during the pause A. As described above, the control circuit 100 may determine the amount of change in force (ΔFa) based on input from one or more force sensors 122, for example, and then query the database 550 for values of a duration (D) of the pause B corresponding to the amount of change in force (ΔFa).

Further to the above, the control circuit 100 implements a third pause (pause C). In the illustrated example, a starting point(S) of the pause B, and a duration (D), of the pause C is selected from the database 550 based on an amount of change in force (ΔFa) detected during the pause A and/or the amount of change in force (ΔFb) detected during the pause B, for example.

In accordance with the present disclosure, the control circuit 100 may assign weights to changes to a parameter associated with multiple previous pauses based on where and/or when the previous pauses were implemented along the firing stroke. The assigned weights may then contribute in determining a characteristic of the future pause. The weights can, for example, be assigned based on where and/or when the previous pauses were implemented along the firing stroke. In the illustrated example, a first weight is assigned for the amount of change in force (ΔFa) and a second weight is assigned to the amount of change in force (ΔFb). In accordance with the present disclosure, the weight value may depend on the temporal relation between a previous pause and a future pause.

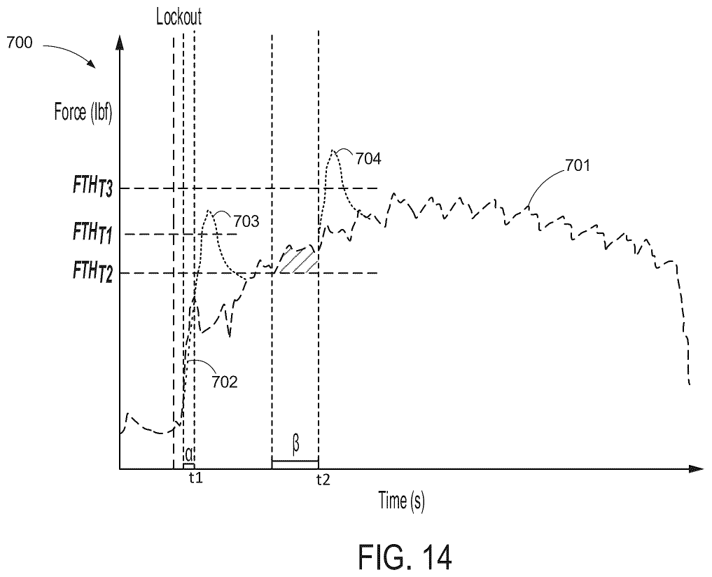

FIG. 14 is a graph 700 illustrating two non-threshold based pauses of a tissue treatment event by the surgical stapling system 5 (FIG. 4), in accordance with the present disclosure. As indicated above, a previous non-threshold-based pause can inform a future non-threshold based pause, in the same tissue treatment event, as illustrated in FIG. 14, or in a future tissue treatment event.

Graph 700 depicts firing force on the x-axis and time on the y-axis. In the illustrated example, a pause to the tissue treatment event is implemented at t₁ based on the behavior of firing force curve 701 during a time period a at an initial portion of the tissue treatment event. The pause at t₁ prevents the firing force from reaching force threshold (FTH_{T1}). Dashed curve 703 outlines a behavior of the firing force without the pause at t₁.

In the example illustrated in FIG. 14 together with FIG. 4, the pause at t₁ is implemented based on a slope 702 of the firing force curve 701 during the time period a. The control circuit 100 determines the slope 701 based on input from the force sensor 122, and determines whether to stop the tissue treatment event based on the value of the slope 702. In accordance with the present disclosure, the decision to pause the tissue treatment event may be based on a database, equation, and/or table stored in a memory unit such as, for example, the memory 106.

Another pause to the tissue treatment event is implemented at t₂ based on the behavior of firing force curve 701 during and/or after the pause at t₁, and/or the behavior of the firing force curve 701 at a time period β at an intermediate portion of the tissue treatment event. In the illustrated example, the firing force curve 702, while not reaching the force threshold (FTH_{T1}), due to the pause started at t₁, still exceeded a force threshold (FTH_{T2}), which informs an effectiveness of the pause at t₁. The behavior of the firing force during the pause is a factor that is considered in implementing the pause at t₂.

In the illustrated example, the pause at t₂ is implemented based on the behavior of the firing force during the time period β, which is determined by calculating the area under the firing force curve 701 during the time period β, and is further based on the behavior of the firing force after the pause at t₁. Dashed curve 704 outlines a behavior of the firing force without the pause at t₂, where the firing force would have exceeded a force threshold (FTH_{T3}).

In accordance with the present disclosure, the time periods α, β can be predetermined time periods, selected by an algorithm implementing the pauses of the tissue treatment event. The selection can be made based on one or more factors including a staple cartridge characteristic (e.g., size, color, type, length, staple height, staple diameter) and/or tissue characteristics (e.g., thickness, density, impedance), for example. Further, in accordance with the present disclosure, the time periods α, β can be adaptively determined based on the behavior of the firing force during the tissue treatment event, or previous tissue treatment events.

During a surgical stapling procedure, a clinician may operate a surgical stapling system 5 to sequentially fire multiple staple cartridges along a selected tissue resection line to achieve a clinical outcome. For example, in a stomach resection procedure, the clinician may sequentially fire staple cartridges of different characteristics (e.g., size, color, type, length, staple height, staple diameter, staple size) along a selected tissue resection line to remove a portion of the stomach. The staple cartridges can be fired along the tissue resection line in an end-to-end arrangement.

The clinician may examine the tissue to be resected using any suitable imaging technique such as, for example, x-ray, registered magnetic resonance imaging (MRI), and/or computerized tomography (CT) scan. The clinician may then select a suitable tissue resection line, and staple cartridges for sequential firing along the selected tissue resection line. Visual examination, however, has its limitations, and a tissue response to stapling can vary depending on many factors including, for example, patient age, tissue health, and/or tissue type. Moreover, tissue thickness and/or stiffness may vary along the selected tissue resection line, resulting in unexpected tissue responses.

Various methods, devices, and systems are provided for adaptively adjusting operational parameters of the surgical stapling system 5 during a staple cartridge firing based on a tissue response in an earlier phase/zone of the staple cartridge firing. Moreover, various methods, devices, and systems are provided for adaptively adjusting operational parameters of the surgical stapling system 5 during a staple cartridge firing based on a tissue response in one or more previous staple cartridge firings in a surgical procedure involving multiple sequential firings.

In accordance with the present disclosure, the processor 104 may execute various program instructions, which can be stored in a memory circuit such as the memory circuit 106, to implement various algorithms associated with firing a staple cartridge by the surgical stapling system 5. Various aspects of such algorithms, e.g., thresholds, limits, triggers, conditions, pauses, wait time, are adjusted based on information learned from a tissue response in an earlier phase/zone of the staple cartridge firing, and/or based on a tissue response in one or more previous staple cartridge firings in a surgical procedure involving multiple sequential firings, as described in more detail below.

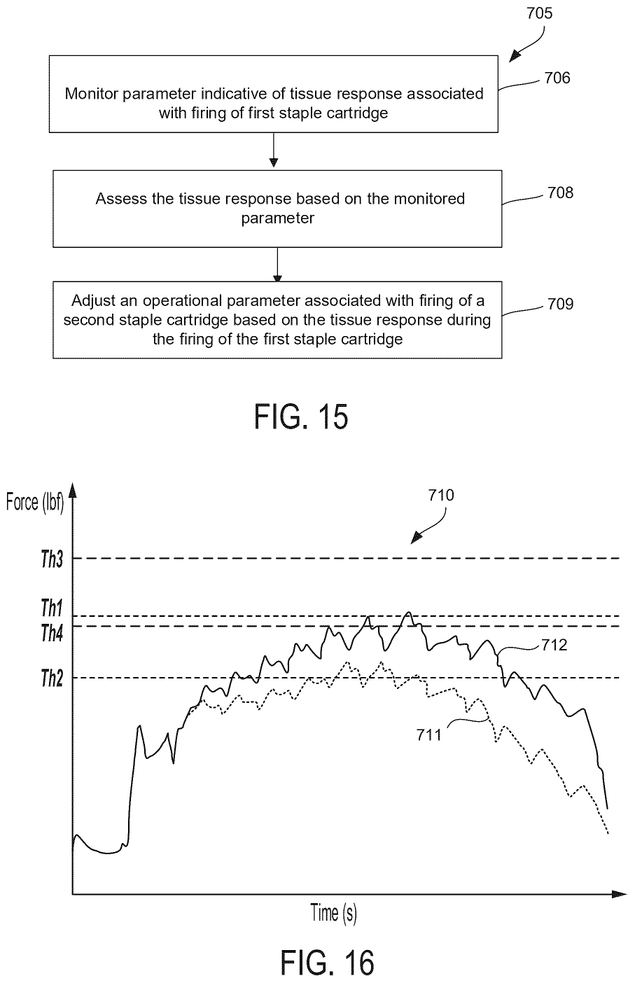

FIG. 15 is flow diagram illustrating a logic configuration of a method 709 for adjusting operational parameters of the surgical stapling system 5 in a surgical procedure that involves a first firing that deploys first staples from a first staple cartridge (e.g. staple cartridge 22) into a first tissue portion, and a second firing that deploys second staples from a second staple cartridge into a second tissue portion adjacent the first tissue portion. The first tissue portion and the second tissue portion reside along a planned tissue resection line, and the first staples and the second staples are deployed in an end-to-end arrangement along the tissue resection line.

One or more firings of staple cartridges can precede the first firing of the first staple cartridge along the tissue resection line. Additionally, the first staple cartridge can be the same as, or different than, the second staple cartridge in color, size, shape, staple height, staple diameter, and/or any other suitable feature.

With reference now to FIG. 15 together with FIG. 4, in accordance with the method 709, the control circuit 100 monitors 706 a parameter indicative of a tissue response associated with the first firing. The control circuit 100 assesses 707 the tissue response based on the monitored 706 parameter, and adjusts 703 an operational parameter associated with the second firing based on the tissue response during the first firing.

In accordance with the present disclosure, the monitored 706 parameter can be a tissue resistance to the advancement of the firing assembly during the first firing. The control circuit 100 can monitor 706 the tissue resistance using one or more of the sensors 120. Additionally, one or more force sensors 122 can measure the force exerted against the drive shaft 40, the sled 50, and/or the firing beam 60, which represents the tissue response during the first firing.

Alternatively, or additionally, a current sensor can be employed together with the control circuit 100 to assess the tissue response during the first firing. In accordance with the present disclosure, the control circuit 100 can be coupled to a current sensor to receive a signal indicative of the current supplied by the power source 111 to the motor of motor assembly 110 during the first firing. A change in the current draw of the motor is representative of a change in the tissue resistance to the advancement of the firing assembly, which is representative of the tissue response during the first firing.

Alternatively, or additionally, the tissue response can be assessed based on a secondary input such as a temperature sensor that measures a temperature of the motor, for example. If the temperature of the motor is higher than a predetermined threshold, the increase in the temperature of the motor is indicative of an overload, or stall, condition.

Further to the above, according to the method 709, the control circuit 100 assesses 707 the tissue response based on the monitored 706 parameter. In accordance with the present disclosure, the control circuit 100 may assess 707 the tissue response by comparing measured values of the monitored 706 parameter to a predetermined threshold. The processor 104 may determine the values based on input from the sensors 120, retrieve the predetermined threshold, and compare the values to the retrieved predetermined threshold. The values can be determined by the control circuit 100 by sampling sensor readings of the parameter.

In accordance with the present disclosure, the predetermined threshold of the parameter can be selected by the processor 104 based on the cartridge type of the first staple cartridge. Different predetermined thresholds can be preset for different staple cartridge types. The control circuit 100 can retrieve an identifier of the first staple cartridge, and query a database stored in the memory 106, for example, for the predetermined threshold based on the retrieved identifier. The database may include a list of identifiers and corresponding predetermined thresholds. Also, in accordance with the present disclosure, the control circuit 100 may communicate wirelessly, or through a wired connection, with a cartridge chip, or memory unit, within the first staple cartridge to retrieve the identifier associated with the first staple cartridge. The identifier may be provided by a clinician through the user interface 140.

Further to the above, the control circuit 100 may store an outcome of the comparison, indicative of the tissue response, for use in the second firing. A tissue response that violates one or more predetermined thresholds of the first staple cartridge can inform the selection of the second staple cartridge. Accordingly, adjusting 703 the operational parameter associated with the second firing may include the control circuit 100 selecting, or at least recommending, a second staple cartridge with different predetermined thresholds than the first staple cartridge, where the tissue response during the first firing indicates a violation of the one or more predetermined of the first staple cartridge.

After completion of the first firing, the control circuit 100 may select, or at least recommend, a staple cartridge type for the second firing based on the stored outcome of the comparison. In accordance with the present disclosure, the control circuit 100 can detect an identifier of the second staple cartridge in a similar manner to that described in connection with the first staple cartridge, and prompt the clinician, for example through the user interface 140, to change to a different staple cartridge type based on the outcome of the comparison during the first firing.

In accordance with the present disclosure, the surgical system 5 can be used in a sleeve gastrectomy, for example, involving multiple sequential firings along a planned tissue resection line. After the first firing on the sleeve using a clinician chosen staple cartridge, the control circuit 100 may determine, based on the tissue response during the first firing, that the tissue thickness is greater than that recommended for the staple cartridge utilized in the first firing. The control circuit 100 may then prompt the clinician through the user interface 140 as to the higher than expected tissue thickness and asks if the clinician wishes to adjust the current thresholds, for example, to accommodate increase during a second firing, change to a cartridge that is more appropriate to the tissue thickness, or to adapt the speed and pauses within the surgical system 5 to compensate for the differences. The control circuit 100 can provide the clinician an interrelationship between firing speeds, pauses, and loads associated with a selected, or recommended, staple cartridge, allowing the clinician to adjust the balance of the such interactive thresholds.

FIG. 16 is a graph 710 illustrating an example of the adaptive behavior of the surgical system 5 during a surgical procedure that involves a sequential firing of multiple staple cartridges. The adaptive behavior comprises a selection, or at least a recommendation, of a second staple cartridge based on a tissue response associated with a first staple cartridge in a sequential firing of the first and second staple cartridges. The X-axis represents firing time, and the Y-axis represents firing force. In the illustrated example, the tissue response is assessed by monitoring the firing force.

Graph 710 illustrates upper (Th1) and lower (Th2) predetermined thresholds associated with the first staple cartridge, and upper (Th3) and lower (Th4) predetermined thresholds associated with the second staple cartridge. Curve line 711 illustrates a tissue response in accordance with the upper (Th1) and lower (Th2) predetermined thresholds associated with a first staple cartridge. Since a suitable tissue response is detected, the control circuit 100 selects, or at least recommends, the first staple cartridge type for the subsequent firing.

In contrast, curve line 712 illustrates a tissue response that violates the upper (Th1) predetermined threshold associated with the first staple cartridge during the first firing. In response, the control circuit 100 selects, or at least recommends, a different staple cartridge type for the second firing, with higher upper (Th3) and lower (Th4) predetermined thresholds. Alternatively, if a clinician elects to use the first staple cartridge type in the second firing, the control circuit 100 overrides the upper (Th1) predetermined threshold, and adjusts 703 other operational parameters of the surgical system 5 such as, for example, motor output, clamp time, and/or firing pauses during the second firing to maintain and/or return the second firing to a level below the upper (Th1) predetermined threshold.

In accordance with the present disclosure, the surgical stapling system 5 may execute the method 709 in a gastric bypass surgery that involves multiple firings along a planned transection line across the stomach. Some clinicians tend to use different staple cartridge types, e.g. different sizes such as a thick reload followed by a thin reload, along the tissue resection line. Others tend to use the same staple cartridge type, e.g. a specific size such as a thick reload or a thin reload, along the tissue resection line. For the latter, the control circuit 100 may execute the method 709 by adjusting 703 a tissue compression parameter and/or a firing parameter based on the tissue response to the thick reload or the thin reload. For example, if the tissue response in a previous firing indicates that the utilized staple cartridge size is too thick for the tissue, the control circuit 100 increases tissue compression time and/or decreases firing speed. In contrast, if the tissue response in a previous firing indicates that the utilized staple cartridge size is too thin for the tissue, the control circuit 100 may decrease tissue compression time and/or increases firing speed.

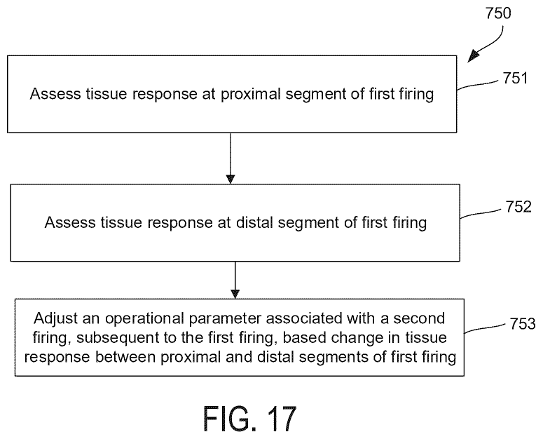

FIG. 17 is a flow diagram illustrating a logic configuration of a method 750 for adjusting a second firing based on a change in the tissue response in predetermined segments of the first firing, wherein the change in the tissue response indicates a change in a tissue characteristic between the predetermined segments. The method 750 is similar in many respects to the method 709. For example, the method 750 also can be executed by the control circuit 100 in a surgical procedure involving multiple sequential firings including a firing of a first staple cartridge and a subsequent firing of a second staple cartridge along a planned tissue transection line.

In accordance with the present disclosure, the control circuit 100 may monitor the same parameter, or parameters, during the method 709 and the method 750. The control circuit 100 may monitor a first parameter during the method 709, and a second parameter, different than the first parameter, in the method 750. The methods 709, 750 can be executed separately or in combination.

As described above, the tissue response depends, at least in part, on characteristics of the tissue such as, for example, tissue thickness and/or stiffness, which can gradually change along a tissue resection line. Accordingly, a change in the tissue response between a proximal segment and a distal segment of a firing along a tissue resection line can be predictive of the tissue response, and characteristics, in a subsequent firing along the tissue resection line.

In the illustrated example, according to the method 750, the control circuit 100 assesses 751 the tissue response at a proximal segment of the first firing, assesses 752 the tissue response at a distal segment of the first firing, and adjusts 753 the operational parameter associated with the second firing based on a change in the tissue response between the proximal and distal segments of the first firing.

In accordance with the present disclosure, the change in the tissue response can be assessed based on values of the parameter detected at the different segments of the first firing. A first value of the parameter, which can be determined at the proximal segment, may be compared to a first predetermined threshold to determine a first delta between the first value and the first predetermined threshold. Similarly, a second value of the parameter, which can be determined at the distal segment, may be compared to a second predetermined threshold to determine a second delta between the second value and the second predetermined threshold. In accordance with the present disclosure, the change in the tissue response can be determined based on the first delta and the second delta. The change in the tissue response can be determined based on a mathematical relation between the first delta and the second delta such as, for example, a ratio of the first delta to the second delta.

Further to the above, the first and second predetermined thresholds can be selected based on the firing positions of one or more components of the firing assembly, during the advancement of the firing assembly along the firing stroke, where the first value and the second value, respectively, are measured. A database (e.g., database 124 FIG. 4), or a table, can store various predetermined thresholds, which represent acceptable, or ideal, upper or lower limits of the parameter at specific firing positions, and corresponding values that represent such firing positions. Alternatively, an equation for calculating the predetermined thresholds at the specific firing positions can be employed. The firing positions can define a variable in the equation.

In either event, the processor 104 employs the database, table, and/or equation to determine the first and second predetermined thresholds of the parameter based on the firing positions. One or more position sensors 121 can provide signals to the processor 104, which may be utilized by the processor 104 to determine the firing positions. In accordance with the present disclosure, the firing positions can be represented as distances from a starting position of the firing assembly. Alternatively, or additionally, the firing positions can be represented as ranges indicative of discrete portions of the firing such as, for example, a proximal portion, an intermediate portion, and/or a distal portion.

In accordance with the present disclosure, the first tissue response at the proximal segment and the second tissue response at the distal segment may indicate a change in tissue stiffness between the proximal segment and the distal segment of the first firing. Said another way, the change in the tissue response between the proximal segment and the distal segment may indicate a change in the tissue stiffness in a proximal-to-distal direction. The control circuit 100 can then adjust 753 one or more operational parameters of the surgical system 5 to address a predicted tissue stiffness in the second firing learned from the change in the tissue response between the proximal segment and the distal segment of the first firing.

If the predicted tissue stiffness is higher than expected, the control circuit 100 can adjust a firing algorithm of the second firing to reduce the firing speed, for example by introducing and/or adjusting one or more pauses during the second firing. Additionally, or alternatively, the control circuit 100 may select, or at least recommend, a different staple cartridge, one more suited for the predicted tissue stiffness. Additionally, or alternatively, the control circuit 100 may adjust or remove one or more predetermined thresholds, and/or changes a clamp, or tissue compression, time associated with to the second firing.

Conversely, if the predicted tissue stiffness is lower than expected, i.e., tissue becomes softer, the control circuit 100 can adjust a firing algorithm of the second firing to increase the firing speed, and/or remove and/or adjust one or more planned pauses during the second firing. Additionally, or alternatively, the control circuit 100 may select, or at least recommend, a different staple cartridge, one more suited for the predicted tissue stiffness. Additionally, or alternatively, the control circuit 100 may adjust or remove one or more predetermined thresholds, and/or changes a clamp, or tissue compression, time associated with the second firing.

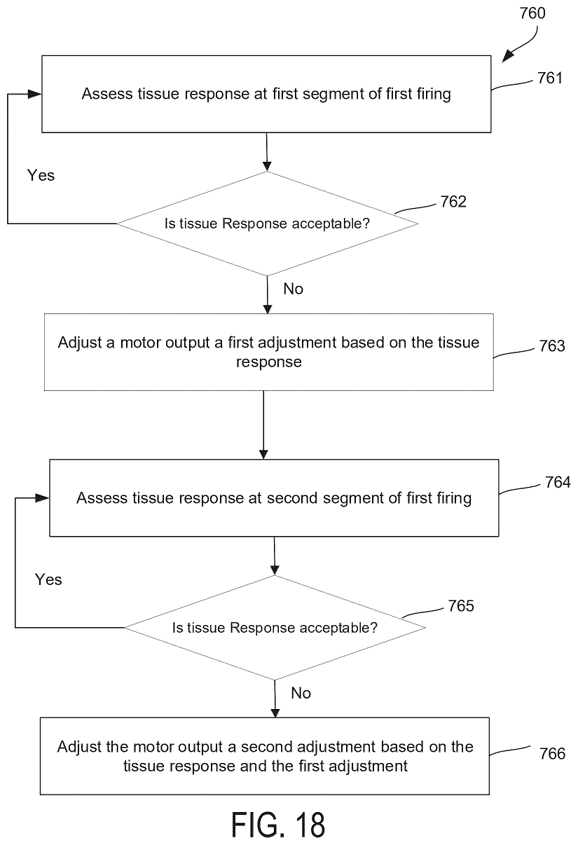

FIG. 18 illustrates a logic configuration of a method 760 similar in many respects to the methods 709, 750. The method 760 can be executed by the control circuit 100 separately, or in combination with the method 709 and/or the method 750. In accordance with the present disclosure, the method 760 may focus on learned triggers for motor control adaptation. With reference to FIG. 18 together with FIG. 4, in the illustrated example, in accordance with the method 760, the control circuit 100 adjusts a firing algorithm based on monitored parameters (e.g., motor current), where the firing algorithm adjustments vary form a first intervention to a second intervention, as a learned response. In accordance with the present disclosure, the variation between the first and second interventions can be based on a failure achieving an expected tissue response such as, for example, a failure to lower a firing force resulting from tissue resistance, as expected after the first intervention.

Like the method 750, the method 760 includes assessing the tissue response at multiple segments of the first firing. In the illustrated example, in accordance with the method 760, the control circuit 100 assesses 761 a first tissue response at a first segment of the first firing and assesses 762 a second tissue response at a segment of the first firing. The details of assessing a tissue response during a firing are similar to those described in connection with the methods 709, 750, and are not repeated herein for brevity.

According to the method 760, the control circuit 100 adjusts 763 a firing algorithm a first adjustment based on the first tissue response, which constitutes a first intervention. According to the method 760, the control circuit 100 determines 762 whether the first tissue response at a first segment of the firing is not acceptable, e.g., the value of the monitored parameter indicative of the tissue response is lower or higher than a predetermined threshold, the control circuit 100 adjusts 763 a firing algorithm a first adjustment based on the first value or based on the delta between the first value and the predetermined threshold. In accordance with the present disclosure, adjusting 763 the firing algorithm may include changing a motor output by, for example, changing a motor voltage parameter such as, for example, pulse-width-modulation (PWM) and/or amplitude.

Following the first adjustment 763, the control circuit 100 continues to assess 764 the tissue response at a second segment of the first firing, distal to the first segment, to determine the efficacy of the first intervention. According to the method 760, the control circuit 100 determines 765 whether a tissue response at the second segment of the firing is also not acceptable, e.g., the value of the monitored parameter indicative of the tissue response is lower or higher than a predetermined threshold, the control circuit 100 further adjusts 766 the firing algorithm a second adjustment, in a learned response, based on the assessed 764 tissue response, and the first adjustment. The second adjustment can, for example, a second adjustment to the motor output by, for example, changing a motor voltage parameter such as, for example, pulse-width-modulation (PWM) and/or amplitude.

Accordingly, the method 750 takes an adaptive approach in selecting the second adjustment, by taking into consideration the first adjustment. Said another way, the second adjustment is based on the tissue response to the first adjustment. For example, if the first adjustment was not effective in addressing the trigger of the first threshold, as can be determined based on the second value, the second delta, or measurements of other parameters relevant to the first firing, the control circuit 100 responds by selecting a more suitable second adjustment. In accordance with the present disclosure, a table, database, and/or equation can be employed to select the second adjustment based on the second value, or the second delta, and the first adjustments.

Further to the above, the learned or adaptive behavior of the control circuit 100 can be implemented automatically or can be conditioned upon a clinician approval receivable through the user interface 140. In accordance with the present disclosure, the control circuit 100 may calculate the second adjustment based on the tissue response and the first adjustment, and prompt the clinician for an approval to implement the second adjustment. If the approval is entered through the user interface 140, the control circuit 100 may then execute the second adjustment. The clinician may be further permitted to modify the second adjustment.

In accordance with the present disclosure, firing algorithm adjustments, in accordance with one more methods (e.g., methods 709, 750, 760) of the present disclosure, can include adjustments to predetermined thresholds. The learned behavior in a first firing can lead to one or more adjustments to one or more predetermined thresholds in a later segment of the first firing, and/or in a second firing that follows the first firing. Such thresholds include global thresholds including, for example, a global force-to-fire threshold that, if reached, the control circuit 100 deactivates the surgical system to avoid potential damage to the surgical system 5.

Such thresholds also include a device-specific force-to-fire threshold that is based on a particular surgical system 5. During final testing of the surgical system 5, where internal loads and frictions are documented, force-to-fire threshold is calculated as a combination of the actual force-to-fire threshold with shaft loads added. Such thresholds also include a cartridge-specific force-to-fire threshold, which are based on various staple cartridge characteristics such as, for example, cartridge size.

Referring still to FIG. 18, a decision to implement a firing algorithm adjustment, such as a predetermined threshold adjustment, in accordance with the method 760, is further based on the location along the firing stroke, where the predetermined threshold is implemented, and the location where the tissue response is assessed. The location can be a specific position, or segment, along the firing stroke, for example. As described above in greater detail, one or more position sensors 121 provide signals to the processor 104, which are utilized by the processor 104 to determine the firing positions. In accordance with the present disclosure, the firing positions can be represented as distances from a starting position of the firing assembly. Alternatively, or additionally, the firing positions can be represented as ranges indicative of discrete segments of the firing such as, for example, a proximal segment, an intermediate segment, and/or a distal segment.

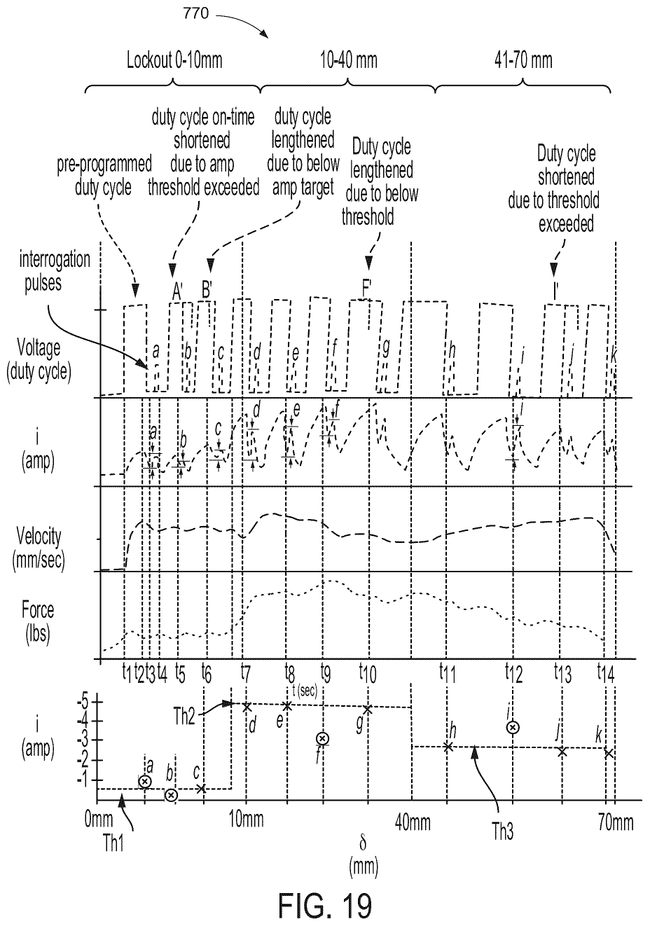

FIG. 19 is a graph 770 illustrating various parameters associated with a staple cartridge firing by the surgical system 5, which are indicative of the tissue response, in accordance with at least one aspect of the present disclosure. The parameters are tracked along various segments of the firing stroke, and include motor voltage, motor current, firing velocity, and force exerted against the firing assembly during firing. The graph 770 further depicts examples of intra-firing adaptive behavior of the surgical system 5, which are based on interrogation pulses that are utilized to assess a tissue response during the firing.

In the illustrated example, the control circuit 100 introduces additional variations, in the form of interrogation pulses (a-k), to improve the adaptive behavior of the surgical system 5. During firing, the motor voltage is typically held constant while being pulse-width modulated for the purpose of delivering power to the motor of the motor assembly 110. The control circuit 100 introduces additional small variations into the motor voltage (dithering) to better understand the response of the measured aspects (motor current/speed/knife location), and the impact on the surgical system 5.

In accordance with the present disclosure, the control circuit 100 may introduce small spikes, e.g., interrogation pulses (a-k, at the end of each PWM cycle (e.g., at t1-t2), which are too small to have a clinical impact on RMS current, but rather create small peak responses in current (e.g., at t3). As illustrated in FIG. 19, the current peak responses are compared to predetermined threshold (e.g., Th1, Th2, Th3) for assessing the tissue response. If the current peak response is below the predetermined threshold, no changes or action may be required. Alternatively, or additionally, the control circuit 100 may increase the next duty cycle time (e.g., B′, F′) to increase firing speed based on the current peak response being below the predetermined threshold.

In contrast, if the current peak response is equal to, or higher than, the predetermined threshold, the control circuit 100 may adjust an operational parameter of the surgical system 5 such as, for example, the next PWM cycle pulse to be shorter in time (e.g., A′, I′). Such adjustment decreases current draw by the motor assembly 110, thereby reducing velocity and/or force applied to the firing assembly, which effectively returns the tissue response to an acceptable value.

In accordance with the present disclosure, the control circuit 100 may begin the firing at 50% duty cycle on the motor assembly 110 to ensure the mechanical integrity of the components of the firing assembly, and/or to test a mechanical cartridge lockout, for example. During this initial segment, the force-to-fire can be monitored, and an initial peak level of the force-to-fire can be determined based on inputs from the force sensor 122, for example, to the control circuit 100. This initial peak can be used to set initial thresholds for the other segments of the firing stroke, in accordance with the method 760, for example.

In accordance with the present disclosure, if the control circuit 100 detects a thick, or dense, tissue based on the initial peak is designated “high,” based on a comparison to an initial predetermined threshold, for example, this can be an indication that the tissue is thick or dense. In response, the control circuit 100 may increase predetermined thresholds for a second segment of the firing stroke. If, however, the initial force reading is low, the control circuit 100 may reduce predetermined thresholds for the second segment of the firing stroke. The increase or decrease can be based on the initial peak, or the delta between the initial peak and the initial predetermined threshold.

As the firing stroke transitions to a third segment, e.g. midpoint to end of cutline, the predetermined thresholds of the third segments can be adjusted based on the initial peak, based on the predetermined thresholds of the second segment, and/or based on the tissue response in the second segment. In accordance with the present disclosure, the thresholds of the third segment can be set higher than the previous segment to ensure the completion of the firing,

Further to the above, upon completion of the firing stroke, the firing assembly is returned by the motor assembly 110 to a home position. The control circuit 100 may remove, or adjust the predetermined thresholds to a maximum, during the return of the firing assembly.

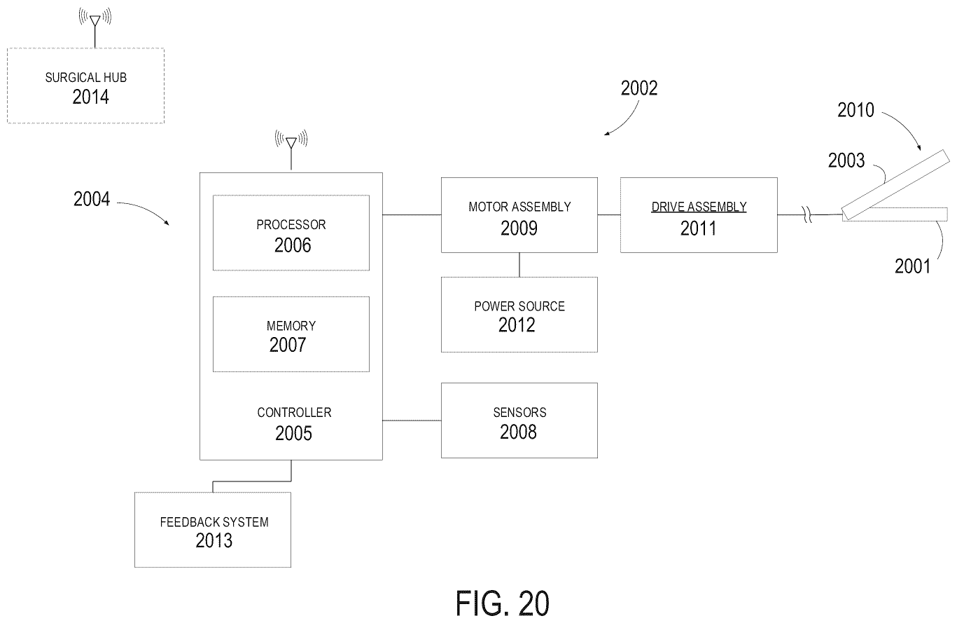

FIG. 20 illustrates a block diagram of a surgical system 2002 for use with one or more surgical instruments, tools, and/or robotic systems in accordance with one or more aspects of the present disclosure. The system 2002 includes a control circuit 2004. The control circuit 2004 includes a microcontroller 2005 comprising a processor 2006 and a storage medium such as, for example, a memory 2007.

A motor assembly 2009 includes one or more motors, driven by motor drivers. The motor assembly 2009 operably couples to a drive assembly 2011 to drive, or effect, one or more motions at an end effector 2010. The drive assembly 2011 may include any number of components suitable for transmitting motion to the end effector 2010 such as, for example, one or more gears, gear sets, gear transmissions with one or multiple selectable gears, linkages, bars, tubes, and/or cables, for example.

One or more of sensors 2008, for example, provide real-time feedback to the processor 2006 about one or more operational parameters monitored during a surgical procedure being performed by the surgical system 2002. The operational parameters can be associated with a user performing the surgical procedure, a tissue being treated, and/or one or more components of the surgical system 2002, for example. The sensors 2008 may comprise any suitable sensor, such as, for example, a magnetic sensor, such as a Hall effect sensor, a strain gauge, an encoder, a position sensor, a force sensor, a pressure sensor, an inductive sensor, such as an eddy current sensor, a resistive sensor, a capacitive sensor, an optical sensor, and/or any other suitable sensor.

Further to the above, in various arrangements, the sensors 2008 may comprise any suitable sensor for detecting one or more conditions at the end effector 2010 including, without limitation, a tissue thickness sensor such as a Hall Effect Sensor or a reed switch sensor, an optical sensor, a magneto-inductive sensor, a force sensor, a pressure sensor, a piezo-resistive film sensor, an ultrasonic sensor, an eddy current sensor, an accelerometer, a pulse oximetry sensor, a temperature sensor, a sensor configured to detect an electrical characteristic of a tissue path (such as capacitance or resistance), or any combination thereof. As another example, and without limitation, the sensors 2008 may include one or more sensors located at, or about, an articulation joint extending proximally from the end effector 2010. Such sensors may include, for example, a potentiometer, a capacitive sensor (slide potentiometer), piezo-resistive film sensor, a pressure sensor, a pressure sensor, or any other suitable sensor type. In some arrangements, the sensor 2008 may comprise a plurality of sensors located in multiple locations in the end effector 2010.

In accordance with the present disclosure, the surgical system 2002 may include a feedback system 2013 which may include one or more devices for providing a sensory feedback to a user. Such devices may comprise, for example, visual feedback devices (e.g., an LCD display screen, a touch screen, LED indicators), audio feedback devices (e.g., a speaker, a buzzer) or tactile feedback devices (e.g., haptic actuators).

The microcontroller 2005 may be programmed to perform various functions such as precise control over the speed and position of the drive assembly 2011. In accordance with the present disclosure, the microcontroller 2005 may be any single-core or multicore processor such as those known under the trade name ARM Cortex by Texas Instruments. Additionally, the main microcontroller 2005 may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHZ, a 32 KB single-cycle SRAM, and internal ROM loaded with StellarisWare® software, a 2 KB EEPROM, one or more PWM modules, one or more QEI analogs, and/or one or more 12-bit ADCs with 12 analog input channels, details of which are available for the product datasheet.

The microcontroller 2005 may be configured to compute a response in the software of the microcontroller 2005. The computed response is compared to a measured response of the actual system to obtain an “observed” response, which is used for actual feedback decisions. The observed response is a favorable, tuned value that balances the smooth, continuous nature of the simulated response with the measured response, which can detect outside influences on the system.

The motor assembly 2009 includes one or more electric motors and one or more motor drivers. The electric motors can be in the form of a brushed direct current (DC) motor with a gearbox and mechanical links to the drive assembly 2011. In accordance with the present disclosure, a motor driver may be an A3941 available from Allegro Microsystems, Inc.

In accordance with the present disclosure, the motor assembly 2009 may include a brushed DC driving motor having a maximum rotational speed of approximately 25,000 RPM. The motor assembly 2009 may include a brushless motor, a synchronous motor, a stepper motor, or any other suitable electric motor. The motor driver may comprise an H-bridge driver comprising field-effect transistors (FETs), for example.

The motor assembly 2009 can be powered by a power source 2012. In accordance with the present disclosure, the power source 2012 may include one or more batteries which may include a number of battery cells connected in series that can be used as the power source to power the motor assembly 2009. Further, in accordance with the present disclosure, the battery cells of the power assembly may be replaceable and/or rechargeable. Additionally, the battery cells may comprise lithium-ion batteries which can be couplable to and separable from the power assembly.

Further to the above, the end effector 2010 includes a first jaw 2001 and a second jaw 2003. At least one of the first jaw 2001 and the second jaw 2003 is rotatable relative to the other during a closure motion that transitions the end effector 2010 from an open configuration toward a closed configuration. In accordance with the present disclosure, a cartridge jaw can be movable relative to a fixed anvil jaw to a clamped position. Additionally, an anvil jaw can be movable relative to a fixed cartridge jaw to a clamped position. Furthermore, an anvil jaw and a cartridge jaw may both be movable relative to each other to a clamped position. The closure motion may cause the jaws 2001, 2003 to grasp tissue therebetween. In accordance with the present disclosure, sensors, such as, for example, a strain gauge or a micro-strain gauge, can be configured to measure one or more parameters of the end effector 2010, such as, for example, the amplitude of the strain exerted on the one or both of the jaws 2001, 2003 during a closure motion, which can be indicative of the closure forces applied to the jaws 2001, 2003. The measured strain can be converted to a digital signal and provided to the processor 2006, for example. Alternatively, additionally, sensors such as, for example, a load sensor, can measure a closure force and/or a firing force applied to the jaws 2001, 2003.

In accordance with the present disclosure, a current sensor can be employed to measure the current drawn by a motor of the motor assembly 2009. The force required to advance the drive assembly 2011 can correspond to the current drawn by the motor, for example. The measured force can be converted to a digital signal and provided to the processor 2006.

In accordance with the present disclosure, strain gauge sensors can be used to measure the force applied to the tissue by the end effector 2010, for example. A strain gauge can be coupled to the end effector 2010 to measure the force on the tissue being treated by the end effector 2010. Additionally, the strain gauge sensors can measure the amplitude or magnitude of the strain exerted on a jaw of an end effector 2010 during a closure motion which can be indicative of the tissue compression. The measured strain can be converted to a digital signal and provided to a processor 2006.

The measurements of the tissue compression, the tissue thickness, and/or the force required to close the end effector on the tissue, as respectively measured by the sensors 2008 can be used by the microcontroller 2005 to characterize the selected position of one or more components of the drive assembly 2011 and/or the corresponding value of the speed of one or more components of the drive assembly 2011. In accordance with the present disclosure, a memory (e.g. memory 2007) may store a technique, an equation, and/or a look-up table which can be employed by the microcontroller 2005 in the assessment.

The system 2002 may comprise wired or wireless communication circuits to communicate with surgical hubs (e.g. surgical hub 2014), communication hubs, and/or robotic surgical hubs, for example. Additional details about suitable interactions between a system 2002 and the surgical hub 2014 are disclosed in U.S. patent application Ser. No. 16/209,423 entitled METHOD OF COMPRESSING TISSUE WITHIN A STAPLING DEVICE AND SIMULTANEOUSLY DISPLAYING THE LOCATION OF THE TISSUE WITHIN THE JAWS, now U.S. Patent Application Publication No. 2019/0200981, the entire disclosure of which is incorporated by reference in its entirety herein.

In accordance with the present disclosure the control circuit 2004 can be configured to implement various processes described herein. The control circuit 2004 may comprise a microcontroller comprising one or more processors (e.g., microprocessor, microcontroller) coupled to at least one memory circuit. The memory circuit stores machine-executable instructions that, when executed by the processor, cause the processor to execute machine instructions to implement various processes described herein. The processor may be any one of a number of single-core or multicore processors known in the art. The memory circuit may comprise volatile and non-volatile storage media. The processor may include an instruction processing unit and an arithmetic unit. The instruction processing unit may be configured to receive instructions from the memory circuit of this disclosure.

Alternatively, in accordance with the present disclosure, the control circuit 2004 can be in the form of a combinational logic circuit configured to implement various processes described herein. The combinational logic circuit may comprise a finite state machine comprising a combinational logic configured to receive data, process the data by the combinational logic, and provide an output.

Alternatively, in accordance with the present disclosure, the control circuit 2004 can be in the form of a sequential logic circuit. The sequential logic circuit can be configured to implement various processes described herein. The sequential logic circuit may comprise a finite state machine. The sequential logic circuit may comprise a combinational logic, at least one memory circuit, and a clock, for example. The at least one memory circuit can store a current state of the finite state machine. In accordance with the present disclosure, the sequential logic circuit may be synchronous or asynchronous. Further, in accordance with the present disclosure, the control circuit 2004 may comprise a combination of a processor (e.g., processor 2006) and a finite state machine to implement various processes herein. Additionally, the finite state machine may comprise a combination of a combinational logic circuit (and the sequential logic circuit, for example.

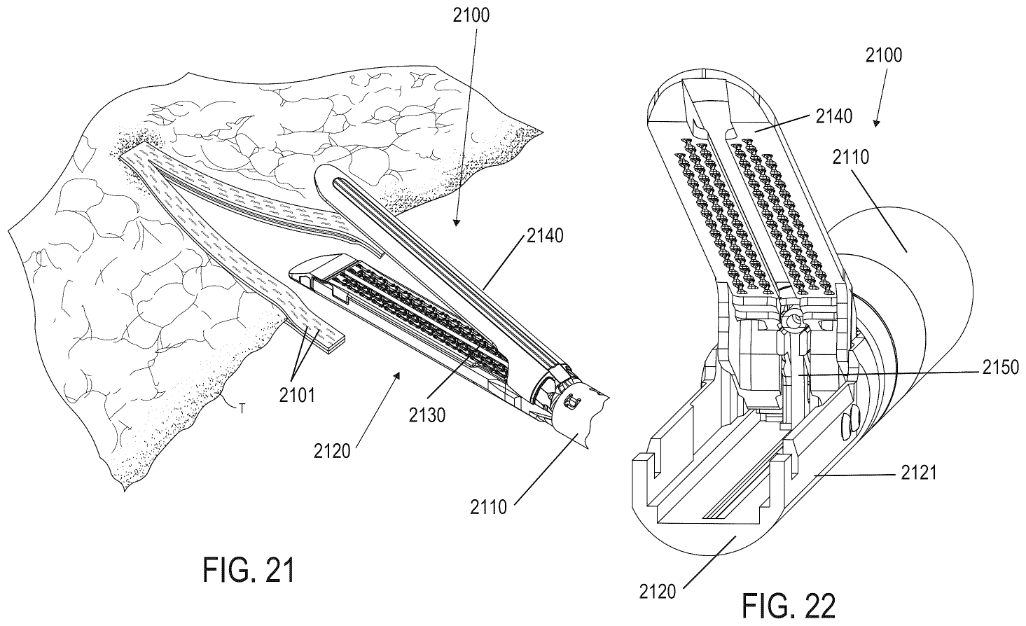

FIGS. 21 and 22 depict a surgical stapling assembly 2100 configured to clamp, staple, and cut patient tissue T during a surgical stapling procedure. As discussed herein, one or more functions of the surgical stapling assembly 2100 can be motor-driven. The surgical stapling assembly 2100 comprises a shaft 2110 and an end effector 2120 extending from the shaft 2110. The end effector 2120 comprises a cartridge channel jaw 2121 and an anvil jaw 2140 movable relative to the cartridge channel jaw 2121 to clamp tissue therebetween during a clamping stroke. In accordance with the present disclosure, the cartridge channel jaw 2121 can be movable in addition to, or in lieu of, the anvil jaw 2140. The end effector 2120 further comprises a replaceable staple cartridge 2130 configured to be installed into the cartridge channel jaw 2121. The replaceable staple cartridge 2130 comprises a plurality of staples 2101 removably stored therein and configured to be ejected from the replaceable staple cartridge 2130 during a staple firing stroke. In accordance with the present disclosure, the staple cartridge 2130 may not be replaceable. A disposable loading unit may comprise a shaft and an end effector attachable to a control interface. Additionally, in accordance with the present disclosure, the entire cartridge channel jaw 2121 may be replaceable.

The surgical stapling assembly 2100 further comprises a firing driver 2150 actuatable through the end effector 2120 by a drive assembly such as the drive assembly 2011, for example. The firing driver 2150 can comprise any suitable firing driver such as, for example, a distal I-beam head, discussed in greater detail below. The firing driver 2150 is configured to push a sled of the replaceable staple cartridge 2130 from an unfired position to a fired position. During distal translation of the sled within the replaceable staple cartridge 2130, the sled is configured to sequentially lift a plurality of staple drivers with staples 2101 supported thereon. As the drivers are lifted toward the anvil jaw 2140, the drivers are configured to eject the staples 2101 from a plurality of staple cavities and against the anvil jaw 2140.

In accordance with the present disclosure, the sled can be part of the firing driver. Any suitable combination of firing components can be considered the firing driver.

In accordance with the present disclosure, moving the anvil jaw 2140 into a clamped position to clamp tissue between the anvil jaw 2140 and the replaceable staple cartridge 2130 may be performed by a closure driver. The closure driver may be separate from the firing driver 2150 and may be actuatable independently of the firing driver. Alternatively, the closure driver may not be separate from the firing driver 2150, and the clamping, or closing, motion may be performed by the firing driver 2150. Opposing jaw-camming pins of a distal I-beam head of the firing driver 2150 are configured to cam the anvil jaw 2140 into a clamped position as the firing driver 2150 is actuated distally through an initial clamping stroke, or motion. In addition to moving the anvil jaw 2140 from an unclamped position to a clamped position during a clamping stroke, the opposing jaw-camming pins are configured to control a tissue gap distance between the anvil jaw 2140 and the replaceable staple cartridge 2130 during the staple firing stroke by limiting the separation of the cartridge channel jaw 2121 and the anvil jaw 2140 during the staple firing stroke with the opposing jaw-camming pins. One of the jaw-camming pins is configured to engage the cartridge channel jaw 2121 and one of the jaw-camming pins is configured to engage the anvil jaw 2140. Discussed in greater detail herein, this clamping action provided by the opposing jaw-camming pins induces clamping forces on the firing driver 2150 and other drive components.

In accordance with the present disclosure, many different forces can be transmitted through the surgical stapling assembly 2100 and these forces can induce loads on the firing driver 2150. These loads can include, but are not limited to, jaw-camming loads experienced during the staple firing stroke as a result of the interaction between the jaws 2121, 2140 and the jaw-camming pins of the firing driver 2150, tissue loads experienced during the staple firing stroke as a result of the interaction between a cutting edge, or knife, of the firing driver 2150 or firing assembly generally, for example, and the patient tissue T, and/or staple firing loads experienced during the staple firing stroke as a result of the firing of the staples 2101 by the firing driver 2150. In accordance with the present disclosure, these loads experienced by the firing driver 2150 can be transmitted through the drive assembly, or drive train, which drives the firing driver 2150, to the prime mover of the drive train such as the motor, for example.

The loads experienced by the firing driver 2150 can affect the overall performance of the staple firing stroke performed by the surgical stapling assembly 2100. Various components of a drive assembly are provided which, in accordance with the present disclosure, may be utilized in a surgical stapling drive assembly to help increase the efficiency, control, and/or reliability, for example, of a surgical stapling drive stroke.

In accordance with the present disclosure, one or more components of the drive assembly, or drive train, may be designed so as to optimize one or more characteristics of the drive train for a drive stroke such as, for example, a surgical stapling drive stroke. Such characteristics may include inertial mass and/or storable kinetic energy, for example. These characteristics can be utilized by a control circuit (e.g., control circuit 2004) to further optimize the drive stroke, as discussed in greater detail below.

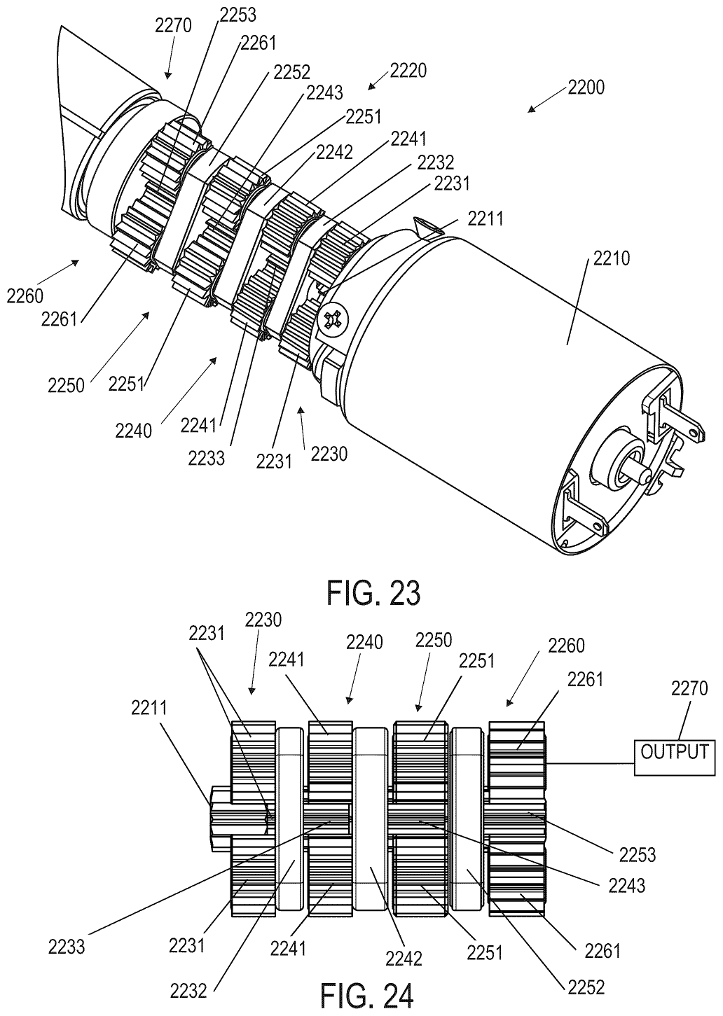

FIGS. 23 and 24 depict a drive assembly 2200 for use with a surgical stapling assembly such as the surgical stapling assembly 2100, for example. In accordance with the present disclosure, the drive assembly 2200 may be coupled to and configured to drive the firing driver 2150 through a drive stroke. The firing driver 2150 may comprise a firing beam having a first jaw-camming portion and a second jaw-camming portion. Additionally, the drive assembly 2200 may be controlled by a control circuit such as those described herein. The drive assembly 2200 comprises a motor 2210 and a gear train 2220 driven by the motor 2210. The drive assembly 2200 is configured to drive an output 2270. In accordance with the present disclosure, the output 2270 may be couplable to the firing driver 2150, for example. Any suitable drive driver can be coupled to the drive assembly 2200. The motor 2210 may comprise any suitable type of motor such as, for example, an electric motor. The motor 2210 may comprise a brushed or brushless DC motor. The drive stroke may comprise an initial clamping stroke to approximate the jaws and, subsequently, a staple firing stroke to staple and cut tissue. Alternatively, the drive stroke may only comprise a staple firing stroke to staple and cut tissue.

The motor 2210 comprises an output pinion gear 2211 driven by the motor 2210 and configured to impart drive motions to the gear train 2220. The gear train 2220 comprises a plurality of gear sets 2230, 2240, 2250, and 2260. The first gear set 2230 comprises a plurality of input planet gears 2231 meshed with the output pinion gear 2211, a carrier 2232 by which the input planet gears 2231 are supported, and an output gear 2233 supported by the carrier 2232. The output pinion gear 2211 is meshed with and configured to drive the planet gears 2231 which are directly coupled to the carrier 2232. Thus, the planet gears 2231, the carrier 2232, and the output gear 2233 all rotate together when driven by the output pinion gear 2211. The second gear set 2240 comprises a plurality of input planet gears 2241 meshed with the output gear 2233, a carrier 2242 by which the input planet gears 2241 are supported, and an output gear 2243 supported by the carrier 2242. The output gear 2233 is meshed with and configured to drive the planet gears 2241 which are directly coupled to the carrier 2242. Thus, the planet gears 2241, the carrier 2242, and the output gear 2243 all rotate together when driven by the output gear 2233. The third gear set 2250 comprises a plurality of input planet gears 2251 meshed with the output gear 2243, a carrier 2252 by which the input planet gears 2251 are supported, and an output gear 2253 supported by the carrier 2252. The output gear 2243 is meshed with and configured to drive the planet gears 2251 which are directly coupled to the carrier 2252. Thus, the planet gears 2251, the carrier 2252, and the output gear 2253 all rotate together when driven by the output gear 2243. The output gear set 2260 comprises a plurality of planet gears 2261 meshed with the output gear 2253. The output gear set 2260 is configured to drive the output 2270 to drive a drive driver such as, for example, the firing driver 2150.

Any suitable gear ratio or ratios can be employed within the gear train 2220. In accordance with the present disclosure, each gear stage may employ between about a 5:1 and a 10:1 gear ratio, for example. Each gear stage may reduce the output speed by about 5:1, 6:1, and/or 7:1, for example. In accordance with the present disclosure, if the input speed of the motor 2210 is 20,000 RPM, for example, the output speed of the output 2270 may be reduced by 5:1 at each gear stage. Multiple gear stages can provide a compound gear ratio. Additionally, a lesser gear ratio may be utilized closer to the motor 2210 so as to conserve rotational speed at the first gear stage from the motor 2210. Further, in accordance with the present disclosure, greater gear ratios which are different than the lesser gear ratio can be used downstream of the motor 2210 so as to reduce the speed of the output 2270 as desired. Discussed in greater detail below, the conservation of rotational speed of the gear set(s) closer to the motor 2210 increases the ability of the drive train 2220 to produce and conserve angular momentum to overcome possible stall conditions, as discussed in greater detail below. In accordance with the present disclosure, output speed and/or torque of the output 2270 may be controlled by a motor control circuit (e.g., control circuit 2004) by varying input voltage and/or current to the motor 2210. Load on the motor 2210 can be monitored and utilized to further control the motor 2210.

In accordance with the present disclosure, gear trains for various surgical instruments can include any number of suitable number of gear stages. A surgical stapling assembly, such as the surgical stapling assembly 2100, for example, can include three gear stages and can complete a full firing stroke (about 2.5 inches, for example) in about three seconds with a load up to about 200 lbs. Alternatively, a circular surgical stapler can include five gear stages, and can complete a full firing stroke (about 0.5 inches, for example) in about 2-5 seconds with a load up to about 600 lbs. The number of gear stages may depend on the required firing load, duration of stroke, and/or length of stroke, for example. As the firing load increases during a firing stroke, the potential for motor stall increases. Increasing the inertial properties of one or more of the gear stages can increase the amount of momentum generated during the firing stroke in the gear train and, under potential stall-inducing loads, can help the gear train push through the potential stall-inducing loads without stalling. In accordance with the present disclosure, the gear stage which rotates with the highest speed (closest to the motor, for example) can be modified so as to increase the inertial properties thereof and, because inertia is proportional to the mass and rotational velocity of the gear stage, the gear stage closest to the motor can provide the most momentum within the gear train.

In accordance with the present disclosure, a housing ring gear may be employed with the drive assembly 2200. Such a housing ring gear can be fixed relative to the gear sets 2230, 2240, 2350, and 2260 and meshed with the planet gears 2231, 2241, 2251, and 2261. In accordance with the present disclosure, a shiftable gear may be provided to switch between different gears during a staple firing stroke.

In accordance with the present disclosure, the first gear set 2230 may spin at a rotational speed faster than all of the subsequent gear sets. Furthermore, the first gear set 2230 may comprise increased inertial properties such as for example, inertial mass and moment arms, relative to subsequent gear sets so as to increase the amount of rotational kinetic energy, or angular momentum, produced by the first gear set 2230. Discussed in greater detail below, the material selection and geometry of each individual gear set can be selected based on the location of the gear set relative to the output 2270 and/or the motor 2210 and/or based on the nominal rotational speed of the gear set determined by the gear ratio relative to the motor 2210. In accordance with the present disclosure, materials and/or geometries of one or more gear sets rotating with a greater velocity closer to the motor 2210 may be selected so as to prioritize the generation and/or storability of kinetic energy within the gear sets positioned closer to the motor 2210. Further, in accordance with the present disclosure, materials and/or geometries of one or more gear sets rotating with a lesser velocity further downstream of the motor 2210 may be selected so as to prioritize manufacturing costs and/or loading properties, for example. Additionally, one or more gear sets further downstream in the gear train 2220 may be comprised of plastic while one or more gear sets closer to the motor 2210 may be comprised of metal.

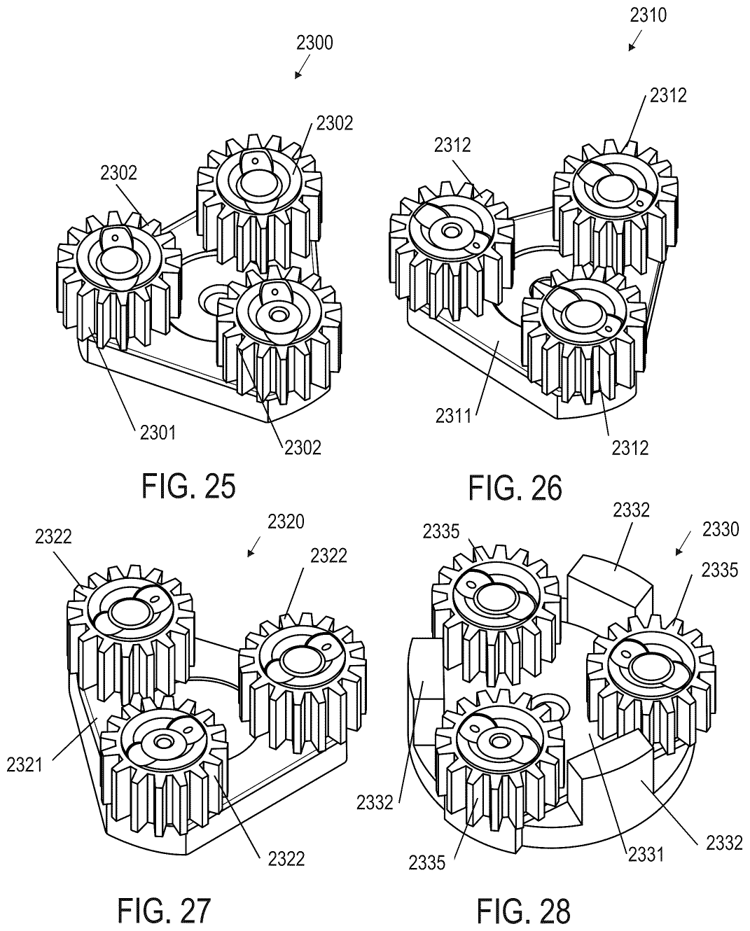

Turning now to FIGS. 25-28, different gear sets 2300 (FIG. 25), 2310 (FIG. 26), 2320 (FIG. 27), and 2330 (FIG. 28) are illustrated. In accordance with the present disclosure, each gear set 2300, 2310, 2320, 2330 may comprise materials, geometries, and/or components with different masses, sizes, and/or geometries thus changing the inertial properties of each gear set 2300, 2310, 2320, 2330. The drive assemblies disclosed herein may employ any combination of the gear sets 2300, 2310, 2320, and 2330. The combination of gear sets selected for a drive assembly can impact the overall inertial properties of the drive assembly. Each gear set may have specific inertial properties optimized for a surgical stapling drive stroke. Further, the inertial properties of each gear set may be selected based on the relative position of the gear set within a gear train.

In accordance with the present disclosure, the inertial properties may be selected based on a nominal operating speed of the gear set. As discussed above, a gear set having the greatest inertia may be selected for the first gear set coupled to the motor 2210 so as to generate a greater amount of kinetic energy with the fastest spinning gear set. For example, the gear set(s) closer to the motor 2210 can be driven at higher speeds than the gear sets further downstream of the motor 2210. Because of the higher nominal operating speed of the gear sets closer to the motor, these gear sets can include materials and design features so as to increase their inertial mass. Increasing the inertial mass of the faster-spinning gear sets can be employed to increase the overall inertia of the drive assembly 2200. The gear sets further downstream from the motor 2210 may be spun at lower nominal operating speeds and thus, increasing their inertial mass may minimally increase the overall inertia of the drive assembly 2200. In accordance with the present disclosure, all of the gear sets may be designed so as to increase the overall inertia of the drive assembly 2200. Additionally, the gear sets operating at lower nominal operating speeds may be selected so as to reduce manufacturing costs and/or reduce load properties.

The gear set 2300 comprises a carrier 2301 and planet gears 2302 rotatably mounted to the carrier 2301. In accordance with the present disclosure, a first material may be utilized for the carrier 2301 and a second material different than the first material may be utilized for the planet gears 2302. The first material may be lighter, as represented by mass in a given volume, than the second material. In accordance with the present disclosure, the first material may comprise a metal material and the second material may comprise a plastic material.

The gear set 2310 comprises a carrier 2311 and planet gears 2312 rotatably mounted to the carrier 2311. In accordance with the present disclosure, the carrier 2311 and the planet gears 2312 may comprise the same material. The material of the carrier 2311 and the planet gears 2312 may comprise a plastic material. Additionally, the inertial mass of the gear set 2300 may be more than the inertial mass of the gear set 2310.

The gear set 2320 comprises a carrier 2321 and planet gears 2322 rotatably mounted to the carrier 2321. In accordance with the present disclosure, the material of the carrier 2321 may comprise a plastic material. The material of the planet gears 2322 may comprise a hybrid material including at least two different materials. One of the materials may comprise tungsten. Additionally, the inertial mass of the gear set 2320 may be more than the inertial mass of the gear sets 2300, 2310. While plastic materials and metal materials are provided as examples of suitable materials for varying inertial mass of various components of the drive assembly 2200. It is readily understood that any suitable materials with different densities can be utilized.

The gear set 2330 comprises a carrier 2331 and planet gears 2335 rotatably mounted to the carrier 2331. As can be seen in FIG. 28, the carrier 2331 comprises outer pillars 2332. In accordance with the present disclosure, the outer pillars 2332 may further increase the inertial mass of the carrier 2331 as compared to the carriers without the outer pillars 2332. The material of the carrier 2331 may comprise multiple materials such as, for example, metal and plastic materials, where a central portion of the carrier 2331 may comprise a plastic material and an outer portion of the carrier 2331 including the pillars 2332 may comprise a metal material. The material of the planet gears 2335 may comprise the same or different materials than the carrier 2331.

Referring still to FIG. 28, the mass of the carrier 2331 is increased and dispersed further outward to increase the storable kinetic energy of the carrier 2331. The size, mass, and/or location of the pillars 2332 can be specifically selected so as to increase the inertial properties of the carrier 2331 and, thus, the gear set 2330. In accordance with the present disclosure, the gear set 2330 may be the first gear set driven by the motor so as to rotate the gear set 2330 with the maximum rotational velocity in the gear train and, thus, generate a maximum amount of momentum within the gear train. Any of the carries disclosed herein may comprise any suitable shape such as triangular and/or circular, for example. The pillars 2332 may be situated closer to the perimeter of the carrier 2331 than the center of the carrier 2331 to increase the ability of the gear set 2330 to store kinetic energy.

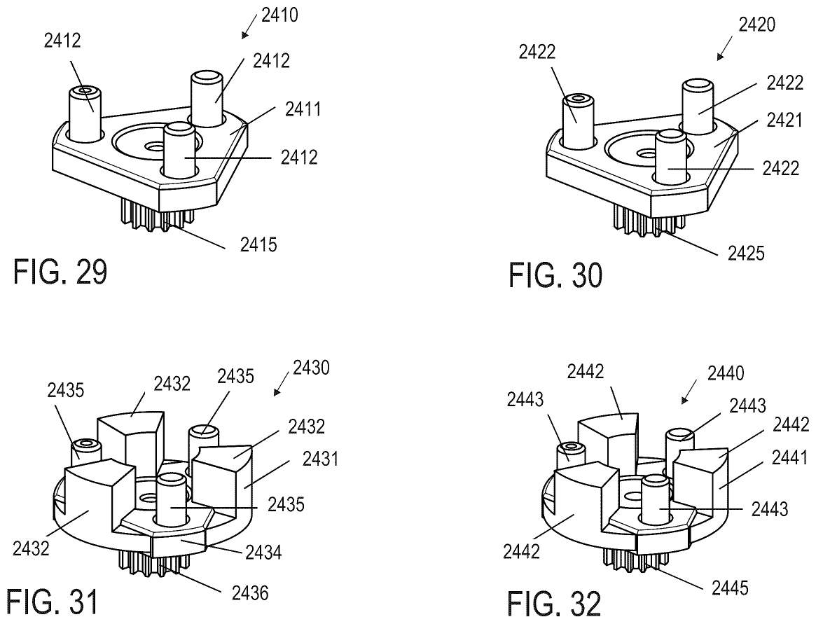

FIGS. 29-32 depict carriers 2410 (FIG. 29), 2420 (FIG. 30), 2430 (FIG. 31), and 2440 (FIG. 32). In accordance with the present disclosure, each carrier 2410, 2420, 2430, 2440 may comprise materials with different inertial masses and/or different geometries thus affecting the inertial properties thereof. The drive assemblies disclosed herein may employ any combination of the carriers 2410, 2420, 2430, and 2440 in a drive train thereof.

The carrier 2410 comprises a primary body portion 2411 and a pinion 2415 fixedly attached to the primary body portion 2411 such that the primary body portion 2411 and the pinion 2415 rotate together. The primary body portion 2411 comprises a plurality of mounting posts 2412 extending therefrom each of which is configured to rotatably support a planet gear thereon. The carrier 2410 comprises a first inertia value. In accordance with the present disclosure, the carrier 2410 may comprise a Nylon material, for example.

The carrier 2420 comprises a primary body portion 2421 and a pinion 2425 fixedly attached to the primary body portion 2421 such that the primary body portion 2421 and the pinion 2425 rotate together. The primary body portion 2421 comprises a plurality of mounting posts 2422 extending therefrom each of which are configured to rotatably support a planet gear thereon. The carrier 2420 comprises a second inertia value. In accordance with the present disclosure, the carrier 2420 can be cast with a steel material, for example. The first inertia value of the carrier 2410 may be less than the second inertia value of the carrier 2420.

The carrier 2430 comprises a primary body portion 2431, a secondary body portion 2434, and a pinion 930 fixedly attached to the primary body portion 2431 and the secondary body portion 2434 such that the primary body portion 2431, the secondary body portion 2434, and the pinion 2436 rotate together. The primary body portion 2431 comprises a plurality of secondary posts 2432 extending therefrom. In accordance with the present disclosure, the secondary posts 2432 can increase the inertial properties such as for example, the inertial mass, of the carrier 2430 relative to a carrier without secondary posts. The secondary body portion 2434 comprises a plurality of mounting posts 2435 each of which is configured to rotatably support a planet gear thereon. The carrier 2430 comprises a third inertia value.

In accordance with the present disclosure, the carrier 2430 may comprise a steel material and a Nylon material. The primary body portion 2431 may be cast with a steel material and the secondary body portion 2434 may comprise a Nylon material. The pinion 2436 may be part of the primary body portion 2431 and may also be made of a Nylon material. Alternatively, the pinion 2436 may be part of the secondary body portion 2434 and may also be cast with a steel material. Additionally, the third inertia value may be greater than the first inertia value of the carrier 2410 and the second inertia value of the carrier 2420.

The carrier 2440 comprises a body portion 2441 and a pinion 2445 fixedly attached to the body portion 2441 such that the body portion 2441 and the pinion 2445 rotate together. The body portion 2441 comprises a plurality of mounting posts 2443 extending therefrom and a plurality of secondary posts 2442 extending therefrom. In accordance with the present disclosure, the secondary posts 2442 can increase the inertial properties such as for example, the inertial mass, of the carrier 2440 relative to a carrier without secondary posts. Each mounting post 2443 is configured to rotatably support a planet gear thereon. The carrier 2440 comprises a fourth inertia value. The carrier 2440 can be entirely cast, or otherwise formed, with a steel material. Additionally, the fourth inertia value may be greater than the first inertia value of the carrier 2410, the second inertia value of the carrier 2420, and the third inertia value of the carrier 2430.

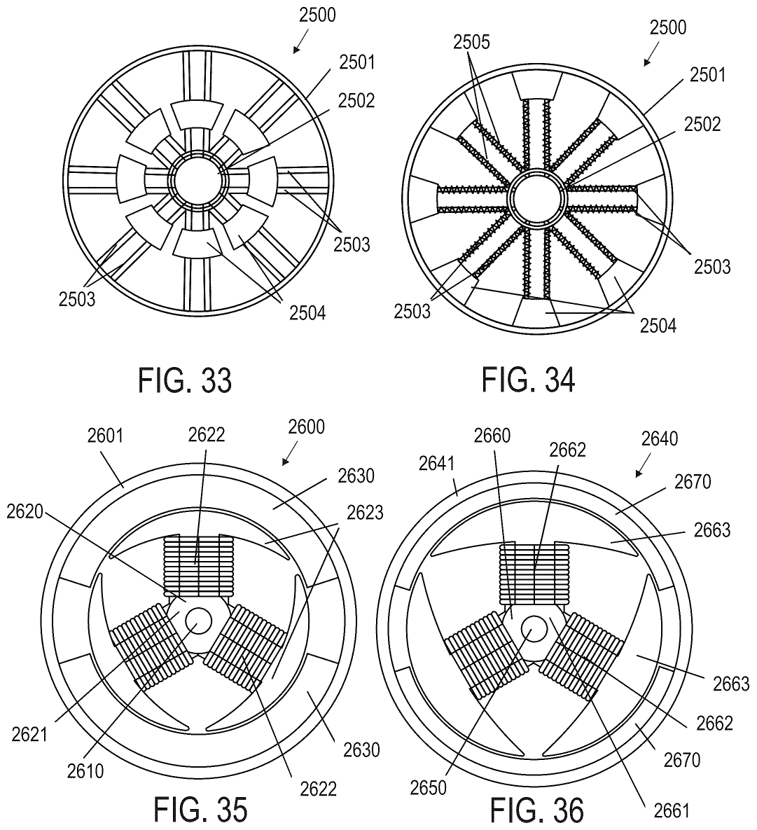

Various other components of a drive assembly 2200 can be selected for altering the inertial properties of the drive assembly of a surgical stapling device, for example. FIGS. 33-34 depict a flywheel, disc, or rotor, 2500 configured to increase the ability of a drive assembly for a surgical instrument to store rotational kinetic energy of a drive assembly by increasing the rotational inertia of the drive assembly. In accordance with the present disclosure, the flywheel 2500 may be mounted to a gear train (e.g., gear train 2220) within a drive assembly (e.g., drive assembly 2200) so as to increase the amount of storable kinetic energy during a drive stroke.

In accordance with the present disclosure, the flywheel 2500 may be passively actuated between a plurality of different configurations so as to provide different levels of inertia in each of the configurations. Alternatively, the flywheel 2500 may be actively actuated manually and/or automatically by a control circuit (e.g., control circuit 2004) between a plurality of different configurations so as to provide different levels of inertia. Additionally, the flywheel 2500 may be configured to utilize conservation of momentum to store rotational kinetic energy during a drive stroke.

The flywheel 2500 comprises an inner hub portion 2501, an outer rim portion 2502, and a plurality of struts, or arms, 2503 connecting the inner hub portion 2501 and the outer rim portion 2502. In accordance with the present disclosure, the inner hub portion 2501 can be mounted to a drive shaft of a drive assembly (e.g., drive assembly 2200). The flywheel 2500 can be mounted to the drive assembly near the motor before the gear box. Alternatively, the flywheel 2500 can be mounted to the drive assembly within the gear box. Additionally, the flywheel 2500 can be mounted to the drive assembly downstream, or distal, of the gear box.

The struts 2503 each comprise a radially-extending arm mounted to the inner hub portion 2501 and outer rim portion 2502 and can be oriented in a plurality of arm pairs. The flywheel 2500 further comprises masses 2504 slidably mounted to the struts 2503 such that the masses 2504 can be positioned near the inner hub portion 2501 as the flywheel 2500 rotates and the masses 2504 can be positioned near the outer rim portion 2502 as the flywheel 2500 rotates. The masses 2504 are configured to increase the rotational inertia of a drive assembly during a firing stroke by providing masses which are movable away from the center of rotation to increase the moment of inertia of the flywheel 2500 and, thus, the drive assembly.

In accordance with the present disclosure, the masses 2504 can be passively actuated between an inner position near the inner hub portion 2501 (FIG. 33) and an outer position near the outer rim portion 2502 (FIG. 34). In the illustrated example, springs 2505 are provided so as to hold the masses 2504 near the inner hub portion 2501 during little or no rotation of the flywheel 2500 but allow the masses 2504 to move radially outwardly toward the outer rim portion 2502 as the rotational velocity of the flywheel 2500 increases. This can reduce the amount of startup torque required by the motor of the drive assembly (e.g., drive assembly 2200) to initiate rotation the drive assembly but maximize rotational kinetic energy once the drive assembly is spinning at a nominal operating speed, for example.

In the instance of passive actuation of the masses 2504, the springs 2505 may comprise any suitable preselected spring constant selected so as to allow the masses 2504 to move radially outwardly as the flywheel 2500 increases rotational velocity to a nominal rotational velocity. During a decrease in velocity, the rotational kinetic energy stored in the flywheel 2500 is directed through, or released into, the drive assembly (e.g., drive assembly 2200) in an effort to reduce the possibility of a stall condition. As the speed of the flywheel 2500 decreases, the masses 2504 may be pulled back in closer to the inner hub portion 2504 to prepare for the next drive stroke by way of the springs 2505. In accordance with the present disclosure, the spring constant of the springs 2505 may be selected so as to control the position of the masses 2504 at different rotational speeds of the flywheel 2500. Further, in accordance with the present disclosure, the masses 2504 may not be pulled in closer to the inner hub portion 2504 until the flywheel 2500 fully stops rotating or nearly fully stops rotating, for example.

In accordance with the present disclosure, each spring 2505 may comprise a different spring constant so as to control the release of the masses 2504 toward the outer rim portion 2502. Such a configuration can allow for varied levels of inertia during a drive stroke. The different spring constants would require different rotational velocities of the flywheel 2500 to deploy each mass 2504 toward the outer rim portion 2502. In accordance with the present disclosure, the different rotational velocities required to deploy each mass to achieve a specific inertia level can be known and can be pursued by a control circuit (e.g., control circuit 2004) accordingly. For example, if a detected load on a motor is increasing at a predetermined rate that indicates a potential stall condition is imminent, the control circuit can be configured to automatically increase the rotational speed of the flywheel 2500 so as to generate more kinetic energy within the drive assembly. This additional kinetic energy can help push through the potential stall condition. If the load falls below a predetermined threshold after the potential stall condition is detected, the rotational speed of the flywheel 2500 can be reduced to move one or more masses 2504 back toward the inner hub portion 2501 and reduce the inertia level of the drive assembly when high stored kinetic energy is not necessary and, in accordance with the present disclosure, less desirable. If the kinetic energy stored in a drive assembly is too high, the magnitude of undershoot error and/or overshoot error of the drive stroke can increase. For example, a target velocity of a drive driver may be overshot and undershot by a larger margin with the unnecessary, increased stored kinetic energy than if the increased stored kinetic energy was not present in the drive assembly.

In accordance with the present disclosure, the masses 2504 can be actuated automatically by a control circuit (e.g., control circuit 2004) between the inner positions (FIG. 33) and the outer positions (FIG. 34). Additionally, a control circuit can automatically deploy one or more masses 2504 to their outer positions and/or inner positions to pursue the desired amount of stored kinetic energy in the drive assembly at any given time during the drive stroke. Any suitable actuation method can be utilized. A linear actuator such as a solenoid, for example, may be provided for each mass 2504 so as to be able to control each mass 2504 individually thereby providing several different levels of rotational inertia within the drive assembly.

FIGS. 35 and 36 depict different motor configurations configured to contribute different levels of inertia to a drive assembly. In accordance with the present disclosure, the motors illustrated in FIGS. 35 and 36 may comprise DC motors. The motors can be brushed or brushless. FIG. 35 depicts a motor 2600 comprising a motor housing 2601, an output shaft 2610, a rotor 2620 connected to the output shaft 2610, and stator magnets 2630 all housed within the motor housing 2601. The rotor 2620 comprises an inner hub portion 2621, coil winding arms 2622 extending outwardly from the inner hub portion 2621, and outer rotor extensions 2623 extending outwardly from the coil winding arms 2622.

FIG. 36 depicts a motor 2640 comprising a motor housing 2641, an output shaft 2650, a rotor 2660 connected to the output shaft 2650, and stator magnets 2670 all housed within the motor housing 2641. The rotor 2660 comprises an inner hub portion 2661, coil winding arms 2662 extending outwardly from the inner hub portion 2661, and outer rotor extensions 2663 extending outwardly from the coil winding arms 2662.

As can be seen in FIGS. 35 and 36, the outer rotor extensions 2663 comprise a larger volume than the outer rotor extensions 2623. In accordance with the present disclosure, the outer rotor extensions 2663 may comprise a greater mass and/or weight than the outer rotor extensions 2623. Similarly, the stator magnets 2630 may comprise a greater volume than the stator magnets 2670. The motor 2640 may have a greater inertia than the motor 2600. Additionally, the motor 2640 can be configured to generate and store more rotational kinetic energy for use within a surgical instrument drive assembly than the motor 2600.

Different inertial properties of components of a drive assembly can vary the ability of the drive assembly to store mechanical energy during a drive stroke within the drive assembly. In accordance with the present disclosure, more inertial mass of a gear stage of a gear box, for example, can increase the rotational kinetic energy of the drive assembly during a drive stroke. An inertial mass of one or more gear box components may be selected to provide sufficient kinetic energy during the drive stroke to overcome a stall condition of the motor caused by the external application of a predicted stall-induced load on the drive element. Additionally, increasing the moment arm of the planet gears of a gear stage can increase the inertia of the gear stage and, thus, the drive assembly. Increasing the mass of one or more of the planet gears can increase the inertia of the gear stage. This increase in rotational energy of a gear stage can result in an increased ability of the drive assembly to generate and/or conserve angular momentum. The increased ability to conserve angular momentum, or store rotational energy, can help reduce the possibility of motor stall, for example, discussed in greater detail below.

The drive assemblies and systems provided herein are configured to increase the amount of load absorbable within the system without stalling the motor. In accordance with the present disclosure, the increased inertial properties of the gear train may allow the systems to overcome force variations beyond a peak motor input torque (maximum torque available by the motor) owing to the stored kinetic energy in the system.

In accordance with the present disclosure, a control circuit (e.g., control circuit 2004) of a surgical instrument coupled to a motorized drive train which controls a drive stroke of the surgical instrument can utilize inertial characteristics of a drive train to help automatically reduce the likelihood of motor stall during the drive stroke.

In accordance with the present disclosure, the control circuit 2004 can be configured to monitor one or more parameters of the drive assembly during a drive stroke and the stored kinetic energy of the drive assembly in an effort to detect an imminent motor stall condition. The control circuit 2004 is configured to compare, or evaluate, the monitored one or more parameters and the stored kinetic energy of the drive assembly to determine if motor stall is imminent.

The parameters can include any suitable parameter or combination of parameters of the drive assembly. In accordance with the present disclosure, the parameters can include various motor parameters such as, for example, motor velocity, motor current, motor acceleration, motor load, motor efficiency, motor heat, and/or voltage sag, etc. The parameters can include various gear box parameters such as, for example, rotational velocity, rotational acceleration, and/or stored kinetic energy of one or more gear stages. Additionally, the parameters can include various aspects of a drive member, or driver, such as, for example, load on the drive member (which can be induced by stapling forces caused by ejecting staples from a staple cartridge and forming the staples against the anvil, clamping forces caused by clamping tissue between the jaws and/or controlling a tissue gap distance between the jaws during a staple firing stroke, and/or tissue-cutting forces caused by the interaction between a tissue-cutting knife of the drive member and the tissue during the staple firing stroke), velocity of the drive member, and/or acceleration of the drive member.

Any of the parameters or combination of parameters discussed herein can be utilized in determining that a motor stall event is imminent. A reduction in speed of the drive member, rapid deceleration of the drive member, and/or increased motor current and/or torque, for example, can all be signs of an imminent motor stall condition. A reduction in gear stage speed can also indicate motor stall is imminent. In accordance with the present disclosure, an increase in motor heat, motor torque, and/or motor current can indicate that a motor stall event is imminent. A combination of parameters may be monitored by the control circuit 2004 to detect an imminent motor stall condition. Additionally, more than one parameter of a drive assembly may be monitored and only when every monitored parameter indicates that a motor stall condition is imminent does the control circuit 2004 determine that a motor stall condition is imminent.

In accordance with the present disclosure, one or more of the parameters can be configured to be monitored over a period of time and analyzed to determine the rate at which the parameter is changing, for example. The rate of change of the parameters can be evaluated by the control circuit 2004 during a staple firing stroke and, when one or more particular parameters changes a predetermined amount, or at a predetermined threshold rate of change, over a predetermined period of time, the control circuit 2004 can utilize this information in determining an imminent motor stall condition. For example, if current increases rapidly at a predetermined threshold rate of increase, this can be utilized to determine that a motor stall condition is imminent.

In accordance with the present disclosure, the control circuit 2004 can detect an imminent motor stall condition based on multiple parameters, as discussed above. The control circuit 2004 may separately compare values, which can be determined based on sensor signals received from sensors that monitor such parameters, to a predetermined threshold associated with each parameter. Alternatively, the imminent motor stall condition can be a function of a number of parameters weighed diffidently in a predetermined equation for detecting the imminent motor stall condition.

As discussed herein, the control circuit 2004 is further configured to monitor and/or determine a kinetic energy stored in the drive assembly during a staple firing stroke, for example, to evaluate alongside, or compare with, the monitored parameter. If the determined stored kinetic energy of the drive assembly is below a predetermined kinetic energy threshold for a given stall condition, which would indicate that the motor is likely to stall unless an adjustment is made to the drive stroke, an adjustment can be made to the drive stroke in an effort to overcome the stall condition and completely prevent the motor from stalling. In accordance with the present disclosure, the adjustment can include making a motor adjustment which increases the kinetic energy of the drive assembly to push through, or overcome, the imminent stall condition.

The kinetic energy stored in the drive assembly can be determined in any suitable manner such as, for example, by monitoring the rotational speed of the motor and/or a particular gear stage, for example. In accordance with the present disclosure, the control circuit 2004 can calculate the kinetic energy stored in the drive assembly using the known values of drive assembly such as for example, the mass of the gear box components (planet gears and carriers), and a variable parameter (parameter which changes during the firing stroke) of the gear box component (speed of a particular gear stage). The control circuit 2004 can be configured to sum the kinetic energy storage of every component in the drive assembly to determine the total stored kinetic energy in the drive assembly at any given time during the firing stroke.

As the stored kinetic energy increases and decreases during the drive stroke, the ability of the drive assembly to overcome a stall condition increases and decreases. For example, if the drive member is moving relatively slowly during the drive stroke, a relatively low amount of stored kinetic energy can be present within the drive assembly. Therefore, the ability of the drive assembly to overcome a potential motor stall event decreases. Given this decrease in stored kinetic energy, the control circuit 2004 is configured to adjust the sensitivity of the trigger, or parameter, thresholds of the monitored parameters which would require, or trigger, a motor adjustment to overcome a stall condition. For example, as the stored kinetic energy of the drive assembly decreases, the tolerance for stall-related variations of the drive assembly is decreased because less kinetic energy is stored within the drive assembly lowering the ability of the drive assembly to overcome a stall condition. As another example, as the stored kinetic energy of the drive assembly increases, the tolerance for stall-related variations of the drive assembly is increased because more kinetic energy is stored within the drive assembly increasing the ability of the drive assembly to overcome a stall condition.

In accordance with the present disclosure, the control circuit 2004 can be further configured to utilize a kinetic energy threshold during a drive stroke. The kinetic energy threshold may be predetermined, or preset. Additionally, the kinetic energy threshold may be adjusted in real time by the control circuit 2004 during the drive stroke based on one or more parameters of the drive stroke. Different kinetic energy thresholds may be set for different stages of the drive stroke. The stages may include, for example, a lockout stage where an I-beam is either locked out or defeats the lockout as the I-beam moves through the lockout stage, an initial clamping stage where the I-beam engages opposing jaw-camming channels defined in the jaws as the jaws initially clamp on tissue, a tissue cutting and stapling stage where the I-beam ejects staples, cuts tissue, and holds the jaws clamped during the stapling and cutting of tissue, and/or an ending stage after the firing of staples.

The kinetic energy threshold can be preset for each stage corresponding to the anticipated stored kinetic energy which may be required to overcome a potential stall condition during each stage of the drive stroke. For example, the lockout stage may require a relatively low kinetic energy. This may be due to the fact that there is little-to-no jaw-camming forces acting on the I-beam pins, no cutting of tissue, and no stapling of tissue, for example. Thus, the likelihood of motor stall may be much less likely during the lockout stage of the drive stroke. Because motor stall is unlikely and/or because the load, for example, experienced during the lockout stage is low, a relatively low kinetic energy threshold can be set by the control circuit 2004 for the lockout stage.

In accordance with the present disclosure, the tissue cutting and stapling stage may involve the greatest loads on the drive assembly. In accordance with the present disclosure, a relative high kinetic energy threshold may be set to ensure that a maximum amount of available stored kinetic energy of the drive assembly is being provided during the tissue cutting and stapling stage so that, in the event of detecting a possible motor stall condition, a maximum amount of kinetic energy is stored in the drive assembly to overcome the motor stall condition.

In accordance with the present disclosure, the kinetic energy threshold may be utilized by the control circuit 2004 to ensure that the drive assembly is actuated to provide a stored kinetic energy which meets and/or exceeds the kinetic energy threshold during a particular stage of the drive stroke. The control circuit 2004 can be configured to compare the determined stored kinetic energy of the drive assembly with the kinetic energy threshold and initiate a motor control adjustment should the stored kinetic energy of the drive assembly fall below the kinetic energy threshold, for example, in an effort to maintain a stored kinetic energy that meets and/or exceeds the kinetic energy threshold.

In accordance with the present disclosure, a comparison of the kinetic energy threshold and the stored kinetic energy of the drive assembly may be utilized to adjust a sensitivity of one or more parameter thresholds indicative of motor stall. Such parameter thresholds are monitored parameter values of the drive stroke which may trigger a motor adjustment. The parameter thresholds may comprise a range of monitored parameter values where, for example, when the monitored parameter falls outside of the range, a motor adjustment is triggered. Alternatively, a parameter threshold may not comprise a range but, rather, a single value where, for example, when the monitored parameter exceeds, or falls below, the parameter threshold, a motor adjustment may be made. A motor adjustment may be made to, ultimately, prevent motor stall. This can be achieved by adjusting torque output of the motor, speed output of the motor, acceleration output of the motor, etc. For example, a motor adjustment may include increasing the speed of the motor to a maximum speed for a short duration in an effort to serve as a kinetic buffer during the potential stall event. In accordance with the present disclosure, the motor adjustment may be held until a stall condition is no longer detected. A multitude of quick speed bursts may be provided by the control circuit 2004 in an attempt to overcome the potential stall condition.

As discussed herein, the sensitivity, or tolerance, of the parameter threshold can be adjusted in real time based on the stored kinetic energy of a drive assembly during a drive stroke. In other words, with less stored, or storable, kinetic energy, a parameter threshold can be adjusted to be more sensitive such that a more marginal variation of the monitored parameter would trigger a motor adjustment. This can be a result of the greater stall risk associated with less stored, or storable, kinetic energy. Similarly, with more stored, or storable, kinetic energy, the parameter threshold can be adjusted to be less sensitive so that a greater variation of the monitored parameter is permitted prior to triggering a motor adjustment. This can be a result of the lower stall risk associated with more stored, or storable, kinetic energy. In accordance with the present disclosure, the sensitivity may be adjusted based on the amount of stored kinetic energy. Additionally, the sensitivity may be based on the comparison of the amount of stored kinetic energy at a given moment and the kinetic energy threshold.

For example, if the stored kinetic energy of the drive assembly is much greater than the kinetic energy threshold, the parameter threshold can be set a first, less conservative, sensitivity. If the stored kinetic energy of the drive assembly is much lower than the kinetic energy threshold, the parameter threshold can be set at a second, more conservative, sensitivity.

In accordance with the present disclosure, there may be increased risk of motor stall in a device without an adaptable sensitivity adjustment of the parameter thresholds. Assume the parameter threshold is not adjusted as the speed of the drive element, for example, varies through the drive stroke, and the parameter threshold is set at a 10% increase in load thereby initiating a motor adjustment at a 10% increase in load at any given drive element speed. Should the load increase 10%, a motor adjustment is made. This may be adequate at higher, more nominal drive element speeds, where a 10% increase in load may not necessarily stall the motor. However, at a lower speed, a 10% increase in load may cause a motor stall because of the lower storable kinetic energy. Thus, the control circuit (e.g., control circuit 2004) described herein would adjust the parameter threshold to a more sensitive load variation such as, for example, 5% at lower speeds. The parameter threshold may be adjusted fluidly with the monitored parameter of the drive assembly (drive element speed, in this instance). This can allow for a more adaptable motor control algorithm that can provide a surgeon with varying drive element speed while minimizing the risk of motor stall no matter what speed the surgeon, or a surgical robot, is actuating the drive element.

In accordance with the present disclosure, the control circuit 2004 can be configured to switch between a first drive configuration of the gear box to provide a first inertia value and a second drive configuration of the gear box to provide a second inertia value. The first drive configuration may include a slower speed but more torque while the second drive configuration may include an increased speed but less torque. The drive configurations may be switched between automatically by the control circuit 2004 or manually by a clinician. In accordance with the present disclosure, a solenoid may be provided to switch between different drive configurations. The first drive configuration provides a first amount of storable kinetic energy while the second drive configuration provides a second amount of storable kinetic energy which is greater than the first amount of storable kinetic energy. The drive configuration can be chosen by the control circuit 2004 depending on the minimum kinetic energy threshold of the current stage of the drive stroke. Additionally, the drive configuration can be chosen by the control circuit 2004 depending on stored kinetic energy of the drive assembly at a given time. For example, as the stored kinetic energy falls, the drive configuration may switch to the higher torque, lower speed configuration.

In accordance with the present disclosure, a target speed for the drive element may be selected based on the desired stored kinetic energy for the stage of the drive stroke. If motor stall risk is relatively low, a lower target speed may be selected. If motor stall risk is relatively high, a higher target speed may be selected to ensure a maximum amount of storable kinetic energy. Tissue thickness and/or clamping load can affect the velocity of the drive element and thus storage of kinetic energy in the drive assembly. Thicker tissue may increase the load on the drive element and decrease the speed of the drive element. This can reduce the stored kinetic energy in the drive assembly. Thinner tissue may decrease the load on the drive element and maintain a consistently higher speed of the drive element. This can increase the amount of stored kinetic energy in the drive assembly.

As discussed herein, motor adjustments can be made by the control circuit 2004 in an effort to overcome a potential stall condition. The motor adjustments can include any suitable adjustment such as those described herein. In accordance with the present disclosure, motor input voltage and/or current can be adjusted. A drive configuration of a gear box can be adjusted to change the amount of available kinetic energy to overcome the potential stall condition. Once a motor stalls, it may be significantly more difficult for a motor to restart a drive stroke. Motor stall may require reversing the drive element before re-advancing the drive element through the rest of the drive stroke. Such a motion may introduce a degree of unpredictability in forming the rest of the staples and/or cutting tissue through the rest of the drive stroke. The increased load on the drive element for restarting a drive element after a motor stall can present unnecessary stress and strain on drive assembly components. Additionally, motor stall may introduce voltage sag in a battery source for the motor and affect the integrity of the battery for future use. Motor stall may damage the motor, mechanical drive train components, gear box gears, etc. Providing a motor-adjustment when detecting a potential stall condition can provide a kinetic buffer to reduce the likelihood of motor stall.

Reducing motor stall of a surgical stapling instrument can increase longevity of the instrument and its components (battery, motor, etc.), increase the reliability of a surgical stapling and cutting drive stroke (ensure proper staple formation and clean tissue-cutting), increase confidence in surgeon to perform each drive stroke, reduce time of the drive stroke and, thus, the surgical stapling operation.

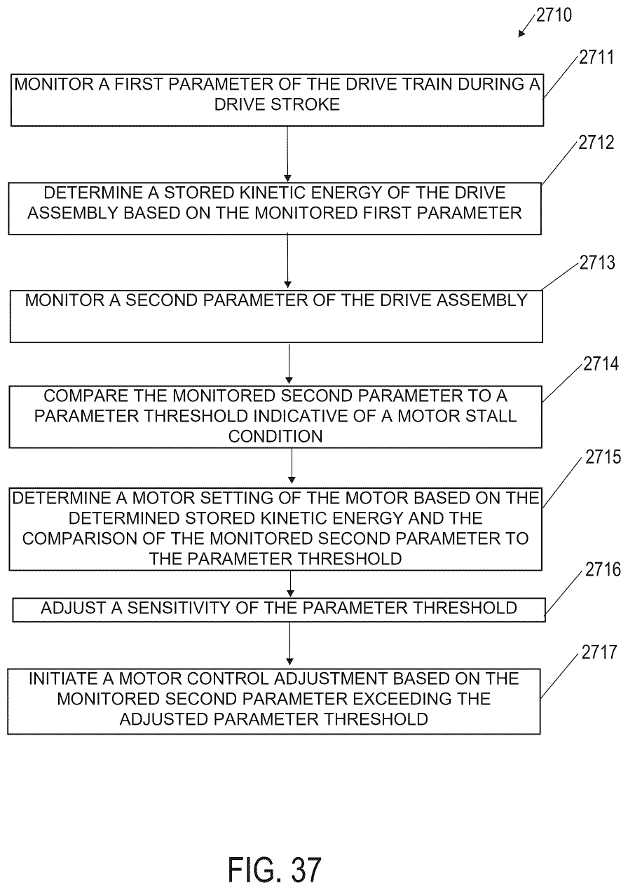

FIG. 37 is a logic flow chart depicting a process 2710 executable by a control circuit, such as the control circuit 2004 illustrated in FIG. 20, for example, for controlling the motor of a drive assembly of a surgical stapling system such as those disclosed herein. The control circuit 2004 is coupled to a motor assembly 2009 including a motor, a drive element (e.g., firing beam), and a drive train connecting the motor and drive element. The drive train is actuatable by the motor to move the drive element through a drive stroke. In accordance with the present disclosure, the drive stroke may comprise a lockout stage, an initial clamping stage, a tissue cutting and stapling stage, and an ending stage. The control circuit 2004 is usable with the drive assembly, drive components, and surgical instrument components described herein.

The control circuit 2004 is configured to monitor 2711 a first parameter of the drive assembly during the drive stroke. As discussed herein, the first parameter may include any suitable parameter or combination of parameters indicative of the stored kinetic energy of the drive assembly. Such a parameter can include, for example, an output speed of the motor. The control circuit 2004 is further configured to determine 2712 a stored kinetic energy of the drive assembly based on the monitored 2711 first parameter. As discussed herein, determining the stored kinetic energy of the drive assembly can be achieved by utilizing known values of the drive assembly such as, for example, inertial mass, gear set moment arms, etc., and utilizing the monitored first parameter such as, for example, output speed of the drive train. This information can be used to calculate the total stored kinetic energy at any given moment during the drive stroke. In accordance with the present disclosure, the stored kinetic energy of the drive assembly may vary over time owning to external factors such as tissue load, for example, and/or owing to controllable inputs such as, for example, motor voltage/speed.

The control circuit 2004 is further configured to monitor 2713 a second parameter of the drive assembly during the drive stroke. In accordance with the present disclosure, the first parameter and the second parameter may be different. The second parameter may comprise tissue-induced load on the drive element, for example. Additionally, the monitored second parameter may be utilized to help detect a potential, or imminent, stall condition during the drive stroke. In the example of tissue-induced load on the drive element, a certain tissue load magnitude can indicate a potential stall condition. A threshold rate of increase of tissue load on the drive element can indicate a potential stall condition. As discussed herein, a combination of parameters can be utilized to help detect and/or alert of a potential stall condition. For example, in addition to tissue load on the drive element, drive element output speed can also be monitored and when the drive element speed and the tissue-induced load on the drive element meet a predetermined criteria, a potential stall condition can be indicated.

The control circuit is further configured to compare 2714 the monitored second parameter to a parameter threshold indicative of a motor stall condition. Such a parameter threshold may indicate a need for motor adjustment to prevent the motor from stalling. As discussed herein, the parameter threshold can include an acceptable percentage change in the monitored second parameter or the surpassing of a straight threshold value of the monitored second parameter, for example, before a motor adjustment is suggested or even necessary to prevent motor stall.

The control circuit 2004 is further configured to determine 2715 a motor setting of the motor based on the determined stored kinetic energy and the comparison of the monitored second parameter to the parameter threshold. The motor setting can include any suitable motor setting. In accordance with the present disclosure, the control circuit 2004 may determine to keep the motor setting the same. Additionally, the control circuit 2004 may determine that the stored kinetic energy of the drive assembly is adequate to overcome the potential stall condition with the current motor setting. The control circuit 2004 may adjust the motor setting such as, for example, increasing the speed of the motor, to increase kinetic energy in an effort to overcome the potential stall condition.

The control circuit 2004 is further configured to adjust 2716 a sensitivity of the parameter threshold indicative of a motor stall condition. In accordance with the present disclosure, the sensitivity of the parameter threshold may be a tolerance of acceptable variation of the monitored second parameter relative to the parameter threshold that indicates a motor stall condition. For example, as the stored kinetic energy increases, the control circuit 2004 can set a more tolerant parameter threshold to allow for more load on the firing beam before making a motor adjustment, for example, with the understanding that the drive assembly contains enough stored kinetic energy to overcome the increased load, or increased parameter threshold. On the other hand, the control circuit 2004 can set a more stringent parameter threshold, or tighten the tolerance of the parameter threshold, so that as the stored kinetic energy decreases, for example, even a slight load on the firing beam may indicate a motor stall condition.

In accordance with the present disclosure, the sensitivity of the parameter threshold may be based on the determined 2712 stored kinetic energy of the drive assembly. As discussed herein, the sensitivity of the parameter threshold can be represented by the magnitude of change of the monitored second parameter which would require a motor adjustment to prevent motor stall. In other words, the tolerance of change of the monitored second parameter before a motor adjustment is initiated can be decreased as the stored kinetic energy decreases. The tolerance of change of the monitored second parameter before a motor adjustment is initiated can be increased as the stored kinetic energy increases. As discussed herein, more stored kinetic energy can allow for a greater variance of the monitored second parameter before initiating a motor adjustment to prevent motor stall because the drive assembly has more kinetic energy to serve as a kinetic buffer through an imminent stall condition.

In accordance with the present disclosure, the tolerance level of variance of the monitored second parameter before initiating a motor adjustment may be tightened as the stored kinetic energy decreases. Similarly, the tolerance level of variance of the monitored second parameter before initiating a motor adjustment can be widened as the stored kinetic energy increases. The tolerance level, or sensitivity, may be adjusted from a 10% acceptable increase threshold of tissue-induced load on the drive element for a first kinetic energy to a 5% acceptable increase threshold of tissue-induced load on the drive element for a second kinetic energy where the second kinetic energy is lower than the first kinetic energy.

In accordance with the present disclosure, the motor control adjustment can be initiated 2717 based on the monitored second parameter exceeding the adjusted parameter threshold. Any suitable motor control adjustment can be initiated such as those disclosed herein. For example, the speed of the motor may be increased to a maximum level in an effort to increase the kinetic energy and, thus, stored kinetic energy to provide a kinetic buffer through the imminent stall condition.

While stall conditions are generally indicated as the events to be prevented, other drive assembly-related events can be monitored and used as the triggering event for sensitivity adjustments, motor control adjustments, and/or stored kinetic energy adjustments. For example, battery integrity of a battery powering a motor can be monitored and kinetic buffers can be utilized to help reduce the load on the battery over time. In accordance with the present disclosure, motor heat may effect motor efficiency over time and can be used to trigger kinetic buffers so as to help reduce motor heat during the life of the motor and/or during a single drive stroke, for example, of the motor.

While surgical stapling drive strokes are generally described in connection with the drive assemblies, control circuits, and processes disclosed herein, it can be appreciated that the drive assemblies, control circuits, and processes can be employed with a separate closure motor and/or closure drive assembly.

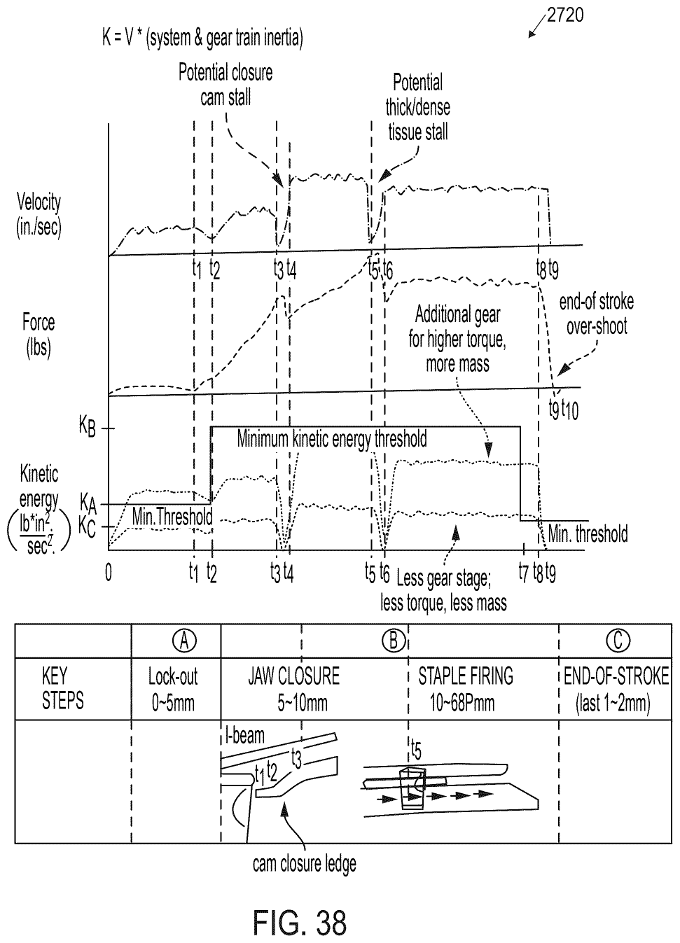

FIG. 38 is graph 2720 illustrating the velocity, force (load), and kinetic energy of an example drive stroke of a surgical stapling instrument such as those disclosed herein. As can be seen in FIG. 38, potential stall conditions are encountered at t3 and t5. These stall conditions can be overcome utilizing the control circuits, drive assemblies and components, and/or control circuit processes disclosed herein. The kinetic energy of multiple gear sets within a gear box of the drive assembly is also depicted in the graph 2720. As can be seen in the graph 2720, the kinetic energy of each gear set is different during different stages of the drive stroke. A minimum kinetic energy threshold is utilized and is different for various stages of the drive stroke. A control circuit (e.g., control circuit 2004) is configured to monitor the kinetic energies of each gear set relative to the minimum kinetic energy and, while also monitoring for potential stall conditions, ensure that a proper amount of kinetic energy in the drive assembly is maintained during the drive stroke provide a sufficient kinetic buffer through the stall events.

Still referring to FIG. 38, the minimum kinetic energy threshold may be set at its highest during the part of the drive stroke where the jaws are clamped, tissue is cut, and tissue is stapled. Maximum kinetic energy storage may be preferred during this period. As discussed herein, one or more other parameters are configured to be monitored during the drive stroke. These parameters are compared with a parameter threshold and, when the parameter threshold is met, an adjustment is made to the motor (maximize motor output speed, for example) to maintain kinetic energy through a potential stall event. The control circuit 2004 is further configured to adjust a sensitivity of the motor-adjustment trigger thresholds in real time during the drive stroke. In accordance with the present disclosure, the sensitivity of the parameter thresholds may be much greater (a more stringent tolerance) at lower speeds during the drive stroke whereas the sensitivity of the parameter thresholds may be much lower (a less stringent tolerance) at higher speeds during the drive stroke.

The minimum kinetic energy threshold is also configured to be adjusted based on a zone within which the drive element is positioned during the drive stroke. As discussed herein, maintaining a particular kinetic energy through particular sections of the drive stroke can increase reliability and predictability of the drive stroke reducing the possibility of motor stall, for example. A system which simply increases the motor speed to a maximum for example ahead of a potential stall condition may be undesirable. In accordance with the present disclosure, maximizing motor speed during the staple firing and tissue-cutting part of the drive stroke may be okay while maximizing motor speed during the tissue clamping part of the drive stroke may clamp tissue too quickly or apply too much pressure to tissue compared to the desired levels of the user. Thus fine tuning the kinetic buffer can ensure proper amounts of speed and force are applied during specific parts of the drive stroke without overcompensating, for example, ahead of a potential stall condition.

In accordance with the present disclosure, a potential stall condition near, at, or after the staple firing and tissue-cutting part of the stroke may not be as important to buffer as a potential stall condition during other parts of the stroke. The control circuit 2004 can be configured to adjust the motor control program so as to not ram the drive element into the end of the end effector in the event of detecting a potential stall condition.

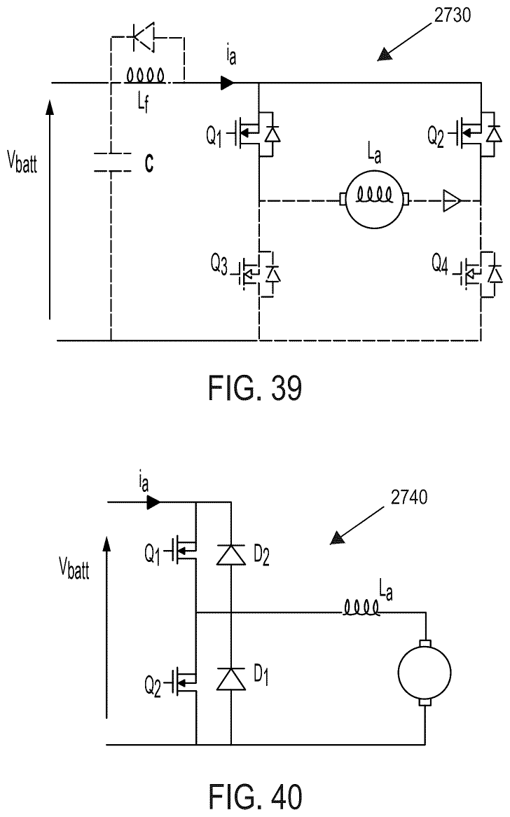

In at least one instance, energy in a drive train of a motorized surgical instrument can be harvested to recharge a battery in a system where a motor of the surgical instrument is powered by an onboard battery. In at least one instance, regenerative braking can employed by the motorized surgical instrument by converting the motor into a generator which can be used to recover kinetic energy and use the recovered energy immediately and/or store the recovered energy in a capacitor and/or a battery of the motorized surgical instrument, for example. Such regenerative breaking can be employed at any suitable point during a stroke of the motorized surgical instrument. For example, regenerative breaking can be employed at or near the end of the cut line, before, during, and/or after a fault condition (hitting an existing staple line in the tissue, for example, triggering a current spike), in between motor pulses and/or during pauses in motor control signals (use “off” portion of PWM signal to recharge battery using regenerative braking), and/or during retraction portion of the stroke. Dynamic braking regeneration can be used to charge a boosting circuit which can be used to overcome, or push through, a tough portion of tissue, for example. One example of a power recovery circuit 2730 can be seen in FIG. 39. Another example of a power recovery circuit 2740 can be seen in FIG. 40.

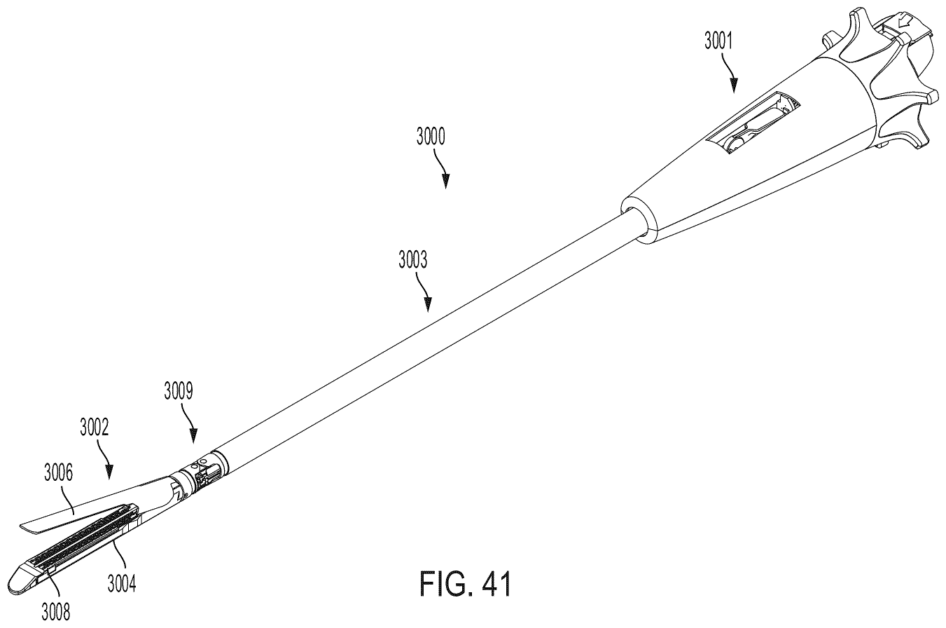

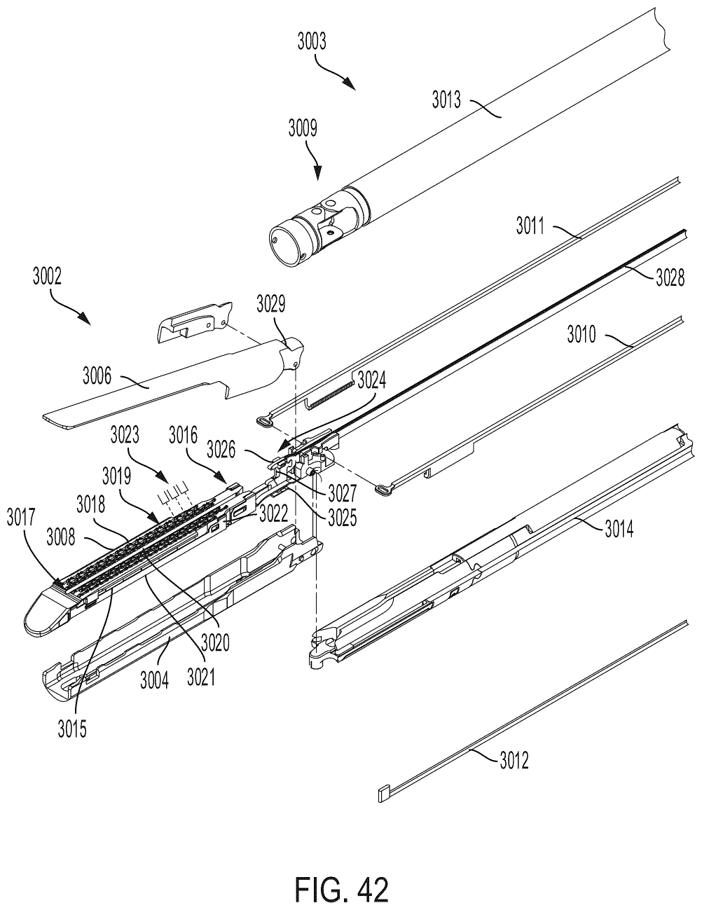

FIGS. 41 and 42 illustrate a surgical stapling system comprising a shaft assembly 3000 and an end effector 3002 extending from the shaft assembly 3000. The shaft assembly 3000 comprises an attachment portion 3001 and a shaft 3003 extending distally from the attachment portion 3001. The attachment portion 3001 is configured to be attached to a handle of a surgical instrument and/or the arm of a surgical robot, for example.

The end effector 3002 comprises a first jaw 3004 and a second jaw 3006. The first jaw 3004 comprises a staple cartridge 3008 insertable into and removable from the first jaw 3004; however, other embodiments are envisioned in which a staple cartridge is not removable from, or at least readily replaceable from, the first jaw 3004. The second jaw 3006 comprises an anvil configured to deform staples ejected from the staple cartridge 3008. The second jaw 3006 is pivotably coupled to the first jaw 3004 such that the second jaw 3006 is pivotable relative to the first jaw 3004 between an open position, where the tip of the second jaw 3006 is space apart from the first jaw 3004 (see FIG. 41) and a closed position, where the tip of the second jaw 3006 is adjacent the first jaw 3004 to capture tissue between the first jaw 3004 and the second jaw 3006; however, other embodiments are envisioned in which the first jaw 3004 is pivotable relative to the second jaw 3006.

The surgical stapling system comprises an articulation joint 3009 configured to permit the end effector 3002 to be rotated, or articulated, relative to the shaft 3003. The end effector 3002 is rotatable about an articulation axis extending through the articulation joint. Some embodiments may omit the articulation joint 3009. The shaft assembly 3000 comprises cooperating articulation rods 3010, 3011 configured to articulate the end effector 3002 relative to the shaft 3003 about the articulation joint 3009. The shaft assembly 3000 comprises an articulation lock bar 3012 configured to prevent rotation of the end effector 3002, an outer shaft tube 3013 configured to house internal components of the shaft assembly 3000, and a spine portion 3014 configured to provide structure support to the shaft assembly 3000.

The staple cartridge 3008 comprises a cartridge body 3015 including a deck 3018 extending between a proximal end 3016 and a distal end 3017. In use, the staple cartridge 3008 is positioned on a first side of tissue to be stapled and the anvil 3006 is positioned on a second side of the tissue. The anvil 3006 is moved toward the staple cartridge 3008 to compress and clamp the tissue against the deck 3018. Thereafter, staples 3023 removably stored in the cartridge body 3015 are deployed into the tissue. The cartridge body 3015 comprises a plurality of staples removably stored in a plurality of staple cavities 3019 defined within the cartridge body 3015. The staple cavities 3019 are arranged in six longitudinal rows. Three rows of staple cavities are positioned on a first side of a longitudinal slot 3020 and three rows of staple cavities are positioned on a second side of the longitudinal slot 3020. Other arrangements of staple cavities 3019 and staples may be possible.

The staples 3023 are supported by staple drivers in the cartridge body 3015. Staples supported on staple drivers are disclosed in U.S. Patent Application Publication No. 2021/0059672, which is herein incorporated by reference in its entirety. The drivers are movable between a first, unfired position, and a second, fired, position to eject the staples from the staple cavities 3019. The drivers are retained in the cartridge body 3015 by a retainer 3021 which extends around the bottom of the cartridge body 3015 and includes resilient members 3022 configured to grip the cartridge body 3015 and hold the retainer 3021 to the cartridge body 3015. The drivers are movable between their unfired positions and their fired positions by a sled. The sled is movable between a proximal position adjacent the proximal end 3016 and a distal position adjacent the distal end 3017. The sled comprises a plurality of ramped surfaces configured to slide under the drivers and lift the drivers, and the staples supported thereon, toward the anvil. In accordance with the present disclosure, the staples may not be supported by staple drivers, but rather, the staples may include integral drive surfaces that are directly engaged by the sled to lift the staples, examples of which are described in U.S. Patent Application Publication No. 2015/0173756, which is herein incorporated by reference in its entirety.

The sled is moved distally by a firing driver exemplified as a firing bar 3024 configured to contact the sled and push the sled toward the distal end 3017. The longitudinal slot 3020 defined in the cartridge body 3015 is configured to receive the firing driver 3024. The anvil 3006 also includes a slot configured to receive the firing driver 3024. The firing driver 3024 comprises a first cam 3025 which engages the first jaw 3004 and a second cam 3026 which engages the second jaw 3006. As the firing driver 3024 is advanced distally, the first cam 3025 and the second cam 3026 can control the distance, or tissue gap, between the deck 3018 of the staple cartridge 3008 and the anvil 3006. The firing driver 3024 also comprises a knife 3027 configured to incise the tissue captured intermediate the staple cartridge 3008 and the anvil 3006. The knife 3027 is desirably positioned at least partially proximal to the ramped surfaces to eject the staples ahead of the knife 3027. The shaft assembly 3000 comprises a firing bar 3028 attached to the firing driver 3024 and is configured to drive the firing driver through the staple cartridge 3008. In accordance with the present disclosure, the firing bar 3028 may comprise a plurality of laminated strips. More details of the shaft assembly 3000 are disclosed in U.S. patent application Ser. No. 15/385,887 entitled METHOD FOR ATTACHING A SHAFT ASSEMBLY TO A SURGICAL INSTRUMENT AND, ALTERNATIVELY, TO A SURGICAL ROBOT, which is herein incorporated by reference in its entirety.

In accordance with the present disclosure, the anvil 3006 may be moved from the open position to the closed position using a closure system that is controlled separately from the firing driver 3024, where the firing driver 3024 is considered to be a part of a firing system that is separate and distinctly operable from the closure system. Further, in accordance with the present disclosure, the anvil 3006 may comprise a ramp 3029 on a proximal end thereof and the closure system may comprise a closure member, such as an outer shaft tube 3013, that can be movable distally to engage the ramp 3029 and cam the anvil 3006 to the closed position. In the closed position, the first cam 3025 and the second cam 3026 of the firing driver 3024 translate distally and maintain the anvil 3006 in the closed position. To transition the anvil 3006 to the open position, the closure member may be retracted proximal and the anvil 3006 may be biased to the open position by springs positioned within the end effector 3002. The anvil 3006 may include a tab and the closure member may define an aperture at the distal end thereof which engages the tab as the closure member moves proximally, thereby positively transitioning the anvil 3006 to the open position. Exemplary closure systems and closure members are disclosed in U.S. Patent Application Publication No. 2021/0059672, the entire disclosure of which is hereby incorporated by reference herein.

In accordance with the present disclosure, the firing driver 3024 may move the anvil 3006 from the open position to the closed position. The anvil 3006 includes a ramp that extends from a wall defining the slot in the anvil 3006 and that is engaged by the firing driver 3024 during a first portion of the stroke of the firing driver 3024 to move the anvil 3006 to the closed position. At the end of the first portion of the stroke, the firing driver 3024 can continue advancing distally through a second portion of the stroke to deploy staples from the staple cartridge 3008 and incise tissue captured by the end effector 3002. Exemplary firing drivers that close the anvil and fire staples are disclosed in U.S. Pat. No. 11,160,551.

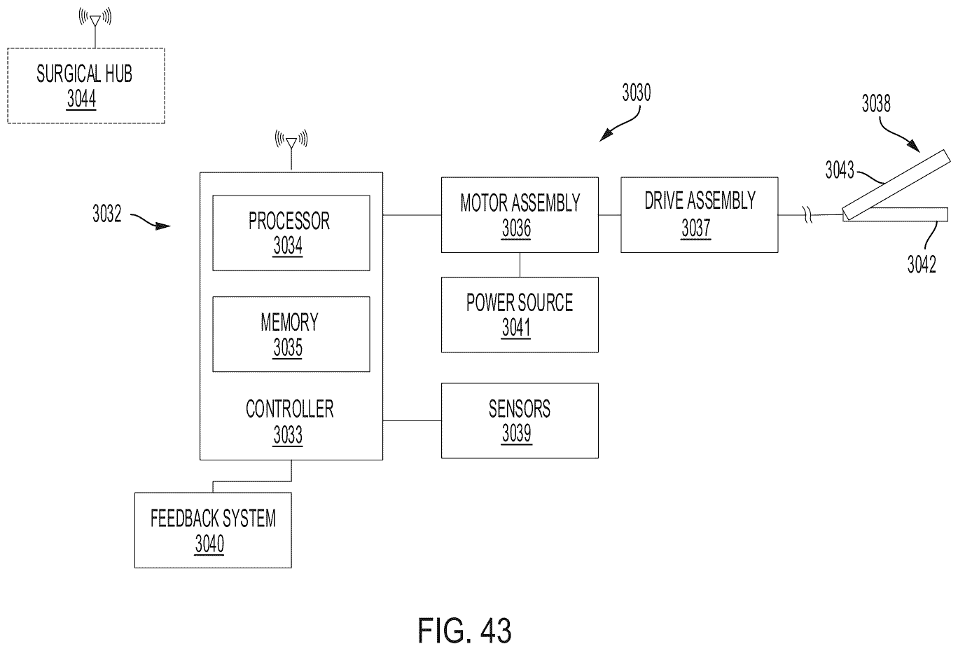

FIG. 43 illustrates a surgical system 3030 for use with one or more surgical instruments, tools, and/or robotic systems in accordance with the present disclosure. The surgical system 3030 includes a control circuit 3032. The control circuit 3032 includes a microcontroller 3033 comprising a processor 3034 and a storage medium such as, for example, a memory 3035.

A motor assembly 3036 includes a motor, driven by a motor driver. The motor assembly 3036 operably couples to a drive assembly 3037 to drive, or effect, motion at an end effector 3038, similar to the end effector 3002 shown in FIGS. 41 and 42. The drive assembly 3037 may include any number of components suitable for transmitting motion to the end effector 3038 such as, for example, one or more linkages, bars, tubes, and/or cables, for example. In accordance with the present disclosure, the drive assembly 3037 can drive a firing driver and/or a closure member.

A sensor(s) 3039, for example, provides real-time feedback to the processor 3034 about an operational parameter monitored during a surgical procedure being performed by the surgical system 3030. The operational parameter can be associated with a user performing the surgical procedure, a tissue being treated, and/or one or more components of the surgical system 3030, for example. The sensor 3039 may comprise one or more than one suitable sensor, such as, for example, a magnetic sensor, such as a Hall effect sensor, a strain gauge, a pressure sensor, a force sensor, an inductive sensor, such as an eddy current sensor, a resistive sensor, a capacitive sensor, an optical sensor, a current sensor, a voltage sensor, and/or any other suitable sensor.

The sensor(s) 3039 may comprise one or more than one suitable sensor for detecting one or more than one condition at the end effector 3038 including, without limitation, a tissue thickness sensor such as a Hall Effect Sensor or a reed switch sensor, an optical sensor, a magneto-inductive sensor, a force sensor, a pressure sensor, a piezo-resistive film sensor, an ultrasonic sensor, an eddy current sensor, an accelerometer, a pulse oximetry sensor, a temperature sensor, a sensor configured to detect an electrical characteristic of a tissue path (such as capacitance or resistance), or any combination thereof. As another example, and without limitation, the sensor(s) 3039 may include a sensor located at, or about, an articulation joint, similar to articulation joint 3009, extending proximally from the end effector 3038. Such sensors may include, for example, a potentiometer, a capacitive sensor (slide potentiometer), piezo-resistive film sensor, a pressure sensor, a pressure sensor, or any other suitable sensor type. In accordance with the present disclosure, the sensor(s) 3039 may comprise a plurality of sensors located in multiple locations in the end effector 3038.

In accordance with the present disclosure, the system 3030 may include a feedback system 3040 which may include a device for providing sensory feedback to a user. Such devices may comprise, for example, visual feedback devices (e.g., an LCD display screen, a touch screen, LED indicators), audio feedback devices (e.g., a speaker, a buzzer) or tactile feedback devices (e.g., haptic actuators).

The microcontroller 3033 may be programmed to perform various functions such as precise control over the speed and position of the drive assembly 3037. In accordance with the present disclosure, the microcontroller 3033 may be any single-core or multicore processor such as those known under the trade name ARM Cortex by Texas Instruments. Further, in accordance with the present disclosure, the main microcontroller 1933 may be an LM4F230H5QR ARM Cortex-M4F Processor Core, available from Texas Instruments, for example, comprising an on-chip memory of 256 KB single-cycle flash memory, or other non-volatile memory, up to 40 MHZ, a prefetch buffer to improve performance above 40 MHz, a 32 KB single-cycle SRAM, and internal ROM loaded with StellarisWare® software, a 2 KB EEPROM, one or more PWM modules, one or more QEI analogs, and/or one or more 12-bit ADCs with 12 analog input channels, details of which are available for the product datasheet.

The microcontroller 3033 may be configured to compute a response in the software of the microcontroller 3033. The computed response is compared to a measured response of the actual system to obtain an “observed” response, which is used for actual feedback decisions. The observed response is a favorable, tuned value that balances the smooth, continuous nature of the simulated response with the measured response, which can detect outside influences on the system.

The motor assembly 3036 includes one or more than one electric motor and one or more than one motor driver. The electric motor may be a brushed direct current (DC) motor with a gearbox and mechanical links to the drive assembly 3037. In accordance with the present disclosure, a motor driver may be an A3941 available from Allegro Microsystems, Inc.

In various forms, the motor assembly 3036 includes a brushed DC driving motor having a maximum rotational speed of approximately 25,000 RPM. The motor assembly 3036 may include a brushless motor, a cordless motor, a synchronous motor, a stepper motor, or any other suitable electric motor. The motor driver may comprise an H-bridge driver comprising field-effect transistors (FETs), for example. Those skilled in the art will appreciate that an amount of power any motor produces is determined solely by the voltage applied to the motor and the current drawn by the windings of the motor.

The motor assembly 3036 can be powered by a power source 3041. In accordance with the present disclosure, the power source 3041 may include one or more than one battery to power the motor assembly 3036. A battery may include a number of battery cells connected in series. The battery cells may be replaceable and/or rechargeable. Additionally, or alternatively, the battery cells can be lithium-ion batteries coupleable to and separable from the power assembly.

The end effector 3038 includes a first jaw 3042 and a second jaw 3043. At least one of the first jaw 3042 or the second jaw 3043 is rotatable relative to the other during a closure motion that transitions the end effector 3038 from an open configuration to a closed configuration. The closure motion may cause the jaws 3042, 3043 to grasp tissue therebetween. In accordance with the present disclosure, sensors, such as, for example, a strain gauge or a micro-strain gauge, can be configured to measure a parameter of the end effector 3038, such as, for example, the amplitude of the strain exerted on one or both of the jaws 3042, 3043 during a closure motion, which can be indicative of the closure forces applied to the jaws 3042, 3043. The measured strain is converted to a digital signal and provided to the processor 3034, for example. Alternatively or additionally, sensors such as, for example, a load sensor, can measure a closure force and/or a firing force applied to the jaws 3042, 3043. In accordance with the present disclosure, the sensors may comprise a first sensor to measure a first force on a firing driver 3024 during a firing stroke, and a second sensor to measure a second force on a closure member, such as the outer shaft tube 3013, during a closure stroke. The processor 3034 can receive these force measurements and determine a relationship therebetween, such as a distribution ratio of the force exerted on the firing driver 3024 and the outer shaft tube 3013.

In accordance with the present disclosure, a current sensor can be employed to measure the current drawn by a motor of the motor assembly 3036. The force required to advance the drive assembly 3037 can correspond to the current drawn by the motor, for example. The measured force is converted to a digital signal and provided to the processor 3034.

In accordance with the present disclosure, a strain gauge sensor can measure the force applied to the tissue by the end effector 3038. The strain gauge sensor can be coupled to the end effector 3038 to measure the force on the tissue being treated by the end effector 3038. The strain gauge sensor can measure the amplitude or magnitude of the strain exerted on a jaw of an end effector 3038 during a closure motion, which can be indicative of the tissue compression. The measured strain is converted to a digital signal and provided to a processor 3034.

The measurements of the tissue compression, tissue thickness, and/or force required to close the end effector on the tissue, as respectively measured by the sensors 3039 can be used by the microcontroller 3033 to characterize the selected position and/or corresponding value of the speed of one or more than one component of the drive assembly 3037. In accordance with the present disclosure, a memory 3035 can store instructions, an equation, and/or a lookup table which can be employed by the microcontroller 3033 in the assessment of position and speed on the one or more than one component of the drive assembly 3037.

The surgical system 3030 may comprise wired or wireless communication circuits to communicate with surgical hubs (e.g., surgical hub 3044), communication hubs, and/or robotic surgical hubs, for example. Additional details about suitable interactions between a surgical system 3030 and the surgical hub 3044 are disclosed in U.S. patent application Ser. No. 16/209,423 entitled METHOD OF COMPRESSING TISSUE WITHIN A STAPLING DEVICE AND SIMULTANEOUSLY DISPLAYING THE LOCATION OF THE TISSUE WITHIN THE JAWS, now U.S. Patent Application Publication No. 2019/0200981, the entire disclosure of which is herein incorporated by reference in its entirety.

The control circuit 3032 can be configured to implement various processes described herein. In accordance with the present disclosure, the control circuit 3032 may comprise a microcontroller 3033 comprising processor 3034 (e.g., microprocessor) coupled to a memory circuit 3035. The memory circuit 3035 stores machine-executable instructions that, when executed by the processor 3034, cause the processor 3034 to execute machine instructions to implement various processes described herein. The processor 3034 may be a single-core or multicore processor. The memory circuit 3035 may comprise volatile or non-volatile storage media. The processor 3034 may include a central processing unit (CPU) and an arithmetic unit. The CPU may be configured to receive instructions from the memory circuit 3035.

Alternatively, the control circuit 3032 can be a combinational logic circuit configured to implement various processes described herein. The combinational logic circuit may comprise a finite state machine comprising a combinational logic configured to receive data, process the data by the combinational logic, and provide an output.

Alternatively, the control circuit 3032 is a sequential logic circuit configured to implement various processes described herein. The sequential logic circuit may comprise a finite state machine. The sequential logic circuit may comprise a combinational logic, at least one memory circuit, and a clock, for example. The at least one memory circuit can store a current state of the finite state machine. In accordance with the present disclosure, the sequential logic circuit may be synchronous or asynchronous. The control circuit 3032 may comprise a combination of a processor (e.g., processor 3034) and a finite state machine to implement various processes herein. The finite state machine may comprise a combination of a combinational logic circuit (and the sequential logic circuit, for example.

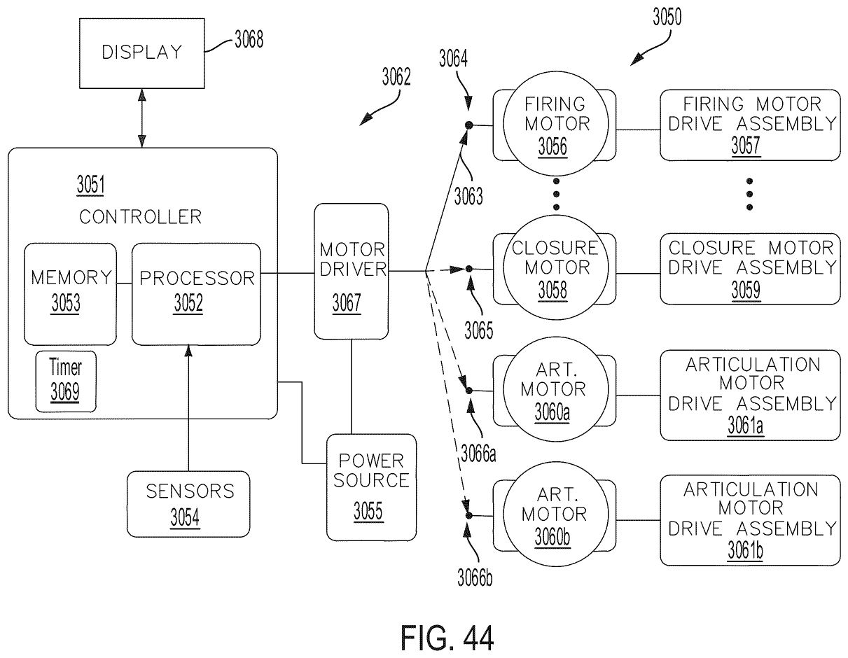

FIG. 44 is a block diagram of a surgical system 3050 for use with one or more surgical instruments, tools, and/or robotic systems in accordance with the present disclosure. The surgical system 3050 is similar in many respects to the surgical system 3030, which are not repeated herein at the same level of detail for brevity. For example, like the surgical system 3030, the surgical system 3050 includes a control circuit comprising a microcontroller 3051 comprising a processor 3052 and a memory 3053, a sensor 3054, and a power source 3055, which are similar, respectively, to the microcontroller 3033, the processor 3034, the memory 3035, and the power source 3041. Additionally, the surgical system 3050 includes a plurality of motors and corresponding driving assemblies that can be activated to perform various functions.

In accordance with the present disclosure, a first motor can be activated to perform a first function, a second motor can be activated to perform a second function, a third motor can be activated to perform a third function, a fourth motor can be activated to perform a fourth function, and so on. Additionally, in accordance with the present disclosure, the plurality of motors can be individually activated to cause firing, closure, and/or articulation motions in an end effector, such as end effector 3002 or end effector 3038, as examples. The firing, closure, and/or articulation motions can be transmitted to the end effector through a shaft assembly, such as shaft assembly 3000, for example.

The surgical system 3050 may include a firing motor 3056 operably coupled to a firing motor drive assembly 3057, which can be configured to transmit firing motions, generated by the motor 3056, to the end effector The firing motions including, for example, displacement of the firing bar 3028 and firing driver 3024. The firing motions generated by the motor 3056 may deploy the staples from the staple cartridge 3008 into tissue captured by the end effector, and/or advance the firing driver the knife 3027 to cut the captured tissue. The firing driver may be retracted by reversing the direction of the motor 3056.

The surgical system 3050 may include a closure motor 3058 operably coupled to a closure motor drive assembly 3059 configured to transmit closure motions, generated by the motor 3058, to the end effector. In particular, the closure motions displace a closure member, such as outer shaft tube, to close an anvil and compress tissue between the anvil and the staple cartridge. The closure motions may cause the end effector to transition from an open configuration to an approximated, or closed, configuration to grasp tissue, for example. The end effector may be transitioned to an open position by reversing the direction of the motor 3058.

The surgical system 3050 may include one or more than one articulation motors 3060a, 3060b, operably coupled to respective one or more than one articulation motor drive assemblies 3061a, 3061b configured to transmit articulation motions, generated by the motors 3060a, 3060b, to the end effector. The articulation motions may cause the end effector to articulate relative to a shaft, for example. In accordance with the present disclosure, the first articulation motor 3060a may drive a first articulation bar, such as articulation rod 3010, to rotate the end effector in a first direction and the second articulation motor 3060b may drive a second articulation bar, such as articulation bar 3011, to rotate the end effector in a second direction opposite the first direction.

The surgical system 3050 may include a plurality of motors configured to perform various independent functions. In accordance with the present disclosure, the plurality of motors of the surgical instrument or tool can be individually or separately activated to perform one or more than one function while the other motors remain inactive. The articulation motors 3060a, 3060b can be activated to cause the end effector to be articulated while the firing motor 3056 remains inactive. Alternatively, the firing motor 3056 can be activated to fire the plurality of staples, and/or to advance the cutting edge, while the articulation motors 3060a, 3060b remains inactive. The closure motor 3058 may be activated simultaneously with the firing motor 3056 to cause the closure member and the firing driver to advance distally at the same time, or in an overlapping fashion, as described in more detail herein below.

The surgical system 3050 may include a common control module 3062 which can be employed with a plurality of motors of the surgical instrument or tool. The common control module 3062 may accommodate one of the plurality of motors at a time. For example, the common control module 3062 can be coupleable to and separable from the plurality of motors of the robotic surgical instrument individually. A plurality of the motors of the surgical instrument or tool may share the common control module 3062. A plurality of motors of the surgical instrument or tool can be individually and selectively engaged with the common control module 3062. The common control module 3062 can be selectively switched from interfacing with one of a plurality of motors of the surgical instrument or tool to interfacing with another one of the plurality of motors of the surgical instrument or tool.

The common control module 3062 can be selectively switched between operable engagement with the articulation motors 3060a, 3060b and operable engagement with either the firing motor 3056 or the closure motor 3058. In the example illustrated in FIG. 44, a switch 3063 can be moved or transitioned between a plurality of positions and/or states. In a first position 3064, the switch 3063 may electrically couple the common control module 3062 to the firing motor 3056; in a second position 3065, the switch 3063 may electrically couple the common control module 3062 to the closure motor 3058; in a third position 3066a, the switch 3063 may electrically couple the common control module 3062 to the first articulation motor 3060a; and in a fourth position 3066b, the switch 3063 may electrically couple the common control module 3062 to the second articulation motor 3060b, for example. Separate common control modules 3062 can be electrically coupled to the firing motor 3056, the closure motor 3058, and the articulation motor 3060a, 3060b at the same time. The switch 3063 may be a mechanical switch, an electromechanical switch, a solid-state switch, or any suitable switch.

Each of the motors 3056, 3058, 3060a, 3060b may comprise a torque sensor to measure the output torque on the shaft of the motor. The force on an end effector may be sensed in any conventional manner, such as by force sensors on the outer sides of the jaws or by a torque sensor for the motor actuating the jaws.

As illustrated in FIG. 44, the common control module 3062 may comprise a motor driver 3067, which may comprise one or more H-Bridge FETs. The motor driver 3067 may modulate the power transmitted from a power source 3055 to a motor coupled to the common control module 3062 based on input from a microcontroller 3051 (the “controller”), for example. In accordance with the present disclosure, the microcontroller 3051 can be employed to determine the current drawn by the motor, for example, while the motor is coupled to the common control module 3062.

The processor 3052 may control the motor driver 3067 to control the position, direction of rotation, and/or velocity of a motor that is coupled to the common control module 3062. In accordance with the present disclosure, the processor 3052 can signal the motor driver 3067 to stop and/or disable a motor that is coupled to the common control module 3062.

The memory 3053 may include program instructions for controlling each of the motors of the surgical system 3050 coupleable to the common control module 3062. For example, the memory 3053 may include program instructions for controlling the firing motor 3056, the closure motor 3058, and the articulation motors 3060a, 3060b to cause the processor 3052 to control the firing, closure, and articulation functions in accordance with inputs from algorithms or control programs of the surgical instrument or tool.

A mechanism and/or sensor(s) 3054 can be employed to alert the processor 3052 to the program instructions that should be used in a particular setting. For example, the sensor(s) 3054 may alert the processor 3052 to use the program instructions associated with firing, closing, and articulating the end effector. In accordance with the present disclosure, the sensor(s) 3054 may comprise position sensors to sense the position of the switch 3063, for example. Accordingly, the processor 3052 may use the program instructions associated with firing the I-beam of the end effector upon detecting, through the sensors 3054 for example, that the switch 3063 is in the first position 3064; the processor 3052 may use the program instructions associated with closing the anvil upon detecting, through the sensors 3054 for example, that the switch 3063 is in the second position 3065; and the processor 3052 may use the program instructions associated with articulating the end effector upon detecting, through the sensors 3054 for example, that the switch 3063 is in the third or fourth position 3066a, 3066b. In accordance with the present disclosure, the controller 3051 can communicate with a display 3068, which can be similar to feedback system 3040, to provide feedback to a user. In addition, the display 3068 can include an input interface such that a user can provide input for controlling the surgical system 3050. The controller 3051 can include a timer 3069 to measure elapsed time.

Referring also to FIGS. 41 and 42, a surgical system can include a closure system and a firing system. The closure system includes a closure member, such as the outer shaft tube 3013, that is movable from a proximal position toward a distal position during a closure stroke. The closure member is driven between the proximal position and the distal position by the closure motor 3058; however, other embodiments are envisioned where the closure member is driven between the proximal position and the distal position by a manual-drive system that includes a closure trigger manually operable by a clinician. The surgical system comprises an end effector 3002 comprising a first jaw 3004, and a second jaw 3006, that is rotatable relative to the first jaw between an open position and a closed position. In use, the closure member translates toward the distal position and engages the second jaw, such as on a ramp on the proximal end thereof. The closure member applies a closure force to the second jaw to rotate the second jaw toward the closed position. In accordance with the present disclosure, the surgical system may include a power source 3041, 3055, to power the closure motor.

The firing system includes a firing driver 3024, that is movable from a proximal, unfired position, toward a distal, fired position, during a firing stroke to deploy staples stored in a staple cartridge 3008, and to incise tissue captured by the end effector with a knife 3027. The firing driver is driven between the proximal, unfired position and the distal, fired position by a firing motor 3056. The firing driver includes a first cam 3025 and a second cam 3026 to engage the first jaw and the second jaw, respectively, during the firing stroke to apply a closure force to the end effector to maintain the second jaw in the closed position. In accordance with the present disclosure, a power source 3041, 3055 may power the firing motor.

The surgical system includes a control system 3033 or controller 3051, as examples, to actuate the closure motor and firing motor to drive the closure member and the firing driver, respectively, through their respective strokes. The surgical system includes a voltage sensor 3039, to sense a voltage potential of the power source.

During use, the closure motor and firing motor draw current and consume power from the power source to drive the closure member and firing driver, respectively, through their respective strokes. As the motor draws current and consume power from the power source, the voltage potential of the power source is loaded and the voltage drops, or sags, causing the power output of the motors to drop. It is desirable to minimize power source voltage drop over the closure stroke/firing stroke of the closure member/firing driver, respectively, to increase the power output of the power source to the respective motors over their respective strokes. It is desirable to maximize the short inconsistent current draw from the power source by the motor during use thereof.

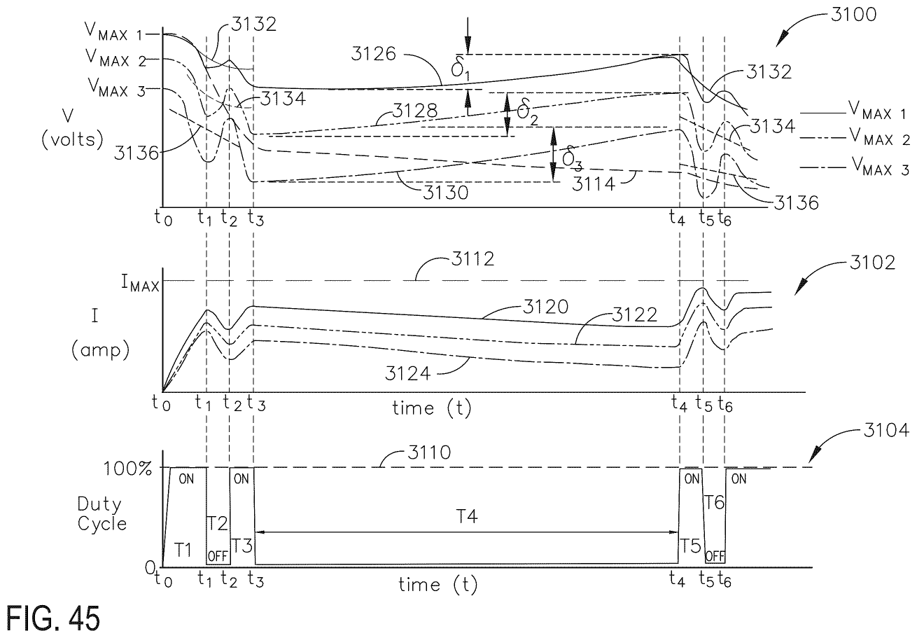

Voltage drop can be minimized by way of pulse width modulation and micro-recoveries during each “off” period of the pulses. Referring now to FIG. 45, graphs 3100, 3102, 3104 illustrate exemplary firing strokes of a firing driver through a staple cartridge. Graph 3100 illustrates the voltage potentials of power sources over time. Graph 3102 illustrates the currents sourced by the respective power sources over time. Graph 3104 illustrates the duty cycle of the respective power sources over time.

In accordance with the present disclosure, the control system may implement a firing algorithm, which may cause a firing motor 3056, to draw current from a power source having a maximum (or peak) voltage potential V_{MAX1} and drive a firing driver 3024 through a firing stroke. The algorithm implements a duty cycle 3110 of 100% (e.g., where the motor is held in an “on” state), as shown in graph 3104. Based on the algorithm, the current 3112 drawn by the motor from the power source is held constant, or substantially constant, at IMAX and the voltage potential 3114 of the power source drops from the maximum voltage potential V_{MAX1} to a minimum voltage potential at the end of the firing stroke.

In accordance with the present disclosure, the control system may implement an adaptive firing algorithm that causes the firing motor to drive the firing driver with adaptive pulse width modulation to diminish power source voltage drop over the firing stroke. Further, in accordance with the present disclosure, graphs 3100, 3102, 3104 may illustrate three exemplary firing strokes of a firing driver using the adaptive firing algorithm using three different power sources—a first power source having a maximum voltage potential of V_{MAX1}, a second power source having a voltage potential of V_{MAX2}, and a third power source having a voltage potential of V_{MAX3}. The first power source includes cells that each have a voltage potential of V_{cell1} that collectively form the first power source. The second power source includes cells that each have a voltage potential of V_{cell2} that collectively form the second power source. The third power source includes cells that each have a voltage potential of V_{cell3} that collectively form the third power source.

As shown in graphs 3100, 3102, 3104, at to, the control system implements the adaptive firing algorithms, which causes the firing motor to transition from an “off” state, in which the motor does not drive, or ceases to drive, the firing driver to prevent it from moving toward the fired position, to an “on” state, in which the motor drives the firing driver toward the fired position, for a first period T1. During the first period T1, the current 3120 drawn by the motor from the first power source increases from 0 to a first maximum current I_{MAX1} and the voltage potential 3126 applied to the motor from the first power source drops from the maximum voltage potential V_{MAX1} to a first lower voltage potential. Similarly, the current 3122 drawn by the motor from the second power source increases from 0 to a first max current I_{MAX2} and the voltage potential 3128 applied to the motor from the second power source drops from the maximum voltage potential V_{MAX2} to a first lower voltage potential. Similarly, the current 3124 applied to the motor from the third power source increases from 0 to a first max current I_{MAX3} and the voltage potential 3130 applied to the motor from the third power source drops from the maximum voltage potential V_{MAX3} to a first lower voltage potential. In accordance with the present disclosure, the control system can detect, or measure, the currents drawn by the motors and voltages applied to the motors using a current sensor and a voltage sensor, respectively. It will be appreciated that the motor can drive the firing driver in either a forward direction (proximal to distal) or a backward direction (distal to proximal).

In accordance with the present disclosure, T1 may be a predetermined period that may be stored in a memory 10035, and may be retrievable by the control system. Further, in accordance with the present disclosure, T1 may be based on the implemented duty cycle. Alternatively, or additionally, T1 may be a variable period. Alternatively, or additionally, T1 may be based on a rate at which the voltage potential drops from the maximum voltage potential Alternatively, or additionally, T1 may be based on the maximum voltage potential dropping a predetermined amount. Alternatively, or additionally, T1 may be based on the maximum voltage potential dropping to a predetermined lower voltage potential.

At time t₁, the algorithm automatically causes the motor to transition to the “off” state for a second period T2, during which time the current 3122, 3122, 3124 drawn by the motor from the power sources drops from the first max current I_{MAX1} to a first lower current and the voltage potential 3126, 3128, 3130 of the power sources recovers from the first lower voltage potential to a first recovered voltage potential that is less than the maximum voltage potentials of the respective power sources.

In accordance with the present disclosure, T2 may be a predetermined period that may be stored in a memory 10035, and may be retrievable by the control system. Alternatively, or additionally, T2 may be based on the implemented duty cycle. Alternatively, or additionally, T2 may be a variable period. Alternatively, or additionally, T2 may be based on T1. Alternatively, or additionally, T2 may be based on a magnitude of the voltage potential drop over T1. Alternatively, or additionally, T2 may be based on a rate at which the voltage potential dropped over T1. Alternatively, or additionally, T2 may be based on the time required for the power source to recover a threshold amount of voltage potential from the first dropped voltage potential. Alternatively, or additionally, T2 may be based on a rate at which the voltage potential recovers from the first dropped voltage potential. In accordance with the present disclosure, T2 may be different than T1. Alternatively, or additionally, T2 may be the same as T1

At time t₂, the algorithm automatically causes the motor to transition to the “on” state for a third period T3, during which time the current 3122, 3122, 3124 drawn by the motor from the power sources increases from the first lower current to a second maximum current that is greater than the first maximum current I_{MAX1} and the voltage potential 3126, 3128, 3130 applied to the motor from the power sources drops from the first recovered voltage potential to a second lower voltage potential that is less than the first lower voltage potential.

In accordance with the present disclosure, T3 may be a period stored in a memory 10035, and may be retrievable by the control system. Alternatively, or additionally, T3 may be based on the implemented duty cycle. In accordance with the present disclosure, T3 may be a variable period. Alternatively, or additionally, T3 may be based on T1 or T2, or a combination thereof. Alternatively, or additionally, T3 may be based on a rate at which the voltage potential recovered during T2. Alternatively, or additionally, T3 may be based on a magnitude of the voltage potential recovered during T2. Alternatively, or additionally, T3 may be based on a rate at which the voltage drops from the first recovered voltage potential. Alternatively, or additionally, T3 may be based on the voltage potential dropping a predetermined amount from the first recovered voltage potential. Alternatively, or additionally, T3 may be based on the voltage potential dropping to a predetermined voltage potential from the first recovered voltage potential. Alternatively, or additionally, T3 may be different than T1 and/or T2. Alternatively, or additionally, T3 may be the same as T1 and/or T3.

At time t₃, the algorithm automatically causes the motor to transition to the “off” state for a fourth period T4, during which time the current 3122, 3122, 3124 drawn by the motor from the power sources drops from the second maximum current I_{MAX2} to a second lower current that is less than the first lower current and the voltage potential 3126, 3128, 3130 of the power sources recovers from the second lower voltage potential to a second recovered voltage potential that is less than the first recovered voltage potential.

In accordance with the present disclosure, T4 may be a predetermined period that may be stored in a memory 10035, and may be retrievable by the control system. Alternatively, or additionally, T4 may be a variable period. Alternatively, or additionally, T4 may be based on T1, T2, or T3, or any combination thereof. Alternatively, or additionally, T4 may be based on a magnitude of the voltage potential drop over T1 or T3, or a combination thereof. Alternatively, or additionally, T4 may be based on a rate at which the voltage potential dropped over T1 or T3, or a combination thereof. Alternatively, or additionally, T4 may be based on the time required for the power source to recover a threshold amount of voltage potential from the second dropped voltage potential. Alternatively, or additionally, T4 may be based on a rate at which the voltage potential recovers from the second dropped voltage potential or a rate at which the voltage potential recovered from the first dropped voltage potential, or a combination thereof. Alternatively, or additionally, T4 may be different than T1, T2, and/or T3. Alternatively, or additionally, T4 may be the same as T1, T2 and/or T3. Alternatively, or additionally, T4 may be about 10 to 50 times larger than T2. As shown in graph 3100, during the fourth period T4, the motor is maintained in an “off” state for a period that allows the power sources to recover a δ amount. Specifically, the motor remains in the “off” state for the fourth period T4 such that the first power source recovers a first amount δ₁, the second power source recovers a second amount δ₂, and the third power source recovers a third amount δ₃.

At time t₄, the algorithm automatically causes the motor to transition to the “on” state for a fifth period T5, during which time the current 3122, 3122, 3124 drawn by the motor from the power sources increases from the second lower current to a third maximum current I_{MAX3} and the voltage potential 3126, 3128, 3130 applied to the motor from the power sources drops from the second recovered voltage potential to a third lower voltage potential.

In accordance with the present disclosure, T5 may be a predetermined period that is stored in a memory 10035, and is retrievable by the control system. Further, in accordance with the present disclosure, T5 may be based on the implemented duty cycle. Alternatively, or additionally, T5 may be a variable period. Additionally, in accordance with the present disclosure, T5 may be based on T1 T2, T3, or T4, or any combination thereof. Alternatively, or additionally, T5 may be based on a rate at which the voltage potential recovered during T2 or T4, or a combination thereof. Alternatively, or additionally, T5 may be based on a magnitude of the voltage potential recovered during T2 or T4, or a combination thereof. Alternatively, or additionally, T5 may be based on a rate at which the voltage drops from the second recovered voltage potential or a rate at which the first recovered voltage potential dropped, or a combination thereof. Alternatively, or additionally, T5 may be based on the voltage potential dropping a predetermined amount from the second recovered voltage potential. Alternatively, or additionally, T5 may be based on the voltage potential dropping to a predetermined voltage potential from the second recovered voltage potential. Alternatively, or additionally, T5 may be different than T1, T2, T3 and/or T4. Alternatively, or additionally, T5 may be the same as T1, T2, T3 and/or T4.

At time t₅, the algorithm automatically causes the motor to transition to the off state for a sixth period T6, during which time the current 3122, 3122, 3124 applied to the motor from the power sources drops from the third maximum current I_{MAX3} to a third lower current and the voltage potential 3126, 3128, 3130 of the power sources recovers from the third lower voltage potential to a third recovered voltage potential.

In accordance with the present disclosure, T6 may be a predetermined period that may be stored in a memory 10035, and may be retrievable by the control system. Alternatively, or additionally, T6 may be based on the implemented duty cycle. Alternatively, or additionally, T6 may be a variable period. Alternatively, or additionally, T6 may be based on T1, T2, or T3, T4, or T5, or any combination thereof. Alternatively, or additionally, T6 may be based on a magnitude of the voltage potential drop over T1, T3, or T5, or any combination thereof. Alternatively, or additionally, T6 may be based on a rate at which the voltage potential dropped over T1, T3, or T5, or any combination thereof. Alternatively, or additionally, T6 may be based on the time required for the power source to recover a threshold amount of voltage potential from the third dropped voltage potential. Alternatively, or additionally, T6 may be based on a rate at which the voltage potential recovers from the third lower voltage potential, a rate at which the voltage potential recovered from the first lower voltage potential, or a rate at which the voltage potential recovered from the second lower voltage potential, or any combination thereof. In accordance with the present disclosure, T6 may be different than T1, T2, T3, T4 and/or T5. Alternatively, or additionally, T6 is the same as T1, T2, T3, T4, and/or T5.

At time t₆, the algorithm automatically causes the motor to transition to the on state for a seventh period T7, during which time the current 3122, 3122, 3124 applied to the motor from the power sources increases from the third lower current and the voltage potential 3126, 3128, 3130 applied to the motor from the power source drops from the third recovered voltage potential. During the seventh period t₇, the firing driver reaches the distal fired position of the firing stroke and the firing algorithm ceases.

As shown, the algorithm is adaptive and reacts to changes in the voltage potential over the firing stroke of the firing driver. Based on the adaptive nature of the algorithm and the “micro-recoveries” of the voltage potential during the firing stroke, the overall macro-recovery curves 3132, 3134, 3136 of the voltage potentials over time are substantially higher compared to when the motor is left “on” (duty cycle of 100%) for the entire firing stroke, or the firing algorithm uses a constant pulse width modulation algorithm. In addition, the algorithm implemented by the control system implements a variable length pulse width modulation that has variable on/off times based the voltage drops and recovers over the firing stroke.

The first power source, which has a first maximum voltage potential V_{MAX1}, experiences less modulation during the algorithm than the second power source, which has a second maximum voltage potential V_{MAX2} less than the first maximum voltage potential V_{MAX1}. Similarly, the second power source, which has a second maximum voltage potential V_{MAX2}, experiences less modulation during the algorithm than the third power source, which has a third maximum voltage potential V_{MAX3} less than the second maximum voltage potential V_{MAX2}. In accordance with the present disclosure, referring to graph 3100, during the second recovery period T4, the first power source recovers δ1, the second power source recovers δ2 which is more than δ1, and the third power source recovers δ3 which is more than δ1 and δ3.

In accordance with the present disclosure, utilizing pulse width modulation can lead to a retardation of heat creation that would result in a locked rotor condition. Further, in accordance with the present disclosure, a larger powered accumulator of a lower ultimate pulse width modulation limit may be utilized to momentarily unstick a locked motor. The motor may reverse direction to result in backward motion of the firing driver and then may reverse direction again to restart forward motion of the firing driver to get through a portion of tissue where mechanical work needed. Alternatively, or additionally, the motor can reverse direction to result in backward motion of the firing driver and then reverse direction again to re-build up dynamic inertia of the firing driver.

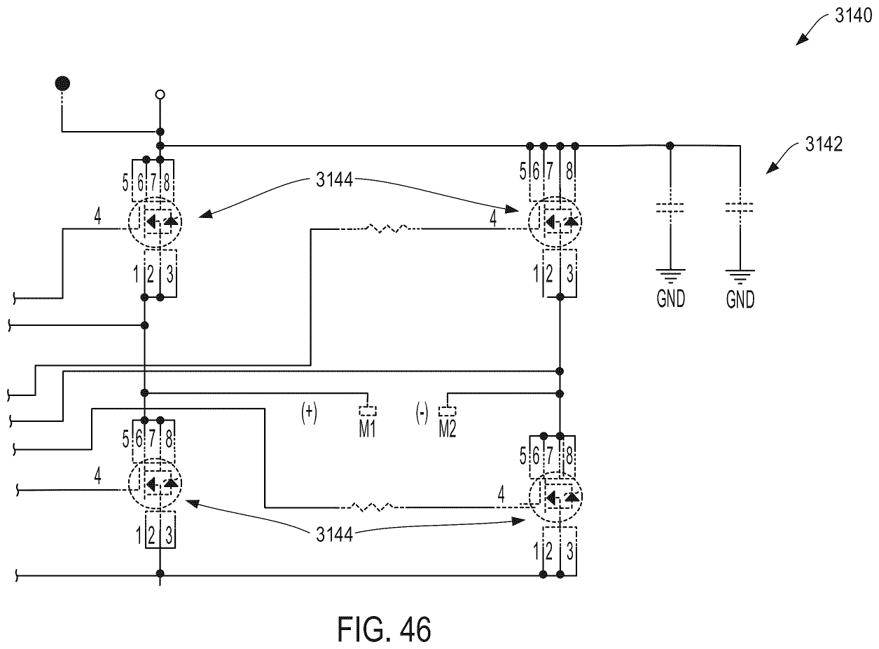

In accordance with the present disclosure, voltage drop can be minimized by accumulating power within the system. Additionally, in accordance with the present disclosure, accumulation of power within the system can be accomplished using a circuit 3140, illustrated in FIG. 46, that includes capacitors 3142 is series with field effect transistors 3144 (FETs). Utilizing the circuit 3140 with capacitors in series with FETs 3144, adds power to the system when the voltage potential of the power source begins to drop. The capacitors may provide a short term power boost, such as 1-5% of the total drive, for handling overload force conditions. Additionally, in accordance with the present disclosure, the capacitors may help during in-rush or small calcified areas of the tissue that cause spikes in the force required to fire the firing driver. It should be understood that the circuit 3140 is just one illustrative embodiment with capacitors in series with FETs 3144 and that various other circuits are contemplated by the present disclosure.

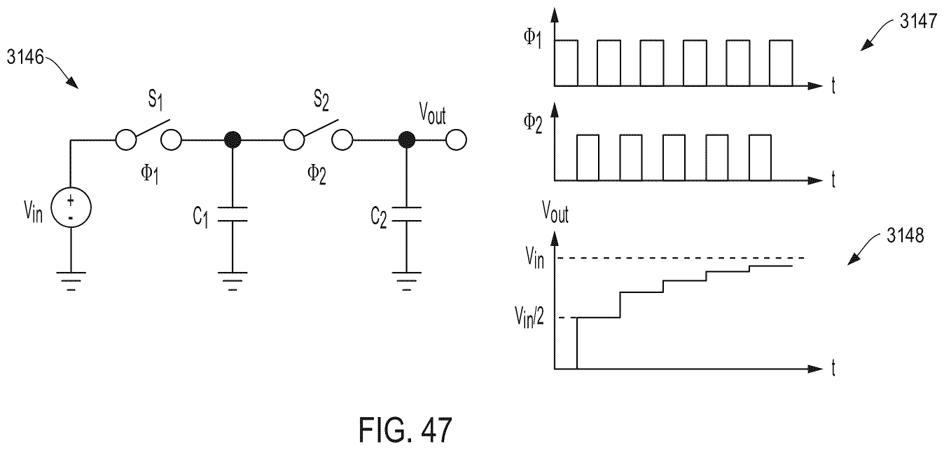

In accordance with the present disclosure, accumulation of power within the system may be accomplished by using a switching capacitor circuit 3146, illustrated in FIG. 47, which is a discrete-time circuit that exploits the charge transfer in and out of a capacitor as controlled by switches. The switching activity 3147 is controlled by non-overlapping clocks such that the charge transfer in and out is well defined and deterministic. The switching capacitor circuit 3146 provides a stepwise voltage output 3148 to the motor to provide an energy boost during the firing stroke.

In accordance with the present disclosure, accumulation of power within the system may be accomplished using capacitors plus pulse width modulation signals that are additive. A duty cycle of the motor may be selected such that the capacitors are re-charged after each drain, which reduces, or diminishes, power source voltage drop. Additionally, or alternatively, the duty cycle may be selected between 30-50%. A selected balance between pulse withdrawn activations and the capacitor sizing leads to a reduction, or elimination, or voltage sag from the power source.

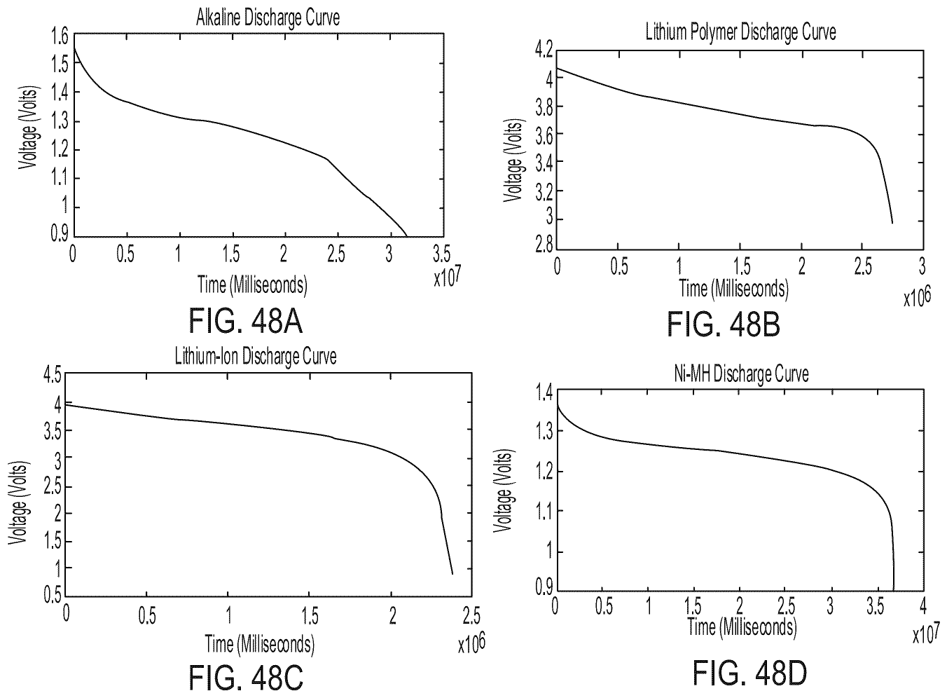

In accordance with the present disclosure, accumulation of power within the system may be based on the chemistry of the system. Additionally, or alternatively, accumulation of power within the system may be based on the duty cycle of the power source, the chemistry of the power source, or the power source chemistry recovery, or a combination thereof. For example, a material for a power source may be selected based on the observed power source voltage drop under load over time. FIGS. 48A-48D illustrate loaded power source voltage drop over time of several power sources utilizing different battery materials. FIG. 48A illustrates a discharge curve over time for a power source utilizing alkaline battery cells that includes a maximum voltage potential of ˜1.6V. FIG. 48B illustrates a discharge curve over time for a power source utilizing lithium polymer battery cells that includes a maximum voltage potential of ˜4.1V. FIG. 48C illustrates a discharge curve over time for a power source utilizing lithium-ion battery cells that includes a maximum voltage potential of ˜4V. FIG. 48D illustrates a discharge curve over time for a power source utilizing nickel-metal hydride (Ni-MH) battery cells that includes a maximum voltage potential of ˜1.35V. Based on the observed voltage drops over time, a user can select an appropriate power source for a desired surgical outcome.

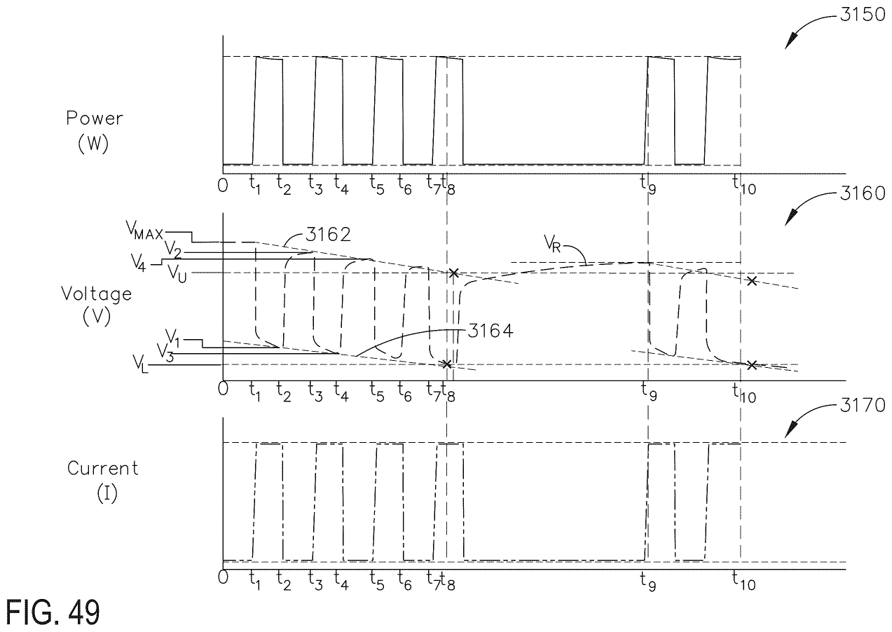

In accordance with the present disclosure, accumulation of power within the system may be based on motor power consumption, chemistry of the power source, or recovery of the power source, or a combination thereof. The control system can monitor the voltage potential of the power source over the course of a firing stroke in order to determine if the firing stroke should be paused to allow the power source to recover. Graphs 3150, 3160, 3170 shown in FIG. 49 show an exemplary firing stroke of a firing driver through a staple cartridge. Graph 3150 illustrates the power output from a power source over time. The power source may comprise a CR123A primary lithium battery. Graph 3160 illustrates the voltage potential of the power source over time. Graph 3170 illustrates the current output of the power source over time. As shown in graph 3160, the power source has a maximum voltage potential of V_{MAX}.

Based on the firing stroke power source, a control system comprising a controller 3033, 3051, sets a power source upper threshold V_{U}, a power source lower threshold V_{L}, and a power source recovery threshold V_{R}. In accordance with the present disclosure, the control system may interrogate the power source to determine a type of the power source. Based on the determination, the control system retrieves the power source upper threshold V_{U}, the power source lower threshold V_{L}, and the power source recovery threshold V_{R} from a memory. Alternatively, the power source upper threshold V_{U}, the power source lower threshold V_{L}, and the power source recovery threshold V_{R} are user defined. Alternatively, the power source upper threshold V_{U}, the power source lower threshold V_{L}, and the power source recovery threshold V_{R} are based on the maximum voltage potential of the power source. Alternatively, the power source upper threshold V_{U}, the power source lower threshold V_{L}, and the power source recovery threshold V_{R} are a predefined percentage of the maximum voltage potential of the power source. Alternatively, the power source upper threshold V_{U}, the power source lower threshold V_{L}, and the power source recovery threshold V_{R} are the same regardless of the power source used by the motor.

At time t₁, the control system initiates an adaptive firing algorithm based on a user providing an input to the control system. In accordance with the present disclosure, the adaptive firing algorithm may be similar to other adaptive firing algorithms discussed elsewhere herein. The adaptive firing algorithm may be stored in a memory 3053, and executable by a processor 3052. In accordance with the present disclosure, the user may provide the input to the control system via an input interface at display 3068. Based on the initiation of the adaptive firing algorithm, the adaptive firing algorithm applies a first duty cycle to the motor.

Based on the initiation of the adaptive firing algorithm, at t₁, the motor is transitioned to an on state for a first period according to the first duty cycle, causing the motor to draw power from the power source to drive a firing driver, such as firing driver 3024, toward its fired position. During the first period (from t₁ to t₂), the power source applies a maximum current I_{MAX} to the motor and the voltage potential of the power source drops from V_{Max} to a first lower voltage potential V₁. In accordance with the present disclosure, the control system may detect, or measure, the applied currents and voltages using a current sensor and a voltage sensor, respectively.

At time t₂, the control system detects, via the voltage sensor, that the voltage potential of the power source has dropped to a first dropped voltage potential V₁. In accordance with the present disclosure, the control system may store the first dropped voltage potential V₁ in the memory for subsequent evaluations. The control system compares the first lower voltage potential V₁ to the power source lower threshold V_{L} to determine if the first lower voltage potential V₁ is above or below the power source lower threshold V_{L}. Based on the control system determining that the first lower voltage potential V₁ is above the power source lower threshold V_{L}, the firing algorithm maintains the first duty cycle. Based on the control system determining that the first lower voltage potential V₁ has reached or dropped below the power source lower threshold V_{L}, the control system adjusts the firing algorithm. As shown in graph 3160, since the first dropped voltage potential V₁ is determined to be above the power source lower threshold V_{L}, the motor is transitioned to an “off” state for a second period according to the first duty cycle. During the second period (from t₂ to t₃), the voltage potential of the power source recovers to a first recovered voltage potential V₂.

At time t₃, the control system detects, via the voltage sensor, that the voltage potential of the power source has recovered to a first recovered voltage potential V₂. In accordance with the present disclosure, the control system may store the first recovered voltage potential V₂ in the memory for subsequent evaluations. The control system compares the first recovered voltage potential V₂ to the power source upper threshold V_{U} to determine if the first recovered voltage potential V₂ is above or below the power source upper threshold V_{U}. Based on the control system determining that the first recovered voltage potential V₂ is above the power source upper threshold V_{U}, the firing algorithm maintains the first duty cycle. Based on the control system determining that the first recovered voltage potential V₂ has reached or dropped below the power source upper threshold V_{U}, the control system adjusts the firing algorithm, as will be discussed in more detail below. As shown in graph 3160, since the first recovered voltage potential V₂ is determined to be above the power source upper threshold V_{U}, the motor is transitioned to the on state for a third period according to the first duty cycle. During the third period (from t₃ to t₄), the power source supplies the maximum current I_{MAX} to the motor and the voltage potential of the power source drops to a second dropped voltage potential V₃.

In accordance with the present disclosure, the control system may evaluate the two data points (e.g., the maximum voltage potential V_{Max} and the first recovered voltage potential V₂) and may project 3162 an anticipated recovered voltage potential over time utilizing the first duty cycle. Based on the projected voltage drop over time, the control system predicts a time that the recovered voltage potential of the power source is expected to fail to reach the power source upper threshold V_{U} prior to a later transition of the motor to an “on” state. In accordance with the present disclosure, the surgical system may comprise a display 3068, and the control system may display the predicted time on the display.

In accordance with the present disclosure, the control system may adjust the duty cycle of the firing algorithm based on the prediction. Additionally, based on the prediction, the control system may adjust the algorithm to change the duty cycle of the motor so as to maintain the recovered voltage potential above the power source recovery threshold V_{U} prior to subsequent transitions of the motor to the on state for the remainder of the firing stroke. In accordance with the present disclosure, based on the prediction, the control system may adjust the duty cycle to increase a length of the “off” pulses of the duty cycle. Additionally, or alternatively, based on the prediction, the control system may adjust the duty cycle to increase a length of the “on” pulses of the duty cycle. Further, in accordance with the present disclosure, based on the prediction, the control system may adjust the duty cycle to increase a length of the “on” pulses and the “off” pulses of the duty cycle.

At time t₄, the control system detects, via the voltage sensor, the drop in voltage potential of the power source to a second lower voltage potential V₃. The control system can store the second lower voltage potential V₃ in the memory for subsequent evaluations. The control system compares the second lower voltage potential V₃ to the power source lower threshold V_{L} to determine if the second lower voltage potential V₃ is above the power source lower threshold V_{L}. Based on the control system determining that second lower voltage potential V₃ is above the power source lower threshold V_{L}, the firing algorithm maintains the first duty cycle. Based on the control system determining that second lower voltage potential V₃ has reached or dropped below the power source lower threshold V_{L}, the control system adjusts the firing algorithm. Since the second lower voltage potential V₃ is determined to be above the power source lower threshold V_{L}, the motor is transitioned to an “off” state for a fourth period according to the first duty cycle as shown in graph 3160. During the fourth period (from t₄ to t₅), the voltage potential of the power source recovers to a second recovered voltage potential V₄.

In accordance with the present disclosure, the control system may evaluate the two data points, the first lower voltage potential V₁ and the second lower voltage potential V₃, and may project 3164 an anticipated voltage potential drop over time utilizing the first duty cycle. Based on the projected voltage drop over time, the control system predicts a time that the voltage potential of the power source is expected to drop below the power source lower threshold V_{L} during a subsequent “on” state of the motor. In accordance with the present disclosure, the control system may display the predicted time on the display. Additionally, or alternatively, the control system may display both predicted times (predicted time that power source will fail to recover to the power source upper threshold V_{U} and predicted time that power source will drop below the power source lower threshold V_{L}) to inform a user as to which event is expected to occur first.

In accordance with the present disclosure, the control system may adjust the duty cycle of the firing algorithm based on the prediction. Additionally, in accordance with the present disclosure, based on the prediction, the control system may adjust the algorithm to control the duty cycle of the motor to maintain the lower voltage potential above the power source lower threshold V_{L} for the remainder of the firing stroke. Further, in accordance with the present disclosure, based on the prediction, the control system may adjust the duty cycle to increase a length of the “off” pulses of the duty cycle. Additionally, in accordance with the present disclosure, based on the prediction, the control system may adjust the duty cycle to increase a length of the “on” pulses of the duty cycle. Further, in accordance with the present disclosure, based on the prediction, the control system may adjust the duty cycle to increase a length of the “on” pulses and the “off” pulses of the duty cycle.

As shown in graph 3160, from time t₄ to t₆, the control system maintains the first duty cycle to transition the motor to the “off” state (t₄ to t₅), the “on” state (t₅ to t₆), and the “off” state (t₆ and t₇). At each time point, the control system compares the detected voltage potential to a respective threshold (power source upper threshold V_{U} and power source lower threshold V_{L}) to determine if the first duty cycle should be maintained. At each time point, the control system stores the detected voltage potential in the memory and uses the data points to update the respective projections 3162, 3164 and predictions.

As shown in graph 3160, at time t₇, the motor is transitioned to the “on” state for a period according to the first duty cycle, which causes the voltage potential to drop to a third lower voltage potential. During the “on” state, as also seen in graph 3160, the voltage potential of the power source drops below the power source lower threshold V_{L}. In accordance with the present disclosure, despite dropping below the power source lower threshold V_{L}, the firing algorithm can maintain the motor in the on state according to the first duty cycle to allow the motor to finish the “on” pulse. Further, in accordance with the present disclosure, upon detecting the voltage potential dropping below the power source lower threshold V_{L}, the control system can transition the motor to the “off” state, cutting short the “on” pulse, to allow the power source to recover for a recovery period.

In the firing algorithm, the control system compares the third lower voltage potential to the power source lower threshold V_{L} to determine if the third lower voltage potential is above or below the power source lower threshold V_{L}. Since the third lower voltage potential is determined by the control circuit to have dropped below the power source lower threshold V_{L}, the control system transitions the motor to the “off” state and maintains the motor in the “off” state for a recovery period. In accordance with the present disclosure, the recovery period can correspond to the time required for the voltage potential of the power source to recover from the third lower voltage potential to the power source recovery threshold V_{R}. Further, in accordance with the present disclosure, the recovery period may be longer than the “off” pulse for the first duty cycle. As shown in graph 3160, at time t₉, based on the control system detecting that the voltage potential of the power source has reached the power source recovery threshold V_{R}, the control system transitions the motor to the on state and resumes applying the firing algorithm with the first duty cycle. In accordance with the present disclosure, based on the control system detecting that the voltage potential of the power source has reached the power source recovery threshold V_{R}, the control system may transition the motor to the on state and may apply a second duty cycle that is different than the first duty cycle.

While FIG. 49 illustrates a scenario where the voltage potential drops below the power source lower threshold V_{L} during an on-pulse, it should be understood that the control system operates in a similar manner when the voltage potential fails to reach the power source upper threshold V_{U} during an off-pulse. In accordance with the present disclosure, based on the voltage potential failing to reach the power source upper threshold V_{U} during the off-pulse, the control system may maintain the motor in the “off” state for a recovery period to allow the voltage potential of the power source to reach the power source recovery threshold V_{R} prior to re-implementing the first duty cycle. Based on the control system detecting that the voltage potential of the power source has reached the power source recovery threshold V_{R}, the control system transitions the motor to the “on” state and resumes applying the firing algorithm with the first duty cycle. In accordance with the present disclosure, based on the control system detecting that the voltage potential of the power source has reached the power source recovery threshold V_{R}, the control system may transition the motor to the “on” state and applies a second duty cycle that is different than the first duty cycle.

After re-implementing the first duty cycle, the control system continues to compare the detected voltage potentials to the respective thresholds at each on and off pulse to determine if the first duty cycle should be maintained for the remainder of the firing stroke. As shown in graph 3160, at time t₁₀, the firing stroke of the firing driver ends before either thresholds are reached or dropped below, and therefore, no additional recovery periods are needed. However, it should be understood that, had a respective threshold been reached or dropped below, the control system would operate in a similar manner as described above, in which the motor is maintained in an off state for a recovery period to allow the voltage potential of the power source to recover to the power source recovery threshold V_{R}.

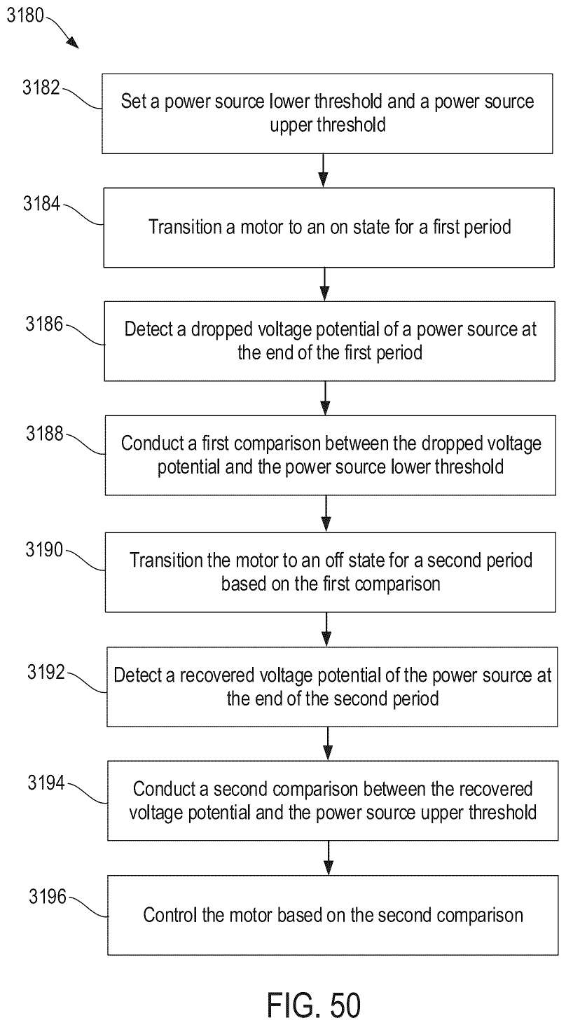

FIG. 50 shows a method 3180 for controlling a surgical system, according to the present disclosure. The method 3180 comprises setting 3182 a power source lower threshold and a power source upper threshold. In accordance with the present disclosure, a control system comprising a controller 3033, 3051, can set a power source lower threshold V_{L} and a power source upper threshold V_{U}. The method 3180 may include setting a power source recovery threshold, such as power source recovery threshold V_{R}. Further, in accordance with the present disclosure, the control system may set the thresholds by retrieving the thresholds from a memory.

The method 3180 comprises transitioning 3184 a motor to an “on” state for a first period. In accordance with the present disclosure, the control system may implement a first duty cycle which turns the motor on for a first period according to the duty cycle. The motor may comprise a firing motor 3056 that drives a firing driver 3024 toward a fired position to deploy staples 3023 from a staple cartridge 3008 when the motor is in an “on” state.

The method 3180 comprises detecting 3186 a dropped voltage potential of a power source at the end of the first period. The control system can interrogate a voltage sensor 3039 to determine the voltage potential of a power source 3041, 3055 that powers the motor. During the “on” state of the motor, the voltage potential of the power source drops from a maximum, or recovered, voltage potential to a lower voltage potential.

The method 3180 comprises conducting 3188 a first comparison between the lower “dropped” voltage potential and the power source lower threshold. In accordance with the present disclosure, the control system may compare the lower “dropped” voltage potential to the power source lower threshold to determine if the lower “dropped” voltage potential is above or below the power source lower threshold.

The method 3180 comprises transitioning 3190 the motor to an “off” state for a second period based on the first comparison. In accordance with the present disclosure, based on the lower “dropped” voltage potential being above the power source lower threshold, the control system may maintain the current duty cycle of the motor and allows the motor to remain “off” for a period according to the duty cycle. Additionally, in accordance with the present disclosure, based on the dropped voltage potential reaching or falling below the power source lower threshold, the control system may transition the motor to the “off” state for a recovery period, which may be longer than the current duty cycle time. The recovery period may correspond to the time required for the voltage potential of the power source to recover to the power source recovery threshold.

The method 3180 comprises detecting 3192 a recovered voltage potential of the power source at the end of the second period. In accordance with the present disclosure, once the second period has elapsed, the control system may interrogate the voltage sensor to determine the voltage potential of the power source. As discussed elsewhere herein, during the “off” state of the motor, the voltage potential of the motor rises, or recovers, from a lower “dropped” voltage potential.

The method 3180 comprises conducting 3194 a second comparison between the recovered voltage potential and the power source upper threshold. The control system can compare the recovered voltage potential to the power source upper threshold to determine if the lower “dropped” voltage potential has reached, or is below, the power source upper threshold.

The method 3180 comprises controlling 3196 the motor based on the second comparison. In accordance with the present disclosure, based on the recovered voltage potential reaching the power source upper threshold, the control system may allow the motor to maintain the current duty cycle of the motor, and thus, may turn the motor back to the “on” state after the second period has elapsed. Alternatively, based on the recovered voltage potential failing to reach the power source upper threshold, the control system may maintain the motor in the “off” state to allow for additional recovery of the motor. Additionally, in accordance with the present disclosure, the control system may maintain the motor in the “off”′ state after the second period for a recovery period, which may correspond to the time required for the voltage potential of the power source to recover to the power source recovery threshold.

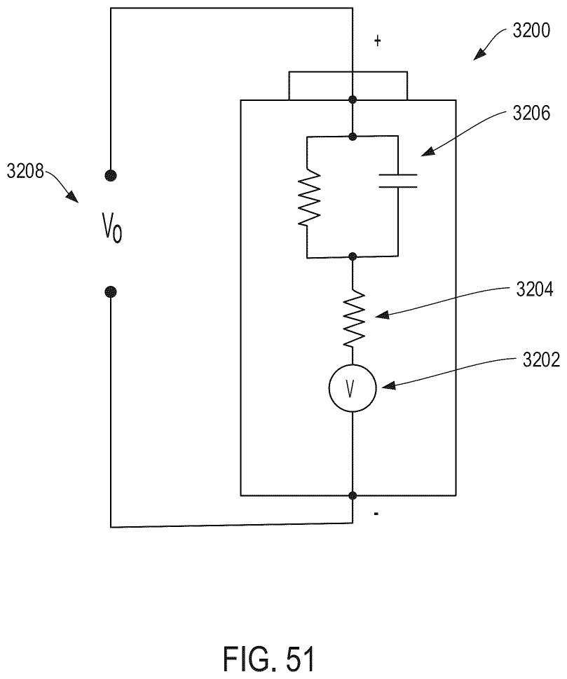

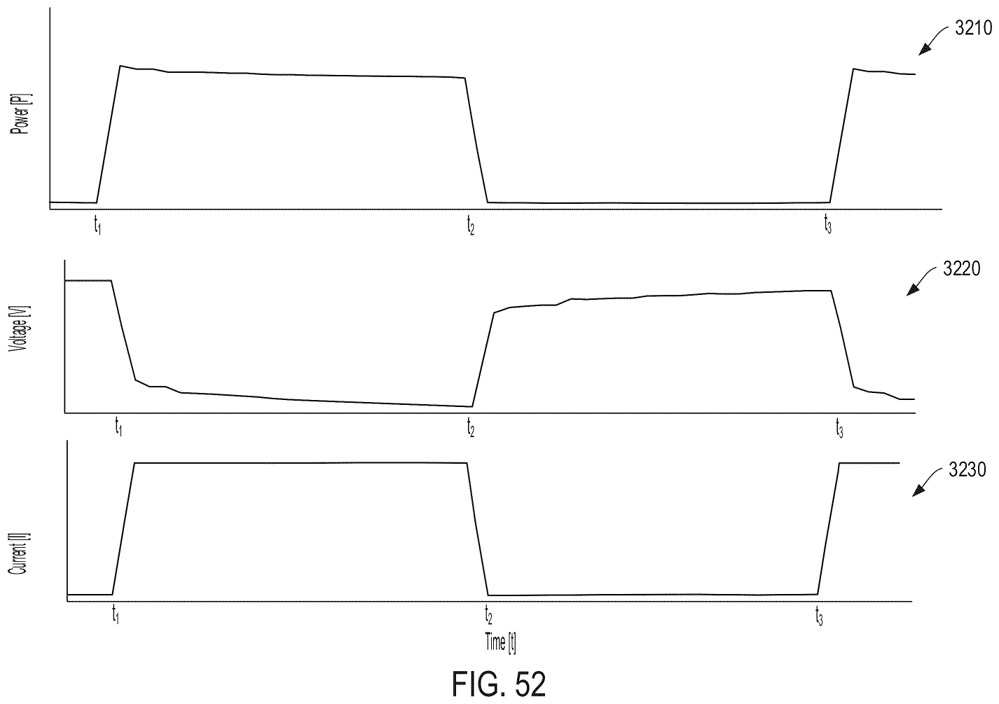

FIG. 51 shows a battery 3200, according to the present disclose. The battery 3200 comprises a circuit including a voltage source 3202 with a voltage potential of V_{Max}, a resistor 3204, a resistor-capacitor (RC) circuit 3206 in series with the voltage source 3202 and the resistor 3204, and a voltage output V₀ 3208 in series with the battery 3200. FIG. 52 illustrates graphs 3210, 3220, 3230 of an exemplary firing stroke of a firing driver where the battery 3200 provides power to a firing motor that drives the firing driver.

At time t₁, a control system comprising a controller 3033, 3051 applies a firing algorithm, which causes the firing motor to transition to an “on” state and draw current from the battery 3200 to drive the firing driver toward the distal position. From time t₁ to t₂, as shown in graphs 3210, 3220, 3230, the voltage output 3208 of the battery 3200 drops from Vₘₐₓ to V₁, the current drawn by the motor increases and is maintained at I_{Max}, and the power consumed by the motor increases to P_{Max} and drops to P₁. At time t₂, the firing algorithm transitions the motor to the “off” state, causing the motor to stop drawing current from the battery 3200. From time t₂ to t₃, based on the chemistry and the circuitry of the battery 3200, the voltage potential of the battery 3200 first sharply rises and then steadily rises to a first recovered voltage V_{R}.

In accordance with the present disclosure, the period from t₂ to t₃ may be a variable period. Additionally, or alternatively, the period from t₂ to t₃ may be a predetermined period. Additionally, or alternatively, the period may be based on an applied duty cycle to the motor. Additionally, or alternatively, the period from t₂ to t₃ may be based on a magnitude of the voltage potential drop from t₁ to t₂. Additionally, or alternatively, the period from t₂ to t₃ may be based on a rate at which the voltage potential dropped from t₁ to t₂. Additionally, or alternatively, the period from t₂ to t₃ may be based on the time required by the battery 3200 to recover a threshold amount of voltage potential from V₁. The time from t₂ to t₃ may be based on a rate at which the voltage potential recovers from V₁.

At time t₃, the firing algorithm transitions the firing motor to the “on” state again such that the motor draws current from the battery 3200 to resume driving the firing driver toward the fired position.

Voltage drop is minimized by accumulating power within the system. In accordance with the present disclosures, accumulation of power within the system may be accomplished by storing energy during off states of the power source. An “off” state may comprise an “off” pulse of a pulse width modulation signal. Further, additionally, or alternatively, an “off” state may comprise a state in between firing strokes of a firing driver between the conclusion of a first firing stroke and the start of a second firing stroke. In accordance with the present disclosure, energy may be stored in the system using capacitors. Energy stored within a capacitor is given by the classic equation U=0.5 (C) (V²), where U is the capacitor energy, C is the capacitance of the capacitor, and V is the voltage. Based on this equation, the same capacitor charged to a higher voltage will store exponentially more energy within a system.

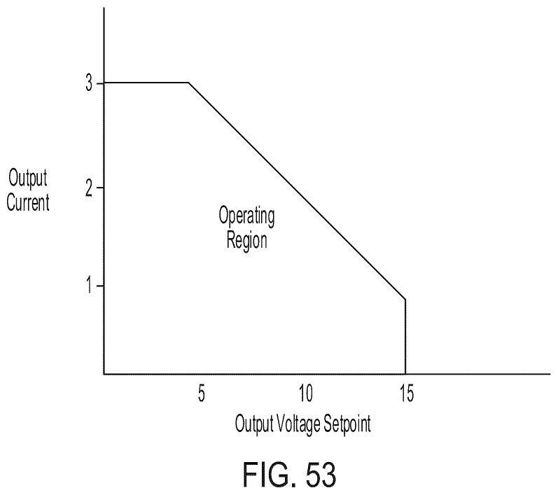

The control system can comprise a voltage convertor to control voltage that is applied to the motor from the power source, which reduces power source voltage drop. A constraint of many voltage converter methodologies is a limitation of load curves. Total output power is fixed for variable voltages, which impacts the current available for sourcing. FIG. 53 illustrates a load curve profile for a 15 W power source. Utilizing a voltage converter, a direct bearing on what the system can deliver is known depending on a set the input voltage.

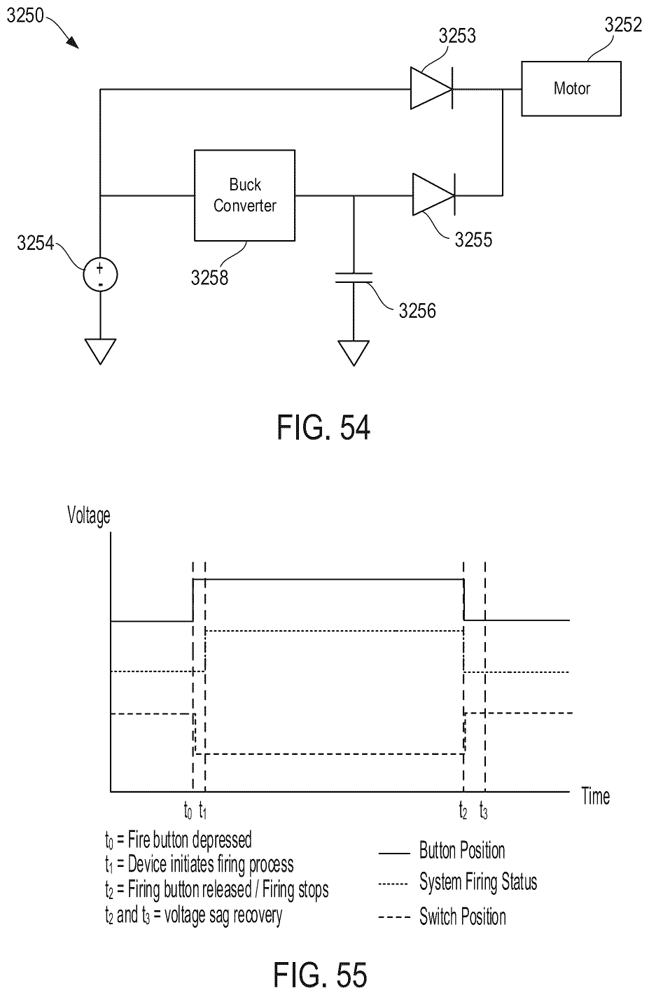

In accordance with the present disclosure, power can be accumulated in the system using a storage capacitor. The storage capacitor may be selectively connected to the motor using active or passive components (e.g., via Diode OR'ing) to allow for very low impact on inrush capabilities. Further, in accordance with the present disclosure, referring to FIG. 54, a circuit 3250 that includes a motor 3252, a power source 3254 that provides power to the motor 3252, and a diode 3253 between the power source 3254 and the motor 3252. The circuit 3250 includes a storage capacitor 3256 in electrical communication with the motor 3252, a buck converter 3258 that steps down voltage from the power source 3254 to the storage capacitor 3256, and a diode 3255 between the storage capacitor 3256 and the motor 3252.

In operation, during an “off” state of the motor 3252, current flows from the power source 3254 to the storage capacitor 3256 via the buck converter 3258 to charge the storage capacitor 3256. When the motor 3252 transitions to an “on” state to drive a firing driver, current flows from the storage capacitor 3256 and the power source 3254 to the motor 3252 to mitigate motor 3252 inrush current. After the initial transition to the “on” state, the current flows from the power source 3254 to the motor 3252 to drive the firing driver. When the motor 3252 is again transitioned to the “off” state, the current once again flows from the power source 3254 to the storage capacitor 3256 via the buck converter 3258 to charge the storage capacitor 3256.

In accordance with the present disclosure, the storage capacitor 3256 may have a much lower internal resistance when compared to the power source 3254. Accordingly, the voltage drop associated with the storage capacitor 3256 will be much smaller than the power source 3254 voltage drop. The storage capacitor 3256 will aid in keeping the voltage potential to the motor 3252 high, which provides the motor 3252 with more power to complete the firing stroke.

In accordance with the present disclosure, a variable output DC/DC converter can be placed in series with a switching circuit, such as conventional mechanical switch, or electronic switch such as a MOSFET, as examples. Based on the motor being in an “off” state, the DC/DC converter will change its output to a higher voltage to charge capacitors and store energy. A diode OR will drop the voltage to the appropriate level.

Referring now to FIG. 55, a graph 3260 is provided that illustrates button position of a surgical system, a switch position, and firing status of a motor over time. At time t₀, a user actuates a button of the surgical system at an input interface at display 3068. Based on the actuation, the switch transitions from an open state to a closed state to electrically couple a storage capacitor to the motor, which causes the capacitor to apply a voltage to the motor. At time t₀, an international delay is introduced, using the aforementioned switching circuit, which provides a slight delay before the motor draws current from the power to supply power to the motor and begin driving the firing driver. In accordance with the present disclosure, the slight delay may provide inrush current mitigation. After the intentional delay, at time t₁, the motor transitions to an “on” state to drive the motor. At time t₂, the power source ceases providing power to the motor to transition the motor to the “off” state, the switch transitions back to the “off”′ state, and the button returns to an unactuated state. Based on the changes, the power source recovers voltage potential and the capacitor is refilled in preparation for a subsequent firing cycle.

In accordance with the present disclosure, accumulation of power within the system can be accomplished by managing power accumulation available within the power source. For instance, due to motor inrush current, the current drawn by the motor from the power source at the beginning of any firing stroke will be substantial. This places a significant limitation on the energy storage capabilities for subsequent firings of the motor. Utilizing a starting consistent operational parameter allows the power source to “recover” and accumulate energy in between firing strokes to provide a more consistent firing outcome over multiple firing strokes of the firing driver. Accordingly, it is desirable that a user of the system be aware of the power accumulation of the power source prior to initiating a subsequent firing stroke in order to understand that the device may perform less than normal unless a sufficient amount of power has been recovered/re-accumulated.

In accordance with the present disclosure, a control system comprising controller 3033, 3051, can monitor an amount of energy accumulated in a power source 3055 after the conclusion of the firing strokes of a firing driver 3024. For instance, a user may provide a first firing input to the control system at an input interface of display 3068. Based on receiving the first firing input, the control system controls a firing motor 3056 to drive the firing driver through a first firing stroke from the proximal, unfired position to the distal, fired position.

Based on the conclusion of the first firing stroke, the control system interrogates a sensor to monitor an amount of power that is re-accumulating within the power source over time. In accordance with the present disclosure, the sensor may comprise a voltage sensor 3054 and the control system may monitor the voltage potential of the power source over time using the voltage sensor. The control system displays the monitored amount of power on a display 3068 to notify the user of the amount of power within the power source. In accordance with the present disclosure, the control system may display the maximum power of the power source on the display. Based on visual cues, a user can determine whether to proceed with a second firing stroke of the firing driver, or wait an additional period prior to the initiation of the second firing stroke to allow for additional power accumulation in the power source.

In accordance with the present disclosure, the control system may set a recovery threshold of the power source. The recovery threshold may correspond to a threshold amount of energy required of the power source to accomplish a subsequent firing stroke. Alternatively, the recovery threshold may correspond to a threshold amount of energy required to allow the firing driver to operate in the same manner as in the first firing stroke. Alternatively, the recovery threshold may be stored in a memory and retrievable by the control system. Alternatively, the recovery threshold may be user defined. Alternatively, the recovery threshold may be a predefined percentage of the maximum voltage potential of the power source. Alternatively, the recovery threshold may comprise a recovery voltage potential of the power source.

In accordance with the present disclosure, the control system may display the recovery threshold on the display to enable the user to visually compare the monitored amount of power of the power source to the recovery threshold. Based on the visual comparison, a user can choose to wait until the power has reached the recovery threshold until actuating a second firing stroke of the firing driver. The user also can choose to actuate the second firing stroke of the firing driver prior to the monitored power reaching the recovery threshold; however, the displayed power will indicate to the user that the system will perform less than normal as the power source was not allowed to re-accumulate to the recovery threshold.

In accordance with the present disclosure, the control system may include a firing lockout to prevent the user from performing another firing stroke until a sufficient amount of energy has been re-accumulated in the power source. Further, in accordance with the present disclosure, the control system may compare the monitored power to the recovery threshold based on a user providing a second firing input to the input interface. Based on the monitored power being less than the recovery threshold, the control system ignores the second firing input and prevents the motor from driving the firing driver through the firing stroke because a sufficient amount of energy has not been re-accumulated in the power source. The control system issues a notification on a display 3068 indicating that a sufficient amount of energy has not been re-accumulated and that additional time is required until the subsequent firing stroke can be initiated. Based on the monitored power reaching or exceeding the recovery threshold, the control system allows the firing stroke to commence and controls the motor to drive the firing driver through the subsequent firing stroke. In accordance with the present disclosure, the control system may display a notification on the display to inform the user that the firing system is available for actuation based on the monitored energy reaching the recovery threshold.

In accordance with the present disclosure, the control system may include a firing lockout to prevent the user from performing another firing stroke until a threshold period has elapsed from the conclusion of a previous firing stroke. The threshold period may be fixed. Further, in accordance with the present disclosure, the power source may comprise an RC circuit and the RC time constant dictates the threshold period. Based on the RC time constant, the control system prevents a user from initiating a subsequent firing stroke for a fixed period to guarantee that the capacitor has had sufficient time to recharge over the period equal to the RC time constant. The threshold period may be variable. Additionally, in accordance with the present disclosure, based on system characterization, and known RC time constant recharge rates, the firing lockout may lock out the user from actuating the firing system for a variable period based on a calculation of predicted energy storage in the power source. Based on the threshold period elapsing, the control system displays a notification on the display to inform the user that the firing system is available for actuation and allows the user to initiate the subsequent firing stroke.

The control system can monitor time, intensity, and/or duration of power draw from the power source during use thereof. Based on the monitored parameters, the control system controls when, and how much, subsequent power draws are allowed or enabled. Such control allows for electrolyte recovery in the power source, as well as allows for heat dissipation of the cells of the power source, which improves the output capacity for the next power usage of the power source, such as during a subsequent firing stroke of the firing driver.

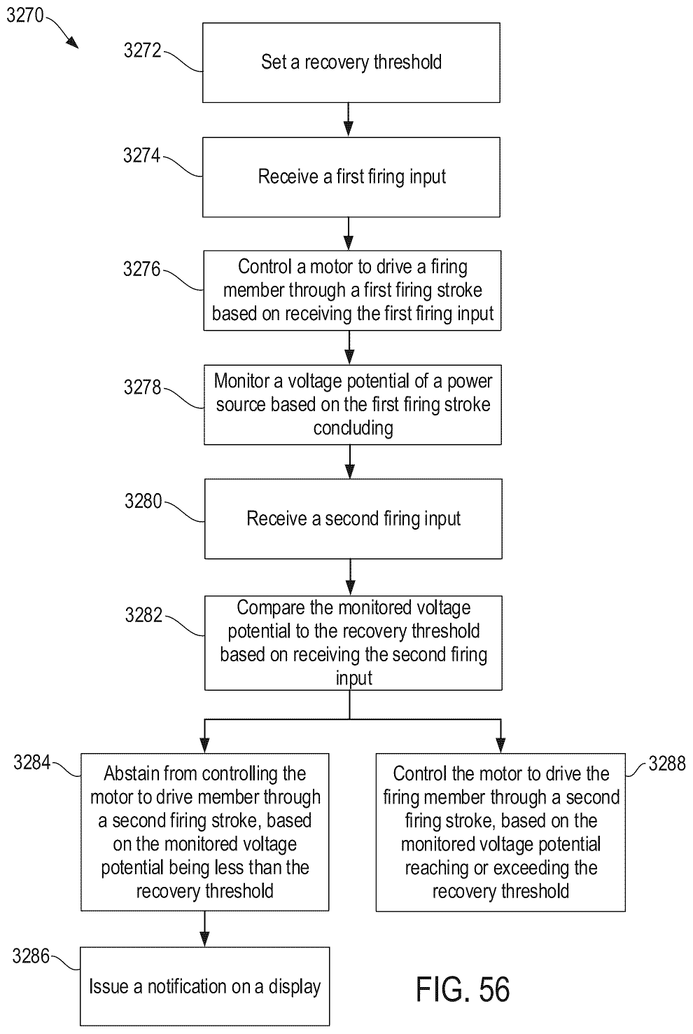

FIG. 56 illustrates a method 3270 for controlling a surgical system, according to the present disclosure. The method 3270 comprises setting 3272 a recovery threshold. In accordance with the present disclosure, a control system comprising a controller 3033, controller 3051 can set a recovery threshold, as described elsewhere herein, such as by retrieving the recovery threshold from a memory. The recovery threshold may comprise a voltage potential threshold associated with a power source 3055.

The method 3270 comprises receiving 3274 a first firing input. A user provides a first firing input to a control system comprising a controller 3033, 3051 at an input interface of display 3068 in order to perform a first firing stroke of the firing driver.

The method 3270 comprises controlling 3276 a motor to drive a firing driver through a first firing stroke based on receiving the first firing input. Based on receiving the first firing input, the control system can control a firing motor 3056 to drive a firing driver 3024 through a first firing stroke, from the proximal, unfired position to the distal, fired position.

The method 3270 comprises monitoring 3278 a power source voltage potential at the end of conclusion the first firing stroke. The motor draws current from a power source 3055 to drive the firing driver through the firing stroke and a voltage sensor 3039 senses the voltage potential of the power source over time. The control system interrogates the voltage sensor after the firing stroke has concluded to determine the dropped voltage potential of the power source and to monitor the voltage recovery of the power source over time. In accordance with the present disclosure, the method may comprise displaying the monitored voltage potential on a display 3068 to enable a user to visually track the voltage recovery of the power source over time.

The method 3270 comprises receiving 3280 a second firing input. In accordance with the present disclosure, a user may provide the second firing input to a control system comprising a controller 3033, 3051 at the input interface in order to perform a second firing stroke of the firing driver.

The method 3270 comprises comparing 3282 the monitored voltage potential to the recovery threshold based on receiving the second firing input. In accordance with the present disclosure, based on the user attempting to initiate a second firing stroke of the firing driver by providing a second firing input to the input interface, the control system may compare the monitored voltage potential to the recovery threshold in order to determine if the firing driver should be allowed to advance through a second firing stroke.

The method 3270 comprises abstaining 3284 from controlling the motor to drive the firing driver through a second firing stroke based on the monitored voltage potential being less than the recovery threshold. In accordance with the present disclosure, based on the control system determining that the monitored voltage has not yet reached the recovery threshold, the control system may ignore the second firing input and may abstain from advancing the firing driver through the second firing stroke with the motor. The method 3270 comprises issuing 3286 a notification on a display. In accordance with the present disclosure, the control system may transmit a signal to the display to display a notification that informs the user that the power source has not yet reached the recovery threshold, and therefore, the second firing stroke cannot be completed at this time.

The method 3270 comprises controlling 3288 the motor to drive the firing driver through a second firing stroke based on the monitored voltage potential reaching or exceeding the recovery threshold. In accordance with the present disclosure, based on the monitored voltage potential reaching or exceeding the recovery threshold, the control system may control the motor to drive the firing driver through the second firing stroke. The method may comprise issuing a notification on the display based on the monitored voltage potential reaching the recovery threshold. Accordingly, a user is notified that the power source has recovered a sufficient amount of energy and that a second firing stroke can now be completed.

In accordance with the present disclosure, the method 3270 optionally may further comprise monitoring an elapsed period based on the first firing stroke concluding, comparing the elapsed period to a recovery time period based on receiving the second firing input, and performing an action, such as abstaining from driving the motor or controlling the motor to drive the firing driver through a second firing stroke, based on the comparison.

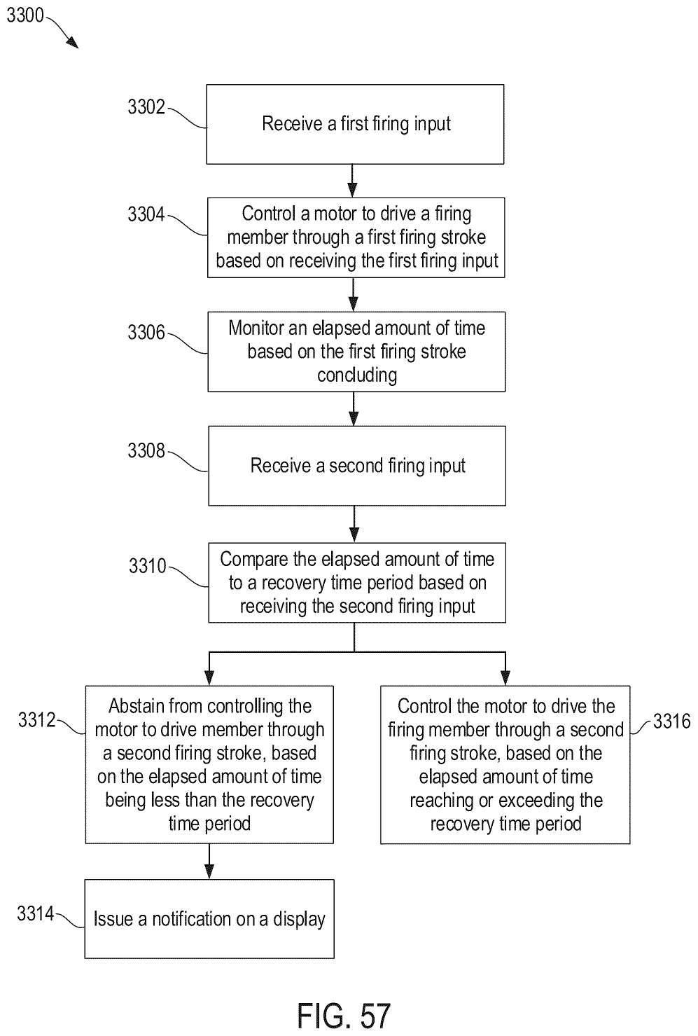

FIG. 57 shows a method 3300 for controlling a surgical system, according to the present disclosure. The method 3300 comprises receiving 3302 a first firing input. A user provides the first firing input to a control system comprising a controller 3033, 3051 at an input interface of display 3068 to initiate a first firing stroke of the firing driver.

The method 3300 comprises controlling 3304 a motor to drive a firing driver through a first firing stroke based receiving the first firing input. Based on receiving the first firing input, the control system can control a the firing motor 3056 to drive a firing driver 3024 through a first firing stroke from the proximal, unfired position to the distal, fired position.

The method 3300 comprises monitoring 3306 an elapsed period based on the first firing stroke concluding. In accordance with the present disclosure, based on the firing driver reaching the distal, fired position, the control system may initiate a timer, such as timer 3069, in order to measure an elapsed period from the conclusion of the firing stroke. As described elsewhere herein, after the conclusion of the firing stroke, a power source, such as power source 3055, that powers the motor begins to re-accumulate energy in preparation for a subsequent firing stroke.

The method 3300 comprises receiving 3308 a second firing input. In accordance with the present disclosure, a user can provide the second firing input to a control system, such as controller 3033 or controller 3051, at the input interface in order to perform a second firing stroke of the firing driver.

The method 3300 comprises comparing 3310 the elapsed period to a recovery time period based on receiving the second firing input. In accordance with the present disclosure, based on the user attempting to initiate a second firing stroke of the firing driver by providing the second firing input to the input interface, the control system may compare the elapsed period to the recovery time period in order to determine if the firing driver should be allowed to advance through a second firing stroke. By comparing the elapsed period to the recovery threshold, the control system determines if a sufficient amount of energy has been re-accumulated by the power source in order to complete the second firing stroke. In accordance with the present disclosure, the recovery period may be a predetermined period stored in a memory.

Alternatively, or additionally, the recovery period may be a predetermined period input by a user at the input interface. Alternatively, or additionally, the recovery period may be based on the RC time constant of the power source. Alternatively, or additionally, the recovery period may be a variable period. In accordance with the present disclosure, based on the known RC time constant recharge rate of the power source, the control system can predict the time required before a subsequent firing stroke can be completed and the recovery period comprises this predicted period. Further, in accordance with the present disclosure, the recovery period may be a variable period based on the recharge rate of the power source.

The method 3300 comprises abstaining 3312 from controlling the motor to drive the firing driver through a second firing stroke based on the elapsed period being less than the recovery time period. Based on the control system determining that the elapsed period has not yet reached the recovery time period, the control system can ignore the second firing input and abstains from advancing the firing driver through the second firing stroke with the motor. The method 3300 comprises issuing 3314 a notification on a display. In accordance with the present disclosure, the control system may transmit a signal to the display to display a notification informing the user that the recovery time period has not yet elapsed and therefore, the second firing stroke cannot be completed at this time. The control system may display a countdown to inform a user as to how long until the subsequent firing stroke can be performed.

The method 3300 comprises controlling 3316 the motor to drive the firing driver through a second firing stroke based on the elapsed time reaching or exceeding the recovery time period. In accordance with the present disclosure, based on the elapsed period reaching or exceeding the recovery time period, the control system can control the motor to drive the firing driver through the second firing stroke. Further, in accordance with the present disclosure, the method may comprise issuing a notification on the display based on the elapsed period reaching the recovery time period. Accordingly, a user is notified that the power source has recovered a sufficient amount and that a second firing stroke can now be completed.

In accordance with the present disclosure, the method 3300 optionally may further comprise monitoring a power source voltage potential at the end of conclusion the first firing stroke, comparing the monitored voltage potential to the recovery threshold based on receiving the second firing input, and performing an action, such as abstaining from driving the motor or controlling the motor to drive the firing driver through a second firing stroke, based on the comparison.

In accordance with the present disclosure, voltage drop may be minimized by adjusting the configuration of the battery cells in the power source. Adjusting the configuration of the battery cells may comprise adjusting the number and/or configurations of the cells within the battery to minimize voltage drop.

Balancing the cells of the battery affects the impact of the battery output performance. The number of streams affects the impact of the battery output performance. In accordance with the present disclosure, voltage drop can be minimized by selectively placing some, or all, cells in parallel with one another. Alternatively, voltage drop can be minimized by selectively placing some, or all, cells in series with one another. Alternatively, voltage drop can be minimized by selectively placing some cells in series and some cells in parallel with one another. Paralleling cells increases the capacity of the output of the power source. Placing the cells in series increases the voltage of the output since series cells have internal resistance that are additive. Accordingly, a user can selectively place the battery cells in series and/or parallel based on the desired outcome. Alternatively, the combination of the battery cell chemistries may enhance the overall performance of the battery. For instance a secondary cell may be utilized in combination with a primary cell, where the secondary cell is used to handle the circuit inrush current. In accordance with the present disclosure, the control circuit can selectively switch between the primary cell and the secondary cell based on which of the cells is better suited for a desired need.

In accordance with the present disclosure, the control system may actively control the number of cells used during a firing stroke and makes adjustments “on the fly”. The control system may set a voltage drop threshold and may monitor the battery voltage drop during the firing stroke. Based on the voltage potential reaching or dropping below the voltage drop threshold, the control system changes a state of the battery to actively switch additional cells into the circuit to add additional power. In accordance with the present disclosure, the control system may utilize a first number of cells during an initial portion of a firing stroke. Based on the voltage potential reaching or dropping below the voltage drop threshold, the control circuit changes the state of the power source such that a second number of cells which is more than the first number of cells are utilized. The control system may complete this transition using switching circuitry to place additional cells in series and/or parallel with the first number of cells depending on a desired need. Additionally, in accordance with the present disclosure, based on the rate of change of the voltage drop, the control system may determine whether or not to switch from a primary power source configured to provide a first voltage potential to a second power source configured to provide a second voltage potential different than the first voltage potential.

In accordance with the present disclosure, the surgical system can include an auxiliary battery pack and the control system switches to the auxiliary battery pack if the primary power source voltage potential drops below a threshold level. Additionally, in accordance with the present disclosure, the control system can couple the auxiliary battery pack to the primary battery pack to enhance the power output of the system to a higher level than was previously available. For instance, the control system may interrogate a force sensor to determine if more power is necessary to complete a firing stroke. Based on the determination, the control circuit couples the auxiliary battery pack to the primary battery pack to provide the necessary power to complete the firing stroke.

In accordance with the present disclosure, the power source may comprise a battery that includes cells comprised of lithium ion. Alternatively, the cells may comprise lithium cobalt cells (NCA). Alternatively, the cells may comprise lithium nickel manganese cobalt cells (NMC). Alternatively, the cells may comprise lithium nickel cobalt aluminum cells (NCA). Alternatively, the cells may comprise lithium iron phosphate cells (LFP). Alternatively, the cells may comprise lithium manganese spinel cells (LMO). Alternatively, the cells may comprise lithium titanate cells (LTO). Alternatively, the cells may comprise lithium cobalt oxide cells (LCO).

During operation of a surgical stapling system, heat is generated by the motor, the motor controller, the power source, or other components, for example. As these components heat up, the system operates less efficiently than if the components were maintained at cooler temperatures. These increasing temperatures ultimately affect the amount of force output by the surgical system. Accordingly, it would be desirable to dissipate heat from heat sensitive components to other portions of the surgical system to reduce the heat impacts during heavy usage of the surgical stapling system.

In one embodiment, the surgical stapling system includes a heat sink to extract heat from the heat generating components of the surgical stapling instrument. In one aspect, the heat sink enables the motor to operate at an improved consistent internal temperature and operate more efficiently. In some embodiments, the heat sink removes heat via convection or radiation heat transfer to equalize the internal ambient temperature of the surgical stapling system.

In one aspect, in a surgical stapling system, heat is generated at the gearbox of the motor, the power source (such as a battery), the printed circuit board (PCB) power supplies, and other electrical components of the PCB. In some embodiments, the motor controller is configured to accommodate the heat generated by various components. For instance, in some embodiments, the MOSFETs in the H-bridge of the motor controller are selected such that they can handle, at least, two times more heat dissipation than a standard surgical stapling instrument. In some embodiments, the motor controller comprises an SOT-23 MOSFET, which has a small outline and low power dissipation. In some embodiments, the motor controller comprises a D-pack MOSFET, which has a larger outline than an SOT-23 MOSFET, an additional thermal capacity, and an addition heat sinking capacity with a metal plate on the backside. In one aspect, active or passive cooling components, such as a heat sink, allows for a compact surgical stapling system form factor making it more cost effective due, in part, to the ability of using “smaller” MOSFETS and components that is achievable by keeping the temperatures cool under active control.

In one aspect, during calibration of the surgical stapling system, a baseline internal resistance of the H-bridge is calculated to optimize field performance of the system as the instrument heats up. A heating transfer function of the system, determined by passing current through the H-bridge while the motor is powered in an unloaded state and the instrument is cooled, via active or passive heat components, can be adjusted based on a determined position along the heating/resistance curve. In various embodiments, the surgical system comprises MOSFET with a built-in temperature sensor to sense internal temperature of the system. In some embodiments, the MOSFET comprises an integrated temperature sensor and is known as a TEMPFET. In some embodiments, the surgical system comprises a sensor external to the MOSFET that is utilized to make adjustments to the surgical system based on the sensed temperatures of the internal components.

In various embodiments, the surgical system comprises an active cooling system to control the internal temperature of the surgical system. In some embodiments, the active cooling system is configured to drive the RDS (ON) resistance of the H-bridge MOSFETS below the current ambient temperature, which would drive the RDS (ON) resistance of the MOSFET lower than the level associated with the ambient surrounding device temperature. In one aspect, these small adjustments to the component temperature help drive additional power to the motor and not being burned up in heat.

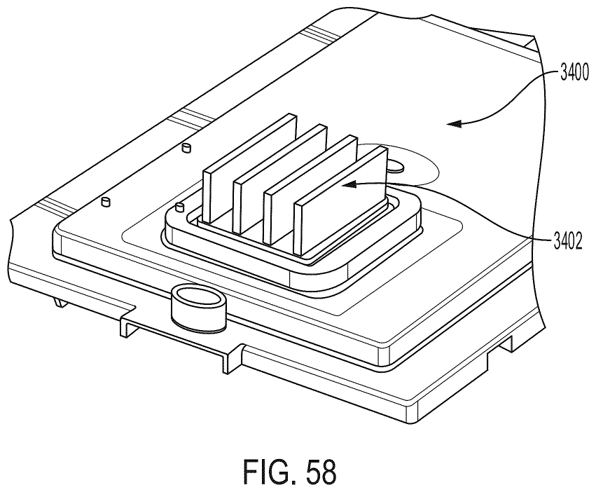

In various embodiments, shrouds are mechanically and electrically integrated with the PCB to utilize the additional surface area and materials to control the temperature of the PCT. FIG. 58 shows a PCB 3400 that includes an exposed heat sink 3402 comprising a plurality of fins. The exposed heat sink controls the internal temperature of the surgical system.

As the temperature of the power source or the motor increases, the surgical instrument becomes less efficient. Accordingly, it would be desirable to cool the power source and/or motor during operation of the surgical instrument.

In various embodiments, the power source is cooled using forced air from one of the motors. In some embodiments, the motor vents push air to the internals of the power source for additional cooling. In some embodiments, pass through vents are molded into the power source housing to allow for additional flow across the cells of the power source.

In various embodiments, the power source is sealed and filled with a paraffin wax type of material. As the cells temperature increases, the paraffin melts, which will draw heat away from the cells. Once the cell returns to normal operating temperature, the wax re-solidifies.

In various embodiments, the cells of the power source are wrapped with a heat sinking material. The heat sinking material is integrated with the power source shrouds to increase the surface area and give the material access to the “outside” ambient temperature.

In various embodiments, the surgical instrument comprises a thermopile that coverts thermal energy into electrical energy. In one aspect, the thermopile absorbs heat generated by the surgical device and converts this heat to electrical energy. In some embodiments, this electrical energy is used to power an active cooling element, such as cooling fan. In some embodiments, this electrical energy is used to recharge the power source. In some embodiments, energy harvesting technology is employed to harvest heat generated by the power source to produce power. This power is utilized to power an auxiliary cooling system or recharge the power source itself.

In various embodiments, the temperature of the motor is controlled by using the air escaping from the spinning rotor of the motor. In some embodiments, the shrouds are designed such that the escaped air is redirected to flow directly across the motor. In various embodiments, the temperature of the motor is controlled by physically attaching metal portions of the motor directly to the coated shrouds comprised of a thermally conductive material to increase the surface area to dissipate heat. In various embodiments, the temperature of the motor is controlled by wrapping the metal motor can with a circular heat sink to increase the surface area of the motor. In addition, in some embodiment, forced air is added to increase the heat transfer of the system.

In various embodiments, the temperature of the motor can be controlled by replacing the internal gear material from plastic to metal. The metal material being a better heat conductor than plastic would aid in pulling a substantial amount of heat out of the gearbox and the motor. In one aspect, changing a component (gear) that is in direct contact with the heat source (motor) to one having a heat transfer characteristics, can yield a higher heat transfer and greater cooling. In one embodiment, the gear can be comprised of a high carbon filled material.

During a surgical stapling procedure, a clinician may operate a powered surgical stapling instrument 5 to sequentially fire multiple staple cartridges along a selected tissue resection line to achieve a clinical outcome. For example, in a stomach resection procedure, the clinician may sequentially fire staple cartridges of different characteristics (e.g., size, color, type, length, staple height, staple diameter, staple size) along a selected tissue resection line to remove a portion of the stomach. The staple cartridges can be fired along the tissue resection line in an end-to-end arrangement.

The clinician may examine the tissue to be resected using any suitable imaging technique such as, for example, x-ray, registered magnetic resonance imaging (MRI), and/or computerized tomography (CT) scan. The clinician may then select a suitable tissue resection line, and staple cartridges for sequential firing along the selected tissue resection line. Visual examination, however, has its limitations, and a tissue response to stapling can vary depending on many factors including, for example, patient age, tissue health, and/or tissue type. Moreover, tissue thickness and/or stiffness may vary along the selected tissue resection line, resulting in unexpected tissue responses.

Various methods, devices, and systems are provided for adaptively adjusting operational parameters of the powered surgical stapling instrument 5 during a staple cartridge firing based on a tissue response in an earlier phase/zone of the staple cartridge firing. Moreover, various methods, devices, and systems are provided for adaptively adjusting operational parameters of the powered surgical stapling instrument 5 during a staple cartridge firing based on a tissue response in one or more previous staple cartridge firings in a surgical procedure involving multiple sequential firings.

In accordance with the present disclosure, the processor 104 can execute various program instructions, which can be stored in a memory circuit such as the memory circuit 106, to implement various algorithms associated with firing a staple cartridge by the powered surgical stapling instrument 5. Various aspects of such algorithms, e.g., thresholds, limits, triggers, conditions, pauses, wait time, are adjusted based on information learned from a tissue response in an earlier phase/zone of the staple cartridge firing, and/or based on a tissue response in one or more previous staple cartridge firings in a surgical procedure involving multiple sequential firings, as described in more detail below.

In accordance with the present disclosure, the control circuit 100 and the motor assembly 110 may have different voltage levels. The control circuit 100 may have a first voltage level that is different than the voltage of the motor assembly 110. This mitigates in transit loss of voltage or current through motor control systems of the surgical instrument. In transit loss occurs during the transit of power from the power source to the motor assembly. In accordance with the present disclosure, the motor assembly 110 may have an “H-bridge” portion that operates at a voltage level that is different than at least one other portion of the circuit. The voltage of the control circuit 100 may be higher than the voltage within the motor assembly 110 to minimize resistive losses of the transit of the power from the power source to the motor assembly. Alternatively, the voltage of the control circuit 100 may be lower than the motor assembly 110, causing higher motor efficiency.

Transmissibility improvements lead to an increase in power transfer and higher motor efficiency. In accordance with the present disclosure, power transfer can be optimized by architectural improvements. For example, a reduction of intermediate control elements within the control circuit 100 reduces overall power consumption. Distance, resistance, conductor shape, and shape all effect electrical current capacity and as a result overall power consumption. For example, resistances are between 80 mOhms to 170-200 mOhms to reduce power consumption.

In addition, parasitic losses and losses to other forms of energy, such as magnetic, capacitive, and heat, occur in the surgical instrument. Heat dissipation is a form of loss that occurs in a circuit due to proximity or enclosure constraints to other electronics. Minimization of these losses is necessary for extending battery life and efficient power transfer to the motor. With a set battery voltage the present disclosure is directed to maximizing the power delivered to the motor system. For example, the present disclosure is directed to capturing as much of the magnetic field as possible, using flux rings and running the motor at its peak spots on the motor curve. Since each cell within the battery has some internal resistance, by minimizing the current being pulled by the device, this lowers the losses caused by the internal battery resistance (heat from the battery). The cell heating can also negatively impact the battery performance.

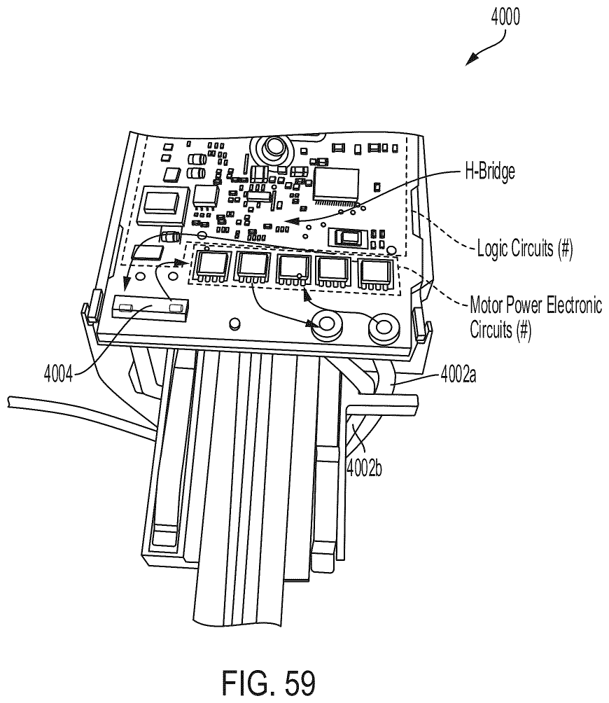

FIG. 59 is an example of a control circuit 4000, in accordance with the present disclosure. The control circuit 4000 is one implementation of the control circuit 100 shown in FIG. 4. As illustrated in FIG. 59, the control circuit 4000 comprises motor first and second leads 4002a, 4002b to couple the control circuit 4000 to the motor (not shown in FIG. 59, see motor 4012 in FIG. 61). In accordance with the present disclosure, the control circuit 4000 may comprise a battery 4004.

In accordance with the present disclosure, the overall current path may be minimized as the circuit components are closely spaced to minimize losses. The current path is through the positive (+) terminal of the battery 4004 to the H-Bridge, to the motor, and to the negative (−) return terminal of the battery 4004. Adjacent circuit components also lose power through induced capacitive coupling and inductive coupling.

The control circuit 4000 is disposed on a printed circuit board (PCB). On the PCB, power electronics 4001 are separated from the logic circuits 4003, such as the digital electronics or electrical conductors carrying data signals to physically and electrically isolate noisy power signal traces from digital/data traces. Accordingly, the digital traces are free from unwanted noise or interference caused by electric, magnetic, or thermal sources.

In accordance with the present disclosure, to minimize parasitic losses in the control circuit 4000, the voltage source to power the control circuit may be unpaired or decoupled from the voltage source to power the motor. This technique enables different power sources to be used. For example, a 24V battery to power a 12V motor uses a step-down circuit at the motor. The 24V battery then has less electrical losses while in storage than a 12V battery. This minimizes parasitic losses. Additional losses occur due to the skin effect in electrical conductors during pulse width modulation (PWM). For example, in electromagnetism, the skin effect refers to the tendency of an alternating electric current (AC) to become distributed within an electrical conductor such that the current density is largest near the surface of the electrical conductor and decreases exponentially with greater depths in the conductor. Thus, in AC circuits, power transfer is mostly through skin transfer while in DC circuits power transfer occurs mid-electrical conductor.

Motor efficiency is a measurement of how much of the electrical energy applied to a motor is converted to mechanical energy. Much of the remaining energy is converted into heat, which can cause a motor to burn out if the motor is operated at a torque and/or revolutions per minute (RPM) where the motor efficiency is very low. Motor heating is directly related to output torque of the motor system. Thus, it is important to control motor generated heat. One way to control motor generated heat is to use a flux ring to envelop the motor.

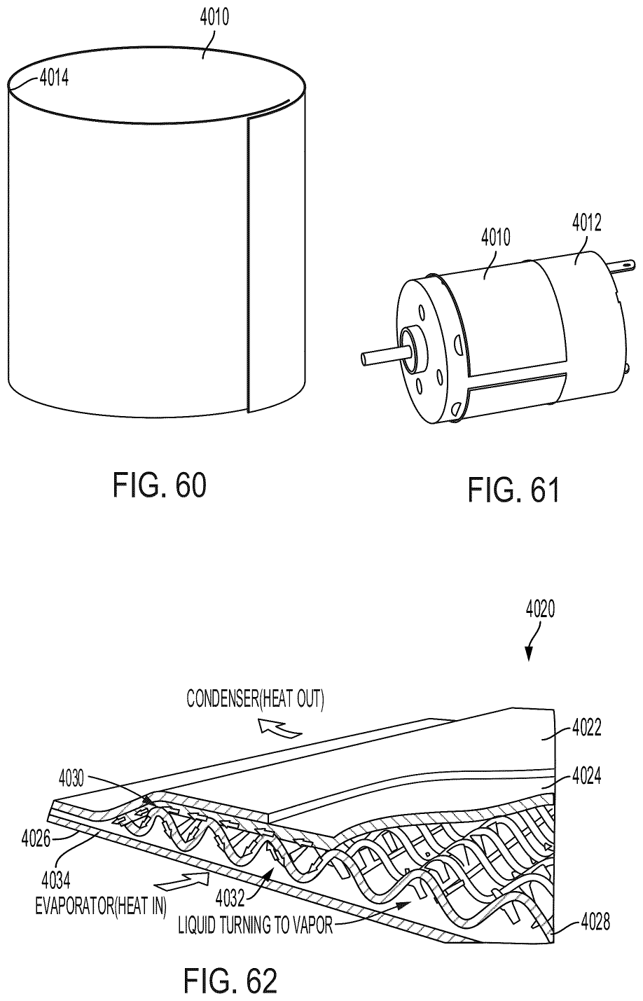

FIG. 60 is an example of a flux ring 4010, in accordance with the present disclosure. The flux ring 4010 is a ferrous metal ring that increases the efficiency of the magnetic field on a motor by lowering the RPM per volt and reducing the amperage draw of the motor. The flux ring 4010 also conducts additional heat away from the motor. Increasing the thickness of the flux ring 4010 enables additional heat to be conducted away from the motor.

FIG. 61 is an illustration of the flux ring 4010 disposed around a motor 4012, in accordance with the present disclosure. The flux ring 4010 is slidably disposed on the motor 4012. The flux ring 4010 changes the magnetic flux characteristics of the motor 4012 and impacts the performance of the motor 4012. The flux ring 4010 disposed around the motor 4012 advantageously reduces the power consumption of the motor 4012 and extends the life of the battery.

FIG. 62 illustrates a vapor chamber 4020 to remove heat from the motor 4012, in accordance with the present disclosure. The flux ring 4010 shown in FIG. 61 may comprise the vapor chamber 4020. The vapor chamber 4020 comprises a condenser 4022, an evaporator 4026, a wick 4024 coupled to the condenser provides a liquid return path. A cavity 4032 is defined between the wick 4024 and the evaporator 4026 to accommodate fluid within the cavity 4032. Coils 4028 are disposed in the cavity 4032. The liquid returned from the wick 4024 is converted into a vapor within the cavity 4032. The evaporator 4026 is to pull heat from the motor. In accordance with the present disclosure, the evaporator 4026 may be in physical contact with the motor (not shown) from which the vapor chamber is to remove heat.

As heat is introduced to the evaporator 4026, from a motor coupling side 4034 of the vapor chamber 4020, the working fluid within the vapor chamber 4020 turns to vapor which moves to areas of lower pressure. In accordance with the present disclosure, the evaporator 4026 can be in contact with one side of the motor 4012 through the motor coupling side 4014. In another example, the evaporator 4026 is in contact with an open side of the flux ring 4010. The condenser 4022, usually a finned structure, cools the vapor such that the vapor condenses back to a liquid which is absorbed by the wick 4024 and returned via capillary action to the heat source area.

In accordance with the present disclosure, the vapor chamber 4020 can be part of the flux ring 4010 shown in FIGS. 60 and 61 to conduct heat away from the motor 4012 (FIG. 61). The vapor chamber 4020 forms a cylindrical shape. The vapor chamber 4020 may be a copper heat pipe. Further, in accordance with the present disclosure, the vapor chamber 4020 may define a shape that conforms to the outer housing or shape of a motor.

Additional techniques to minimize system parasitic losses and cool system components by removing the heat (energy) from the system components include removing heat from the H-bridge and the transistors (e.g., MOSFET devices) by way of cooling mechanisms including heat sinks, heat pipes, or vapor chambers.

Other contributing sources of heat include heat energy generated by motor vibrations when the motor is out of specification, e.g., when the motor is being operated above its operating conditions. When the motor vibrates, the motor gearbox may encounter tooth loading, which will add heat energy to the system. One solution is to optimize the impact angle for high loading conditions. During low loading conditions the efficiency is lower but it allows higher outputs during critical high loading conditions of the instrument firing process.

Another solution is to replace plastic gears with metal gears. The loading characteristics of plastic gears change the optimized or efficiency of the contact to the mating gear. The loading profile would “steal” output power due to the gear requiring more energy to rotate. If the gears are metal the contact point would not deflect which would keep the power input required lower than if plastic gears are used.

Other sources of heat energy that can be mitigated include electromagnetic interference (EMI) and electromagnetic compatibly (EMC). This includes adding effective EMI/EMC shielding and filtering to the surgical instrument to simultaneously improve EMI/EMC immunity and reduce electromagnetic emissions, while minimizing risk.

Moreover, motor efficiency can be optimized by tuning the motor drive configurations to compensate for resistance changes or losses to the power supply voltage level. One method for compensating the power supply voltage applied to the motor is through continuous control variation of one or more H-Bridge transistors by operating at least one of the transistors in the linear operating region. Operating the H-Bridge transistors in the linear region provides additional control of the current flowing to the motor. The linear region could be used to lower the current flowing to the motor, which in turn lowers the speed and reduces energy consumption that contributing to the system heat energy losses.

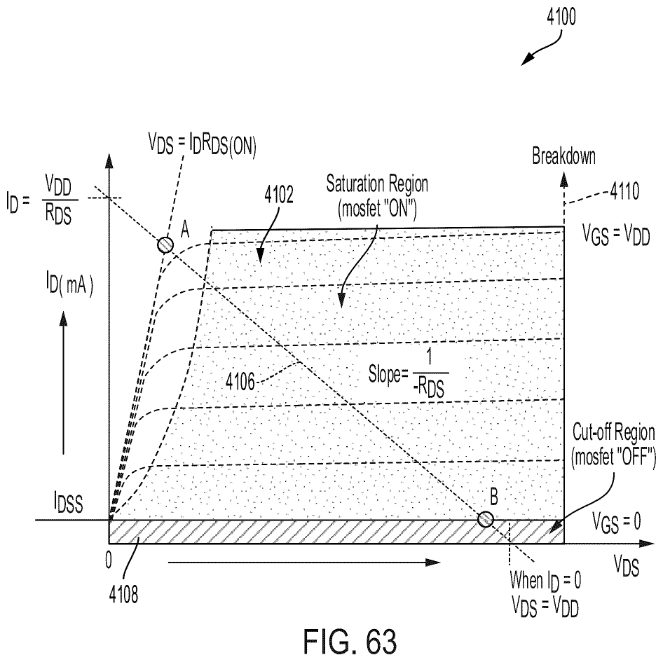

Before describing techniques for operating a motorized surgical instrument, such as the powered surgical stapling instrument 5 shown in FIGS. 1-4, by operating the H-Bridge transistors in the linear region, the disclosure turns to FIG. 63. FIG. 63 is a graph 4100 illustrating characteristics of typical MOSFET devices. The graph 4100 shows Drain to Source voltage (V_{DS}) vs Drain Current (I_{D}) of a typical MOSFET device. The saturation region 4102 occurs when V_{GS}>V_{TH} which creates a maximum current. The load line 4106 has two points, A and B, which illustrate the voltage and current at the cut-off region and the saturation region 4102.

In the saturation region 4102, the MOSFET device will be biased so that the maximum gate voltage is applied to the MOSFET device resulting in channel resistance R_{DS} being as small as possible with maximum drain current flowing through the MOSFET device. Therefore for enhancement type MOSFET devices the conductive channel is open and the device is switched “ON.”

The cut-off region 4108 occurs when V_{GS}<V_{TH}. The MOSFET device operates as an open switch. The breakdown voltage 4110 is shown on graph 4120. By applying a suitable drive voltage to the gate of a MOSFET device, the resistance of the drain-source channel, R_{DS(on)} can be varied from an “OFF-resistance” of many hundreds of kilo-Ohms, effectively acting as an open circuit, to an “ON-resistance” of less than 1Ω, effectively acting as a short circuit.

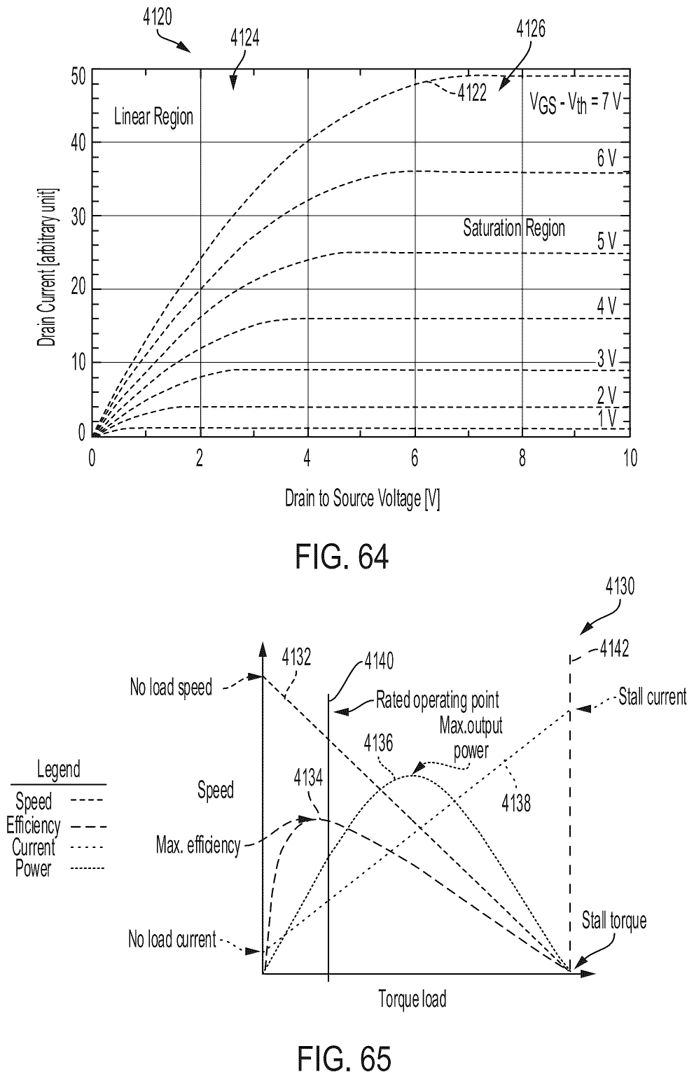

FIG. 64 illustrates a graph 4120 of characteristic Drain to Source Voltage (V_{DS}) vs Drain Current (I_{D}) curves for a MOSFET device operating at different values of Gate to Source Voltage (V_{GS}). The primary current flow through the MOSFET device is controlled by the voltage applied to one of the terminals rather than by a control current flow through any part of the device. When the drain voltage is increased to the saturation voltage, V_{SAT} the current through the device becomes controlled solely by the gate voltage under drain saturation conditions. The linear region 4124 and saturation region 4126 are separated by the upward curving parabola 4122.

FIG. 65 is a Torque Load vs Speed graph 4130 illustrating an example of characteristics of operating a motorized surgical instrument. The modulation of the current being delivered to the motor impacts the output force the motor can apply. There is a direct relationship between speed 4132 and torque.

For example, where there is no load, the speed of the motor is at a maximum. For example, when the motor is stalled 4142, there is a maximum current being applied to the motor, but no torque is being created and the rotor does not rotate while the motor is stalled. The speed and efficiency are also zero. The rated operating point is between the no load and stall conditions. The speed 4132, efficiency 4134, current 4138, and power 4136 lines all intersect the rated operating point 4140 of the motor. Rated operating point 4140 intersects all the lines for a specific load applied to the motor.

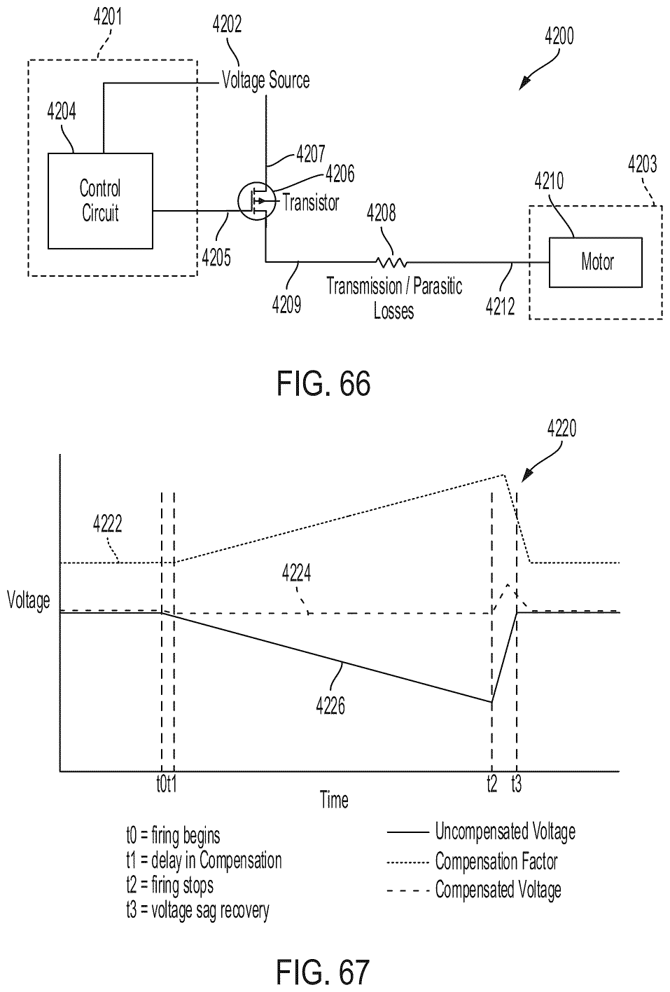

FIGS. 66-69 hereinbelow will now be described in combination with the powered surgical stapling instrument 5 and the control circuit 100 shown in FIGS. 1-4. For example, as described hereinbelow, the control circuits 4204, 4304, 4404 are example implementations of the control circuit 100 of the powered surgical stapling instrument 5 shown in FIG. 4. With reference to FIGS. 66-69 together with FIGS. 1-4, the power source 111 (FIG. 4) is divided into two circuit portions. A first portion applies power to the control circuit 100 components and a second circuit portion applies power to the motor assembly 110 (FIG. 4). In FIGS. 66-69, the two circuit portions are shown as first circuit portions 4201, 4301, 4401 and second circuit portions 4203, 4303, 4403. The first circuit portions 4201, 4301, 4401 comprises the voltage source 4202, 4302, 4402 and the control circuit 4204, 4304, 4404 and the second circuit portion 4203, 4303, 4403 comprises the motor 4210, 4310, 4410. Aspects of FIGS. 59-62 can be implemented in combination with FIGS. 66-69 to increase motor efficiency, such as a flux ring or vapor chamber disposed on the motor of FIGS. 66-69.

FIG. 66 is an example of a motor control system 4200 of a powered surgical instrument to control a motor 4210 of the powered surgical instrument, in accordance with the present disclosure. The motor control system 4200 of the powered surgical instrument comprises a voltage source 4202, a transistor 4206 coupled to the voltage source 4202, and a control circuit 4204. The transistor 4206 can be a MOSFET, BJT, JFET, FET, NPN transistor, PNP transistor, or any suitable transistor. The powered surgical instrument comprises a first circuit portion 4201 operable at a first voltage level and a second circuit portion 4203 operable at a second voltage level 4212, shown as the voltage applied to the motor 4210. For example, the voltage source 4202 is configured to supply the second voltage level to the second circuit portion 4203, such that the motor receives a voltage level similar to the voltage source 4202.

In accordance with the present disclosure, the motor control system 4200 can be located within the powered surgical instrument, such as, for example, the powered surgical stapling instrument 5 shown in FIGS. 1-4. Additionally, in accordance with the present disclosure, the second voltage level 4212 can be different from the first voltage level of the voltage source 4202. The voltage source 4202 of the powered surgical instrument supplies the first voltage level to power electrical components in the first circuit portion 4201. A boost circuit (not shown) can be coupled between the voltage source 4202 and control circuit 4204 to boost the voltage level of the voltage source to the first voltage level, such that the first voltage level is higher than the voltage level of the voltage source 4202. A higher voltage to the control circuit 4204 allows the control circuit 4204 to draw less current from the voltage source 4202. This allows the motor 4210 to receive more power. A first current carrying terminal 4207 of the transistor 4206 is coupled to the voltage source 4202 and a second current carrying terminal 4209 of the transistor 4206 is coupled to the motor 4210 via transmission/parasitic losses 4208. The control circuit 4204 is coupled to the control terminal 4205 of the transistor 4206 to control the conductivity of the transistor 4206 between the first and second current carrying terminals 4207, 4209.

With reference back to FIG. 66, the transistor 4206 is to output the voltage to be applied to the motor 4210 taking into account the transmission/parasitic losses 4208. The transistor 4206 output current between the first and second current carrying terminals 4207, 4209 is controlled by the control circuit 4204 to compensate for the transmission/parasitic losses 4208.

In accordance with the present disclosure, when the voltage source 4202 is higher than the input voltage rating of the motor 4210, the control circuit 4204 may step the voltage source 4202 voltage down to a voltage usable by the motor 4210 by controlling the conductivity of the transistor 4206 between the first and second current carrying terminals 4207, 4209.

For example, in accordance with the present disclosure, the control circuit 4204 may set the transistor 4206 in the linear mode (as discussed above in connection with FIGS. 63-64) to control the output voltage of the transistor 4206 to set a current to the motor 4210 to power the motor 4210. The transistor 4206 may be located between the first and second circuit portions.

In accordance with the present disclosure, the control circuit 4204 may set the transistor 4206 in the linear mode (as discussed above in connection with FIGS. 63-64). Additionally, in accordance with the present disclosure, when the transistor 4206 is a MOSFET device, the linear mode of a MOSFET device is shown in FIGS. 63-64. Linear mode operation refers to the current saturation region in the output characteristics. The drain current (I_{DS}) is nearly independent of the drain to source voltage (V_{DS}) for a given gate to source (V_{GS}) voltage in the linear region. The drain current depends then directly on the V_{GS} voltage of the MOSFET.

In accordance with the present disclosure, the control circuit 4204 can control the output voltage of the transistor 4206 at the second current carrying terminal 4209 of the transistor 4206 to a second voltage level by applying a stored profile to the control terminal 4205 of the transistor 4206. The stored profile stores a compensation factor to compensate for voltage drops of the voltage source 4202 during a firing operation of the powered surgical instrument driven by the motor 4210. The control circuit 4204 adjusts for the voltage drop and increases the voltage applied to the motor 4210 such that the voltage applied to the motor is similar to the voltage level of the voltage source 4202. In accordance with the present disclosure, the control circuit 4204 can set a current for the motor 4210 by controlling the control terminal 4205 of the transistor 4206 to adjust the conductivity of the transistor 4206 and apply a desired transistor voltage at the second current carrying terminal 4209 of the transistor 4206 that is compensated for the transmission/parasitic losses 4208 such that the second voltage level 4212 is suitable for efficiently operating the motor 4210 during the firing process.

In accordance with the present disclosure, any voltage drop can be ‘pre-compensated’ when the fire-trigger of the powered surgical instrument is activated. Pre-compensation may be based on known profiles of inrush current for a given motor 4210. For example, each powered surgical instrument includes a memory to store motor profiles. The powered surgical instruments are individually calibrated to meet a predetermined performance based on predetermined parameters. In accordance with the present disclosure, the compensation parameter values may be physically stored in a potentiometer circuit or in a memory circuit of the control circuit 4204, such as for example, an EEPROM (electrically erasable programmable read only memory). Further, in accordance with the present disclosure, the potentiometer can be controlled by the control circuit 4204 to adjust the second voltage level 4212 applied to the motor 4210. One example of a stored voltage profile during a firing operation is described below in connection with FIG. 67.

FIG. 67 is a graph 4220 illustrating an example stored profile of voltage as a function of time during a firing stroke. With reference now to FIG. 67 together with FIG. 66, the control circuit 4204 stores a profile similar to the profile depicted in the graph 4220. The control circuit 4204 adjusts the second voltage level 4212 based on a stored profile of the motor 4210. The stored profile includes the uncompensated voltage 4226, the compensation factor 4222, and the compensated voltage 4224.

To determine the profile in calibration, the second voltage level 4212 applied to the motor 4210 is measured and compared to a desired voltage to be applied to the motor 4210. A compensation factor is determined based on the difference between the applied voltage and the desired voltage. The control circuit 4204 stores the compensation factor 4222.

For example, the first circuit portion 4201 comprises the control circuit 4204 and the second circuit portion 4203 comprises the motor 4210. The transistor 4206 may separate the first circuit portion 4201 and second circuit portion 4203. The motor 4210 receives the second voltage level 4212. The control circuit 4204 receives the first voltage level. In accordance with the present disclosure, the first voltage level may be a compensated voltage, compensated by the compensation factor 4222, such that the first voltage level is higher than the second voltage level.

In accordance with the present disclosure, the first voltage level—the voltage at the voltage source 4202—may be higher than the second voltage level 4212 applied to the motor 4210. Alternatively, the first voltage level—the voltage at the voltage source 4202—may be lower than the second voltage level 4212 applied to the motor 4210.

The control circuit 4204 also minimizes or maximizes the power applied to the motor 4210 and stores algorithms to modulate the power applied to the motor 4210. For example, the first several firings of the powered surgical instrument will encounter a higher force to fire and will require more power applied to motor 4210 to advance the drive shaft 40. Based on the device cycle number, the control circuit 4204 increases the output power and monitors the power throughout the cycles. The control circuit 4204 counts the number of cycles and stores the device cycle number. The control circuit 4204 also determines the second voltage level 4212 to apply to the motor based on the stored number of cycles for the device. The control circuit 4204 also stores the output power of the prior cycles and determines a desired output power to the motor 4210, at least based in part, on prior output power.

In accordance with the present disclosure, the increase in output power from the voltage source 4202 may be controlled by changing the second voltage level 4212, e.g., the voltage applied to the motor 4210. By way of example, if the motor control system 4200 uses an 18V voltage source 4202, e.g., battery, to power the control circuit 4204 and the motor 4210, during the initial firing cycle the control circuit 4204 applies the full 18 volts to the motor 4210. As the load increases, the second voltage level 4212 applied to the motor 4210 is adjusted to the voltage necessary to complete the firing cycle. If the first several firing were at an increased load—higher than a predetermined threshold, the control circuit 4204 continues to apply the full 18 volts to the motor 4210. If the initial firings are lower than the threshold, the motor 4210 is throttled down to a lower voltage that better matches the loads on the end effector 20 (FIGS. 1 and 4). The control circuit 4204 stores the prior loads on the end effector 20 for the prior firings and determines a suitable voltage level to be applied to motor 4210 to operate properly under the loads, at least based in part on the prior loads.

In another example, during a motor 4210 stall condition, the control circuit 4204 raises the second voltage level 4212 applied to the motor 4210 incrementally to attempt to return the firing beam 60/knife (FIG. 1) without the use of a mechanical bailout. The control circuit 4204 stores differing thresholds for firing and return to minimize damage and risk. The control circuit 4204 determines the condition of the motor 4210 and determines the second voltage level 4212 to apply to the motor 4210 based on the condition of the motor 4210. In addition, the control circuit 4204 controls the decay of the H-bridge drive circuit. A slow decay of the H-bridge drive circuit increases the efficiency of the motor 4210. A faster decay allows for faster braking.

In accordance with the present disclosure, the motor 4210 may be a bipolar or unipolar stepper motor to operate outside of the pull-out and pull-in torque of the motor 4210, which causes the motor 4210 and the attached drive system to vibrate.

In accordance with the present disclosure, the transistor 4206 can be replaced with a DC/DC power converter. The DC/DC power converter drops a higher voltage provided by the voltage source 4202 down to a lower second voltage level 4212 for operating the motor 4210. In accordance with the present disclosure, the transistor 4206 may be a MOSFET device. Further, in accordance with the present disclosure, the transistor 4206 may be a BJT device.

In accordance with the present disclosure, a higher second voltage level 4212 can be utilized to drive the motor 4210, such as a 14V second voltage level 4212 for a 12V battery voltage source 4202. An unknown amount of voltage drop, such as 1V for example, will occur due to transmission/parasitic losses 4208. The transistor 4206 can then be used in the linear region to compensate the remaining overhead voltage down to second voltage level 4212 that is acceptable for the motor 4210. The control circuit 4204 controls the voltage drop of the transistor 4206 by controlling the conductivity of the transistor 4206 through the control terminal 4205.

In accordance with the present disclosure, the control circuit 4204 can monitor the voltage drop and dynamically compensates the voltage drop. The voltage drop is due to voltage and battery sag.

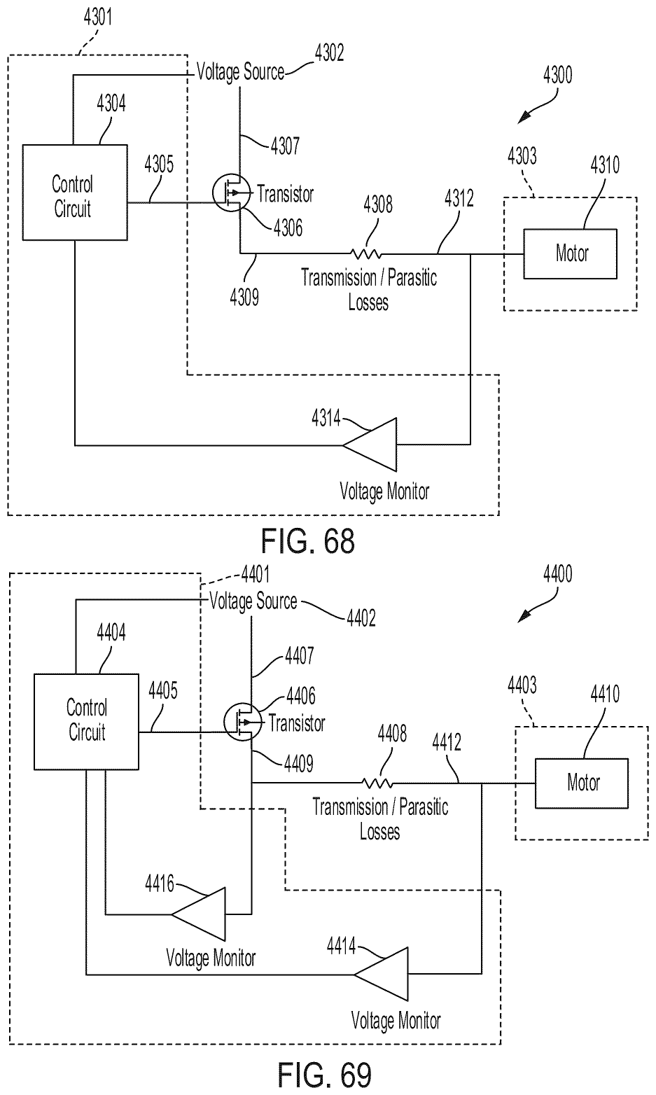

FIGS. 68 and 69 illustrate alternative implementations of the motor control system 4200 shown in FIG. 66. In accordance with the present disclosure, as illustrated in FIGS. 68 and 69 the control circuit 4304, 4404 may use an algorithm and active monitoring of the second voltage level 4312, 4412 applied to the motor 4310, 4410 to provide the status of the second voltage level 4312, 4412 being applied to the motor and to compensate the second voltage level 4312, 4412 to a desired voltage level.

With reference now to FIG. 68, there is shown a motor control system 4300 of a powered surgical instrument, in accordance with the present disclosure. The powered surgical instrument is similar to the powered surgical stapling instrument 5 shown in FIGS. 1-4. The motor control system 4300 is an example circuit implementation for controlling the motor 4310 of the powered surgical instrument. In accordance with the present disclosure, the circuits can be contained within a housing of the powered surgical instrument. The motor control system 4300 is similar to the motor control system 4200 shown in FIG. 66, and for conciseness and clarity of disclosure similar components will not be described in detail. In accordance with the present disclosure, the motor control system 4300 can be located within a housing of the powered surgical instrument. The motor control system 4300 comprises a first circuit portion 4301 operable at a first voltage level-supplied by the voltage source 4302 and a second circuit portion 4303 operable at a second voltage level 4312. In accordance with the present disclosure, the second voltage level 4312 may be different from the first voltage level supplied by the voltage source 4302. The voltage source 4302 of the powered surgical instrument supplies the first voltage level to power electronic/electrical components in the first circuit portion 4301, such as the control circuit 4304. A first conduction terminal 4307 of the transistor 4306 is coupled to the voltage source 4302. A second conduction terminal 4309 of the transistor 4306 is coupled to the motor 4310 via transmission/parasitic losses 4308. The control circuit 4304 is coupled to a control terminal 4305 of the transistor 4306.

The motor control system 4300 comprises a voltage monitor circuit 4314 coupled to the second conduction terminal 4309 of the transistor 4306, the voltage monitor circuit 4314 measures the voltage applied to the motor 4310 and provides the voltage as feedback to the control circuit 4304. In accordance with the present disclosure, the control circuit 4304 can receive the second voltage level 4312 applied to the motor 4310 as measured by the voltage monitor circuit 4314 and may adjust the second voltage level 4312 based on the measured voltage. In accordance with the present disclosure, the control circuit 4304 may compare a predetermined value of the second voltage level 4312 to the voltage applied to the motor 4310 as measured by the voltage monitor circuit 4314 and may adjust the second voltage level 4312 based on the comparison. For example, the predetermined value of the second voltage level 4312 is the voltage level of the voltage source 4302.

Compensation can also be used to allow for monitoring. Compensation can be used to allow for both unintentional losses as well as losses due to passive elements (e.g., diodes) and active elements (e.g., other active/powered circuitry).

FIG. 69 is motor control system 4400 of a surgical instrument, in accordance with the present disclosure. The powered surgical instrument is similar to the powered surgical stapling instrument 5 shown in FIGS. 1-4. The motor control system 4300 is an example circuit implementation for controlling the motor 4410 of the powered surgical instrument. The motor control system 4400 is similar to the motor control systems 4200, 4300 shown in FIGS. 66 and 68, and for conciseness and clarity of disclosure similar components will not be described in detail. In accordance with the present disclosure, the motor control system 4400 can be located within a housing of a powered surgical instrument. The motor control system 4400 comprises a first circuit portion 4401 operable at a first voltage level-supplied by the voltage source 4402 and a second circuit portion 4403 operable at a second voltage level 4412. In accordance with the present disclosure, the second voltage level 4412 may be different from the first voltage level supplied by the voltage source 4402. The voltage source 4402 of the powered surgical instrument supplies the first voltage level to power electronic/electrical components in the first circuit portion 4401, such as the control circuit 4404. A first conduction terminal 4407 of the transistor 4406 is coupled to the voltage source 4402. A second conduction terminal 4409 of the transistor 4406 is coupled to the motor 4410 via transmission/parasitic losses 4408. The control circuit 4404 is coupled to a control terminal 4405 of the transistor 4406. The motor control system 4400 comprises a first voltage monitor circuit 4414 coupled to the motor 4410 to measure the input voltage applied to the motor 4410. The voltage monitor circuit 4414 measures the voltage applied to the motor 4410. The motor control system 4400 comprises a second voltage monitor 4416 coupled to the second conduction terminal 4409 of the transistor 4406 to measure the voltage source 4402 voltage.

The differential voltage measured by the first and second voltage monitor circuits 4414, 4416 implement a current monitor to monitor the current supplied to the motor 4410. The control circuit 4404 receives a first voltage measured by the first voltage monitor circuit 4414 and a second voltage monitored by the second voltage monitor 4416 to derive a measured current to the motor 4410 and adjusts the current based on the measured current. In accordance with the present disclosure, the control circuit 4404 can monitor both current and voltage applied to the motor 4410. The control circuit 4404 stores an algorithm in memory and actively monitors both the voltage level supplied by voltage source 4402 as well as the second voltage level 4412 applied to the motor 4410 to provide the status of the current being drawn by the motor 4410. The differential voltage is measured across the transmission/parasitic losses 4408 which acts as a shunt for the purposes of monitoring current supplied to the motor 4410. This value could be characterized and calibrated during the manufacturing process against a known quantity to save the need to use a shunt resistor and therefore save costs. For example, the transmission/parasitic losses 4408 could be characterized during manufacturing and together with the differential voltage measured by the first and second voltage monitor circuits 4414, 4416 implement a current monitor to measure the current supplied to the motor 4410. The control circuit 4404 adjusts the current and voltage applied to the motor 4410 based on the monitored voltage and current.

During a surgical stapling procedure, a clinician may operate a surgical stapling instrument 5 to fire sequentially multiple staple cartridges along a selected tissue resection line to achieve a clinical outcome. For example, in a stomach resection procedure, the clinician may sequentially fire staple cartridges of different characteristics (e.g., size, color, type, length, staple height, staple diameter, staple size) along a selected tissue resection line to remove a portion of the stomach. In operation, the staple cartridges fire along the tissue resection line in an end-to-end arrangement.

The clinician may examine the tissue selected for resection using any suitable imaging technique such as, for example, x-ray, registered magnetic resonance imaging (MRI), and/or computerized tomography (CT) scan. The clinician may then select a suitable tissue resection line, and staple cartridges for sequential firing along the selected tissue resection line. Visual examination, however, has its limitations, and a tissue response to stapling can vary depending on many factors including, for example, patient age, tissue health, and/or tissue type. Moreover, tissue thickness and/or stiffness may vary along the selected tissue resection line, resulting in unexpected tissue responses.

Various methods, devices, and systems are provided for adaptively adjusting operational parameters of the surgical stapling instrument 5 during a staple cartridge firing based on a tissue response in an earlier phase/zone of the staple cartridge firing. Moreover, various methods, devices, and systems are provided for adaptively adjusting operational parameters of the surgical stapling instrument 5 during a staple cartridge firing based on a tissue response in one or more previous staple cartridge firings in a surgical procedure involving multiple sequential firings.

In accordance with the present disclosure, the processor 104 can execute various program instructions, which can be stored in a memory circuit such as the memory circuit 106, to implement various algorithms associated with firing a staple cartridge by the surgical stapling instrument 5. Such algorithms, e.g., thresholds, limits, triggers, conditions, pauses, and/or wait time, can be adjusted based on information learned from a tissue response in an earlier phase/zone of the staple cartridge firing, and/or based on a tissue response in one or more previous staple cartridge firings in a surgical procedure involving multiple sequential firings, as described in more detail below.

Parasitic losses occur within the circuitry of a surgical instrument. Parasitic losses can occur when power is consumed even when the device is powered off. Additionally, parasitic losses can occur when energy is lost during the powering of components in an electrical circuit. Momentary overcoming of the parasitic losses within the surgical instrument allows the instrument to minimize the diversion of power away from the motor and increase the power of the motor. The three main contributors to parasitic losses in power semiconductors are conduction, switching, and blocking (also known as reverse leakage). Conduction losses are the product of current flowing through an electrical component. The ripple current affects the power loss.

In accordance with the present disclosure, the surgical instrument may comprise a control circuit that can actively reduce its draw of power to enable the motor to use the full battery output. Battery powered surgical instruments have control electronics that are capable of operating a motor control circuit while reducing the power draw of the control electronics from the primary power source to such a level that the motor is capable of utilizing nearly all of the outputted power of the battery. The control electronics can have an integrated power accumulator and/or a boost circuit capable of providing the control circuit with a voltage level that is different from the voltage of the primary battery pack. Additionally, the control electronics can be selectively severable from the output power of the primary battery pack temporarily and re-coupleable. The electrical components can be temporarily removed from the primary power source.

In addition, optimization of power supply efficiency for a rated output current optimizes overall circuit efficiency and reduces circuit losses. A power supply with multiple converter circuits may be capable of specifying different loading conditions (voltage and current requirements). The converter circuits can be any of a buck, boost, or buck-boost converter circuit. LDO regulators are more efficient than power supplies at low loading situations. An LDO voltage regulator, for example, optimizes voltage output based on pre-determined loading conditions. The surgical device may switch from a power supply to an LDO regulator during low loads to increase battery life. In switching from one power source to another, additional capacitance holds the voltage rail until the switch is complete.

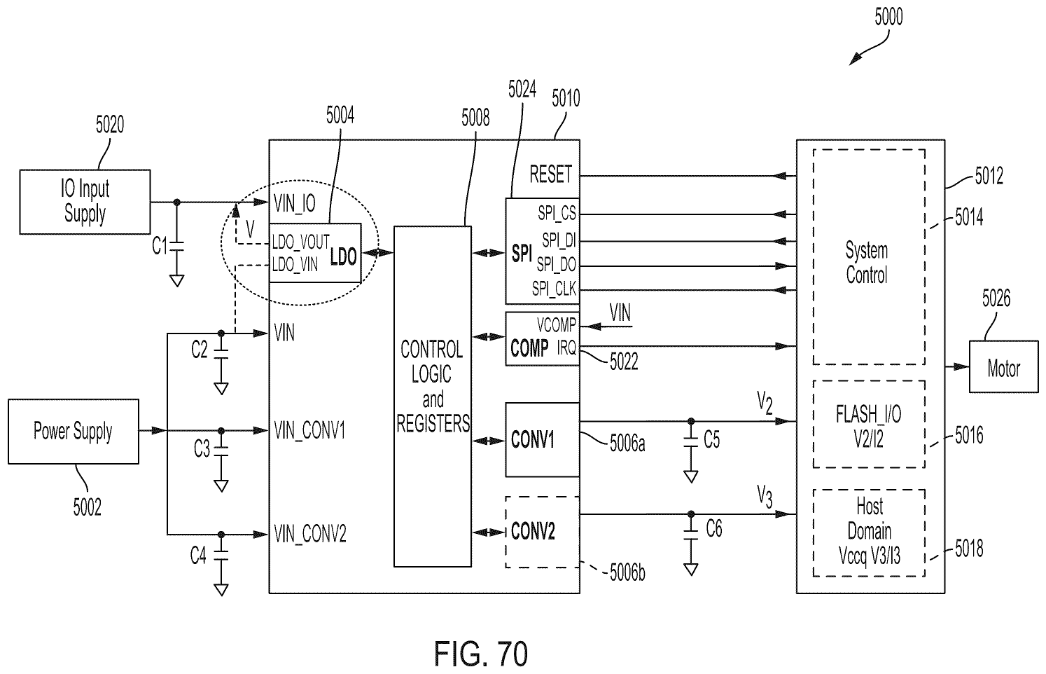

FIG. 70 is a circuit 5000, in accordance with the present disclosure. The circuit 5000 is part of a surgical instrument. The circuit 5000 can be part of the surgical stapling instrument 5 shown in FIGS. 1 and 4, for example. With reference now to FIGS. 1, 4, and 70, the surgical stapling instrument 5 may comprise the circuit 5000. The circuit 5000 comprises a power management circuit 5010 coupled to a control circuit 5012 to reduce or overcome parasitic losses of the surgical stapling instrument 5. The power management circuit 5010 is a power management circuit comprising two configurable, high-efficiency buck regulators for supplying variable voltages. The circuit 5000 reduces the electrical system parasitic losses and running losses including temporarily removing control electronics from the primary power source. The circuit 5000 can enable the motor 5026 to access the full capacity of the power source (e.g., a battery).

The power management circuit 5010 receives input power from a power supply 5002 and an Input/Output (IO) power supply 5020. In the example illustrated in FIG. 70, the power management circuit 5010 comprises a LDO regulator 5004, a plurality of converter circuits 5006a, 5006b, and a control logic and register circuit 5008 coupled between the LDO regulator 5004 and the plurality of converter circuits 5006a, 5006b. The converter circuits 5006a, 5006b can be any of a buck, boost, or buck-boost converter circuit. The power management circuit 5010 may comprise a single converter circuit 5006a. The power management circuit 5010 also comprises a comparator 5022 and a serial peripheral interface 5024 (SPI). The power management circuit 5010 operates cooperatively with the control circuit 5012 to optimize the supply voltage to the motor 5026 and low-power conditions and power saving modes via the SPI 5024. The power management circuit 5010 also supports the LDO regulator 5004 and a programmable interrupt comparator 5022 to monitor VIN. The motor 5026 is coupled to the power supplies of the power management circuit 5010 through the control circuit 5012.

The control circuit 5012 comprises a system control circuit 5114, a flash I/O circuit 5016, and a host domain circuit 5018. The control circuit 5012 communicates with the power management circuit 5010 through the SPI 5024 to control the control logic and register circuit 5008 to select the output voltages V2, V3 supplied by the converter circuit 5006a, 5006b, respectively. In accordance with the present disclosure, the output voltage V2 may be in a range from 1.1V to 3.6V at 1 A and the output voltage V3 is in a range from 0.7V to 1.335V at 1 A. Further, in accordance with the present disclosure, the LDO regulator 5004 may output voltage V1 of approximately 3.0V at 250 mA. The control circuit 5012 may comprise additional control electronics (not shown) that draw power from the power supply 5002. The control circuit 5012 can be one embodiment of the control circuit 100 shown in FIG. 4.

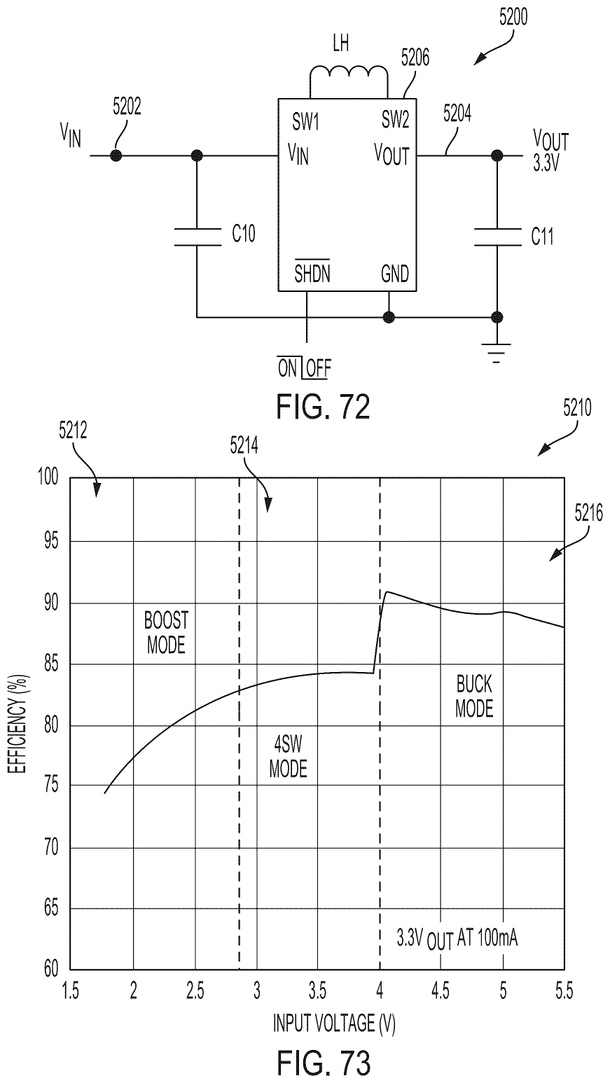

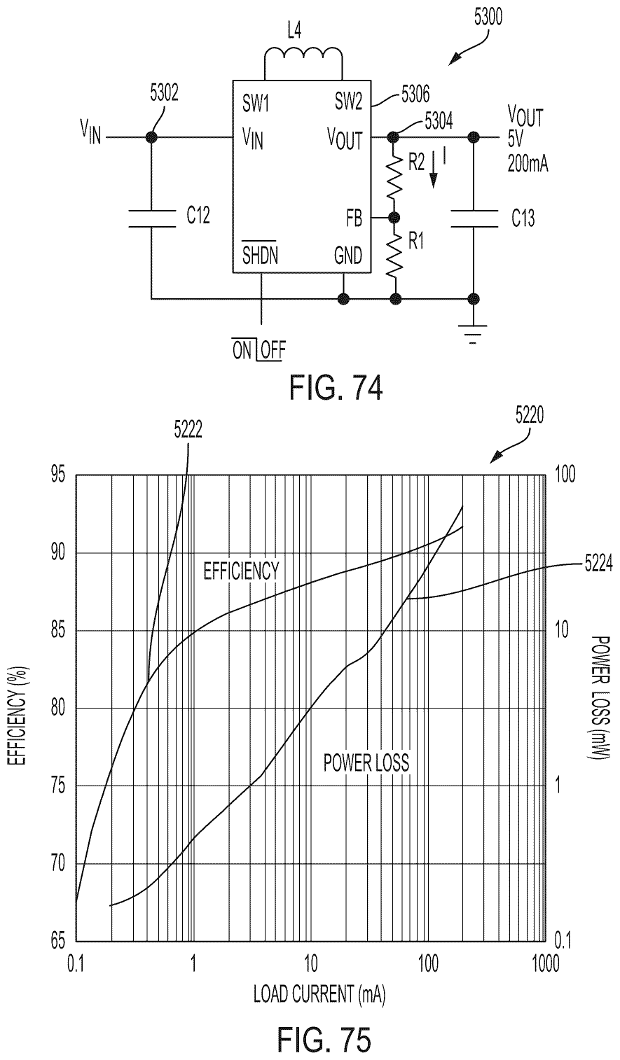

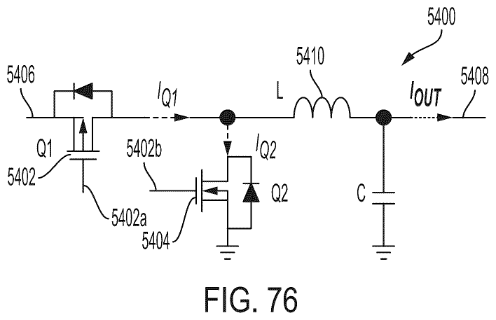

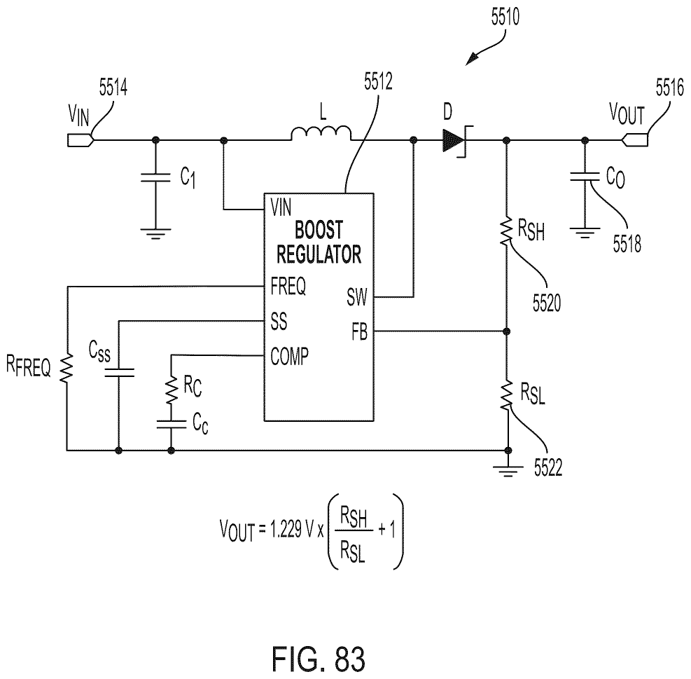

With continued reference to FIGS. 1, 4, and 70, the control circuit 5012 measures the load to the motor 5026. The control circuit 5012 measures the load to the motor 5026 with a voltage monitor (e.g., voltage sensor or voltmeter) and/or a current monitor (e.g., current sensor or ammeter) coupled between the motor 5026 and the power supply. The control circuit 5012 can couple a power source from one of the power supply 5002, the LDO regulator 5004, or the plurality of converter circuits 5006a, 5006b based on the load to the motor 5026 and can cause the motor 5026 to receive power from the selected source. The control circuit 5012 can couple either the regulator 5004 or the converter circuit 5006a based on a pre-determined voltage or current load applied to the motor. The motor 5026 in cooperation with the shaft 10 and the end effector 20 causes a surgical effect. The control circuit 5012 can determine the power source for the motor 5026 based in part on the load to the motor 5026. The selected power source is one of the power supply 5002, the LDO regulator 5004, or the plurality of converter circuits 5006a, 5006b. The load can be one of or both of voltage (V) load of the motor 5026 or a current load (I) of the motor 5026. In accordance with the present disclosure, the converter circuits 5006a, 5006b can be any of a buck (shown in FIG. 76), boost (shown in FIG. 83), or buck-boost (shown in FIGS. 72 and 74) converter circuit.

The control circuit 5012 stores the operating range of the power supply 5002, the LDO regulator 5004, and the plurality of converter circuits 5006a, 5006b. In accordance with the present disclosure, the expected operating range of the power supply 5002, the LDO regulator 5004, and the plurality of converter circuits 5006a, 5006b may dictate the selection of the power source. The operating range includes at least one of the voltage (V) and current (I) output of each power source.

The control circuit 5012 stores the efficiencies of each selectable power source, for example, the power supply 5002, the LDO regulator 5004, and the plurality of converter circuits 5006a, 5006b. In addition, the control circuit 5012 stores the voltage (V) and current (I) output of each power source. The control circuit 5012 may determine the power source based in part on the voltage (V) and current (I) output of each of the selectable power sources.

In accordance with the present disclosure, the control circuit 5012 can couple the power sources of the power management circuit 5010, such as for example, the LDO regulator 5004 and the plurality of converter circuits 5006a, 5006b selected by the control circuit 5012, to the motor 5026. Where at least one of the converter circuits 5006a, 5006b is a boost converter circuit, the control circuit 5012 can be configured to couple the boost converter circuit to the motor 5026 based on the voltage load at the motor 5026 exceeding the pre-determined voltage load. Additionally, where at least one of the converter circuits 5006a, 5006b is a boost converter circuit, the control circuit 5012 can be configured to determine a voltage sag at the motor 5026 and can couple the voltage boost converter circuit to the motor 5026. Alternatively, where at least one of the converter circuits 5006a, 5006b is a buck converter circuit, the buck converter circuit 5012 can be selected based on the current exceeding the pre-determined current load. The regulator 5004 can be a low dropout (LDO) regulator, and the control circuit 5012 can couple the motor 5026 to the LDO regulator based on the voltage load of the motor 5026 being below the pre-determined voltage load.

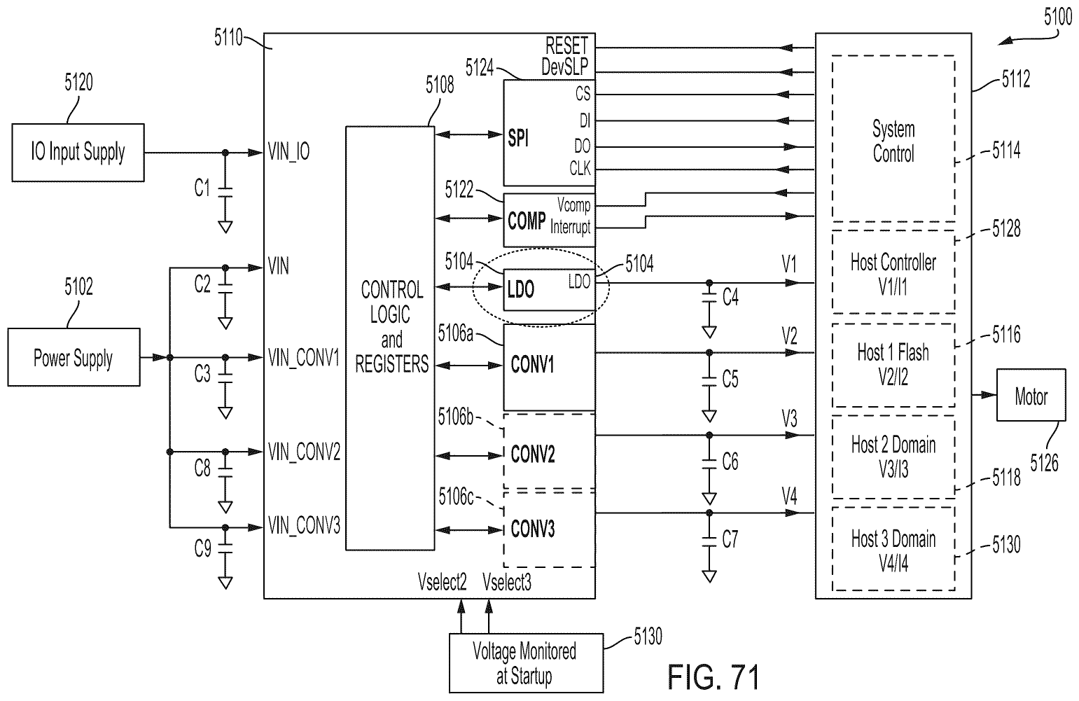

FIG. 71 is a circuit 5100 in accordance with the present disclosure. The circuit 5100 can be part of the surgical stapling instrument 5 shown in FIGS. 1 and 4, for example. With reference now to FIGS. 1, 4, and 71, the surgical stapling instrument 5 may comprise the circuit 5100. The circuit comprises a power management circuit 5110 coupled to a control circuit 5112 to reduce or overcome parasitic losses of the surgical stapling instrument 5. The power management circuit 5110 is an advanced power management unit comprising three configurable, high-efficiency buck regulators for supplying variable voltages. The circuit 5100 reduces the electrical system parasitic losses and running losses including temporarily removing control electronics from the primary power source. The circuit 5100 can enable the motor 5126 to access the full capacity of the power source (e.g., a battery).

Similar to the power management circuit 5010 described in connection with FIG. 70, the power management circuit 5110 shown in FIG. 71 receives input power from a power supply 5102 and an I/O power supply 5120. The power management circuit 5110 comprises a LDO regulator 5104, a plurality of converter circuits 5106a, 5106b, 5106c, and a control logic and register circuit 5108 coupled between the LDO regulator 5104 and the plurality of converter circuits 5106a, 5106b, 5106c. The converter circuits 5106a, 5106b, 5106c can be any of a buck (shown in FIG. 76), boost (shown in FIG. 83), or buck-boost (shown in FIGS. 72 and 74) converter circuit. The power management circuit 5510 may comprise a single converter circuit 5106a. The power management circuit 5110 also comprises a comparator 5122 and a SPI 5124. The power management circuit 5110 operates cooperatively with the control circuit 5112 to optimize the supply voltage to the motor 5126 and low-power conditions and power saving modes via the SPI 5124. The power management circuit 5110 also supports the LDO regulator 5104 and a programmable interrupt comparator 5122 to monitor VIN. The motor 5126 is coupled to the power supplies of the power management circuit 5110 through the control circuit 5112.

The control circuit 5112 comprises a system control circuit 5114, a host controller 5128, a host-1 flash I/O circuit 5116, a host-2 domain circuit 5118, and a host-3 domain circuit 5130. The control circuit 5112 communicates with the power management circuit 5110 through the SPI 5124 to control the control logic and register circuit 5108 to select the output voltages V2, V3, V4 supplied by the converter circuit 5106a, 5106b, 5106c, respectively. In accordance with the present disclosure, the output voltage V2 may be in a range from 1.1V to 3.6V at 1.6 A, the output voltage V3 is in a range from 1.1V to 3.6V at 1 A, and the output voltage V3 is in a range from 0.7V to 1.335V at 1 A. Further, in accordance with the present disclosure, the LDO regulator 5104 may output voltage V1 at approximately 1.2V to 3.1V at up to 250 mA. The control circuit 5112 may comprise additional control electronics (not shown) that draw power from the power supply 5102. The control circuit 5112 can be one embodiment of the control circuit 100 shown in FIG. 4.

With continued reference to FIGS. 1, 4, and 71, the control circuit 5112 measures the load to the motor 5126. The control circuit 5112 measures the load to the motor 5126 with a voltage monitor (e.g., voltage sensor or voltmeter) and/or a current monitor (e.g., current sensor or ammeter) coupled between the motor 5016 and the power supply. The control circuit 5112 can select a power source from one of the power supply 5102, the LDO regulator 5104, or the plurality of converter circuits 5106a, 5106b, 5106c based on the load and can cause the motor to receive power from the selected source. Additionally, the control circuit 5012 can couple either the regulator 5004 or the converter circuit 5006a based on a pre-determined voltage or current load applied to the motor. The motor 5126 in cooperation with the shaft 10 and the end effector 20 causes a surgical effect. The control circuit 5112 can determine the power source for the motor 5126 based in part on the load to the motor 5126. The selected power source is one of the power supply 5102, the LDO regulator 5104, or the plurality of converter circuits 5106a, 5106b, 5106c. The load can be one of or both of voltage (V) load of the motor 5126 or a current (I) load of the motor. The converter circuits 5106a, 5106b, 5106c can be any of a buck, boost, or buck-boost converter circuit.

The control circuit 5112 stores the operating range of the power supply 5102, the LDO regulator 5104, and the plurality of converter circuits 5106a, 5106b, 5106c. In accordance with the present disclosure, the selection of the power source may be based on the expected operating range of the power supply 5102, the LDO regulator 5104, and the plurality of converter circuits 5106a, 5106b, 5106c. The operating range includes at least one of the voltage and current output of each power source.

The control circuit 5112 stores the efficiencies of each selectable power source, for example, the power supply 5102, the LDO regulator 5104, and the plurality of converter circuits 5106a, 5106b, 5106c. In addition, the control circuit 5112 stores the voltage and current output of each power source. The control circuit 5112 can determine the power source based in part on the voltage (V) and current (I) output of each of the selectable power sources.

In accordance with the present disclosure, the control circuit 5112 can couple the power sources of the power management circuit 5110, such as for example the LDO 5104 and the plurality of converter circuits 5106a, 5106b, 5106c selected power source by the control circuit 5112, to the motor 5126. Where at least one of the converter circuits 5106a, 5106b, 5106c is a boost converter circuit, the control circuit 5112 can be configured to couple the boost converter circuit to the motor 5126 based on the voltage load at the motor 5126 exceeding the pre-determined voltage load. Further, where at least one of the converter circuits 5106a, 5106b, 5106c is a boost converter circuit, the control circuit 5112 can be configured to determine a voltage sag at the motor 5126 and couple the voltage boost converter circuit to the motor 5126. Where at least one of the converter circuits 5106a, 5106b, 5106c is a buck converter circuit, the buck converter circuit 5112 may be selected based on the current exceeding the pre-determined current load. The regulator 5104 can be a low dropout (LDO) regulator, and the control circuit 5112 may couple the motor 5126 to the LDO regulator based on the voltage load of the motor 5126 being below the pre-determined voltage load.

The converter circuits 5006a, 5006b described in connection with FIG. 70 and the converter circuits 5106a, 5106b, 5106c described in connection with FIG. 71 can be implemented as the buck-boost converter circuits 5200, 5300 shown in FIGS. 72 and 74, respectively. The buck-boost converter circuits 5200, 5300 are described hereinbelow. FIG. 73 is graphical representation of the operating modes of the control circuit 100, 5012, 5112 shown in FIGS. 4, 70, and 71 selected based on the efficiencies shown in FIG. 75.

FIGS. 72 and 74 illustrate examples of DC/DC converter type buck-boost converter circuits 5200, 5300. The buck-boost converter circuits 5200, 5300 operate from input voltages above, below, or equal to the output voltage. The buck-boost converter circuit 5200 may comprise an input 5202 from a voltage source (not shown) at a first voltage level, a buck-boost DC/DC converter 5206, and an output 5204 at a second voltage level. The buck-boost converter circuit 5200, 5300 can be any one of the converter circuits (5006a, 5006b, 5106a, 5106b, 5106c) of FIGS. 70 and 71

In accordance with the present disclosure, the output 5204 can be coupled to a motor (e.g., the motor 5026, 5126 shown in FIGS. 70-71). The buck-boost converter circuit 5200 outputs the second voltage level to the motor. The second voltage level is controllable by the control circuit (e.g., control circuit 100, 5012, 5112 shown in FIGS. 4, 70, and 71). The control circuit controls the second voltage level applied to the motor. The first voltage level can be different from the second voltage level.

In accordance with the present disclosure, the buck-boost converter circuit 5300 shown in FIG. 74 can be similar to the buck-boost converter circuit 5200 shown in FIG. 72. The buck-boost converter circuit 5300 comprises an input 5302 from a voltage source VIN at a first voltage level, a DC/DC converter type buck-boost converter circuit 5306, and an output 5304 at a second voltage level. The buck-boost converter circuits 5200, 5300 are configured for different input voltage ranges. As shown in FIG. 74, the buck-boost converter circuit 5300 is loaded by R2 and R1 such that there is current (I) through the output 5304 terminal. For example, the buck-boost converter circuits 5200, 5300 are part of surgical stapling instrument 5 shown in FIGS. 1 and 4.

FIG. 73 is a graphical illustration of operational modes of an example DC/DC converter type buck-boost converter circuit such as the unloaded buck-boost converter circuit 5200 shown in FIG. 72, in accordance with the present disclosure. Three modes 5212, 5214, 5216 are depicted in a graph 5210, which represents EFFICIENCY (%) (along the vertical axis) as a function of INPUT VOLTAGE (V) (along the horizontal axis) applied to the buck-boost converter circuit 5200. For example, the boost mode 5212 occurs when the input voltage is approximately 400 mV below the output voltage. The buck mode 5216 occurs when the input voltage is approximately 800 mV above the output voltage. In the 4-Switch mode 5214, the input voltage is between the voltage for the boost mode 5212 and the buck mode 5216. The different modes are based on the input voltage level and the desired output voltage level (second voltage level). The modes vary in efficiency, with buck mode 5216 having the highest efficiency.

By way of example and with reference back to FIGS. 70 and 71, the control circuits 5012, 5112 determine the output voltage to the motor 5026, 5126. The operating modes of the converter circuits 5006a, 5006b, 5106a, 5106b, 5106c are based on the output voltage. The control circuits 5012, 5112 store the operating efficiencies of each operating mode for each of the converter circuits 5006a, 5006b, 5106a, 5106b, 5106c. The control circuit 5012, 5112 chooses the buck-boost converter circuit based on the output voltage and the operating efficiency of the operating mode of the converter circuit for that output voltage. Once the control circuit 5012, 5112 selects the operating mode for a converter circuit, the control circuit 5012, 5112 communicates to the control logic and register circuit 5008, 5108 via the SPI 5024, 5124 of the power management circuit 5010, 5110 to select the desired power supply output for the motor 5026, 5126 and other circuits as described above. For example, the control circuit 5012 may select one of the converter circuits 5006a, 5006b for connection to the motor 5026. For example, the control circuit 5112 may select one of the converter circuits 5106a, 5106b, 5106c to be connected to the motor 5126.

FIG. 75 is a graphical illustration of power loss and efficiency at different load currents for a buck-boost DC/DC converter type buck-boost converter circuit such as the loaded buck-boost converter circuit 5300 shown in FIG. 74, in accordance with the present disclosure. The graph 5220 illustrates EFFICIENCY (%) (along the left vertical axis) as a function of LOAD CURRENT (mA) (along the horizontal axis) and POWER LOSS (mW) (along the right vertical axis) as a function of LOAD CURRENT (mA). As shown, both efficiency 5222 and power loss 5224 increase as the load current increases.

By way of example and with reference back to FIGS. 70 and 71, the control circuits 5012, 5112 measure the load current to determine the efficiency and power loss of the converter circuits 5006a, 5006b, 5106a, 5106b, 5106c. The control circuits 5012, 5112 store the profile of power loss and efficiency for different load currents for each converter circuit 5006a, 5006b, 5106a, 5106b, 5106c. The control circuits 5012, 5112 can store the efficiency and power loss for different load currents for each converter circuit 5006a, 5006b, 5106a, 5106b, 5106c. The control circuits 5012, 5112 determine the power source based on at least one of the power loss, efficiency, or load current. By way of example and with reference back to FIG. 73, the control circuits 5012, 5112 can determine the efficiency based on the stored operating efficiencies of each of the modes for each converter circuits 5006a, 5006b, 5106a, 5106b, 5106c.

FIG. 76 illustrates a basic topology of a buck converter circuit 5400 in accordance with this disclosure. The buck converter circuit 5400 can be any one of the converter circuits (5006a, 5006b, 5106a, 5106b, 5106c) of FIGS. 70 and 71 The buck converter circuit 5400 has both inductor conduction losses and transistor conduction losses. The buck converter circuit 5400 comprises a first transistor 5402 (Q1) and a second transistor 5404 (Q2) coupled between an input 5406 and an output 5408 of the buck converter circuit 5400. A first current (shown in FIG. 78) flows through the first transistor 5402. A second current (shown in FIG. 79) flows through the second transistor 5404.

The input 5406 is coupled to a power source, such as the battery for the surgical instrument. The output 5408 can be coupled to the motor. The control circuit 5012, 5112 (shown in FIGS. 70 and 71) controls the level of the output voltage of the buck converter circuit 5400. The control circuit 5012, 5112 controls the transistors 5402 (Q1), 5404 (Q2) to change the output current I_{OUT} of the buck converter circuit 5400. The control circuit 5012, 5112 applies control signals to the inputs 5402a, 5402b of the transistors 5402 (Q1), 5404 (Q2) to control the current I_{Q1} output by the first transistor 5402 (Q1) and the current I_{Q2} output by the second transistor 5404 (Q2) to control the output current I_{OUT} of the buck converter circuit 5400.

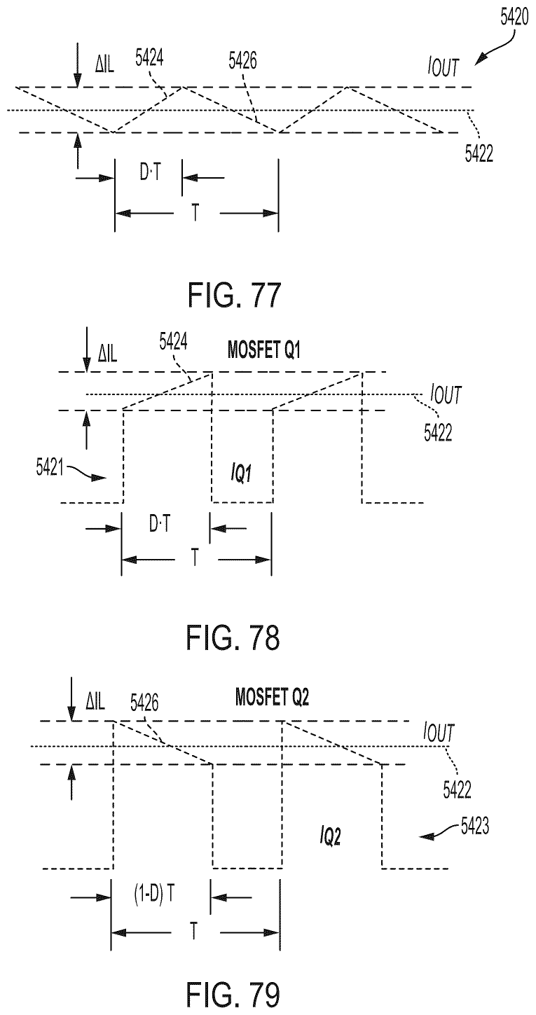

FIG. 77 illustrates the output current of the buck converter circuit 5400 I_{OUT} shown in FIG. 76. FIG. 78 illustrates the current I_{Q1} flowing through the first transistor 5402 (Q1) in FIG. 76. FIG. 79 illustrates the current I_{Q2} flowing through the second transistor 5404 (Q2) in FIG. 76. The current I_{Q1} flowing through the first transistor 5402 (Q1) and the current I_{Q2} flowing through the second transistor 5404 (Q2) produce the ripple current 5420 flowing through the inductor 5410 (L). The output current I_{OUT} 5422 resulting from the inductor L is applied to the motor (e.g., motor 5026, 5126 shown in FIGS. 70 and 71) through the output 5408.

The control circuit 5012, 5112 (shown in FIGS. 70 and 71) switches the first transistor 5402 (Q1) and the second transistor 5404 (Q2) on and off. For example, the control circuit 5012, 5112 turns on the first transistor 5402 (Q1) and turns off the second transistor 5404 (Q2). In the on state, while the first transistor 5402 (Q1) switch is conducting, the current through the inductor 5410 begins increasing linearly, as shown by the slope 5424 (FIGS. 77 and 78) of the first transistor 5402 (Q1) current 5421 (I_{Q1}). At the end of the duty cycle, the control circuit 5012, 5112 switches the first transistor 5402 (Q1) off.

The control circuit 5012, 5112 (shown in FIGS. 70 and 71) switches the second transistor 5404 (Q2) on. At this time, the inductor 5410 (L) continues conducting current and the second transistor 5404 (Q2) conducts current 5423 (I_{Q2}) (FIG. 79). The current through the inductor 5410 (L) begins decreasing linearly as shown by the slope 5426 (FIG. 79). The buck converter circuit waveforms representations of the ripple current 5420, 5421, 5423 shown in FIGS. 77-79 depict this switching action. The ripple current 5420 through the inductor 5410 (L) is shown in FIG. 77. As described above, I_{OUT} is the output current 5422 (FIGS. 77-79) through the output 5408 of the buck converter circuit 5400. For example, the output current 5422 is supplied to the motor of the surgical stapling instrument 5 shown in FIGS. 1 and 4.

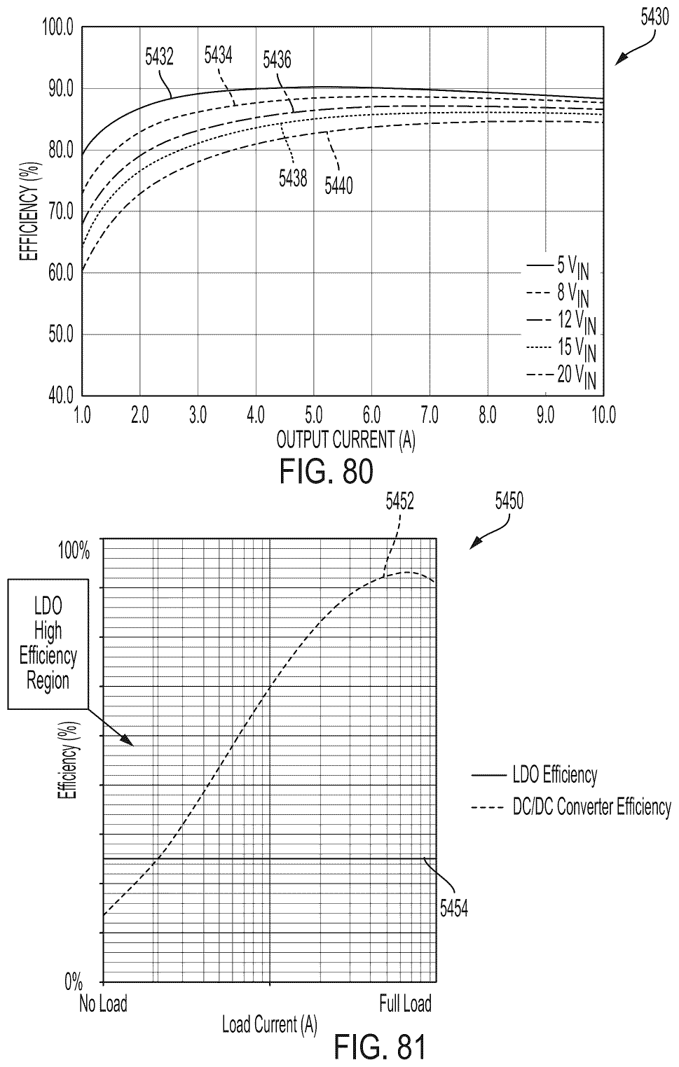

FIG. 80 is a graph 5430 of EFFICIENCY (%) (along the vertical axis) as a function of OUTPUT CURRENT (A) (along the horizontal axis) over a range of output currents of the buck converter circuit 5400 shown in FIG. 76 at discrete voltage values. The graph 5430 shows efficiency of the buck converter circuit 5400 for different input voltage values 5432 (5V), 5434 (8V), 5436 (12V), 5438 (15V), and 5440 (20V) over a range of output current I_{OUT} (1.0 A-10.0 A). As shown by the various plots, the efficiency is greater at lower input voltages. The control circuit 5012, 5112 (shown in FIGS. 70 and 71) stores similar efficiency profiles.

FIG. 81 is a graph 5450 of Efficiency (%) (along the vertical axis) as a function of Load Current (A) over a range of load currents for a LDO regulator, such as the LDO regulator 5004, 5104 (FIGS. 70 and 71) and a DC/DC converter type buck-boost converter circuits 5200, 5300 (FIGS. 72 and 74). As shown in FIG. 81, the efficiency 5452 of the DC/DC converter type buck-boost converter circuits 5200, 5300 increases with load current, whereas the efficiency 5454 of the LDO regulator 5004, 5104 does not change with load current. The efficiency of the LDO regulator 5004, 5104 is greater than the efficiency of the DC/DC converter type buck-boost converter circuits 5200, 5300 at low load currents. The control circuit 5012, 5112 (FIGS. 70 and 71) stores the efficiency of the LDO regulator 5004, 5104 (FIGS. 70 and 71), the converter circuits 5006a, 5006b, 5106a, 5106b (FIGS. 70 and 71), and the DC/DC converter type buck-boost converter circuits 5200, 5300 (FIGS. 72 and 74). The control circuit 5012, 5112 employs the stored efficiencies to determine which power supply is more efficient based on the present load condition of the motor 5026, 5126 (FIGS. 70 and 71). Accordingly, the control circuit 5012, 5112 may employ a current level operating range switching technique to optimize the power supply for its expected operating range to increase the efficiency. Accordingly, depending on the range, a DC/DC converter circuit may be more appropriate than an LDO regulator. Switching losses that occur in the circuits 5000, 5100 (FIGS. 70 and 71) of the surgical stapling instrument 5 (FIGS. 1-4) need to be minimized. To minimize the switching losses of the circuits 5000, 5100 of the surgical stapling instrument 5, the control circuits 5012, 5112 control the turn on/turn off times and the rise/fall times of the transistors of the converter circuits 5006a, 5006b, 5106a, 5106b, 5106c to minimize the switching circuitry losses. The control circuit 5012, 5112 controls the switching frequency to be above the human audible range.

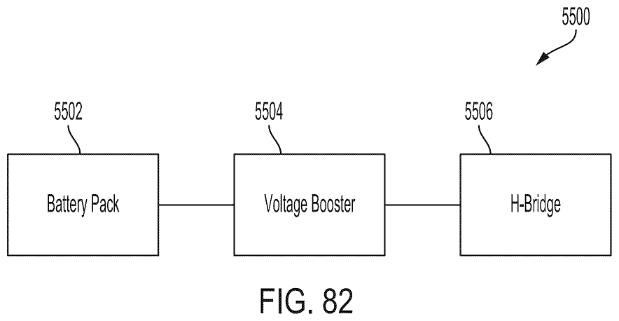

FIG. 82 illustrates a surgical system 5500, in accordance with the present disclosure. The surgical system 5500 is a block diagram of the surgical stapling instrument 5 shown in FIGS. 1-4. The surgical system 5500 comprises a battery pack 5502, an H-bridge 5506, and a voltage boost converter circuit 5504 coupled between the battery pack 5502 and the H-bridge 5506. Inserting the voltage boost converter circuit 5504 between the battery pack 5502 and the H-bridge 5506 may allow the surgical system 5500 to operate at lower voltages, however, the switching efficiency creates system losses. The surgical system 5500 can provide additional voltage to the voltage supplied by the battery pack 5502 to increase the voltage applied to the motor 5026, 5126 (FIGS. 70 and 71). The control circuit 5012, 5112 (FIGS. 70 and 71) can determine if the voltage boost converter circuit 5504 provides additional voltage to the H-bridge 5506. The control circuit 5012, 5112 determines which voltage boost converter circuit can provide additional voltage to the motor 5006, 5126 (FIGS. 70 and 71). Different types of voltage boost converter circuits 5504 are disclosed herein.

FIG. 83 illustrates an example implementation of a voltage boost converter circuit 5510, in accordance with the present disclosure. The voltage boost converter circuit 5510 can be any one of the converter circuits (5006a, 5006b, 5106a, 5106b, 5106c) of FIGS. 70 and 71. The voltage boost converter circuit 5510 produces an output voltage 5516 (V_{OUT}) based on an input voltage 5514 (VIN) that is selectively coupleable to the motor 5026, 5126 (FIGS. 70 and 71). Increasing the capacitance of the output capacitor 5518 (C_{O}) reduces the effort required by the voltage boost converter circuit 5510 to maintain the output voltage 5516 (V_{OUT}) under current loaded conditions. The voltage boost converter circuit 5510 can regulate the output voltage 5516 (V_{OUT}) using current-mode, pulse-width modulation (PWM) control, for example. The voltage boost converter circuit 5510 receives the input voltage 5514 (VIN) and the boost regulator 5512 outputs the output voltage 5516 (V_{OUT}). The control circuit 5012, 5112 (FIGS. 70 and 71) controls the boost regulator 5512 to set the output voltage 5516 (V_{OUT}).

The voltage boost converter circuit 5510 can maintain the output voltage 5516 (V_{OUT}) under high current loads from the motor 5026, 5126 (FIGS. 70 and 71). The control circuit 5012, 5112 (FIGS. 70 and 71) determines motor 5026, 5126 load is greater than a pre-determined threshold and couples the voltage boost converter circuit 5510 output to the motor 5026, 5126. This provides additional voltage to the motor 5026, 5126 to overcome any voltage sag that occurs due to load conditions.

The voltage boost converter circuit 5510 provides several advantages. For example, under heavy loading conditions of the battery pack, such as for example the battery pack 5502 shown in FIG. 82, the battery pack voltage sags considerably. Under considerable voltage sag, the electrical system of the surgical stapling instrument 5 (FIGS. 1-4) can go into a brown out condition. The voltage boost converter circuit 5510 can additionally boost the system voltage to maintain the desired voltage to the motor 5026, 5126 (FIGS. 70 and 71). The voltage boost converter circuit 5510 can be very efficient under high loading conditions.

For example, the electrical system of the surgical stapling instrument 5 (FIGS. 1-4) can incorporate an additional boost regulator that is isolated from the motor 5026, 5126 (FIGS. 70 and 71) subsystem only. This prevents sagging of the voltage applied to the motor. By minimizing the voltage sag, the circuit 5000, 5100 (FIGS. 70 and 71) of the surgical stapling instrument 5 can supply more power to the motor 5026, 5126 based on the desired motor operating curve. The voltage boost converter circuit 5510 can boost the nominal 12V system voltage to a higher voltage level.

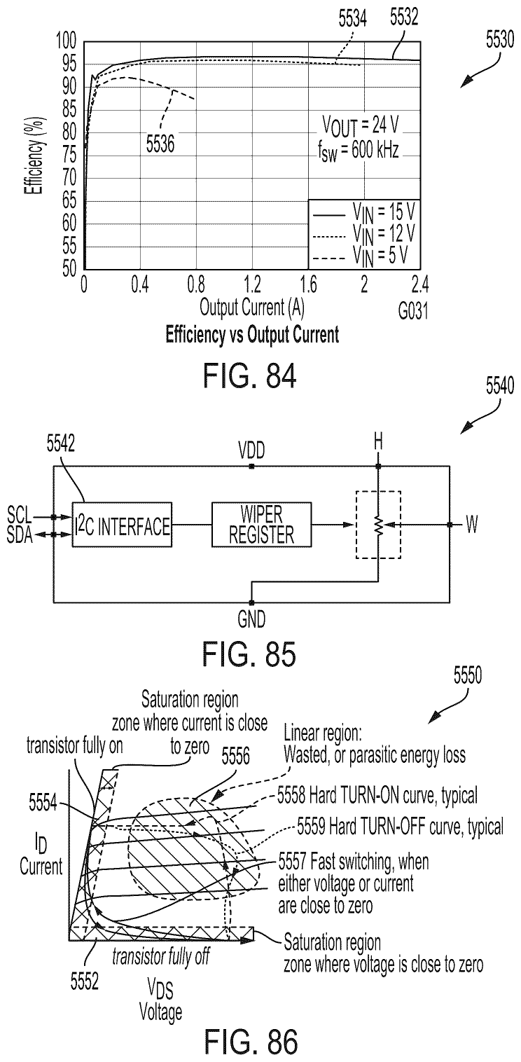

FIG. 84 is a graph 5530 illustrating efficiency (%) as a function of output current (A) for a DC/DC boost regulator. The graph 5530 shows the efficiency of a DC/DC boost regulator at different input voltage (V_{OUT}) levels over a range of output currents. The 5V input voltage 5536 is the least efficient. The 15V input voltage 5532 is more efficient than 12V input voltage 5534.

FIG. 85 is a variable potentiometer 5540, in accordance with the present disclosure. The variable potentiometer 5540 is controlled by the input received at the serial interface circuit 5542 (e.g., an I²C interface circuit) from the control circuit 5012, 5112 (FIGS. 70 and 71). The received input controls wiper registers 5544 to control the potentiometer 5546 and change the resistance of the potentiometer 5546.

In accordance with the present disclosure, the variable resistor 5540 can be adjustable in “real” time based on the system performance by the control circuit 5012, 5112 (FIGS. 70 and 71). The variable potentiometer 5540 can be used as the first resistor 5520 (R_{SH}) and/or the second resistor 5522 (R_{SL}) of the voltage boost converter circuit 5510 (FIG. 83). The resistance of the first resistor 5520 (R_{SH}) and/or the second resistor 5522 (R_{SL}) are then set by the control circuit 5012, 5112 to vary the output voltage 5516 (V_{OUT}) of the voltage boost converter circuit 5510 and thus set the speed of the motor 5026, 5126 (FIGS. 70 and 71).

In accordance with the present disclosure, motor load feedback can be a measured parameter and the resistance values of at least one of the first resistor 5520 (R_{SH}) and the second resistor 5522 (R_{SL}) (FIG. 83) can be adjusted based on at least the load on the motor 5026, 5126 (FIGS. 70 and 71). The potentiometer 5546 can be a dynamic active resistor. The dynamic resistor changes the resistance electronically.

In accordance with the present disclosure, the control circuit 5012, 5112 (FIGS. 70 and 71) can control the converter circuits to dynamically change speed of the motor. Further, the control circuit can switch between static boost circuits to change the speed of the motor. The converter circuits 5006a, 5006b, 5106a, 5106b, 5106c (FIGS. 70 and 71), and specific implementations of such circuits described above, can be controlled by the control circuit 5012, 5112 to lower the voltage applied to the motor 5026, 5126 (FIGS. 70 and 71).

FIG. 86 is a graph 5550 of drain current (I_{D}) as a function of drain-to-source voltage (V_{DS}) for switching a field effect transistor, in accordance with the present disclosure. For MOSFETs, IGBTs, and other transistors, moving these components out of their linear zones as quickly as possible saves energy because the time it takes for the transistor to turn on is all wasted or is a parasitic loss. The graph 5550 illustrates a first saturation region 5552 where the voltage is close to zero. The transistor is off in the first saturation region 5552. The graph 5550 also illustrates a second saturation region 5554 where the current is close to zero. The transistor is on in the second saturation region 5554. The graph 5550 also illustrates a linear region 5556.

Driving the transistor hard out of the linear region and into the saturation region quickly saves energy. In addition, switching between the linear and saturation states can be done as fast as possible to minimize losses. The graph 5550 illustrates the hard turn on curve 5558 and the hard turn off curve 5559. The control circuit 5012, 5112 (FIGS. 70 and 71) controls the speed at which the transistor is switched between the linear and saturation states to minimize losses.

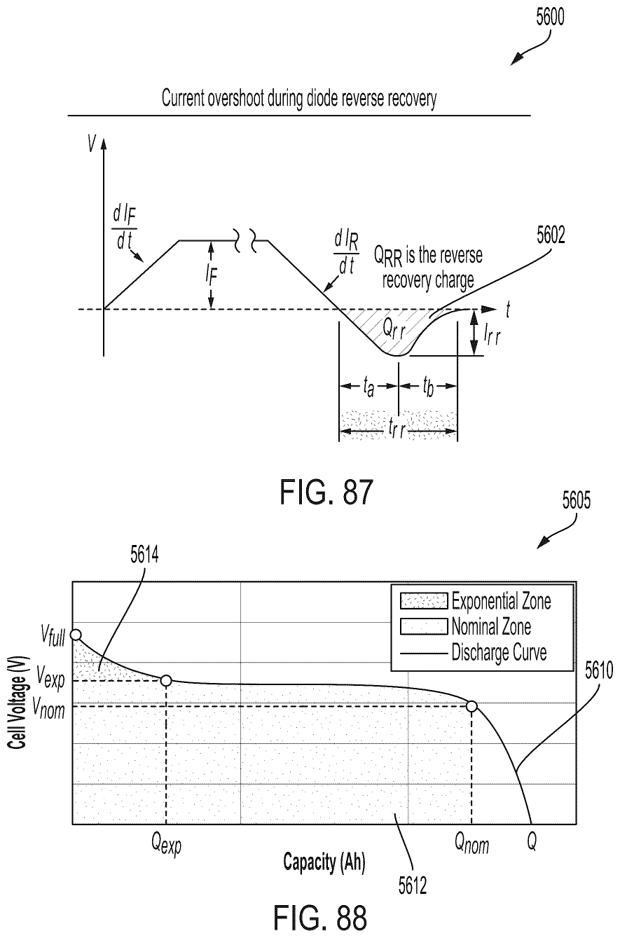

FIG. 87 is a graphical depiction of leakage current losses, in accordance with the present disclosure. The graph 5600 depicts current overshoot during diode reverse recovery period. The graph 5600 shows voltage (V) as a function of time (t) where Q_{RR} is the reverse recovery charge 5602 over period tᵣᵣ. For example, operating an integrated circuit near or above the maximum operating temperature and/or voltage increases the leakage current. The control circuit 5012, 5112 (FIGS. 70 and 71) can control the operation of an integrated circuit to lower the operating temperature based on a measured operating temperature. A temperature sensor can measure the operating temperature of the integrated circuit.

In accordance with the present disclosure, the use of fast recovery diodes may improve switching losses, but increases conduction losses. To reduce losses, the components of the overall electrical circuitry that are not in use should thus be put in a sleep state or turned off. These small currents can add up to a substantial “parasitic” current draw of the power source. The graph 5600 of reverse recovery illustrates that the reverse recovery charge 5602 is based on the current and the required recovery time.

FIG. 88 is a graph 5605 of a battery discharge curve 5610, in accordance with the present disclosure. The graph 5605 depicts the Cell Voltage (V) as a function of Capacity (Ah). The battery discharge curve 5610 illustrates the voltage drop of a battery over the amp hours that the battery is used. The exponential zone 5614 represents the exponential voltage drop of the battery from full voltage to exponential voltage level. The nominal zone 5612 represents the area where the voltage drops from the exponential voltage to the nominal voltage.

FIG. 88 will now be described together with FIGS. 1-4, 71 and 82. In accordance with the present disclosure, to maintain peak power to the motor system, maintaining a maximized potential voltage is a key parameter. As the cell capacity of a battery decreases, the cell voltage also trends downward. Thus, the power applied to the motor is affected by the amp-hours (Ah) that the battery has been used for. Motor power is the product of voltage and current. Battery voltage reduction has a direct impact on the power the system can supply to the motor.

For example, the surgical stapling instrument 5 (FIGS. 1-4) can employ Lithium batteries as its power source. These cells have a capacity of approximately 1500 mAh with a nominal voltage of 3V per cell. The battery pack, such as the battery pack 5502 (FIG. 82), is able to deliver all the required power for up to 12 hours. The current set up requires approximately 50 mA to run. At 12 hours of idle, the system has consumed 50 mA×12 hours=600 mAh. This equates to consuming 40% of the battery capacity in an idle or non-productive manner.

With reference to FIGS. 70 and 71, to minimize these losses, reductions in parasitic losses and power management techniques are required. As previously discussed, to reduce losses, components of the overall electrical circuitry that are not in use should thus be put in a sleep state or turned off. These small currents can add up to a substantial “parasitic” draw of the power source. The circuit 5000, 5100 may comprise a power management circuit 5010, 5110. The circuit 5000, 5100 controls the power to the non-therapeutic tissue sensing circuits.

Further, in accordance with the present disclosure, the power management circuit 5010, 5110 can monitor a sensor or circuit element to determine when to turn on and off the surgical stapling instrument 5 (FIGS. 1-4) and the circuit elements to be energized. The sensor can be at least one of a gyroscope, accelerometer, thermal sensor, pressure sensor, or any other type of sensor to determine activity of the surgical stapling instrument 5 or a timer for a surgical stapling instrument 5 timeout after a pre-determined amount of time without activity. Activity of the surgical stapling instrument 5 includes picking up the surgical stapling instrument 5. The activity is determined by the gyroscope or accelerometer. A user's hand on the surgical stapling instrument 5 can be detected by a pressure or temperature sensor. The orientation of the surgical stapling instrument 5 can be determined by the gyroscope. The motion of the surgical stapling instrument 5 can be determined by the accelerometer. Accordingly, any indication that the surgical stapling instrument 5 is going to be used by a user can be detected by a suitable sensor.

In accordance with the present disclosure, the power management circuit 5010, 5110, under control of the control circuit 5012, 5112, may engage or disengage unused electrical components. For example, the power management circuit is to engage circuit components when the device determines an activity has occurred. The power management circuit is to couple the electrical components of the circuit to a power source to engage the surgical stapling instrument 5. The power management circuit 5010, 5110 disengages when the surgical stapling instrument 5 determines that there has been a lack of activity for a pre-determined amount of time or that the activity has ended. Disengaging may be severing a connection between the unused electrical components and the power source.

In accordance with the present disclosure, the end of activity can be determined by the orientation of the surgical stapling instrument 5 (FIGS. 1-4), the lack of movement of the surgical stapling instrument 5, the lack of a user's hand on the surgical stapling instrument 5, or any indication that the user is done employing the surgical stapling instrument 5. The power management circuit 5010, 5110 is to disengage circuit components from the battery based on the determination that the activity has ended. Disengaging includes disconnecting circuit components from the battery to prevent loss when not in use.

In accordance with the present disclosure, the power management circuit 5010, 5110 can electrically disengage, severs, or disconnect circuit components that are not is use while operating the surgical stapling instrument 5 (FIGS. 1-4). The power management circuit 5010, 5110 may disengage circuit components based on the voltage level of the power source and the load on the motor 5026, 5126.

Further, in accordance with the present disclosure, the power management circuit 5010, 5110 can interact with external mechanisms or features to determine activity of the surgical stapling instrument 5 (FIGS. 1-4). The power management circuit 5010, 5110 engages circuit components based interactions with the external mechanisms. For example, the power management circuit 5010, 5110 can interact with a trocar. The power management circuit 5010, 5110 engages circuit components, which activate the surgical stapling instrument 5. For example, insertion of the surgical stapling instrument 5 into a trocar turns the surgical stapling instrument 5 on and removal of the surgical stapling instrument 5 from the trocar turns the surgical stapling instrument 5 off or to an idle state.

In accordance with the present disclosure, the external feature of the surgical stapling instrument 5 (FIGS. 1-4) can be a staple cartridge 22 (FIGS. 1 and 2). The power management circuit 5010, 5110 determines the insertion of the staple cartridge 22 into a channel 21 (FIG. 1) of the surgical stapling instrument 5. The power management circuit 5010, 5110 engages circuit components based on the insertion of the staple cartridge 22 into the channel 21. The power management circuit 5010, 5110 determines the staple cartridge 22 is empty and disengages circuit components based on the determination.

Further, in accordance with the present disclosure, the external feature can be a device located in the operating room. The power management circuit 5010, 5110 determines that the surgical stapling instrument 5 (FIGS. 1-4) is located in the operating room or that a second device is located in the operating room. The power management circuit 5010, 5110 engages circuit components based on the determination that the surgical stapling instrument 5 is located in the operating room or based on the determination that a second device is located in the operating room. For example, the second device indicates that a surgical procedure is to start. The second device may be another surgical stapling instrument similar to the surgical stapling instrument 5.

In accordance with the present disclosure, the power management circuit 5010, 5110 can interact with devices externally connected through a wireless connection. The externally connected devices inform the power management circuit 5010, 5110 as to its status during the surgical procedure. Additionally, the power management circuit 5010, 5110 can receive signals from an externally connected device coupled to a surgical hub or surgical energy generator hub (e.g., ultrasonic, monopolar or bipolar RF, or any combination of ultrasonic, monopolar or bipolar RF energy). Some of the received signals indicate that there will be a delay before the externally connected device will be required again or that the externally connected device service in the procedure is complete. The externally connected device can be partially or completely turned off based on the signals. Alternatively, the externally connected devices can be partially or completely turned on by the signals. Engaging circuit components may include turning on the externally connected device or the surgical stapling instrument 5 (FIGS. 1-4) and disengaging circuit components.

Further, in accordance with the present disclosure, the power management circuit 5010, 5110 can control the user interface. The power management circuit 5010, 5110 adjusts the brightness or contrast of a display portion of the surgical stapling instrument 5. For example, during a firing stage of the surgical stapling instrument 5, the power management circuit 5010, 5110, controls the display. In accordance with the present disclosure, a user may not look at the display until the firing stage is complete. Accordingly, during the firing stage the display is turned off and the display is turned on after the firing stage.

Further, in accordance with the present disclosure, the power management circuit 5010, 5110 may disengage circuit components while the surgical stapling instrument 5 (FIGS. 1-4) is in a storage or shipping package. The power management circuit 5010, 5110 determines that the surgical stapling instrument 5 has been removed from the storage or shipping package based on input received from various sensors. The power management circuit 5010, 5110 maintains the device in idle or off state until after the storage or shipping package is removed and the surgical stapling instrument 5 is initialized. The sensor can be a pressure sensor to detect a broken vacuum seal in the storage or shipping package. The pressure sensor also can sense that a user is holding the surgical stapling instrument 5. The sensor can be a temperature sensor to detect that the user is holding the device. A switch on the surgical stapling instrument 5 can activate the surgical stapling instrument 5 for the first time and can initialize the surgical stapling instrument 5. Any other suitable sensor or method can be employed to sense removal of the surgical stapling instrument 5 from the storage or shipping package.

Additionally, in accordance with the present disclosure, the power management circuit 5010, 5110 can also control the power management of the communication systems. The power management circuit 5010, 5110 may control the data rate of the communication system. The control circuit 100 (FIG. 4), 5012 (FIG. 70), 5112 (FIG. 71) adjusts the data rate based on at least the power level of the battery. The control circuit, 100, 5012, 5112 adjusts the data rate based on at least the hardware connected to the surgical stapling instrument 5 (FIGS. 1-4).

Additionally, in accordance with the present disclosure, the power management circuit 5010, 5110 may disable the communication systems based on a lack of detecting associated hardware in the operating room. The power management circuit 5010, 5110 determines a lack of hardware in the operation room based on: (1) no detection of devices within a received signal strength indication link range, (2) no devices acting as a preparing advertising beacon, and (3) a lack of audio-based communication, infrared IR based communication, and RFID signals.

Further, in accordance with the present disclosure, the power management circuit 5050, 5110 may not be part of the control circuit 100 (FIG. 4), 5012 (FIG. 70), 5112 (FIG. 71). The power management circuit 5010, 5110 can selectively engage and disengage circuit components based on at least one of the voltage level of the power source, the state of the surgical stapling instrument 5 (FIGS. 1-4), the desired power level to the motor 5026, 5126 (FIGS. 70-71), and the circuit components to be used.

In accordance with the present disclosure, the power management circuit 5010, 5110 can charge a super capacitor. When additional power is required to service the electrical system, the super capacitor can deliver an additional pulse of energy into the system. The energy can be a burst mode of short duration.

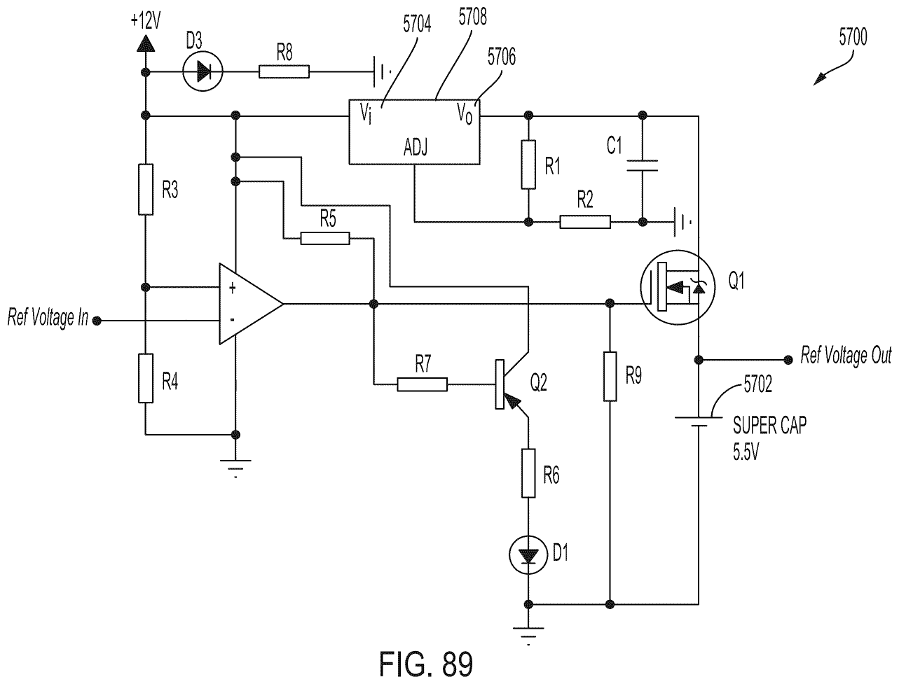

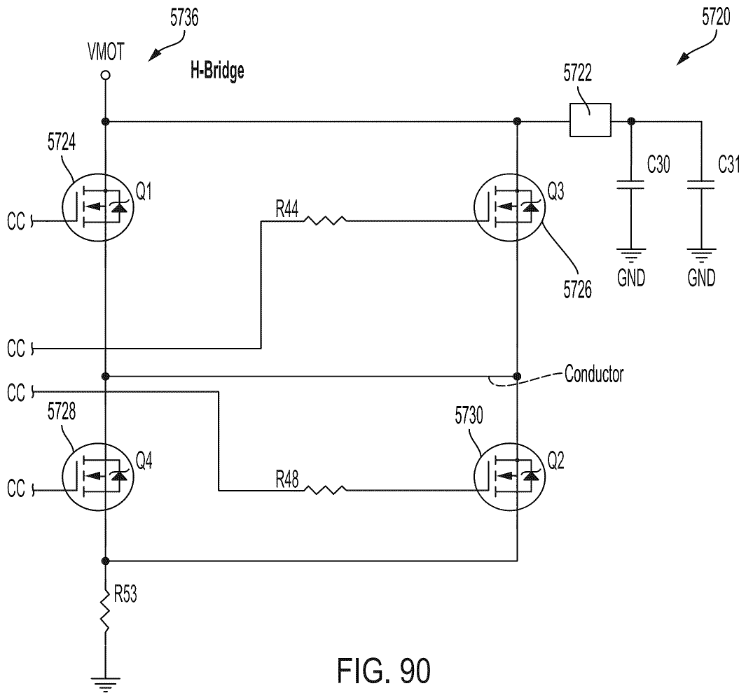

FIG. 89 is a super capacitor charging circuit 5700, according to this disclosure. The super capacitor charging circuit 5700 comprises an adjustable regulator 5708 to charge a super capacitor 5702. The adjustable regulator 5708 comprises an input 5704 to receive a first voltage level and an output 5706 to output a second voltage level to charge the super capacitor 5702. Super capacitors can be integrated into the H-Bridge motor drive circuit 5720 (shown in FIG. 90).

For example, the control circuit 100 (FIG. 4), 5012 (FIG. 70), 5112 (FIG. 71) of the surgical stapling instrument 5 (FIGS. 1-4) detects thick dense tissue within the anvil 24 and the staple cartridge 22 of the end effector 20 (FIGS. 1-4) during a drive cycle of the surgical stapling instrument 5. The control circuit 100 can detect the thick dense tissue by monitoring the voltage, current, and the speed of the knife coupled to the firing beam 60 (FIG. 1). If the monitored parameters begin to fall below a minimum threshold for a successful drive cycle, the control circuit 100, 5012, 5112 triggers the inclusion of a charged super capacitor 5702 into the control circuit 100, 5012, 5112. The stored energy in the super capacitor 5702 is added to the drive system power to increase the output power level above the standard levels. The increased power provided by switching in the super capacitor 5702 provides additional energy to the drive system to complete the drive cycle.

FIG. 90 illustrates a motor drive circuit 5720 with boost circuit connections, in accordance with the present disclosure. As illustrated in FIG. 90, the motor drive circuit 5720 can be an H-bridge. Other drive circuit implementations are contemplated to be within the scope of this disclosure. The motor drive circuit 5720 controls the function of the motor, such as for example, the motor 5026, 5126 (FIGS. 70 and 71). By turning on a first transistor 5724 (Q1) and a second transistor 5730 (Q2) and turning off a third transistor 5726 (Q3) and a fourth transistor 5728 (Q4), the motor is energized in a forward operation. The positive motor lead is connected between the first transistor 5724 (Q1) and the fourth transistor 5728 (Q4). The negative motor lead is connected between the second transistor 5730 (Q2) and the third transistor 5726 (Q3). By turning on the third transistor 5726 (Q3) and the fourth transistor 5728 (Q4) and turning off the first transistor 5724 (Q1) and the second transistor 5730 (Q2), the motor is energized in a backward operation. The voltage applied to the motor is supplied from a power source 5736.

In accordance with the present disclosure, the supercapacitor circuit (FIG. 89) may be coupled to the motor drive circuit 5720 by a switch 5722. The switch 5722 couples the motor drive circuit 5720 and the super capacitor charging circuit 5700. When the switch 5722 is closed, the super capacitor charging circuit 5700 provides additional power to the motor. When the switch 5722 is open, the motor is driven by the power source 5736.

The switch 5722 is controlled by the control circuit 100, 5012, 5112 (FIGS. 4, 70, 71). The control circuit 100, 5012, 5112 monitors any voltage sag of the power source 5736. The control circuit 100, 5012, 5112 determines that the voltage sag is greater than a pre-determined threshold and based on the determination the control circuit 100, 5012, 5112 closes the switch 5722 to supply additional power from the super capacitor 5702 (FIG. 89).

In accordance with the present disclosure, the power boost can also prevent a brownout condition of the electronics for lack of sufficient power to the electronics.

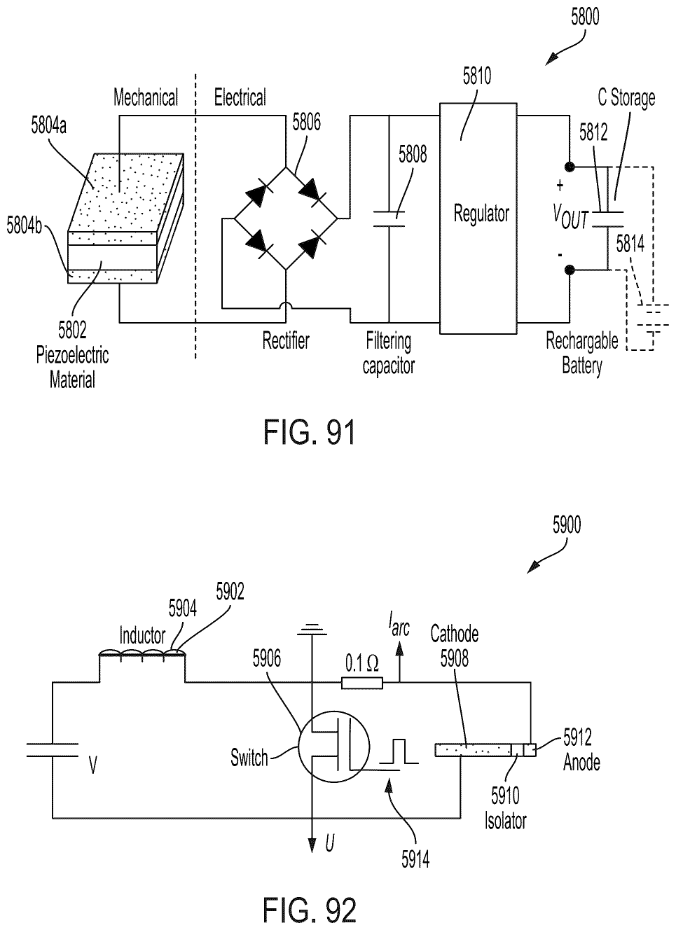

FIG. 91 is a vibration circuit 5800 to harvest energy from vibrations of the surgical stapling instrument 5 (FIGS. 1-4), in accordance with the present disclosure. The vibration circuit 5800 generates and stores power based on the vibration of the surgical stapling instrument 5. The vibration circuit 5800 can harvest energy from the inherent vibrations experienced by the surgical stapling instrument 5 and can store the energy for future uses. Energy harvested from the surgical stapling instrument 5 vibrations also may be used to slowly charge an energy storage system such as a capacitor, rechargeable battery, and the like.

In accordance with the present disclosure, the vibration circuit 5800 may comprise a piezoelectric material 5802 coupled between a first electrode 5804a and a second electrode 5804b. Under mechanical vibration the piezoelectric material 5802 generates an alternating voltage across the first 5804a and second electrodes 5804b. The vibration circuit 5800 also comprises a rectifier 5806 to convert the alternating voltage into a DC output voltage. The vibration circuit 5800 also comprises a filtering capacitor 5808. The vibration circuit 5800 also comprises a regulator 5810 to maintain a fixed output voltage irrespective of input voltage or load conditions. The voltage at the output of the regulator 5810 can be used to charge a storage capacitor 5812 (C_{storage}) or charge a rechargeable battery 5814.

The collected power is stored and can be released if needed by the surgical stapling instrument 5 (FIGS. 1-4). For example, the control circuit 100, 5012, 5112 (FIGS. 4, 70, 71) determines that the desired voltage to the motor is less than the voltage received by the motor. The control circuit 100, 5012, 5112 discharges the stored power in the vibration circuit 5800 based on the determination.

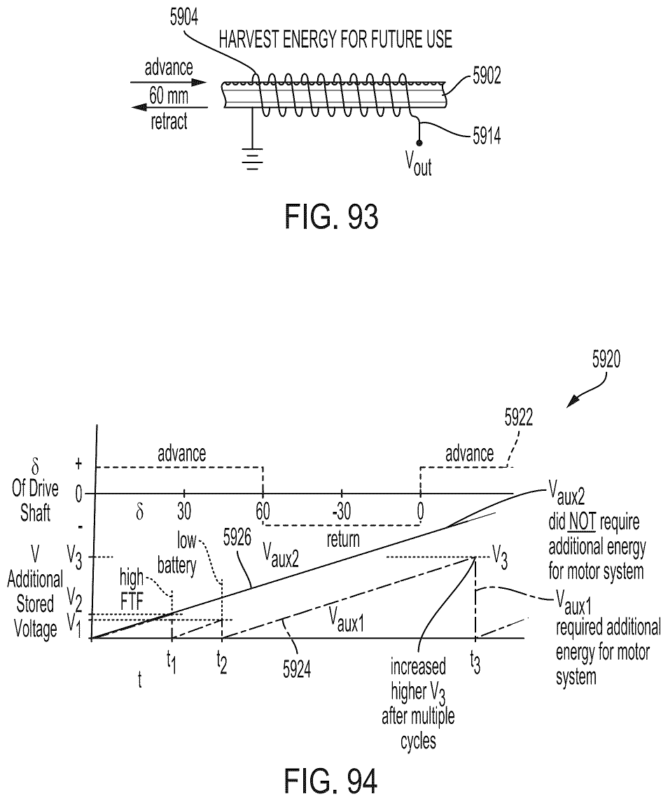

FIGS. 92 and 93 is a circuit 5900 to collect power from the movement of a drive shaft 5902 of the surgical stapling instrument (FIGS. 1-4) such as, for example, the drive shaft 40 (FIGS. 1 and 4), in accordance with the present disclosure.

FIG. 93 is a detailed view of the energy harvesting inductor 5904 showing a coil 5914 surrounding the drive shaft 5902. As illustrated in FIG. 92, the inductor 5904 can be formed by surrounding the drive shaft 5902 with a coil 5914. As the drive shaft 5902 moves back and forth with each firing cycle of the surgical stapling instrument 5, current is in the inductor 5904.

In accordance with the present disclosure, the circuit 5900 can store the energy in the inductor 5904 for future extraction. The collected energy is stored in the inductor 5904 and released if and when it is needed by the surgical stapling instrument 5 (FIGS. 1-4). For example, the control circuit 100, 5012, 5112 (FIGS. 4, 70, 71) determines that the desired voltage to the motor is less than the voltage received by the motor. Accordingly, the control circuit 100, 5012, 5112 discharges the stored energy in the circuit 5900 based on the determination.

FIG. 94 illustrates a graph 5920 of additional energy applied to the system from the circuit 5900, in accordance with the present disclosure. The graph 5920 illustrates the movement of the drive shaft 5902 shown in FIGS. 92-93. A first voltage profile 5924 illustrates the voltage when additional energy is required for the motor system. A second voltage profile 5926 illustrates the voltage when additional energy is not required for the motor. Additional energy is required when the output voltage is below the desired voltage to the motor.

In accordance with the present disclosure, the control circuit 100, 5012, 5112 (FIGS. 4, 70, 71) can separate a distinct battery sub-system from the rest of the circuit components in order to use the sub-system as an auxiliary pulsed power source.

In accordance with the present disclosure, a higher performance battery pack can be used during over stressed firing conditions. For example, a battery cell is at 3.0 volts, whereas other battery cells in the same size at 3.3 to 3.8 volts. The use of four battery cells with a 0.3 to 0.8 volt increase, increases the total voltage by 1.2 to 3.2 volts (10% to 26% increase). This battery system would have a longer life due to the increased initial voltage.

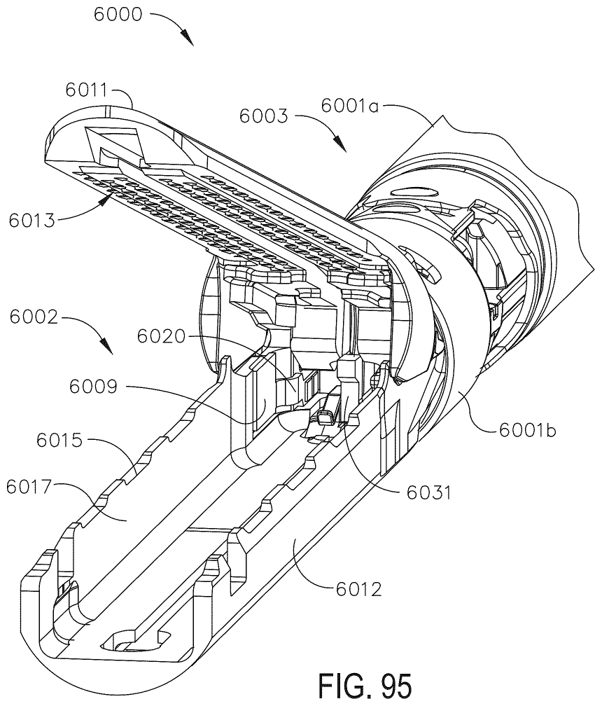

FIG. 95 is a distal perspective view of a surgical instrument 6000, in accordance with the present disclosure. The surgical instrument 6000 includes a shaft 6001 that defines a central longitudinal axis 6005 extending therethrough. The shaft 6001 includes a proximal shaft portion 6001a that can be coupled to a handle in instances where the surgical instrument is handheld. In other instances, the proximal shaft portion 6001a is coupled to a robotic arm for use with a surgical robot, for example.

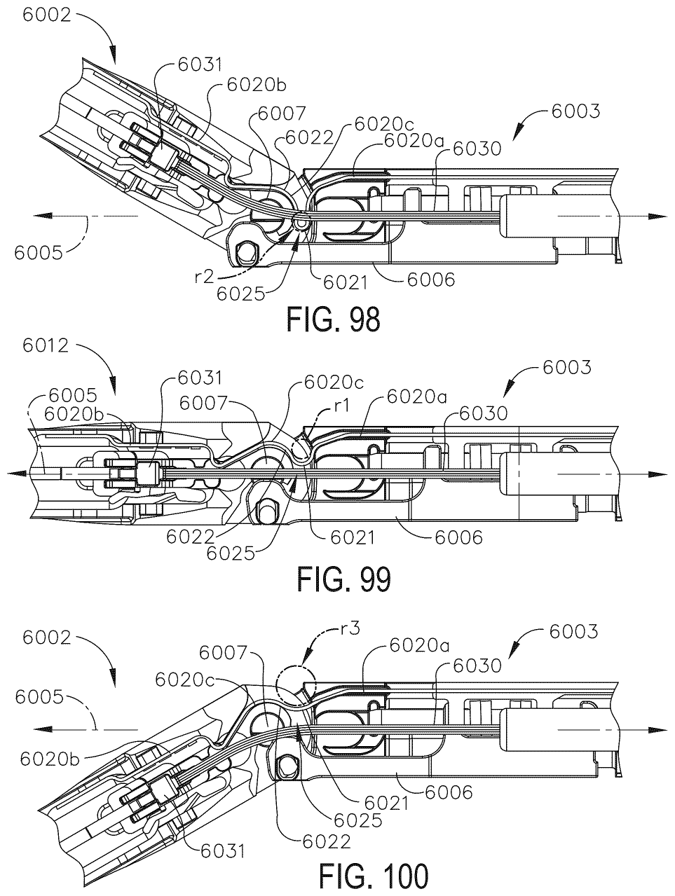

The shaft 6001 further includes a distal shaft portion 6001b coupled to an end effector 6002. An articulation joint assembly 6003 extends between the proximal shaft portion 6001a and the distal shaft portion 6001b. The articulation joint assembly 6003 includes an articulation driver 6006 movable distally to rotate the end effector 6002 from an unarticulated position, as illustrated in FIG. 99, toward a first fully articulated position, as illustrated in FIG. 98, about an articulation joint 6007. Furthermore, the articulation driver 6006 is movable proximally to rotate the end effector 6002 from the unarticulated position, as illustrated in FIG. 99, toward a second fully articulated position, as illustrated in FIG. 100, about the articulation joint 6007. In the illustrated example, the first fully articulated position is on a first side of the unarticulated position, and the second fully articulated position is on a second side of the unarticulated position, opposite the first side, for example.

Further to the above, the end effector 6002 includes a first jaw 6011 and a second jaw 6012 movable relative to the first jaw 6011 to transition the end effector 6002 from an open configuration, as illustrated in FIG. 95, to a closed configuration to grasp tissue between the first jaw 6011 and the second jaw 6012. In the illustrated example, the first jaw 6011 comprises an anvil including staple forming pockets 6013 and the second jaw 6012 includes a longitudinal channel 6015 configured to receive a staple cartridge 6014 including staples deformable against the staple forming pockets 6013 of the anvil. In accordance with the present disclosure, the first jaw 6011 and the second jaw 6012 may be equipped with electrodes configured to deliver therapeutic and/or non-therapeutic energy to the grasped tissue.

In the illustrated example, a drive shaft 6030 extends distally along the longitudinal central axis 6005 in the unarticulated position, and is coupled to a firing beam 6031 movable by the drive shaft 6030 to drive a sequential deployment of staples from the staple cartridge 6014 and to cut the stapled tissue. Additional details are described in U.S. patent application Ser. No. 14/200,111, entitled CONTROL SYSTEMS FOR SURGICAL INSTRUMENTS, filed Mar. 7, 2014, which issued on Apr. 25, 2017 as U.S. Pat. No. 9,629,629, which is hereby incorporated by reference herein in its entirety.

The surgical instrument 6000 further includes a flex circuit 6020 that transmits at least one of data or power through the articulation joint assembly 6003 to the end effector 6002 from a power source or a data source proximal to the articulation joint assembly 6003. In accordance with the present disclosure, the flex circuit 6020 may transmit power to the end effector 6002 for powering an electronics package 6009 that communicates with a chip in the staple cartridge 6014, for example. Additionally, or alternatively, the flex circuit 6020 may define a communication pathway between the chip and a processor of the surgical instrument 6000 positioned proximal to the articulation joint assembly 6003. In accordance with the present disclosure, as illustrated in FIG. 95, the electronics package 6009 can be fixed to a side wall 6017 of the longitudinal channel 6015.

In accordance with the present disclosure, the flex circuit 6020 may comprise floating ends. Additionally, in accordance with the present disclosure, the flex circuit 6020 may comprise fixed ends. Alternatively, in accordance with the present disclosure, the flex circuit 6020 may comprise one floating end and one fixed end. Further, in accordance with the present disclosure, the flex circuit 6020 may include a flexible substrate, and a conductive layer disposed on the flexible substrate.

Translating a flex circuit around an articulation joint requires some manner of accommodation for the different distances spanned by the flex circuit in the unarticulated position and the articulated position. One approach is to provide some manner of strain-relief or allow the flex circuit to change its effective length. A key challenge with strain relief is the number of times the strain relief can be activated and the number of times it can fully recover either due to loss of elasticity, interference due to debris, or fatigue of the copper wiring within the flex circuit. Also, changing the effective length of the flex circuit has its own challenges. The unaccommodated additional length, in the unarticulated position, may cause portions of the flex circuit to extend outside the surgical instrument, or may wrap around, or interfere, with other components.

In accordance with the present disclosure, the present disclosure may present solutions that avoid the forgoing challenges. In accordance with the present disclosure, as illustrated in FIGS. 98-100, the flex circuit 6020 can remain folded in the unarticulated position (FIG. 99), the first articulated position (FIG. 98), and the second articulated position (FIG. 100). The planned folding of the flex circuit maintains the flex circuit 6020 in a relaxed curved state during a full range of articulation of the end effector 6002, while transitioning between different degrees of curvature without fully straightening or needing strain relief. As discussed in greater detail below, the flex circuit 6020 never reaches a fully unfolded configuration throughout the full range of articulation of the end effector 6002, thereby nullifying any need for a refold biasing force.

In the illustrated example, the flex circuit 6020 is offset from the central longitudinal axis 6005, and includes a proximal flex-circuit portion 6020a positioned in the proximal shaft portion 6001a, a distal flex-circuit portion 6020b positioned in the distal shaft portion 6001b, and an intermediate flex-circuit portion 6020c extending between the proximal and distal flex-circuit portions 6020a, 6020b through the articulation joint assembly 6003 in a predefined passageway, as illustrated in FIGS. 98-100. The intermediate flex-circuit portion 6020c includes a proximal segment 6021 folded into a cavity 6025 defined in the articulation joint assembly 6003. In the illustrated example, the cavity 6025 is situated proximal to the articulation joint 6007. The intermediate flex-circuit portion 6020c further includes a distal segment 6022 extending distally from the folded proximal segment 6021. The distal segment 6022 partially wraps around the articulation joint 6007 and, then, extends distally toward the distal flex-circuit portion 6020b.

The folded proximal segment 6021 is tucked in the cavity 6025, and transitions between a first folded configuration, as illustrated in FIG. 99, and a second folded configuration, as illustrated in FIG. 98. In the second folded configuration, the proximal segment 6021 extends across the drive shaft 6030 and across the central longitudinal axis 6005. When the end effector 6002 is returned to the unarticulated position (FIG. 99) from the first articulated position (FIG. 98), the proximal segment 6021 retreats to a less curved, or less folded, configuration but remains in a folded/curved state without straightening out completely, thus, ensuring that the intermediate flex-circuit portion 6020c remains in a relaxed state as the end effector 6002 repeatedly transitions between the unarticulated position (FIG. 99) and the first articulated position (FIG. 98).

Further to the above, the proximal segment 6021 transitions between the second folded configuration, as illustrated in FIG. 99, and a third folded configuration, as illustrated in FIG. 100. I and transitions between a first folded configuration, as illustrated in FIG. 99, and a second folded configuration, as illustrated in FIG. 98. In the second folded configuration, the proximal segment 6021 extends across the drive shaft 6030 and across the central longitudinal axis 6005. When the end effector 6002 is returned to the unarticulated position (FIG. 99) from the first articulated position (FIG. 98), the proximal segment 6021 retreats to a less curved, or less folded, configuration but remains in a folded/curved state without straightening out completely, thus, ensuring that the intermediate flex-circuit portion 6020c remains in a relaxed state as the end effector 6002 repeatedly transitions between the unarticulated position (FIG. 99) and the first articulated position (FIG. 98). When the end effector 6002 is transitioned from the unarticulated position (FIG. 99) to the second articulated position (FIG. 100), the proximal segment 6021 retreats to a less curved, or less folded, configuration but remains in a folded/curved state without straightening out completely, thus, ensuring that the intermediate flex-circuit portion 6020c remains in a relaxed state as the end effector 6002 repeatedly transitions between the unarticulated position (FIG. 99) and the second articulated position (FIG. 100)

Accordingly, the proximal segment 6021 maintains a curved, or folded, state throughout a full range of articulation of the end effector 6002, while experiencing a reduction in curvature as the end effector 6002 moves from the first articulated position (FIG. 98) to the second articulated position (FIG. 100). Said another way, the proximal segment 6021 comprises a first curvature in the first articulated position (FIG. 98) greater than a second curvature of the proximal segment 6021 in the unarticulated position (FIG. 99), and a third curvature in the second articulated position (FIG. 100) less than the second curvature and the first curvature.

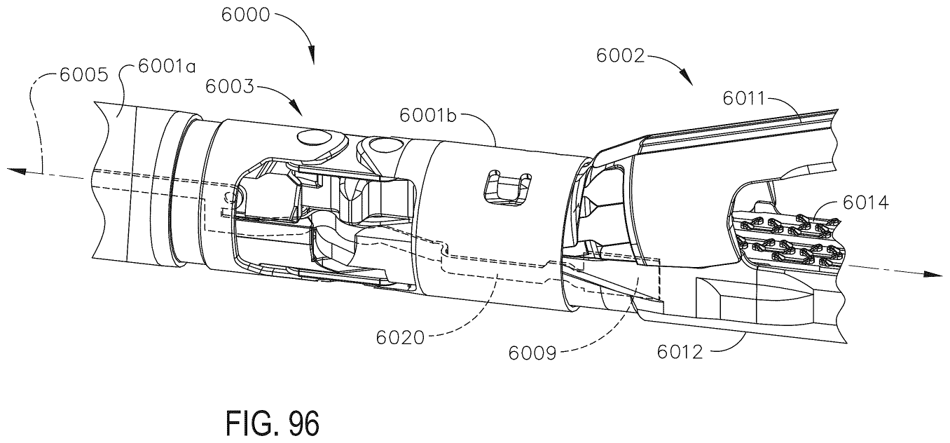

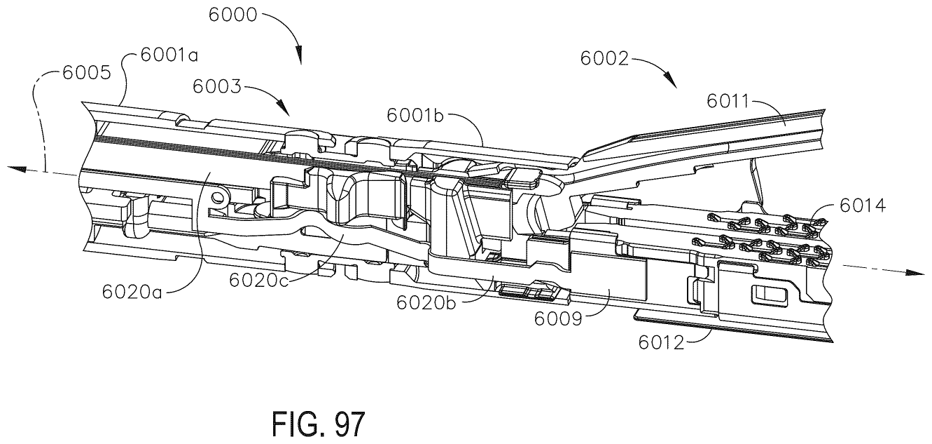

As best illustrated in FIGS. 96 and 97, the flex circuit 6020 comprises a flat ribbon shape generally extending longitudinally alongside the drive shaft 6030, and including a thickness, and a width greater than the thickness. The proximal segment 6022 of the intermediate flex-circuit portion 6020c is formed by folding the flex circuit 6020 about an axis extending along the width, which permits the proximal segment 6021 to be tucked into the cavity 6025. The different curvatures of the proximal segment 6021 comprise different radii of curvatures that extend along the thickness of the proximal segment 6021.

In accordance with the present disclosure, proximal segment 6021 may have a flex radius greater than, or equal to, about 2× the thickness of the flex circuit 6020 throughout a full range of articulation of the end effector 6002. Further, in accordance with the present disclosure, the proximal segment 6021 may have a flex radius selected from a range of about 2× to about 4× the thickness of the flex circuit 6020, for example, throughout a full range of articulation of the end effector 6002. This arrangement ensures an articulation of the end effector 6002 that only causes a change in the tortuous path of the flex circuit 6020 without strain relief or a change in the effective length of the intermediate flex-circuit portion 6002c.

In the illustrated example, the proximal segment 6021 defines a first radius of curvature (r1) in the unarticulated position (FIG. 99), a second radius of curvature (r2) in the first articulated position (FIG. 4), and a third radius of curvature (r3) in the second articulated position (FIG. 100). The radii of curvature (r1, r2, r3) are different from one another, but all permit the proximal segment 6021 to remain within the cavity 6025 as the proximal segment transitions therebetween.

Further to the above, referring primarily to FIGS. 98-100, the length of the proximal segment 6021 changes as the end effector 6002 transitions between the first articulation position (FIG. 98), the unarticulated position (FIG. 99), and the second articulated position (FIG. 100), without fully straitening, or while maintaining a curves/folded state throughout its full range of articulation. The excess length of the proximal segment 6021, which is tucked in the cavity 6025, permits a relaxed transition between the different positions of the end effector without requiring strain relief, and without pulling on the proximal and/or distal flex circuit portions 6002a, 6002b.

In the illustrated example, the proximal segment 6021 enters the cavity 6025 by extending toward the drive shaft 6030, then curves away from the drive shaft 6030. The distal segment 6022, which extends distally from the proximal segment 6021, and partially wraps around the articulation joint 6077, then extends distally toward the end effector 6002. Other pathways and curvatures of the intermediate flex-circuit portion 6002c are contemplated by the present disclosure.

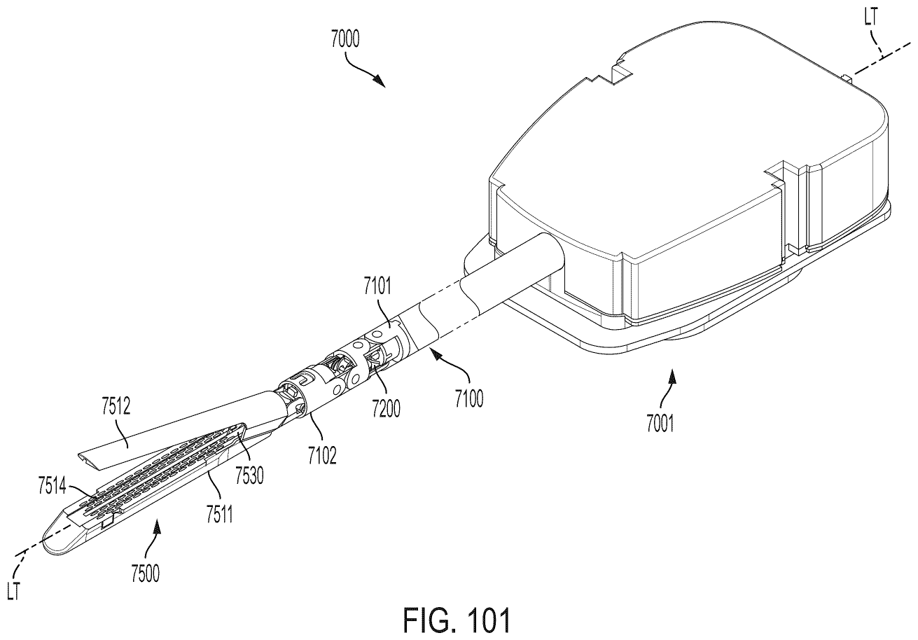

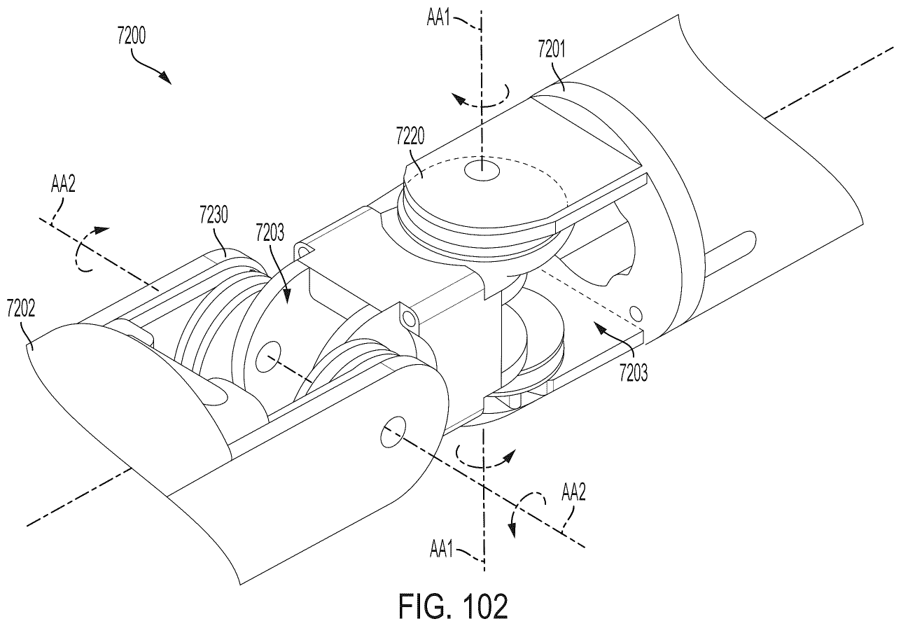

FIG. 101 provides a perspective view of a surgical instrument 7000, in accordance with the present disclosure. The surgical instrument 7000 includes a shaft 7100 and a multi-axis articulation joint assembly 7200. The shaft 7100 defines a longitudinal axis LT. In instances where the surgical instrument 7000 is robotically manipulated, the shaft 7100 can be coupled to a robotic arm or an intermediary mount for a robotic arm, such as, for example, a tool mounting portion 7001, as depicted in FIG. 101. In other instances where the surgical instrument is handheld, the shaft 7100 is coupled to a handle, for example. The shaft 7100 further includes a distal shaft portion 7102. The surgical instrument may further comprise an end effector 7500 coupled to the distal shaft portion 7102 and/or the multi-axis articulation joint assembly 7200.

Further to the above, the multi-axis articulation joint assembly 7200 extends between a proximal shaft portion 7101 and the distal shaft portion 7102. As illustrated in FIG. 102, the multi-axis articulation joint assembly 7200 includes a proximal end 7201, a distal end 7202, and first and second articulation joints 7220, 7230. The multi-axis articulation joint assembly includes a cavity 7203 extending between the proximal end 7201 and the distal end 7202.

In accordance with the present disclosure, when the multi-axis articulation assembly 7200 is in a non-articulated state, the first articulation joint 7220 and the second articulation joint 7230 can be longitudinally separated by a longitudinal distance D and the multi-axis articulation joint assembly 7200 is aligned with the longitudinal axis LT of the surgical instrument 7000. Additional details are described in U.S. patent application Ser. No. 17/032,279, entitled SURGICAL STAPLING INSTRUMENTS WITH ROTATABLE STAPLE DEPLOYMENT ARRANGEMENTS, Sep. 25, 2020, which is hereby incorporated by reference herein in its entirety.

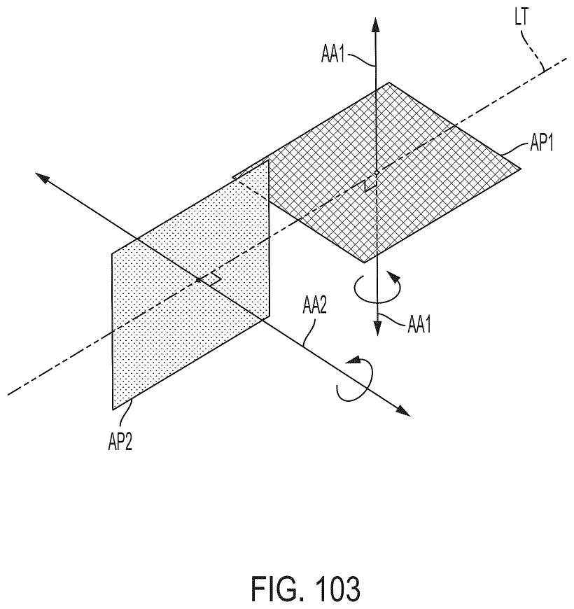

Further to the above, the multi-axis articulation joint assembly 7200 is able to transition from a non-articulated state, as illustrated in FIGS. 101-103, to an articulated state by moving the first articulation joint 7220 and/or the second articulation joint 7230 in articulation planes AP1 and AP2 about articulation axes AA1 and AA2, respectively. As used in the present disclosure in relation to a particular articulation joint in a non-articulated state, an articulation plane is defined as a plane that is perpendicular to the articulation axis of the articulation joint, such that both planes longitudinally extend with the longitudinal axis LT. For example, FIG. 103 provides a geometric representation of the spatial relationships between articulation axes AA1/AA2, articulation planes AP1/AP2, and the longitudinal axis LT. Although FIG. 103 depicts articulation axes AA1 and AA2 as intersecting longitudinal axis LT within respective articulation planes AP1 and AP2, it is envisioned that the articulation axes and/or articulation planes may not be aligned with one another in other implementations with respect to the longitudinal axis LT, such as, for example, with offset articulation joints.

Still referring to FIGS. 101-103, the multi-axis articulation joint assembly 7200 is configured to articulate in multiple planes. In accordance with the present disclosure, the first articulation joint 7220 and the second articulation joint 7230 can be oriented such that the articulation planes AP1 and AP2 are transverse with one another. Said in another way, the articulation joints 7220 and 7230 can be rotationally offset from each other with respect to the longitudinal axis LT. For example, as best illustrated in FIGS. 102-103, the articulation planes AP1 and AP2 are perpendicularly offset from one another in the non-articulated state.

Now referring back to FIG. 101, the distal shaft portion 7102 can be coupled to an end effector 7500 including a first jaw 7511 and a second jaw 7512 movable relative to the first jaw 7511 to transition the end effector 7500 from an open configuration, as illustrated in FIG. 101, to a closed configuration to grasp tissue between the first jaw 7511 and the second jaw 7512. In the illustrated example, the first jaw 7511 includes a staple cartridge 7514 including staples deformable against staple forming pockets of an anvil in the second jaw 7512. In accordance with the present disclosure, the first jaw 7511 and the second 7512 may be equipped with electrodes configured to deliver therapeutic and/or non-therapeutic energy to the grasped tissue. Additionally, a firing beam 7530 for driving a sequential deployment of staples from the staple cartridge 7514 and for cutting the stapled tissue may be coupled to an internal firing shaft extending along the longitudinal axis LT within the surgical instrument 7000. Additional details are described in U.S. patent application Ser. No. 14/200,111, entitled CONTROL SYSTEMS FOR SURGICAL INSTRUMENTS, filed Mar. 7, 2014, which issued on Apr. 25, 2017 as U.S. Pat. No. 9,629,629, which is hereby incorporated by reference herein in its entirety.

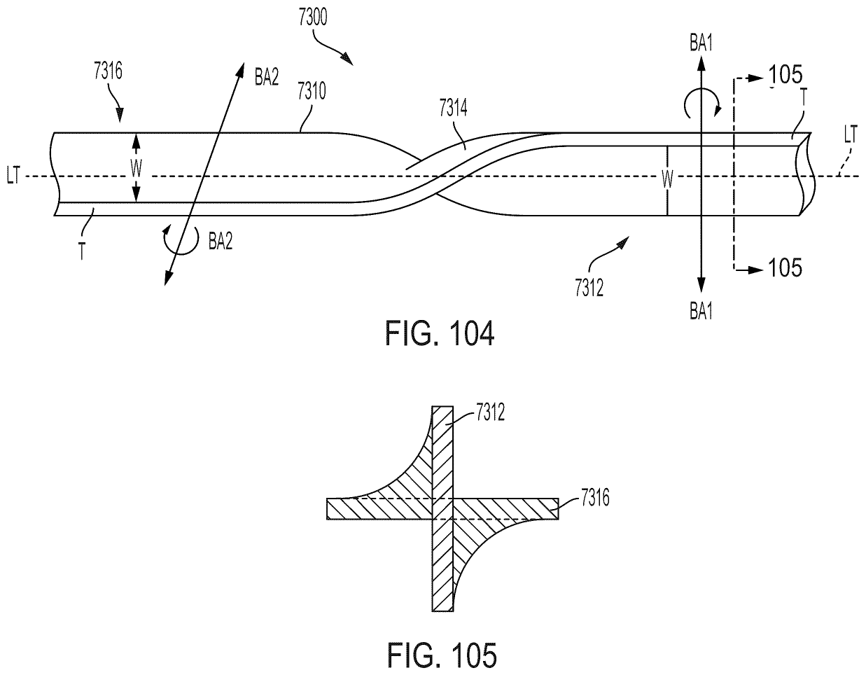

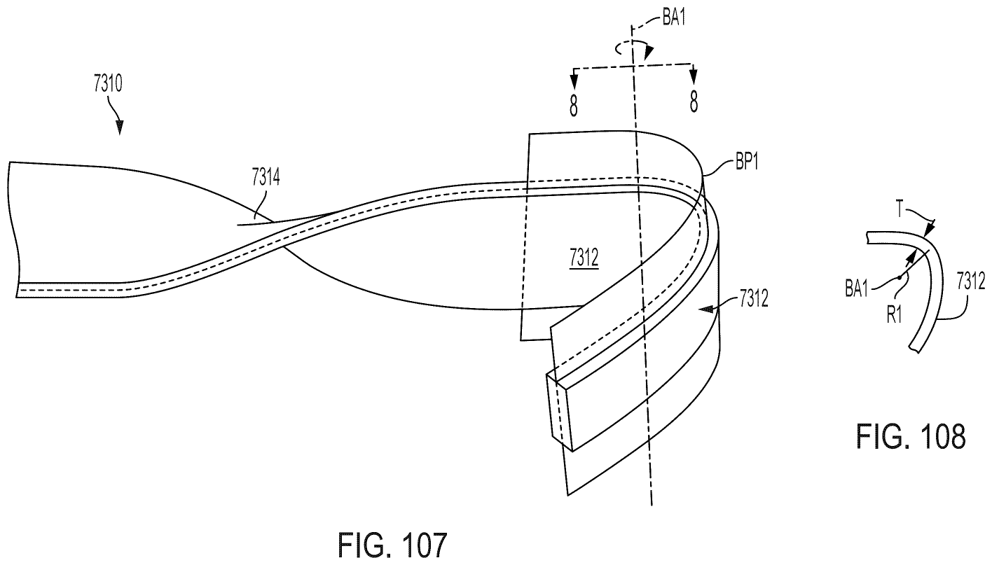

The surgical instrument 7000 further includes a wiring assembly 7300 disposed within the shaft 7100 and the multi-axis articulation joint assembly 7200. The wiring assembly 7300 includes a wiring harness extending through the cavity 7210 of the multi-axis articulation joint assembly 7200 that transmits at least one of data or power through the multi-axis articulation joint assembly 7200 to a further distal component, such as an end effector 7500, from a power source or a data source proximal to the multi-axis articulation joint assembly 7200. In accordance with the present disclosure, the wiring assembly 7300 can transmit power to the end effector 7500 for powering a chip in the staple cartridge 7514, for example. Additionally, or alternatively, the wiring assembly 7300 may define a communication pathway between the chip and a processor of the surgical instrument 7000 positioned proximal to the multi-axis articulation joint assembly 7200. Portions of the wiring assembly 7300, such as a wiring harness 7310, are able to transition between a non-articulated state, such as illustrated in FIGS. 104-106, and articulated states, such as the articulated state illustrated in FIG. 107-108, as described in greater detail below.

Routing a wiring harness through a multi-axis articulation joint requires some manner of accommodation for transitioning between various rotationally offset articulation planes along the length of the multi-axis articulation joint. One accommodation is to incorporate reinforcements, such as strain reliefs or wiring harness junctions, into sections of the wiring harness where any significant amount of flexing and/or bending during an articulation is anticipated. However, the reinforcements themselves may have a limited number of activation cycles before exhibiting a decline in elasticity, interference due to debris, and/or fatigue of copper strands within the wiring harness.

Additionally, there is generally limited space allowed for a wiring harness, and any required movement thereof, within a cavity of a multi-axis articulation joint. Therefore, the use of any reinforcements therein must be balanced with the number of desired conductors and/or geometry thereof to avoid limiting a range of articulation and/or increasing the footprint of the articulation joint. While round cabling can bend in multiple directions, round cables are typically not space efficient and can be limited in allowable bend radius, especially in situations where a multiconductor harness is desirable.

Furthermore, flat wiring harnesses can contain many conductors placed side-by-side spanning the width of the flat wiring harness. This arrangement can be resilient to repeated flexing and/or bending while providing better space efficiency than round cabling. However, flat wiring harnesses are generally limited to flexing about a single linear axis. For example, a flat wiring harness may be resilient to repetitive bends when a thickness thereof is subjected to deformations or bends about a single bend axis parallel to, or extending along, a width of the flat wiring harness with a bend radius extending along the thickness. Said another way, the plane along which a width of the flat wiring harness extends is generally transversely oriented with respect to a plane of rotation. Implementing a flat wiring harness within a multi-axis articulation joint can require multiple flat sections adjoined and reinforced by connector sections should multiple bending planes be required, thereby increasing the overall footprint of the wiring harness. Thus, standard implementations of wiring harnesses through a multi-axis articulation may result in a limited articulation range and/or electrical communication through the wiring harness, and/or a larger articulation joint footprint, each of which is undesirable.

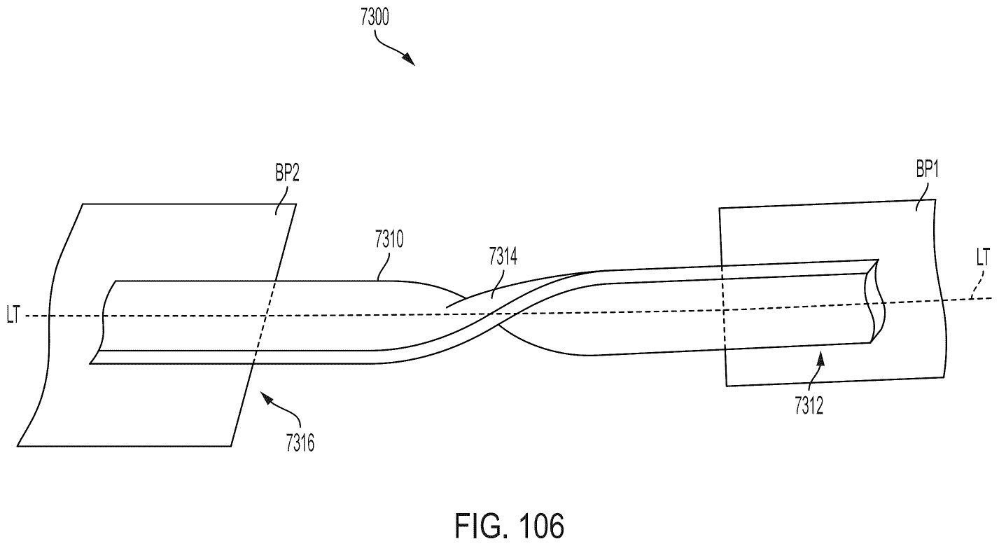

The present disclosure presents solutions that avoid the forgoing challenges. In accordance with the present disclosure, as illustrated in FIG. 104, the wiring assembly 7300 can include a wiring harness 7310 having a cross-sectional profile defined by a thickness T and a width W greater than the thickness T. As best illustrated in FIG. 106, the wiring harness 7310 includes a proximal flat portion 7312 corresponding to a first bending plane BP1 and a distal flat portion 7316 corresponding to a second bending plane BP2, with the bending planes BP1, BP2 transecting the thickness of wiring harness 7310 at the proximal and distal flat portions, respectively. The wiring harness 7300 also includes a preset twist 7314 spanning a longitudinal distance between the proximal flat portion 7312 and the distal flat portion 7316. The preset twist 7314 biases the distal flat portion 7316 into a different orientation than the proximal flat portion 7312. As discussed in greater detail below, the different orientations of the flat portions 7312 and 7316 facilitate a full range of articulation without the need for any auxiliary reinforcement sections.

Now referring to FIGS. 104-108, in the non-articulated state, the proximal flat portion 7312 spans the first articulation joint 7220 and the distal flat portion 7316 spans the second articulation joint 7230 such that the wiring harness 7310 may bend along with the first and/or second articulation joints 7220, 7230 to an articulated state, such as the articulated state illustrated in FIGS. 107-108 where the proximal flat portion 7212 is following a rotated first articulation. As best illustrated in FIGS. 106-107, the proximal flat portion 7312 comprises a first orientation in the non-articulated state such that a first bend radius R1 of the proximal flat portion 7312 in the articulated state extends through the thickness of wiring harness at the proximal flat portion 7312. Likewise, the distal flat portion 7316 comprises a second orientation in the non-articulated state such that a second bend radius R2 of the distal flat portion 7316 in the articulated state extends through the thickness of the wiring harness at the distal flat portion 7316. In accordance with the present disclosure, the first bend radius R1 and the second bend radius R2 may be greater than, or equal to, about 2× the thickness of the respective proximal flat portion and the distal flat portion throughout a full range of articulation of the articulation joints to minimize, or eliminate, overstressing the wiring harness 7310. Thus, the wiring harness 7310 remains unstretched during the full range of articulation such that the positioning of the preset twist 7314 with respect to the proximal end 7201 and/or the distal end 7202 remains the unchanged. Accordingly, this configuration of the wiring harness 7310 maintains the orientation and alignment of the flat portions 7312 and 7316 with the articulation joints 7220 and 7230, thereby minimizing, or eliminating, the need to supplement the harness with reinforcing and/or strain relief sections.

Further to the above, the proximal flat portion 7312 is associated with a first bending plane BP1 and the distal flat portion 7316 is associated with a second bending plane BP2, where the width of the wiring harness at each portion extends along the corresponding bending plane. In the non-articulated state, the bending planes BP1, BP2 intersect respective articulation planes AP1/AP2 and thus, are transversely oriented to their respective articulation planes and to one another. In accordance with the present disclosure, the bending planes BP1/BP2 may intersect the articulation planes AP1/AP2 such that their normal vectors intersect each other perpendicularly. Furthermore, each of the bending planes may curve or deform with the wiring harness, such as during an articulation, as illustrated in FIG. 107.

Further to the above, the preset twist 7314 is configured to accommodate the multiplanar articulations without incorporating additional reinforcements into the wiring harness 7310. In accordance with the present disclosure, the preset twist 7314 can define an angle that corresponds to the rotational offset between the first and second articulation joints 7220 and 7230. In the context of the present disclosure, the angle of the preset twist 7314 can be defined by the rotational offset between the flat portions 7312, 7316 adjoining the preset twist 7314 when viewed from the proximal end 7201 or distal end 7202 in the non-articulated state.

Additionally, the preset twist 7314 is maintained, or partially maintained, throughout a full range of motion of the articulation joint assembly, thereby preserving the relative planar orientations between the flat sections 7312, 7316 and respective articulation planes AP1, AP2 in the non-articulated state. The preset twist 7314 can be integrated into the wiring harness 7310 prior to assembling the surgical instrument. Additionally, the substrate of the wiring harness 7310 may comprise a tough yet flexible polymeric base material, such as, for example, a polyimide-based material, and may be molded to position the preset twist 7314 between the first and second articulation joints 7220/7230 upon routing the wiring harness 7310 through the surgical instrument 7000. Any layers of the wiring harness 7310 may also comprise the polymeric base material and/or another suitable flexible base material having desirable electrical properties. Thus, the wiring harness 7310 does not have to be rearranged and/or stretched from a resting state to provide the preset twist 7314, thereby avoiding any differences in space requirements for the wiring harness 7310 between non-articulated and articulated states. Additionally, the preset twist 7314 obviates the need for a separate biasing member to maintain the orientations of the adjoining flat portions 7312, 7316, thereby minimizing the number of additional components within the multi-axis articulation assembly and issues associated therewith.

In accordance with the present disclosure, the preset twist 7314 can be a permanent twist preformed prior to assembly with the surgical instrument 7000. Further, in accordance with the present disclosure, the preset twist 7314 can be formed by heating the polymeric base material of the wiring harness 7310, twisting the wiring harness to form the preset twist 7314, then allowing the polymeric base material to cool down. Additionally, more than one preset twist 7314 can be defined in the wiring harness 7310. In accordance with the present disclosure, one or more preset bends may be defined in the wiring harness 7310 in addition to, or instead of, the preset twist 7314. The preset twist 7314 can be formed by twisting the wiring harness 7310 to a twist angle that corresponds to a desired angle between articulation planes AP1, AP2, for example.

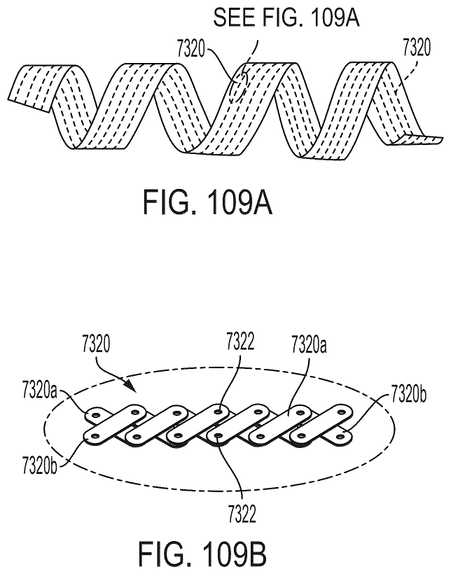

FIGS. 109A-109B illustrate conductive paths 7320 of the wiring harness 7310, in accordance with the present disclosure. The wiring harness 7310 may include conductive paths 7320 embedded within a substrate for transmitting power and/or data. The wiring harness 7310 may comprise multiple layers with at least some of the conductive paths 7320 positioned in closely arranged pairs. For example, as best illustrated in FIG. 109B, a conductive path 7320 can comprise a first group of conductive elements 7320a and a second group of conductive elements 7320b with each group providing separate portions of a conductive path, such as a supply and a return portion. Adjacent conductive elements of a particular group alternate between a top wiring harness layer and a bottom wiring harness layer while being connected with intermediate connectors 7322, each of which span the top and bottom wiring harness layers. In accordance with the present disclosure, the intermediate connectors 7322 can comprise a plated through-hole, a pin, and/or a conductive polymer. The wiring harness 7310 may further include an insulative intermediate layer between a bottom conductive element 7320a and an overlapping top conductive element 7320b to prevent electrical contact between the two groups and/or shielding layers sandwiched around conductive elements 7320a, 7320b. This arrangement can provide benefits such as electromagnetic interference rejection and/or common-mode noise rejection, which are traditionally associated with twisted pair wiring and/or shielded cabling, without using round conductors. Thus, the wiring harness 7310 and conductive elements 7320a, 7320b therein can provide advantages, such as increased space efficiency and flexibility, over other harnesses employing round conductor.

In accordance with the present disclosure, a proximal portion 7340 of the wiring assembly 7300 is housed in a retainer 7103 nested within the shaft 7100. The proximal portion 7340 is similar in many aspects to other wiring harnesses described elsewhere in the present disclosure. Thus, the proximal portion 7340 can have a cross-sectional profile defined by a thickness and a width greater than the thickness, and multiple conductive paths embedded in a tough and resilient polymeric base layer. Additionally, the proximal portion 7340 is in electrical communication with the wiring harness 7310 and/or physically joined therewith. Further, in accordance with the present disclosure, the proximal portion 7340 and the wiring harness 7310 can be routed through the surgical instrument 7000 as a single assembly.

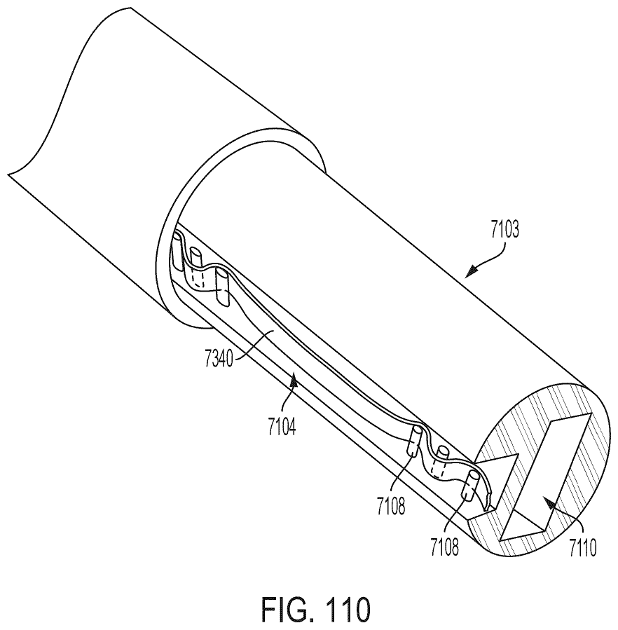

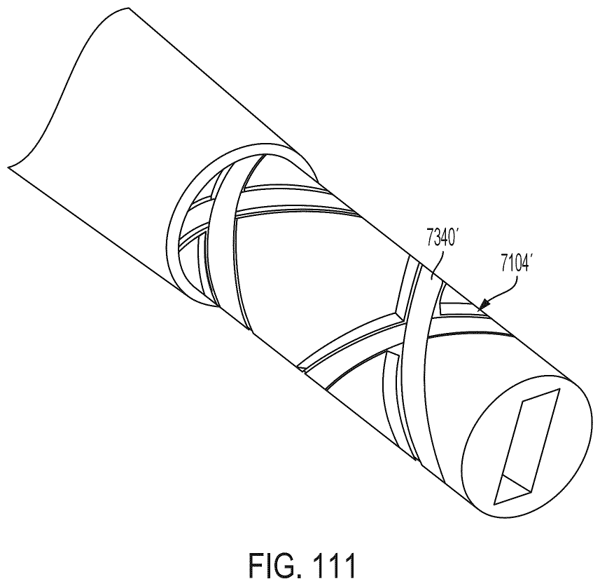

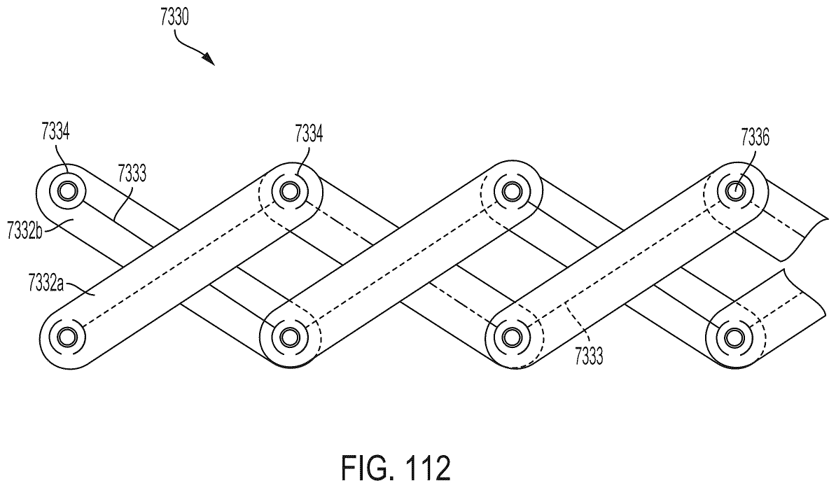

Now referring to FIG. 110, a perspective view of a retainer 7103 is provided, in accordance with the present disclosure. The retainer 7103 is nested within the shaft 7100 and comprises a plastic material. A channel 7104 is defined in an outer surface 7106 of the retainer thereof. Additionally, the channel 7104 can be configured to guide the proximal portion 7340 of the wiring assembly 7300 from a power and/or communication source proximal to the shaft 7100 toward the multi-axis articulation joint assembly 7200. Further, in accordance with the present disclosure, the channel 7104 can span an axial length of the retainer 7103. Additionally, or alternatively, one or more helical channels 7104′ can be defined in the outer surface of the retainer as illustrated in FIG. 112. The retainer 7103 can further comprise an inner channel 7110 to accommodate a longitudinally extending component such as a firing shaft.

The channel 7104 may be supplemented by guiding posts 7108 to maintain an orientation of the portion of the wiring assembly 7300 therein. The guiding posts 7108 may be molded out of the same material as the retainer 7103 and spaced such that they do not impart any friction on the proximal portion 7340 of the wiring assembly or impede any movements thereof which may occur during operation of the surgical instrument 7000. The guiding posts 7108 may be longitudinally offset from each other to facilitate routing the proximal portion 7340 of the wiring assembly therethrough without introducing an amount of curvature into the proximal portion 7340 which would induce binding in the longitudinal direction. Additionally, or alternatively, slots can be molded into the retainer 7103 for routing the wiring assembly therethrough.

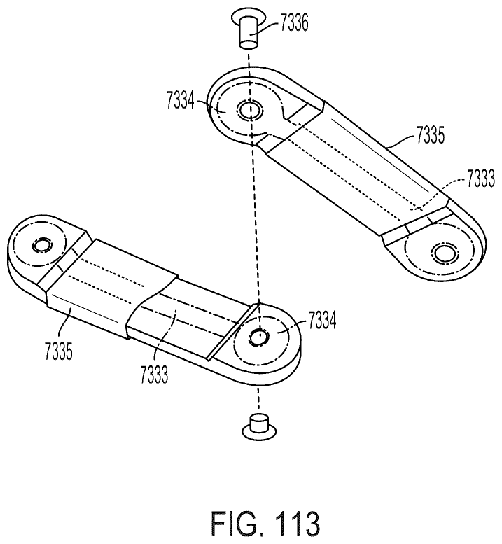

Further to the above, the wiring assembly 7300 may include scissoring portions to facilitate longitudinal expansion in areas of the surgical instrument where any components thereof may be longitudinally displaced during operation. As best illustrated in FIGS. 112-113, scissoring portions 7330 include a first group of linkages 7332a associated with a first electrical path and a second group of linkages 7332b associated with a second electrical path. Each of the linkages 7332a and 7332b includes a wire portion 7333 terminated at each end by circular contacts 7334 for providing a conduction path along the length of a linkage linkable to an adjacent linkage. Additionally, the wire portions 7333 are covered with insulating material 7335 to avoid shorting between the first and second electrical paths when overlapping as shown in FIG. 112. Rivets or pins 7336 join conductive elements of a given group together to maintain electrical continuity along the length of the scissoring portion via circular contacts 7337. The mechanical portion of the linkages 7332a and 7332b comprise a high resiliency polymeric material to withstand any bending forces without fracturing and/or undergoing plastic deformation while maintaining an ability to longitudinally expand. Furthermore, one or more of the linkages 7332a and/or 7332b may be molded with a curved length and/or width to traverse a bend if necessary.

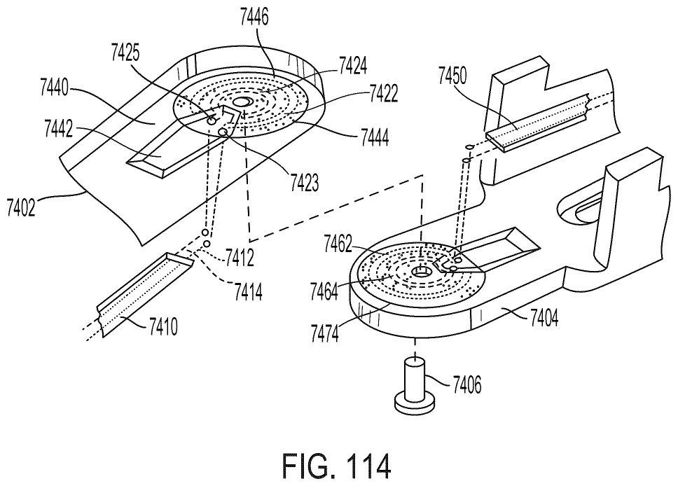

Now referring to FIG. 114, a perspective view of an articulation joint 7400 is provided according to at least one aspect of the present disclosure. The articulation joint 7400 includes a first portion 7402 and a second portion 7404 mechanically coupled by a pin 7406 through holes 7403 and 7405, such that the first portion 7402 and second portion 7404 are rotatable with respect to each other about the pin 7406 in a single plane. The first portion 7402 and/or the second portion 7404 can be comprised of a metallic and/or a plastic material. In various examples, the first portion 7402 includes a first flex circuit 7410 electrically connected to a number of conductive traces which are stationary with respect to the first portion 7402. For example, as illustrated in FIG. 114, when a flex circuit 7410 includes two wires 7412 and 7414 therein, the first wire 7412 can be coupled with a first trace 7422 at connection 7423 and the wire 7414 can be coupled with a second trace 7424 at connection 7425. The first trace 7422 and second trace 7424 are concentrically disposed, with respect to each other, around the hole 7403 on a designated mating area 7444. The flex circuit 7410 and/or connections 7423 and 7425 may lie within and/or on a channel 7442 recessed with respect to an inner surface 7440 of the first portion 7402 to avoid any rotational interference of the first and second portions 7402 and 7404, and/or to avoid any accidental electrical contact of another conductive surface with the connections 7423 and 7425. The first trace 7422 and the second trace 7424 are comprised of a conductive material, such as copper or a conductive plastic, and can be raised with respect to the inner surface 7440 and/or mating area 7444. For example, the first trace 7422 and the second trace 7424 may be formed and/or deposited on the mating area 7444 itself or preformed and adhered to the mating area 7444. In some examples, the mating area 7444 is raised with respect to inner surface 7440. In certain examples, the mating area 7444 may include an insulative coating 7446 underlying, surrounding, and/or separating the traces 7422 and 7424 to minimize any undesirable stray current between the first trace 7422, the second trace 7424, and/or the first portion itself 7402. As described in greater detail below, the second portion 7404 can maintain electrical contact with trace 7422 and 7424 without any shorting between traces and/or components attached therewith during a rotation the first portion 7402 and second portion 7404 around pin 7406.

Still referring to FIG. 114, the second portion 7404 may be configured similarly to the first portion 7402, such that the second portion 7404 is complementary to the first portion 7402. For example, the second portion 7404 can include a second flex circuit 7450, concentrically arranged traces 7462 and 7464, and a second mating area 7474, each of which are similar in many respects to flex circuit 7410, traces 7422 and 7424, and mating area 7444 of the first portion 7402, which are not repeated herein at the same level of detail for brevity. Thus, upon coupling the first portion 7402 and the second portion 7404, the traces 7422 and 7424 respectively contact traces 7462 and 7464, while the individual wires of flex circuits 7410 and 7450 and/or connections therewith remain statically positioned on their respective first and second portions 7402 and 7404 during a rotation of the first portion 7402 and/or second portion 7404. Accordingly, any strain or movement of wires or flex circuits can be avoided during a rotation of the articulation joint 7400 to an articulated state, thereby minimizing a risk of breaking an electrical connection during a surgical procedure.

With an industry shift toward smart cartridges arises a need for an effective transmission of power and/or data between the surgical instrument and disposable smart cartridges. Chips, sensors, and/or other electrical components on the staple cartridges provide tremendous functionality, but require higher power and better communication capabilities. Physical electrical interfaces between the surgical instrument and the staple cartridge are capable of meeting the power/data requirements, but suffer from exposure to saline and/or other bodily fluids during a surgical procedure, which can negatively impact power and/or data transmission.

Power and/or data transmission between the surgical instrument and the staple cartridge can also be accomplished wirelessly using wireless antennas, as described in greater detail below. Nonetheless, size constraints (e.g., diameter of antenna coil is roughly equivalent to the maximum transmission distance) can be quite taxing on the antenna coils' ability to meet the higher power and/or data transmission requirements. Moreover, slight misalignments between the antenna coils can seriously impact the ability of the antenna coils to effectively transmit power and/or data wirelessly therebetween. The present disclosure can provide technical solutions for ensuring effective power and/or data transmission between a surgical instrument (e.g., surgical instrument 8000) and a disposable cartridge (e.g., staple cartridge 8040).

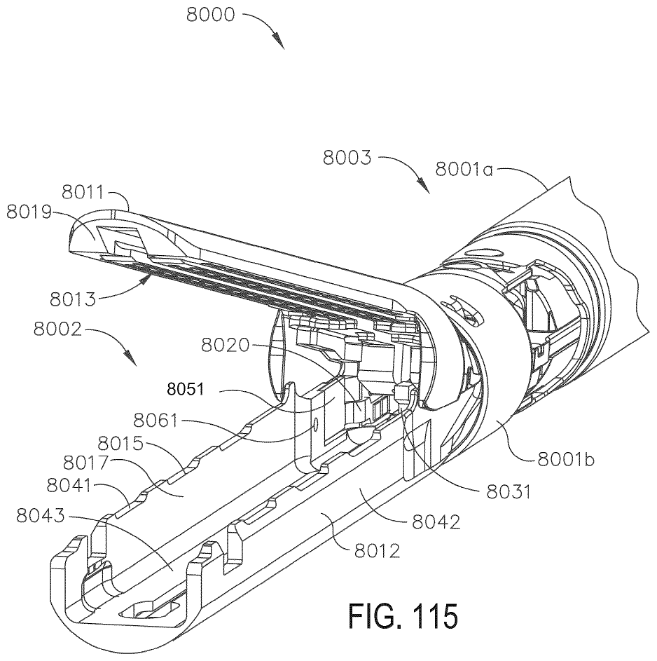

FIG. 115 is a partial perspective view of a surgical instrument 8000, in accordance with the present disclosure. The surgical instrument 8000 includes a shaft 8001 that defines a central longitudinal axis 8005 extending therethrough. The shaft 8001 includes a proximal shaft portion 8001a that can be coupled to a handle if the surgical instrument is handheld. Alternatively, the proximal shaft portion 8001a may include a housing for coupling to a robotic arm of a surgical robot, for example.

The shaft 8001 further includes a distal shaft portion 8001b coupled to an end effector 8002. An articulation joint assembly 8003 extends between the proximal shaft portion 8001a and the distal shaft portion 8001b. The articulation joint assembly 8003 includes an articulation driver movable distally to rotate the end effector 8002 from an unarticulated position toward an articulated position. The articulation joint assembly 8003 can be removed, and the proximal shaft portion 8001a and the distal shaft portion 8001b define a continuous shaft.

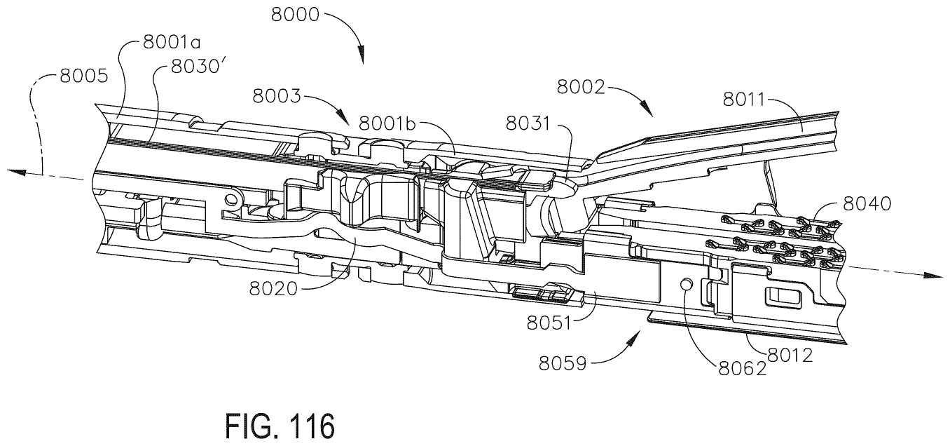

Further to the above, the end effector 8002 includes a first jaw 8011 and a second jaw 8012 movable relative to the first jaw 8011 to transition the end effector 8002 from an open configuration, as illustrated in FIG. 115, to a closed configuration to grasp tissue, for example, between the first jaw 8011 and the second jaw 8012. In the illustrated example, the first jaw 8011 comprises an anvil 8019 including staple forming pockets 8013 and the second jaw 8012 includes a longitudinal channel 8015 designed and sized to receive a staple cartridge 8040, as illustrated in FIG. 116. The staple cartridge 8040 is removably insertable in the longitudinal channel 8015. The staple cartridge 8040 and the longitudinal channel 8015 can be transitioned between an assembled configuration, wherein the staple cartridge 8040 is positioned in the longitudinal channel 8015, and an unassembled configuration where the staple cartridge 8040 is separate from the longitudinal channel 8015. The staple cartridge 8040 includes staples deformable against the staple forming pockets 8013 of the anvil 8019, in the assembled configuration. As described in greater detail below, the staple cartridge 8040 is configured for wireless transmission of power and/or data with the surgical instrument 8000, in an assembled configuration, as illustrated in FIG. 115.

While the present disclosure can be explained in the context of a linear stapler, this should not be construed as limiting. The present disclosure can readily be implemented in other types of surgical instruments that employ smart cartridges.

In the illustrated example, a drive shaft 8030 extends distally along the longitudinal central axis 8005. The drive shaft 8030 is coupled to a firing beam 8031, and is movable by the drive shaft 8030 to motivate a sequential deployment of staples from the staple cartridge 8040, and motivate a cutting of the stapled tissue by a knife on the firing beam 8031, for example. Additional details are described in U.S. patent application Ser. No. 14/200,111, entitled CONTROL SYSTEMS FOR SURGICAL INSTRUMENTS, filed Mar. 7, 2014, which issued on Apr. 25, 2017 as U.S. Pat. No. 9,629,629, which is hereby incorporated by reference herein in its entirety.

The closure of the end effector 8002 can be driven separately, e.g., by a closure tube, from staple firing. A closure tube could motivate one, or both, the jaws 8011, 8012 to move toward the closed configuration prior to, or concurrently with, advancement of the firing beam 8031 to deploy the stapes and cut the tissue grasped by the end effector 8002.

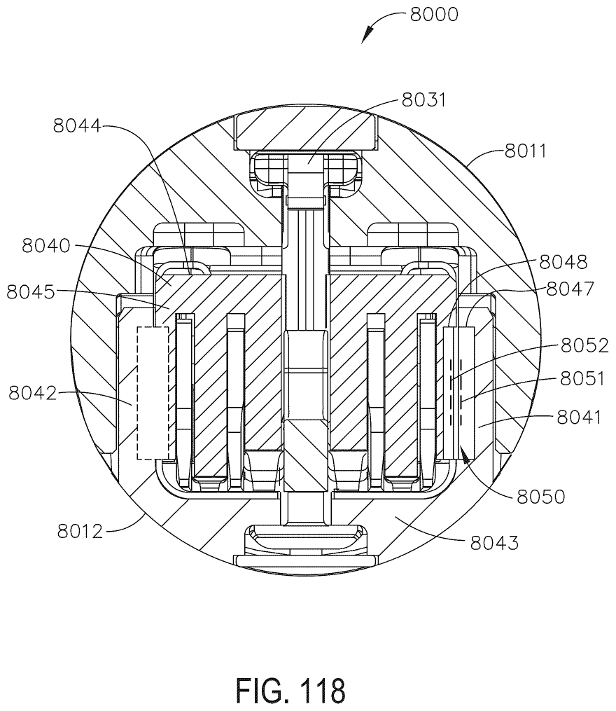

The surgical instrument 8000 further includes a wiring harness 8020 comprising a flex circuit that transmits at least one of data or power through the articulation joint assembly 8003 to the end effector 8002 from a power source or a data source proximal to the articulation joint assembly 8003. The flex circuit can transmit power to the end effector 8002 for powering an electronics package that communicates with a chip in the staple cartridge 8040, for example. A wireless signal-transfer circuit 8050 (FIG. 118) facilitates a wireless transmission of power and/or data between the flex circuit 8020 and the chip of the staple cartridge 8040, as described below in greater detail. Additionally, the flex circuit may define a communication pathway between the chip and a processor and/or a power source of the surgical instrument 8000 positioned proximal to the articulation joint assembly 8003.

The flex circuit can comprise floating ends. The flex circuit can comprise fixed ends. The flex circuit can comprise one floating end and one fixed end. The flex circuit can include a flexible substrate and a conductive layer disposed on the flexible substrate.

The interconnecting wiring harness 8020 can be comprise of twisted wire pairs or a flex circuit with similar shielding or overlapping conductors, for example, in order to minimize the electrical coupling to adjacent metallic components that may cause parasitic losses. The flex circuit may include a shielding layer that can be integrated into some of the layers of the flex circuit, for example, for magnetic coupling mitigation. The flex circuit traces can be overlaid in a twisted pair pattern to minimize the magnetic coupling to external metal and, thereby, minimize the creation of reinforced magnetic fields, for example. The flex circuit can be coiled/twisted down the entirety of the pathway of the wiring harness 8020, which can be the entire, or at least part of the entire, length of the shaft 8001, for example.

The coiled flex circuit allows for longitudinal movement of the flexible circuit. The flex circuit can be put into the twist-curl shape, which allows the flex circuit to have some shape memory. Accordingly, the coiled flex circuit may extend, then retract to an original shape, with articulation of the end effector 8001, for example, without risking damage to flex circuit components.

As best illustrated in FIG. 118, the wireless signal-transfer circuit 8050 includes a channel antenna 8051 and a cartridge antenna 8052. Moreover, the surgical instrument 8000 further includes an aligner 8060 that interlocks the staple cartridge 8040 and the longitudinal channel 8015 in the assembled configuration. The aligner 8060 is adjacent the channel antenna 8051 and the cartridge antenna 8051 in the assembled configuration to maintain a predefined spatial relation between the channel antenna 8051 and the cartridge antenna 8052.

Referring to FIGS. 115, 117, and 118, the longitudinal channel 8015 includes a base 8043, a first wall 8041 and a second wall 8042 extending from the base 8043. The second wall 8042 is spaced apart from the first wall 8041 to accommodate the staple cartridge 8040 therebetween in the assembled configuration. The channel antenna 8051 is housed in a cavity of the first wall 8041. A second channel antenna can be housed in a cavity of the second wall 8042. The channel antenna 8051 can be attached to, and protrudes from, the first wall 8041.

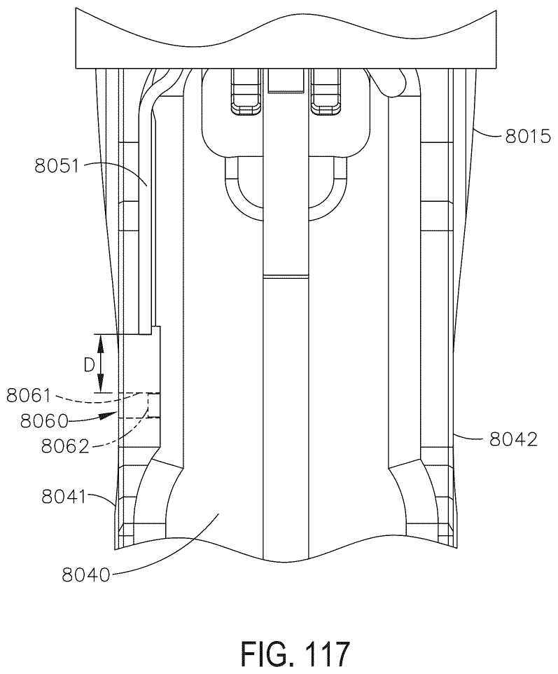

Referring primarily to FIGS. 117 and 118, the staple cartridge 8040 includes a cartridge deck 8044 that includes staple cavities, and a cartridge body 8045 housing staples driven through the staple cavities for deployment into tissue grasped between the staple cartridge 8040 and the staple forming pockets 8013 of the anvil 8019. As illustrated, the cartridge body 8045 can support the cartridge antenna 8052 for positioning against the channel antenna 8051 in the assembled configuration. As best illustrated in FIG. 118, the cartridge antenna 8052 is housed in a cavity in the cartridge body 8045. The cartridge antenna 8052 can be attached to, and protrudes from, the cartridge body 8045.

The aligner 8060 includes a channel alignment component 8061 (FIG. 115) defined in longitudinal channel 8015 and a corresponding cartridge alignment component 8062 (FIG. 116) of the staple cartridge 8040, which cooperates with the channel alignment component 8061 in the assembled configuration to maintain the channel antenna 8051 and the cartridge antenna 8052 in a predefined spatial relation. The aligner 8060 can define an interlocking, or mating, interface between the channel alignment component 8061 and the cartridge alignment component 8062, to maintain the predetermined spatial relation between the antennas 8051, 8052.

As illustrated in FIGS. 117 and 118, the channel alignment component 8061 can be in the form of a cavity, slot, or opening, and the cartridge alignment component 8062 can be in the form of a projection, post, or guide to be received in the channel alignment component 8061 in the assembled configuration. Alternatively, the cartridge alignment component 8062 can be in the form of a cavity, slot, or opening, and the channel alignment component 8061 can be in the form of a projection, post, or guide to be received in the channel alignment component 8061 in the assembled configuration. Other alignment components are contemplated by the present disclosure.

The predetermined spatial relation between the antennas 8051, 8052, as defined by the interlocking interface of the components of the aligner 8060, comprises a translational misalignment tolerance of less than or equal to a predefined distance. The translational misalignment tolerance includes a tolerance in one or more dimensions such as, for example, a longitudinal translational misalignment tolerance, a transverse translational misalignment tolerance, and/or a vertical translational misalignment tolerance. The aligner 8060 is to resist longitudinal, vertical, and/or transverse movement, or sliding, of the staple cartridge 8040 relative to the longitudinal channel 8015 in the assembled configuration, to ensure a proper alignment of the antennas 8051, 8052.

Additionally, or alternatively, the predetermined spatial relation comprises an angular misalignment tolerance of less than or equal to a predefined angle. The interlocking interface of the components of the aligner 8060 resists pitch, yaw, and/or roll movement of the staple cartridge 9040 relative to the longitudinal channel 8015.

The translational misalignment tolerance and/or the angular misalignment tolerance can be selected from a range of about 0.01% to about 10%, for example. Other values and/or ranges are contemplated by the present disclosure. The aligner 8060 can ensure a translational misalignment less than or equal to ±5 mm, ±3 mm, or ±1 mm, for example. The aligner 8060 can ensure an angular misalignment of less than or equal to ±5°, ±3°, or ±1°, for example.

The aligner 8060 can maintain an air-gap distance between the antennas 8051, 8052, in the assembled configuration, of about 4.5 mm. In some aspects, the air-gap distance is any value selected from a range of about 3 mm to about 10 mm, for example.

The alignment components 8061, 8062 can be designed in an offset configuration to resolve a manufacturing-induced misalignment between the channel antenna 8051 and the cartridge antenna 8052 in the assembled configuration. The manufacturing-induced misalignment may be due cartridge shrinkage. The designed offset between the alignment components 8061, 8062 can be tuned to address the manufacturing-induced misalignment.

An important characteristic of the aligner 8060 is its distance from the antennas 8051, 8052 in the assembled configuration. To effectively maintain the predetermined spatial relation between the antennas 8051, 8052, in a manner that ensures efficient/optimal power and/or data transfer therebetween, the aligner 8060 is to be positioned a predetermined distance (D), FIG. 118, from the antennas 8051, 8052. The distance (D) is selected from a range of about 1 mm to about 60 mm. The distance (D) can be selected from a range of about 5 mm to about 30 mm, or about 10 mm to about 20 mm. The distance (D) can be about 15 mm, for example.

The aligner 8060 and the antennas 8051, 8052 are located at a proximal portion 8059 of the end effector 8002, as best illustrated in FIG. 116. Alternatively, the aligner 8060 and the antennas 8051, 8052 can be located at an intermediate portion, or a distal portion, of the end effector 8002. Nonetheless, the position of the aligner 8060 and the antennas 8051, 8052 at the proximal portion reduces the length of the wiring harness 8020 needed to transmit power and/or data to/from the channel antenna 8051, which reduces parasitic losses and/or signal interference.

The aligner 8060 is positioned distal to the antennas 8051, 8052 in the assembled configuration, as illustrated in FIG. 116. Nonetheless, in other configurations, the aligner 8060 can be positioned proximal to the antennas 8051, 8052 in the assembled configuration. In yet other configurations, more than one aligner 8060 can be utilized. For example, in some configurations, an aligner 8060 can be positioned distal to the antennas 8051, 8052, and another aligner can be positioned proximal to the antennas 8051, 8052, such that the antennas 8051, 8052 are positioned between two aligners, in the assembled configuration, for example.

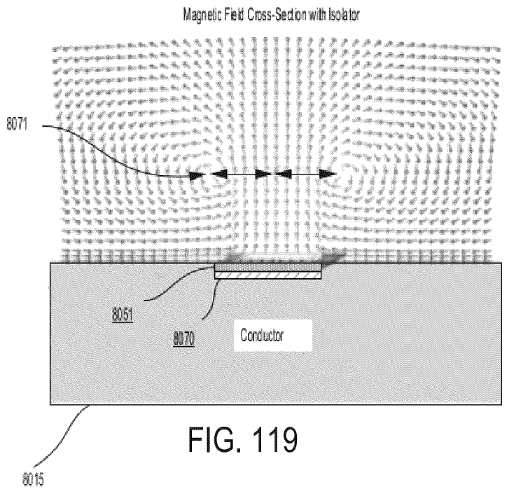

Referring primarily to FIG. 119, one or both of the antennas 8051, 8052 can be disposed onto an intermediate insulative, or non-conductive, layer 8070 to improve efficiency of power and/or data transfer between the antennas 8051, 8052, by reducing parasitic to neighboring surfaces such as, for example, to the longitudinal channel 8015. In the insulative, or non-conductive, layer 8070 focuses a magnetic field pattern 8071 between the antennas 8051, 8052, thereby preventing, or reducing, magnetic field drainage directed in its direction and/or wrapping fields directed in other directions, as illustrated in FIG. 119. The insulative, or non-conductive, layer 8070 may comprise a Urethane foam. The insulative, or non-conductive, layer 8070 may comprise silicone EMI. The insulative, or non-conductive, layer 8070 may comprise a microwave shielding material impregnated with carbon fibers or magnetically loaded, for example. The insulative, or non-conductive, layer 8070 may comprise ECCOSORF™.

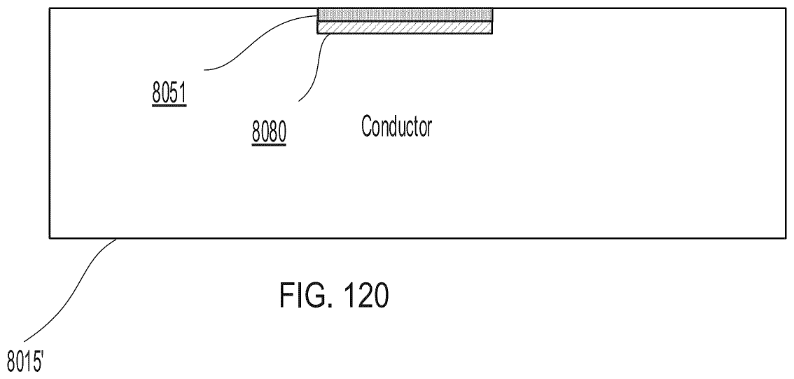

Instead of the insulative, or non-conductive, layer 8070, or in addition thereto, a ferrite shield 8080 can be utilized to focus/manage the magnetic field in the inductive coupling between the antennas 8051, 8052. FIG. 120 illustrates a longitudinal channel 8015′, similar in many respects to the longitudinal channel 8015, wherein a ferrite shield 8080 is disposed between the longitudinal channel 8015′ and the channel antenna 8051. Additionally, or alternatively, a similar ferrite shield can be disposed between the cartridge body 8045 and the cartridge antenna 8052, for example. Additionally, or alternatively, a ferrite shield can be disposed between the antennas 8051, 8052, for example.

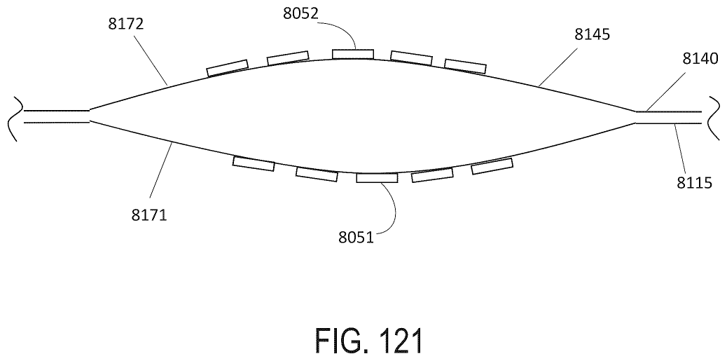

FIG. 121 illustrates alternative longitudinal channel 8115 and staple cartridge 8140 in an assembled configuration. The longitudinal channel 8115 and staple cartridge 8140 are similar in many respects to the longitudinal channel 8015 and staple cartridge 8040, which are not repeated herein for brevity. Alternatively, the surgical instrument 8000 may include the longitudinal channel 8115 instead of the longitudinal channel 8015. The staple cartridge 8140 can be removably received in the longitudinal channel 8115, in the assembled configuration.

The longitudinal channel 8115 and staple cartridge 8140 comprise cavities 8171, 8172, respectively, defined in corresponding outer surfaces thereof, as illustrated in FIG. 121. The cavities 8171, 81712 define curved, or concave, zones, or surfaces, that accommodate the antennas 8051, 8052, respectively. The arcuate disposition of the antennas 8051, 8052 along the curved zones of the cavities 8171, 8172 provides a three-dimensional effect that focuses the transmission field between the antennas 8051, 8052. Alternatively, only the longitudinal channel 8115 or staple cartridge 8140 may comprise a curved antenna disposition, while the other remains flat, or at least substantially flat.

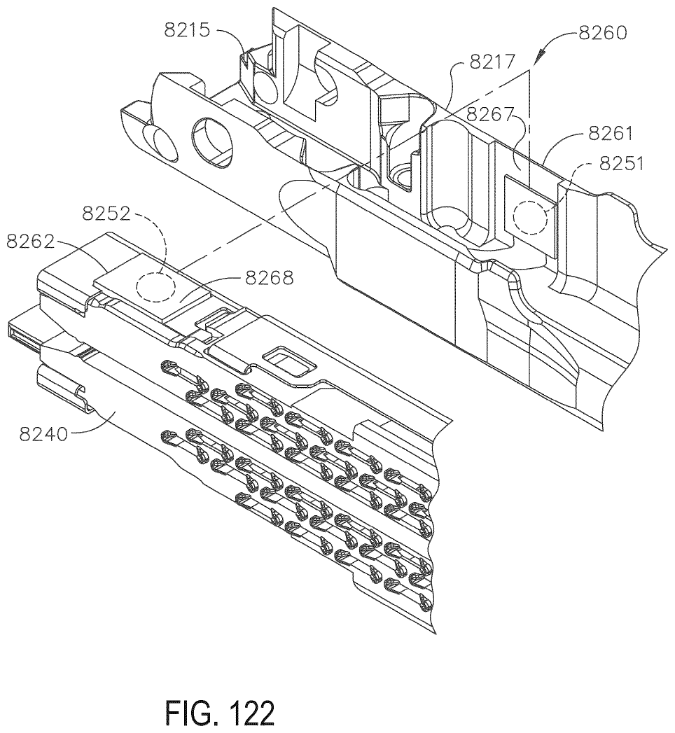

FIG. 122 illustrates alternative longitudinal channel 8215 and staple cartridge 8240 that are similar in many respects to the longitudinal channel 8015 and staple cartridge 8040, which are not repeated herein for brevity. Alternatively, the surgical instrument 8000 may include the longitudinal channel 8215 instead of the longitudinal channel 8015. The staple cartridge 8240 can be removably received in the longitudinal channel 8215, in the assembled configuration.

The longitudinal channel 8215 and staple cartridge 8240 cooperatively form an aligner 8260 that incorporates, or integrates, antennas 8251, 8252 into a channel alignment component 8261 and cartridge alignment component 8262, respectively. As illustrated in FIG. 122, the antennas 8251, 8252 can be positioned on, or embedded in, the channel alignment component 8261 and cartridge alignment component 8262, respectively.

The staple cartridge 8240 is similar in many respects to the staple cartridge 8040, which are not repeated herein for brevity. The cartridge alignment component 8262 is positioned on a cartridge body 8245 of the staple cartridge 8240, and is shaped and sized for a matting engagement with the channel alignment component 8261 defined on a side wall 8217 of the longitudinal channel 8215.

The channel alignment component 8261 includes a depression, or cavity 8267, defined in the side wall 8217. The cartridge alignment component 8262 includes a corresponding projection 8268 for matting engagement with the cavity 8267. The antennas 8251, 8252 reside in the cavity 8267 and projection 8268, respectively. The antennas 8251, 8252 are within sufficiently close proximity for signal transmission therebetween when the projection 8268 is assembled with the cavity 8267.

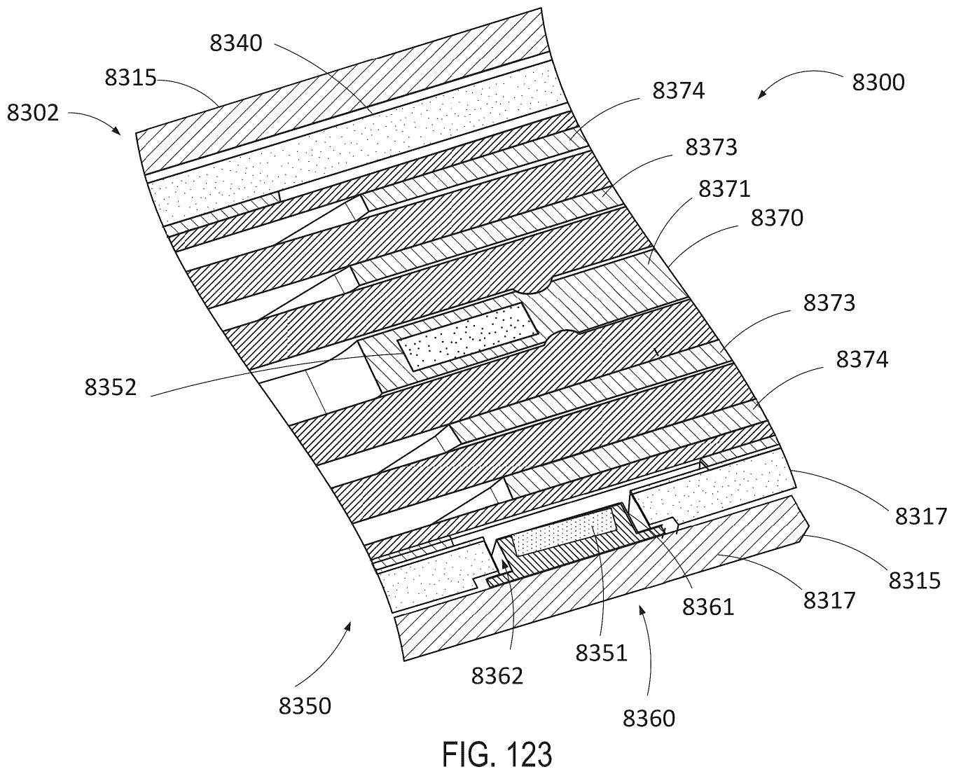

FIG. 123 is a partial cross-sectional view of a surgical instrument 8300 similar in many respects to the surgical instrument 8000, which are not repeated herein for brevity. For example, the surgical instrument 8300 includes a shaft 8001, and may include an articulation joint assembly 8003. The surgical instrument 8300 also includes an end effector 8302 with jaws for grasping tissue therebetween. Moreover, the end effector 8302 includes a longitudinal channel 8315 shaped and sized to retain a staple cartridge 8340.

The staple cartridge 8340 is removably insertable in the longitudinal channel 8315. The staple cartridge 8340 and the longitudinal channel 8315 can be transitioned between an assembled configuration, wherein the staple cartridge 8340 is positioned in the longitudinal channel 8315, and an unassembled configuration where the staple cartridge 8340 is separate from the longitudinal channel 8315. The staple cartridge 8340 includes staples deformable against staple forming pockets of an anvil of the end effector 8302, in the assembled configuration.

The staple cartridge 8340 is configured for wireless transmission of power and/or data with the surgical instrument 8300, in the assembled configuration, as illustrated in FIG. 123. The surgical instrument 8300 includes a wireless signal-transfer circuit 8350 similar in many respects to the wireless signal-transfer circuit 8050, which are not repeated herein for brevity. The wireless signal-transfer circuit 8350 is to transfer data and/or power between the surgical instrument 8300 and the staple cartridge 8340. While the wireless signal-transfer circuit 8050 includes cartridge antenna 8052 defined in the cartridge body of the staple cartridge 8015, the wireless signal-transfer circuit 8350 includes a cartridge antenna 8352 defined in, or housed in, a sled 8370 within the staple cartridge 8340.

The sled 8370 is movable distally from a home position (FIG. 123) during a firing motion to engage and lift staple drivers to eject staples from the staple cartridge 8340 into tissue grasped by the end effector 8302. The sled 8370 is a four-rail sled having a central upright 8371 configured to accommodate the cartridge antenna 8352, a pair of inner rails 8373, and a pair of outer rails 8374. The rails 8373, 8374 are to engage and lift the staple drivers during the firing motion to eject the staples from the staple cartridge 8340. The cartridge antenna 8352 can be housed in one of the rails 8373, 8374, for example.

The surgical instrument 8300 further includes a channel antenna 8351 positioned at a side wall of 8317 of the longitudinal channel 8315, in alignment with the cartridge antenna 8352, while the sled 8370 is in the home position, as illustrated in FIG. 123. An aligner 8360 interlocks the staple cartridge 8340 and the longitudinal channel 8315 in the assembled configuration to ensure a proper alignment between the antennas 8351, 8352 in the home position, for efficient transmission of power and/or data through the wireless signal-transfer circuit 8350.

The aligner 8360 is at a proximal portion of the end effector 8302, and is adjacent the channel antenna 8351 and the cartridge antenna 8352 in the assembled configuration. The aligner 8360 is configured to maintain a predefined spatial relation between the channel antenna 8351 and the cartridge antenna 8352, while the sled 8370 is in the home position.

The aligner 8360 includes a channel alignment component 8361 and a cartridge alignment component 8362 configured for matting engagement with the channel alignment component 8361. The alignment components 8361, 8362 are similar to the alignment components 8261, 8262, respectively. The channel alignment component 8361 protrudes, or outwardly extends, from the side wall 8317, and is configured to house the channel antenna 8351 therein. The cartridge alignment component 8362 defines an opening configured to receive the channel alignment component 8361 in the assembled configuration. Alternatively, the opening can be defined by the side wall 8317, and the protrusion by the cartridge body of the staple cartridge 8315. In any event, the channel alignment component 8361 and corresponding cartridge alignment component 8362, cooperatively maintain the channel antenna 8351 and the cartridge antenna 8352 in a predefined spatial relation.

In various aspects, one or more electrical circuits, e.g., various flex circuits described herein, can be integrated into mechanical and/or structural components of a surgical instrument such as, for example, a handle, a shaft, and/or an end effector. The electrical circuits can be incorporated into the body of the components or their coatings. Various conductors can be incorporated in shrouds of a handle, for example.

In some aspects, anvils and/or longitudinal channels of the present disclosure can be modified to include print traces directly onto metal surfaces of the anvils and/or the longitudinal channels, for example, to minimize space needed for the electrical circuits. The same approach can be applied to coils for wireless communications. The coil can be printed directly on the walls of the longitudinal channels. In fact, the metal channel backing may increase the wireless strength.

In some aspects, the metal components of the shafts and/or the handles described herein can be utilized as ground return for electrical circuits therein. In one example, one or more of the drive/firing bars described herein can be made from a plurality of metal laminates, one or more of which can be used as conductors through the shaft, by applying conductive traces thereto. A dielectric layer must be placed be utilized to separate the laminates from the conductive traces. In some aspects, the laminates can be made out of a non-conductive material to allow the material to be receptive to an electrical conductor. The traces can be made from conductive inks or epoxies.

Conductive traces can also be applied to a handle shroud of a surgical instrument, in accordance with the present disclosure. The surgical instrument can be battery powered, and the battery can be stored within the handle shroud, for example. The conductive traces can be utilized to form power and ground connections from the battery, which can be routed to various portion of the surgical instrument, while occupying minimal space there within. For example, power to the motor could be routed down the handle plastic to the area near the handle end. The plastic cross rib could have the electrical contacts set up such that when the MGB is placed into the shroud, the motor terminals contact the plastic rib in the proper location to be energized.

Various sensors, in accordance with the present disclosure, can be connected for data and/or power transmission through conductive traces disposed in nearby portion of a surgical instrument such as, for example, the shrouds that are designed to wrap and protect the internal frame that carries and locates the mechanical components of the surgical instrument. One or more sensors could be positioned to take advantage of the shrouds proximity to key mechanical components. For example, sensors could be attached to the shroud near trigger such as, for example, a closure trigger. By placing the sensor on the shroud near the closure trigger, a direct measure of the closure trigger location during the closure cycle can be achieved. This direct measure can be correlated to the closure state of the surgical instrument. The shrouds location near the closure trigger would allow the sensor to be positioned perpendicular to the trigger, and ensure a high fidelity signal.

Further to the above, the placement of conductive traces directly on, or near, functional components such as, for example, a motor and/or a motor gear box permits a direct measurement of speed at the motor, or at the motor gearbox. The shroud portions of the handle closely surround the motor gearbox to keep the size of the handle at an optimal size for a proper grip. Accordingly, placement of the sensors and/or the conductive traces on, or near, the shroud portions extending around the motor, or the motor gear box, permits optimal sensor measurements. For example, a motor encoder can be placed directly at the back end of the motor, which permits more accurate speed measures, and reduces feedback error. The conductive traces, which are placed on the surrounding shroud portions, can then connect the motor encoder to a control circuit of the surgical instrument.

In addition to the conductive traces, in place of the conductive traces, a semi rigid wiring harness can be utilized in the shroud portions of the handle, for power and/or data transmission along a fixed semi-rigid track within the shrouds.

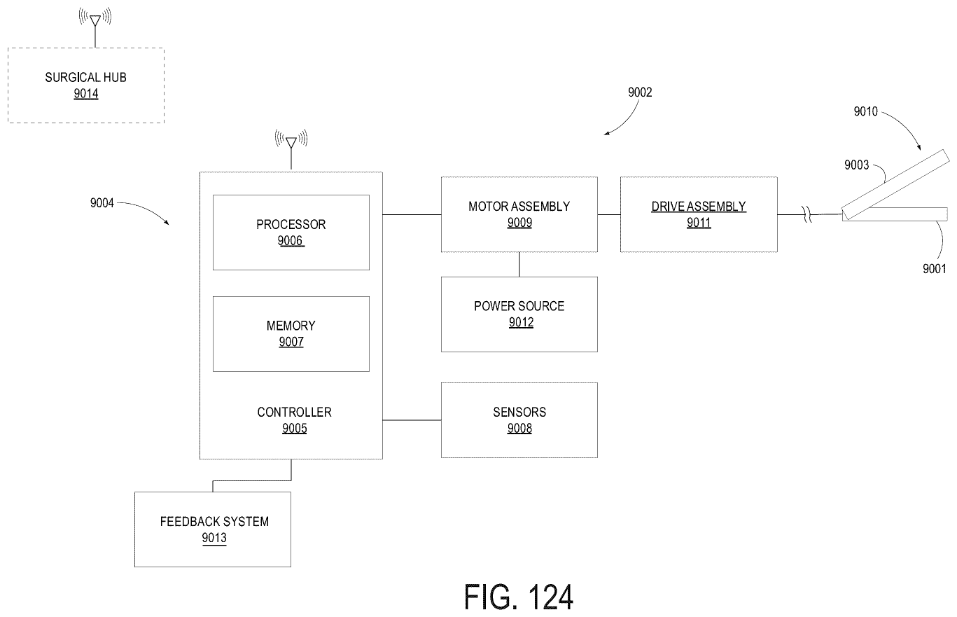

FIG. 124 illustrates a block diagram of a surgical system 9002 for use with one or more surgical instruments, tools, and/or robotic systems in accordance with the present disclosure. The system 9002 includes a control circuit 9004. The control circuit 9004 includes a microcontroller 9005 comprising a processor 9006 and a storage medium such as, for example, a memory 9007.

A motor assembly 9009 includes one or more motors, driven by motor drivers. The motor assembly 9009 operably couples to a drive assembly 9011 to drive, or effect, one or more motions at an end effector 9010. The drive assembly 9011 may include any number of components suitable for transmitting motion to the end effector 9010 such as, for example, one or more gears, gear sets, gear transmissions with one or multiple selectable gears, linkages, bars, tubes, and/or cables, for example.

One or more of sensors 9008, for example, provide real-time feedback to the processor 9006 about one or more operational parameters monitored during a surgical procedure being performed by the surgical system 9002. The operational parameters can be associated with a user performing the surgical procedure, a tissue being treated, and/or one or more components of the surgical system 9002, for example. The sensors 9008 may comprise any suitable sensor, such as, for example, a magnetic sensor, such as a Hall effect sensor, a strain gauge, an encoder, a position sensor, a force sensor, a pressure sensor, an inductive sensor, such as an eddy current sensor, a resistive sensor, a capacitive sensor, an optical sensor, and/or any other suitable sensor.

Further to the above, in accordance with the present disclosure, the sensors 9008 may comprise any suitable sensor for detecting one or more conditions at the end effector 9010 including, without limitation, a tissue thickness sensor such as a Hall Effect Sensor or a reed switch sensor, an optical sensor, a magneto-inductive sensor, a force sensor, a pressure sensor, a piezo-resistive film sensor, an ultrasonic sensor, an eddy current sensor, an accelerometer, a pulse oximetry sensor, a temperature sensor, a sensor configured to detect an electrical characteristic of a tissue path (such as capacitance or resistance), or any combination thereof. As another example, and without limitation, the sensors 9008 may include one or more sensors located at, or about, an articulation joint extending proximally from the end effector 9010. Such sensors may include, for example, a potentiometer, a capacitive sensor (slide potentiometer), piezo-resistive film sensor, a pressure sensor, a pressure sensor, or any other suitable sensor type. In some arrangements, the sensor 9008 may comprise a plurality of sensors located in multiple locations in the end effector 9010, including the staple cartridge, for example.

In accordance with the present disclosure, the surgical system 9002 can include a feedback system 9013 which includes one or more devices for providing a sensory feedback to a user. Such devices may comprise, for example, visual feedback devices (e.g., an LCD display screen, a touch screen, LED indicators), audio feedback devices (e.g., a speaker, a buzzer) or tactile feedback devices (e.g., haptic actuators).

The microcontroller 9005 may be programmed to perform various functions such as precise control over the speed and position of the drive assembly 9011. The microcontroller 9005 may be any single-core or multicore processor such as those known under the trade name ARM Cortex by Texas Instruments. Additionally, the main microcontroller 9005 may be an LM4F230H5QR ARM Cortex-M4F Processor Core, available from Texas Instruments, for example, comprising an on-chip memory of 256 KB single-cycle flash memory, or other non-volatile memory, up to 40 MHZ, a prefetch buffer to improve performance above 40 MHz, a 32 KB single-cycle SRAM, and internal ROM loaded with StellarisWare® software, a 2 KB EEPROM, one or more PWM modules, one or more QEI analogs, and/or one or more 12-bit ADCs with 12 analog input channels, details of which are available for the product datasheet.

The microcontroller 9005 may be configured to compute a response in the software of the microcontroller 9005. The computed response is compared to a measured response of the actual system to obtain an “observed” response, which is used for actual feedback decisions. The observed response is a favorable, tuned value that balances the smooth, continuous nature of the simulated response with the measured response, which can detect outside influences on the system.

The motor assembly 9009 includes one or more electric motors and one or more motor drivers. The electric motors can be in the form of a brushed direct current (DC) motor with a gearbox and mechanical links to the drive assembly 9011. In accordance with the present disclosure, a motor driver may be an A3941 available from Allegro Microsystems, Inc.

In accordance with the present disclosure, the motor assembly 9009 may include a brushed DC driving motor having a maximum rotational speed of approximately 25,000 RPM. Alternatively, the motor assembly 9009 may include a brushless motor, a synchronous motor, a stepper motor, or any other suitable electric motor. The motor driver may comprise an H-bridge driver comprising field-effect transistors (FETs), for example.

The motor assembly 9009 can be powered by a power source 9012. The power source 9012 can include one or more batteries which may include a number of battery cells connected in series that can be used as the power source to power the motor assembly 9009. In accordance with the present disclosure, the battery cells of the power assembly may be replaceable and/or rechargeable. Further, accordance with the present disclosure, the battery cells comprise lithium-ion batteries which can be couplable to and separable from the power assembly.

Further to the above, the end effector 9010 includes a first jaw 9001 and a second jaw 9003. At least one of the first jaw 9001 and the second jaw 9003 is rotatable relative to the other during a closure motion that transitions the end effector 9010 from an open configuration toward a closed configuration. In accordance with the present disclosure, a cartridge jaw may be movable relative to a fixed anvil jaw to a clamped position. Additionally, an anvil jaw may be movable relative to a fixed cartridge jaw to a clamped position. Furthermore, an anvil jaw and a cartridge jaw may both be movable relative to each other to a clamped position. The closure motion may cause the jaws 9001, 9003 to grasp tissue therebetween. In accordance with the present disclosure, sensors, such as, for example, a strain gauge or a micro-strain gauge, can be configured to measure one or more parameters of the end effector 9010, such as, for example, the amplitude of the strain exerted on the one or both of the jaws 9001, 9003 during a closure motion, which can be indicative of the closure forces applied to the jaws 9001, 9003. The measured strain is converted to a digital signal and provided to the processor 9006, for example. Alternatively, additionally, sensors such as, for example, a load sensor, can measure a closure force and/or a firing force applied to the jaws 9001, 9003.

In accordance with the present disclosure, a current sensor can be employed to measure the current drawn by a motor of the motor assembly 9009. The force required to advance the drive assembly 9011 can correspond to the current drawn by the motor, for example. The measured force is converted to a digital signal and provided to the processor 9006.

In accordance with the present disclosure, strain gauge sensors can be used to measure the force applied to the tissue by the end effector 9010, for example. A strain gauge can be coupled to the end effector 9010 to measure the force on the tissue being treated by the end effector 9010. In accordance with the present disclosure, the strain gauge sensors can measure the amplitude or magnitude of the strain exerted on a jaw of an end effector 9010 during a closure motion which can be indicative of the tissue compression. The measured strain is converted to a digital signal and provided to a processor 9006.

The measurements of the tissue compression, the tissue thickness, and/or the force required to close the end effector on the tissue, as respectively measured by the sensors 9008 can be used by the microcontroller 9005 to characterize the selected position of one or more components of the drive assembly 9011 and/or the corresponding value of the speed of one or more components of the drive assembly 9011. In accordance with the present disclosure, a memory (e.g. memory 9007) may store a technique, an equation, and/or a look-up table which can be employed by the microcontroller 9005 in the assessment.

The system 9002 may comprise wired or wireless communication circuits to communicate with surgical hubs (e.g. surgical hub 9014), communication hubs, and/or robotic surgical hubs, for example. Additional details about suitable interactions between a system 9002 and the surgical hub 9014 are disclosed in U.S. patent application Ser. No. 16/209,423 entitled METHOD OF COMPRESSING TISSUE WITHIN A STAPLING DEVICE AND SIMULTANEOUSLY DISPLAYING THE LOCATION OF THE TISSUE WITHIN THE JAWS, now U.S. Patent Application Publication No. 2019/0200981, the entire disclosure of which is incorporated by reference in its entirety herein.

In accordance with the present disclosure, the control circuit 9004 can be configured to implement various processes described herein. The control circuit 9004 may comprise a microcontroller comprising one or more processors (e.g., microprocessor, microcontroller) coupled to at least one memory circuit. The memory circuit stores machine-executable instructions that, when executed by the processor, cause the processor to execute machine instructions to implement various processes described herein. The processor may be any one of a number of single-core or multicore processors known in the art. The memory circuit may comprise volatile and non-volatile storage media. The processor may include an instruction processing unit and an arithmetic unit. The instruction processing unit may be configured to receive instructions from the memory circuit of this disclosure.

Alternatively, the control circuit 9004 can be in the form of a combinational logic circuit configured to implement various processes described herein. The combinational logic circuit may comprise a finite state machine comprising a combinational logic configured to receive data, process the data by the combinational logic, and provide an output.

Alternatively, the control circuit 9004 can be in the form of a sequential logic circuit. The sequential logic circuit can be configured to implement various processes described herein. The sequential logic circuit may comprise a finite state machine. The sequential logic circuit may comprise a combinational logic, at least one memory circuit, and a clock, for example. The at least one memory circuit can store a current state of the finite state machine. In accordance with the present disclosure, the sequential logic circuit may be synchronous or asynchronous. Additionally, the control circuit 9004 may comprise a combination of a processor (e.g., processor 9006) and a finite state machine to implement various processes herein. Furthermore, the finite state machine may comprise a combination of a combinational logic circuit (and the sequential logic circuit, for example.

In accordance with the present disclosure, the staple cartridges described herein are replaceable. The staple cartridge can include one or more electronic systems onboard the staple cartridge. Discussed in greater detail below, one or more of the electronic systems onboard the staple cartridge may be modular and/or replaceable. In accordance with the present disclosure, the electronic systems may be in electrical communication with one or more components of the surgical system 9002. The one or more electronic systems can include, sensor circuits including sensors to measure end effector parameters during a surgical stapling procedure, cartridge-identifying circuits including a circuit with a detectable property to be able to identify one or more properties of the replaceable staple cartridge, and an onboard memory and processor. Additionally, the electronic systems can include a modular electronics package including a PCB, for example, which is attachable to, and detachable from, the replaceable staple cartridge. Furthermore, one or more of the electronic systems may be replaceable with the same or different electronic systems. Such an arrangement can enable a manufacturer or user, for example, to specifically select the desired onboard electronics of the replaceable staple cartridge. For example, different electronics packages may be desired for different types of target tissue. In accordance with the present disclosure, the hardwired electrical pathways between the sensor circuits of the replaceable staple cartridge and the modular electronics package can be modified from electronics package to electronics package.

One or more of the electronic systems of the staple cartridge may also include the method for communicating power and/or data to and/or from the staple cartridge to a control circuit of the overall surgical stapling system (surgical robot, instrument handle, motor control circuits, etc.). In accordance with the present disclosure, power and/or data may be transmitted to and/or from the staple cartridge by way of hardwired connections where physical contact between contacts or connectors of the staple cartridge and contacts or connectors of the cartridge jaw within which the staple cartridge is to be installed is required. In addition to, or in lieu of, the hardwired connections, the staple cartridge and cartridge jaw may each comprise one or more wireless transmission coils to transmit data and/or power to and/or from the staple cartridge.

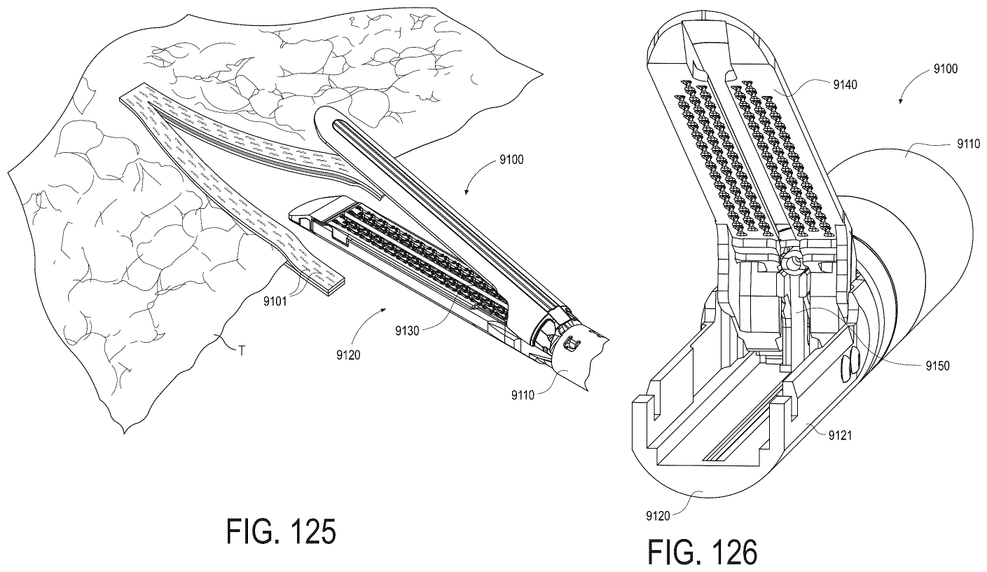

FIGS. 125 and 126 depict a surgical stapling assembly 9100 configured to clamp, staple, and cut patient tissue T during a surgical stapling procedure. As discussed herein, one or more functions of the surgical stapling assembly 9100 can be motor-driven. The surgical stapling assembly 9100 comprises a shaft 9110 and an end effector 9120 extending from the shaft 9110. The end effector 9120 comprises a cartridge channel jaw 9121 and an anvil jaw 9140 movable relative to the cartridge channel jaw 9121 to clamp tissue therebetween during a clamping stroke. In accordance with the present disclosure, the cartridge channel jaw 9121 can be movable in addition to, or in lieu of, the anvil jaw 9140. The end effector 9120 further comprises a replaceable staple cartridge 9130 configured to be installed into the cartridge channel jaw 9121. The replaceable staple cartridge 9130 comprises a plurality of staples 9101 removably stored therein and configured to be ejected from the replaceable staple cartridge 9130 during a staple firing stroke. In accordance with the present disclosure, the staple cartridge 9130 may not be replaceable. Further, in accordance with the present disclosure, a disposable loading unit may comprise a shaft and an end effector attachable to a control interface. Additionally, the entire cartridge channel jaw 9121 may be replaceable.

The surgical stapling assembly 9100 further comprises a firing driver 9150 actuatable through the end effector 9120 by a drive assembly such as the drive assembly 9011, for example. The firing driver 9150 can comprise any suitable firing driver such as, for example, a distal I-beam head, discussed in greater detail below. The firing driver 9150 is configured to push a sled of the replaceable staple cartridge 9130 from an unfired position to a fired position. During distal translation of the sled within the replaceable staple cartridge 9130, the sled is configured to sequentially lift a plurality of staple drivers with staples 9101 supported thereon. As the drivers are lifted toward the anvil jaw 9140, the drivers are configured to eject the staples 9101 from a plurality of staple cavities and against the anvil jaw 9140.

In accordance with the present disclosure, the sled may be part of the firing driver. Any suitable combination of firing components can be considered the firing driver.

In accordance with the present disclosure, moving the anvil jaw 9140 into a clamped position to clamp tissue between the anvil jaw 9140 and the replaceable staple cartridge 9130 can be performed by a closure driver. The closure driver may be separate from the firing driver 9150 and may be actuatable independently of the firing driver. Alternatively, the closure driver may not be separate from the firing driver 9150. In accordance with the present disclosure, the clamping, or closing, motion may be performed by the firing driver 9150. Opposing jaw-camming pins of a distal I-beam head of the firing driver 9150 are configured to cam the anvil jaw 9140 into a clamped position as the firing driver 9150 is actuated distally through an initial clamping stroke, or motion. In addition to moving the anvil jaw 9140 from an unclamped position to a clamped position during a clamping stroke, the opposing jaw-camming pins are configured to control a tissue gap distance between the anvil jaw 9140 and the replaceable staple cartridge 9130 during the staple firing stroke by limiting the separation of the cartridge channel jaw 9121 and the anvil jaw 9140 during the staple firing stroke with the opposing jaw-camming pins. One of the jaw-camming pins is configured to engage the cartridge channel jaw 9121 and one of the jaw-camming pins is configured to engage the anvil jaw 9140.

In accordance with the present disclosure, the replaceable staple cartridge 9130 may comprise onboard electronics. The onboard electronics require power and/or data transmission to and/or from the replaceable staple cartridge. Thus, an electrical interface exists between the replaceable staple cartridge and the cartridge channel jaw 9121. In accordance with the present disclosure, a control circuit (e.g., control circuit 9004) can be electrically coupleable to the onboard electronics of the replaceable staple cartridge 9130. Any suitable transmission technique can be employed. Various examples are described in greater detail below.

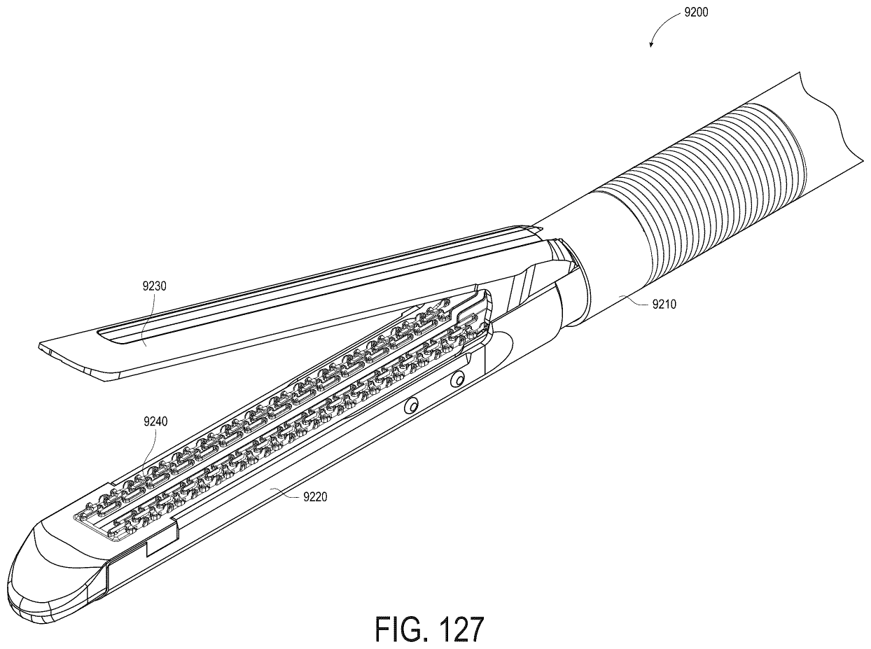

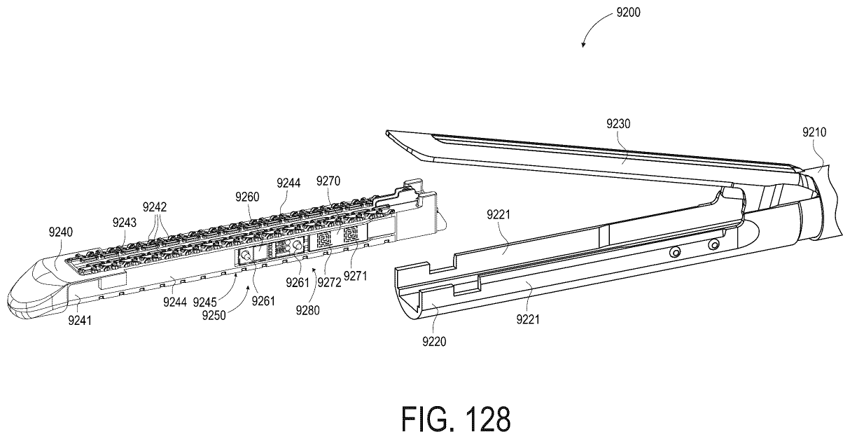

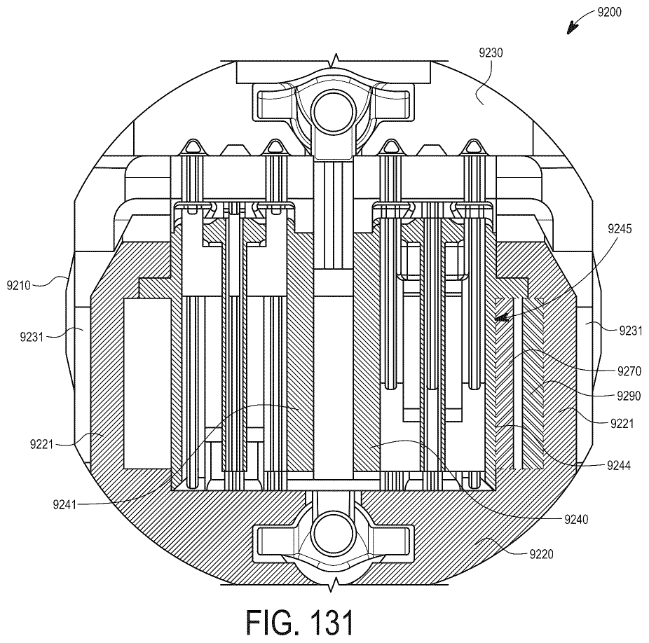

FIGS. 127-131 depict a surgical stapling end effector 9200 comprising a shaft 9210, a cartridge channel 9220, and an anvil 9230 movable relative to the cartridge channel 9220 to clamp tissue therebetween. The surgical stapling end effector 9200 further comprises a replaceable staple cartridge 9240 positioned within the cartridge channel 9220 and containing staples deployable by a firing driver during a firing stroke. The replaceable staple cartridge 9240 comprises a cartridge body 9241 comprising a plurality of staple cavities 9242, a longitudinal slot 9243, and sidewalls 9244. One of the sidewalls 9244 comprises a receptacle 9245. The replaceable staple cartridge 9240 comprises onboard electronics 9250 comprising a modular electronics package 9260 and a wireless transmission interface 9270 configured to transmit one of power and/or data to and/or from the replaceable staple cartridge 9240 to and/or from the control circuit 9004 of the surgical system 9002 with which the surgical stapling end effector 9200 is used.

The onboard electronics, or electronics sub-assembly, 9250 are positioned within the receptacle 9245. The receptacle 9245 can be configured to receive one or more modular electronics packages 9260. In accordance with the present disclosure, when installed in the receptacle 9245, the modular electronics packages 9260 can be electrically coupled with the wireless transmission interface 9270 via electrical traces 9280 positioned within the receptacle 9245. Additionally, in accordance with the present disclosure, additional electrical traces can be employed to connect the receptacle 9245 (and, thus, the electronics package 9260 and/or wireless transmission interface 9270) to various sensors onboard the staple cartridge 9240. The modular electronics package 9260 comprises a PCB including a processer and a memory. However, any suitable modular electronics package 9260 can be employed with any suitable electrical components such as sensors, multiple processors, multiple memories, etc. The modular electronics package 9260 is pinned to the sidewall 9244 via attachment pins 9261. A user may replace the modular electronics package 9260 with a different modular electronics package 9260 by disengaging the pins 9261, removing the modular electronics package 9260, placing a different modular electronics package in place of the modular electronics package 9260, and re-engaging the pins 9261. Each modular electronics package 9260 may comprise attachment pins which can be press fit, or snap fit, into corresponding apertures defined in the receptacle 9245 such that the pins remain a part of the modular electronics package 9260.

In accordance with the present disclosure, the one or more modular electronics packages 9260 can be electrically coupled with onboard electrical circuits of the replaceable staple cartridge 9240 such as, for example, sensor circuits including sensors for measuring one or more end effector parameters, lockout circuits for identifying of the staple cartridge is spent or unspent, cartridge-identifying circuits for identifying the specific type, size, length, and/or color, of the staple cartridge 9240, and/or RFID circuits, etc. The signals received from the various onboard circuits can be communicated to the modular electronics package 9260. In accordance with the present disclosure, the signals received from the various onboard circuits can be communicated to the control circuit 9004 of the surgical system 9002 with which the staple cartridge 9260 is used in addition to, or in lieu of, to the modular electronics package 9260. The onboard circuits can be powered through the onboard electronics package 9260. Additionally, the onboard circuits can receive power directly from the wireless transmission interface 9270. Multiple power sources may be employed. Further, accordance with the present disclosure, one or more of the power sources used can act as backup power sources.

As discussed above, the wireless transmission interface 9270 is configured to transmit data and/or power to the control circuit 9004 of the surgical system 9002. The wireless transmission interface 9270 comprises a proximal transmission coil 9271 and a distal transmission coil 9272. In accordance with the present disclosure, one of the coils may transmit data therethrough and the other coil may transmit power therethrough. The wireless transmission interface 9270 is configured to transmit the power and/or data to and/or from the modular electronics package 9260 through electrical traces 9280. In accordance with the present disclosure, the modular electronics package 9260 may comprise contacts configured to engage the electrical traces 9280 upon installation of the modular electronics pack 9260 into the receptacle 9245.

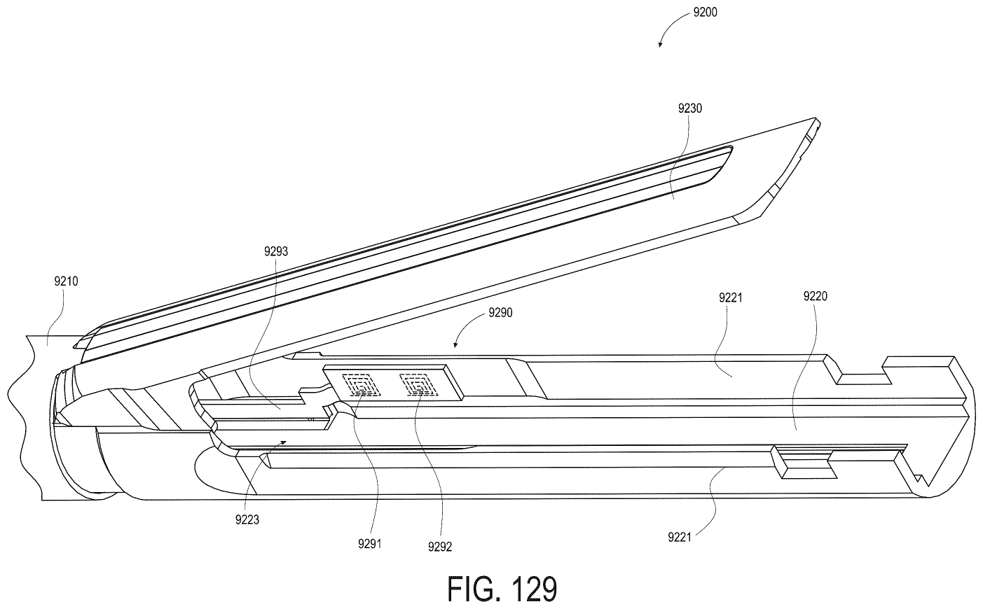

Referring to FIG. 129, the cartridge channel jaw 9220 comprises channel sidewalls 9221. The surgical stapling end effector 9200 further comprises a wireless transmission interface 9290 attached to one of the channel sidewalls 9221. The wireless transmission interface 9290 comprises a proximal transmission coil 9291 and a distal transmission coil 9292. The wireless transmission interface 9290 further comprises a flex circuit 9292 at least partially positioned within a slot 9223 defined in the cartridge channel jaw 9220. Once the replaceable staple cartridge 9260 is properly installed in the cartridge channel jaw 9220, the proximal wireless transmission coils 9271, 9291 are sufficiently aligned to wireless transmit power and/or data therethrough and the distal wireless transmission coils 9272, 9292 are sufficiently aligned to wireless transmit power and/or data therethrough. In accordance with the present disclosure, the control circuit 9004 can be configured to alert a user upon obtaining proper alignment between the coils 9271, 9272, 9291, 9292. Additionally, one pair of the coils may transmit power and one pair of the coils may transmit data. The wireless transmission interfaces 9270, 9290 can comprise any suitable material to host the wireless transmission coils and connect the coils to the outgoing/ingoing electrical pathways (such as the flex circuit 9293 and electrical traces 9280, for example).

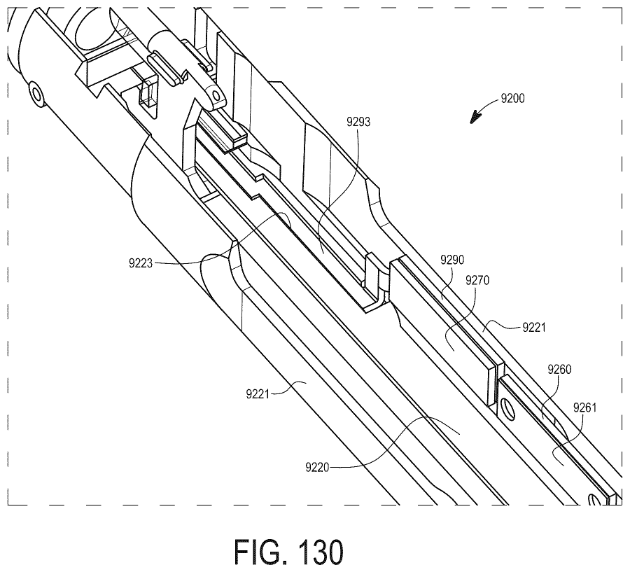

As can be seen in FIG. 130, the flex circuit 9293 is positioned within the slot 9223 of the cartridge channel jaw 9220. The flex circuit 9293 electrically couples the wireless transmission interface 9290 (specifically the coils 9291, 9292) to the control circuit 9004 of the surgical system 9002 to supply the coils 9291, 9292 with data and/or power. The flex circuit 9293 is configured to run proximally toward a surgical instrument control interface such as, for example, a surgical instrument handle and/or robot puck interface, for example.

Described herein are a plurality of onboard electronics of a replaceable surgical staple cartridge. In accordance with the present disclosure, one or more of the onboard electronics can be identifiable, or detectable, by the control circuit 9004 of the surgical system 9002. Certain control algorithms can be selected and/or adjusted based on the specific onboard electronics. For example, a clinician may plan a procedure targeting a specific type of tissue. The specific type of tissue may require a specific length, type, or color staple cartridge. The specific type of tissue may also require a specific firing algorithm. For example, the specific type of tissue may be thinner than other types of tissue and a slower, more delicate, firing and/or clamping control algorithm may be selected by the control circuit 9004.

When the control circuit 9004 selects a specific control algorithm, the onboard electronics can be utilized in a specific predetermined manner. For example, a sensor circuit onboard a replaceable staple cartridge can be utilized to detect a tissue density, for example, regardless of which modular electronics package is installed in the staple cartridge. The measured tissue density can be used during firing to modify the firing control algorithm. However, depending on which modular electronics package (selected for a specific type of tissue) is installed, the firing control algorithm can be modified differently for the same tissue density measurement with a different modular electronics package (associated with a different type of tissue) installed in the stapling end effector. Such an arrangement can allow a user to change the onboard control circuit by swapping the modular electronics package with a different modular electronics package to accommodate the specific procedure within which the staple cartridge is to be used. The selected control circuit may process sensor circuit values differently depending on the type of tissue associated with that modular electronics package.

Changing the modular electronics package may also be performed to update the software, firmware, and/or other control programs/algorithms of the replaceable staple cartridge. In accordance with the present disclosure, the replaceable staple cartridge can be manufactured and the modular electronics package can be updated over time so the physical design of the replaceable staple cartridge need not change overtime as the control algorithms/programs are updated. Upon distributing the replaceable staple cartridge, the most up to date modular electronics package can be easily and quickly installed on the replaceable staple cartridge. New modular electronics packages can be designed to fit in the existing cartridge receptacle.

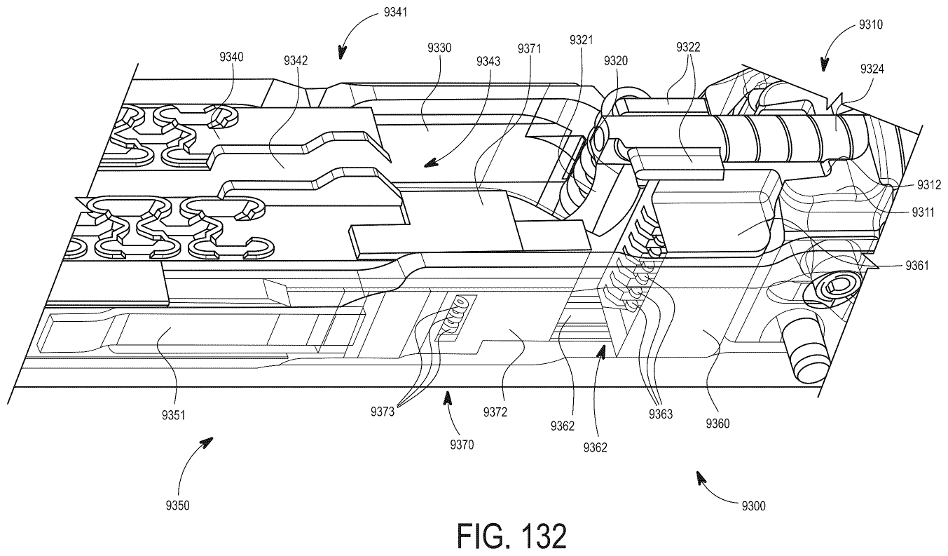

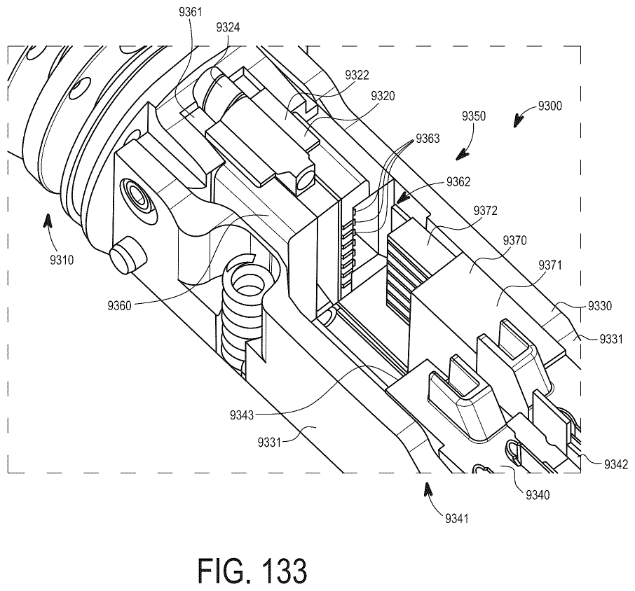

FIGS. 132 and 133 depict a surgical stapling end effector 9300 comprising a shaft assembly 9310 including a channel retainer 9311, a cartridge channel jaw 9330 supported by the channel retainer 9311, and a replaceable staple cartridge 9340 configured to be installed into the cartridge channel jaw 9330. The surgical stapling end effector 9300 further comprises a firing driver 9320 including a cutting edge to cut tissue, anvil cams 9322 to engage the anvil during a firing stroke, and channel cams 9323 to engage the cartridge channel jaw 9330 during the firing stroke. The firing driver 9320 is illustrated in its proximal-most position or, an unfired position.

The replaceable staple cartridge 9340 comprises a proximal end 9341, a slot 9342 configured to receive at least a portion of the firing drive 9320 during the firing stroke, and a proximally-facing side 9343. The replaceable staple cartridge 9340 further comprises an electronic system 9350. The electronic system 9350 may comprise any suitable electronic components such as those disclosed herein. For example, the replaceable staple cartridge 9340 comprises a wireless transmission interface, one or more onboard sensor circuits, one or more onboard cartridge identifier circuits, and/or one or more modular electronics packages. One or more of the electronic components are configured to receive power and/or data from the surgical instrument (surgical robot and/or surgical instrument handle, for example) to which the replaceable staple cartridge 9340 is attached. The electronic system 9350 of the replaceable staple cartridge 9340 further comprises electrical connectors 9360, 9370 through which power and/or data is transferred.

The electrical connector 9360 is positioned within the cartridge channel jaw 9330 and at least a portion of the channel retainer 9311. The electrical connector 9360 comprises a female slot 9362 configured to receive a male end 9372 of the electrical connector 9370. The electrical connector 9360 comprises a body 9361 at least partially positioned within a slot 9312 of the channel retainer 9311. As can be seen in FIGS. 132 and 133, the electrical connector is positioned proximal to the cutting edge 9321 when the firing driver 9320 is in its unfired position. The electrical connector 9360 further comprises a plurality of contacts 9363 positioned within the female slot 9361 and configured to be engaged by a plurality of corresponding contacts 9373 of the electrical connector 9370. The male end 9372 extends proximally from a body 9371 of the electrical connector 9370. As can be seen in FIG. 132, the electrical connector 9370 extends proximally from the proximal-facing side 9343 of the replaceable staple cartridge 9340.

The replaceable staple cartridge 9340 is configured to be slid proximally into the cartridge channel jaw 9330. This distal-to-proximal installation motion allows the male end 9372 of the electrical connector 9370 to be inserted into the female slot 9362 of the electrical connector 9360 when installing the replaceable staple cartridge 9340 into the cartridge channel jaw 9330. Once connected, power and/or data may be transferred through the contacts 9363, 9373. The electronic system 9350 further comprises an electrical cable 9351 connected to the contacts 9373 to carry electrical signals to the various onboard electronics of the replaceable staple cartridge 9340. The various onboard electronics of the replaceable staple cartridge 9340 may receive electrical signals from the electrical connector 9370 individually. As can be seen in FIG. 133, the cartridge channel jaw 9330 comprises channel sides 9331 configured to support the replaceable staple cartridge 9340 during and after installation of the replaceable staple cartridge 9340 into the cartridge channel jaw 9330. The electrical cable 9351 is positioned between the cartridge channel jaw 9330 and a sidewall of the replaceable staple cartridge 9340.

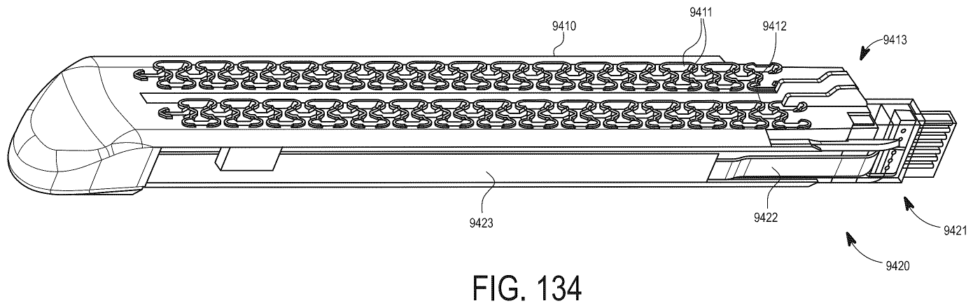

FIG. 134 is a perspective view of a replaceable staple cartridge 9400 for use with a surgical system such as those disclosed herein. The replaceable staple cartridge 9400 is configured to be installed into a cartridge channel jaw of a surgical stapling end effector. The replaceable staple cartridge 9400 comprises a cartridge body 9410 comprising a plurality of staple cavities 9411, a longitudinal slot 9412, and a proximal end 9413. The replaceable staple cartridge 9400 comprises an electrical system 9420 comprising an electrical connector 9421 extending from the proximal end 9413 of the cartridge body 9410, an electrical cable 9422 electrically coupled to the electrical connector 9421, and onboard electronics 9423 electrically coupled to the electrical connector 9421.

The onboard electronics 9423 can comprise any suitable onboard electronics such as those disclosed herein. In accordance with the present disclosure, the onboard electronics 9423 may comprise a modular electronics package, a PCB including a processor and a memory among other electrical components, a wireless transmission interface, one or more sensor circuits electrically coupled to the electrical connector 9421 and/or the modular electronics package, PCB, and/or the wireless transmission interface, and/or one or more cartridge-identifier circuits electrically coupled to the electrical connector 9421 and/or the modular electronics package, PCB, and/or the wireless transmission interface.

In accordance with the present disclosure, one or more modular electronics packages may be capable of being installed into a receptacle of a replaceable staple cartridge. Where multiple modular electronics packages (PCBs, for example) are installed into the receptacle, each modular electronics package is electrically coupled to each other. Additionally, each modular electronics package can be electrically coupled to the control circuit 9004 independently through the electrical connector of the replaceable staple cartridge. In accordance with the present disclosure, the entire receptacle may contain multiple sets of electrical contacts for each component installed in the receptacle. Each set of electrical contacts can be electrically coupled to the wireless transmission interface and/or the electrical connector.

In accordance with the present disclosure, all of the electrical components installed in the receptacle of a replaceable staple cartridge can be powered through a wireless coil of the wireless transmission interface (and thus the corresponding wireless transmission interface of the cartridge channel jaw) and receive and/or transmit data through another wireless coil of the wireless transmission interface (and thus the corresponding wireless transmission interface of the cartridge channel jaw). In accordance with the present disclosure, one or more of the electrical components installed in the receptacle may be battery powered.

In addition to, or in lieu of, receiving power and data through a wireless transmission interface, the electrical components installed in the receptacle of a replaceable staple cartridge are electrically coupled to the surgical instrument through the electrical connector of the replaceable staple cartridge to receive power and/or data therethrough. Such an electrical connection can serve as a redundancy, or back up, path of electrical power and/or data to and/or from the replaceable staple cartridge. In accordance with the present disclosure, some of the electrical components of a replaceable staple cartridge can be electrically coupled to the surgical instrument through the electrical connector, while others can be electrically coupled to the surgical instrument through the wireless transmission interface. Additionally, in accordance with the present disclosure, all of the data transfer may be achieved through the hardwired interface and all of the power transfer may be achieved through the wireless transmission interface. Alternatively, all of the power transfer may be achieved through the hardwired interface and all of the data transfer may be achieved through the wireless transmission interface. In accordance with the present disclosure, certain components of the staple cartridge may receive power through the hardwired interface and certain components of the staple cartridge may receive power through the wireless transmission interface. Alternatively, in accordance with the present disclosure, certain components of the staple cartridge may receive data through the hardwired interface and certain components of the staple cartridge may receive data through the wireless transmission interface.

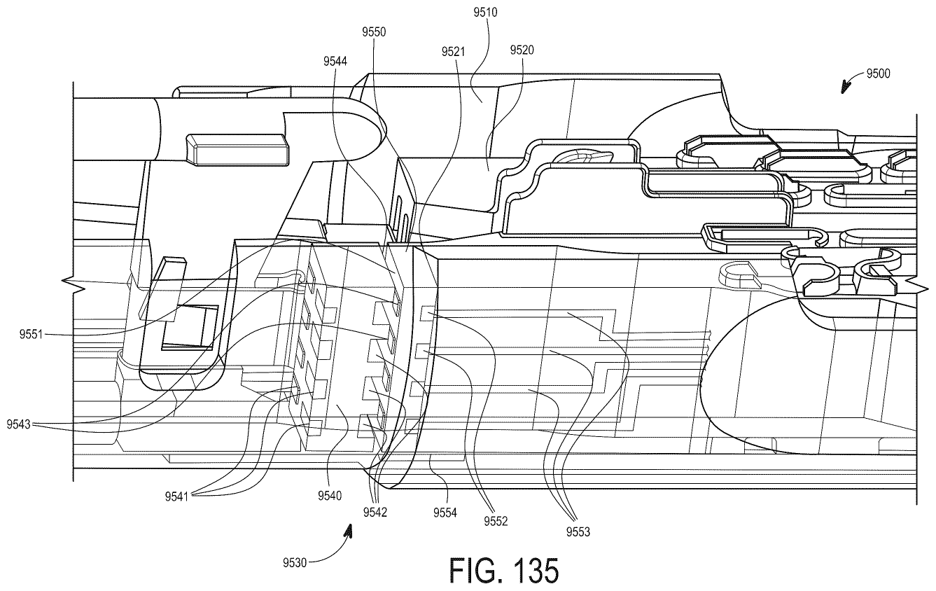

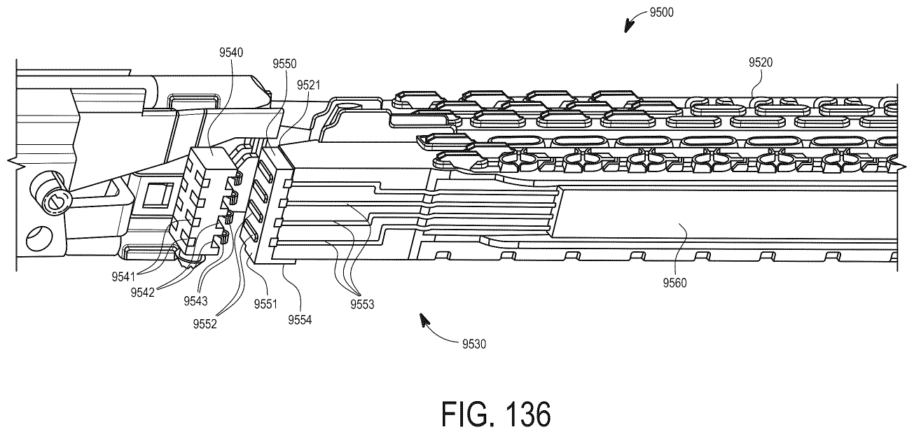

FIGS. 135 and 136 depict a surgical stapling end effector 9500 including a cartridge channel jaw 9510, a replaceable staple cartridge 9520 having onboard electronics 9560, and an electrical connector system 9530 configured to transmit data and/or power between the onboard electronics 9560 and the control circuit 9004 of the surgical system 9002, for example. The electrical connector system 9530 comprises a proximal connector 9540 positioned within the cartridge channel jaw 9510 and a distal connector 9530 mounted to a proximal face 9521 of the replaceable staple cartridge 9520. The proximal connector 9540 comprises proximal contacts 9541 electrically coupled with a cable, for example, to carry electrical signals to and/or from the control circuit 9004 positioned within a surgical instrument handle, shaft, and/or robotic puck, for example. The proximal connector 9540 comprises a distal end 9544 having a plurality of slots 9542 and electrical connectors 9543 extending out of the slots 9542 and electrically coupled to the proximal contacts 9541.

The distal connector 9550 is mounted to the proximal face 9521 and comprises a tab 9554 extending underneath a bottom of a cartridge body of the replaceable staple cartridge 9520. The tab 9554 may serve to support the replaceable staple cartridge 9520 against the cartridge channel jaw 9510 upon installation. In accordance with the present disclosure, the tab 9554 may serve as an alignment aid for aligning the proximal connector 9540 and the distal connector 9550 during and/or after installation of the replaceable staple cartridge 9520 in the cartridge channel jaw 9530. The distal connecter 9550 comprises electrical contacts 9552 extending from a proximal side 9551 of the connector 9550, through the connector 9550, and electrically coupled to electrical traces, or leads, 9553 distal to the connector 9550. In accordance with the present disclosure, the electrical traces 9553 can be positioned within slots, or channels, defined in the cartridge body of the replaceable staple cartridge 9540. The electrical traces 9553 are configured to carry electrical signals to and/or from onboard electronics 9560.

In accordance with the present disclosure, the electrical connectors 9543 may be spring loaded such that, as the replaceable staple cartridge 9520 is installed in the cartridge channel jaw 9510 in a distal-to-proximal direction, the electrical connectors 9542 are able to be engaged by and pushed into the slots, as necessary, by corresponding contacts 9552 upon installation of the replaceable staple cartridge 9520 into the cartridge channel jaw 9510 to affirmatively bias the connectors 9542 into engagement with the contacts 9552.

In accordance with the present disclosure, the one or more onboard electronics discussed herein may be installed into a cartridge channel jaw in addition to, or in place of, onboard electronics installed on a replaceable staple cartridge.

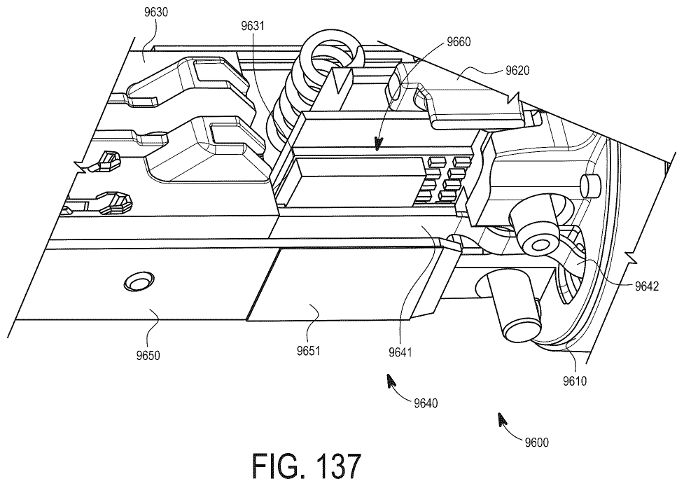

FIG. 137 depicts a surgical stapling assembly 9600 comprising a shaft assembly 9610 and a replaceable staple cartridge 9630. The replaceable staple cartridge 9630 is configured to be installed into a cartridge channel jaw. The surgical stapling assembly 9600 further comprises an electrical system 9640 comprising a PCB 9650 having a wireless transmission interface, or coils, 9651 integrated into the PCB 9650 and a wireless transmission interface, or coil, 9641 configured to transfer power and/or data to and/or from the wireless transmission interface 9651. The wireless transmission interface 9641 is supplied with electrical data and/or power through a cable 9642.

The surgical stapling assembly 9600 further comprises a firing drive 9620 and an electronics assembly 9660 positioned between the replaceable staple cartridge 9630 and the shaft assembly 9610. The electronics assembly 9660 can be configured to receive power and/or data from the wireless transmission interface 9641. In accordance with the present disclosure, the electronics assembly 9660 may comprise onboard sensor circuits configured to measure one or more parameters of the firing driver 9620 during a firing stroke. This information can be transmitted to the PCB 9650 and/or back to a control circuit of the surgical instrument through the cable 9642. As can be seen in FIG. 137, the wireless transmission interface 9651 is proximal to a proximal end 9631 of the replaceable staple cartridge 9630. In accordance with the present disclosure, the PCB 9650 may be attachable to and/or detachable from the replaceable staple cartridge 9630. Additionally, the PCB 9650 may be attachable to and/or detachable from a cartridge channel jaw.

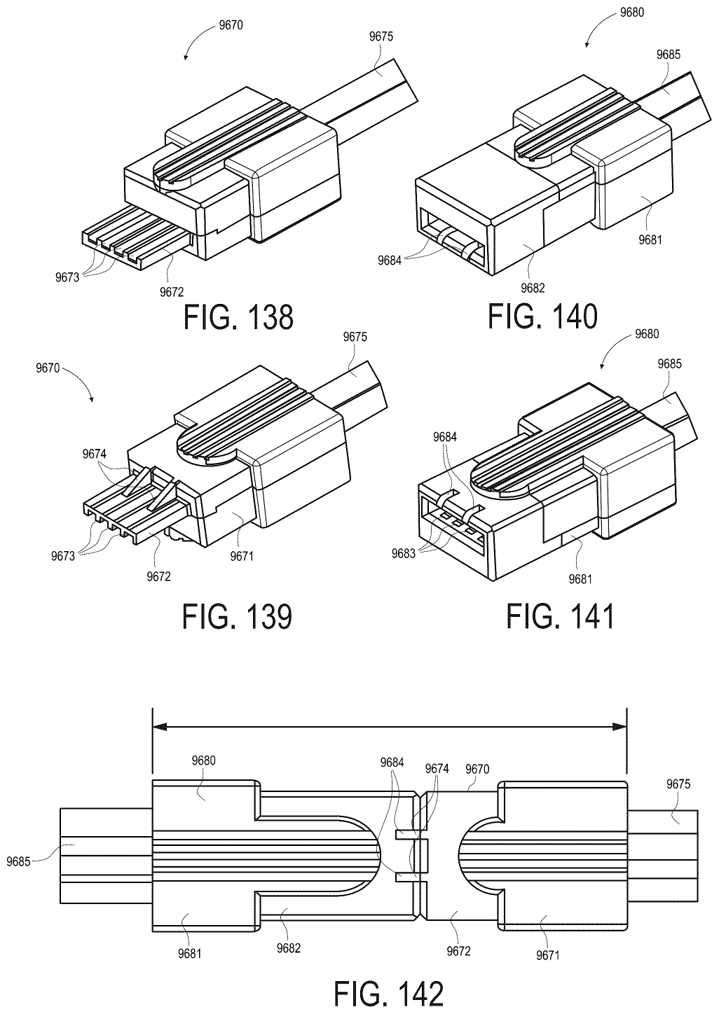

FIGS. 138-142 depict an electrical connector arrangement comprising a male connector 9670 and a female connector 9680 configured to receive the male connector 9670. The connector arrangement can be used with a replaceable staple cartridge and a cartridge channel jaw where one of the connectors 9670, 9680 is attached, or mounted, to the replaceable staple cartridge and the other of the connectors 9670, 9680 is attached, or mounted, to the cartridge channel jaw. In accordance with the present disclosure, the electrical connector connected to the cartridge can be mounted to the bottom of the replaceable staple cartridge near the distal end and the electrical connector can be mounted to the end of the cartridge channel jaw. In such an arrangement, the replaceable staple cartridge can be inserted into the jaws at an angle, brought down at least substantially parallel to the cartridge channel jaw, and pushed fully proximally to seat the cartridge into the jaw which also physically couples the electrical connectors.

The male connector 9670 comprises a connector body 9671 having a male end 9672. Electrical contacts 9673 are exposed on the male end 9672. The male connector 9670 further comprises alignment fins 9674 extending from the connector body 9671 to the male end 9672. In accordance with the present disclosure, the fins 9674 may provide additional support, or rigidity, to the male end 9672. The female connector 9680 comprises a connector body 9681 having a female end 9682. Electrical contacts 9683 are exposed inside of the female end 9682. The female end 9682 further comprises slots 9684 defined in the female end to receive the fins 9674 of the male end 9672 upon complete insertion of the male end 9672 into the female end 9682. As can be seen in FIG. 142, the connectors 9670, 9680 are fully connected where electrical signals from electrical cables 9675, 9685 may be transmitted therebetween by way of the contacts 9673, 9683.

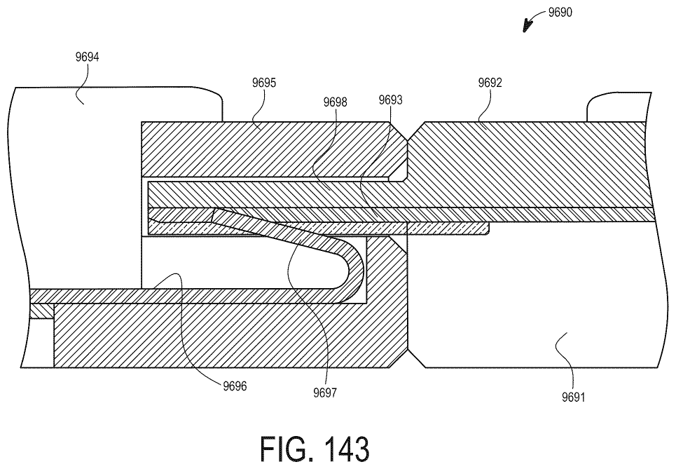

FIG. 143 depicts an electrical connector arrangement 9690 between a cartridge channel jaw 9691 and a replaceable staple cartridge 9694. The cartridge channel jaw 9691 comprises an electrical connector 9692 having a male end 9698 and an electrical contact 9693 exposed on the male end 9698. The replaceable staple cartridge 9694 comprises a female electrical connector 9695 having an electrical contact 9696 exposed inside of the female electrical connector 9695. The electrical contact 9696 has a spring end 9697 configured to engage the electrical contact 9693 of the male end 9698 upon insertion of the male end 9698 into the female electrical connector 9695. In accordance with the present disclosure, the replaceable staple cartridge may comprise the male electrical connector and the cartridge channel jaw may comprise the female electrical connector.

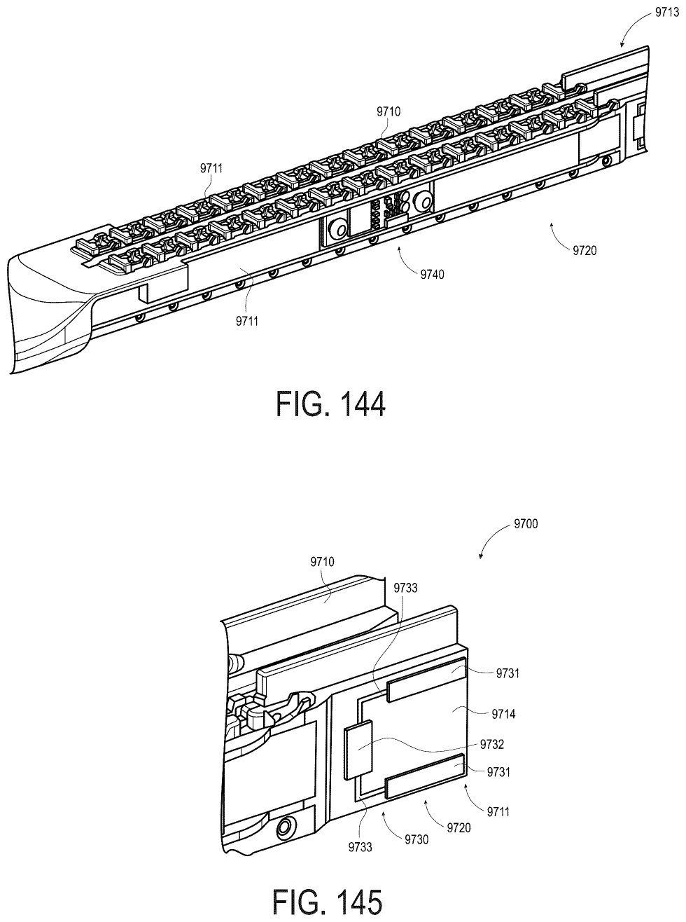

FIGS. 144 and 145 depict a replaceable staple cartridge 9700 for use with one or more of the surgical systems disclosed herein. The replaceable staple cartridge 9700 comprises a cartridge body 9710 having cartridge sides 9711 and a proximal end 9713. The replaceable staple cartridge 9700 further comprises onboard electronics 9720. The onboard electronics 9720 comprise any suitable electronic component disclosed herein and combinations thereof. The onboard electronics 9720 comprise a modular electronics package 9740 replaceably attached to a side 9711 of the cartridge body 9710. The onboard electronics 9720 further comprise a cartridge-identifier circuit 9730 on a side 9714 of the proximal end 9713.

The circuit 9730 is configured to be detectable by a control circuit of a surgical instrument to which the replaceable staple cartridge 9700 is configured to be attached. The circuit 9730 comprises circuit contacts 9731, a resistance element 9732, and electrical traces 9733 electrically coupling the contacts 9731 and the resistance element 9732. The circuit 9730 is configured to be electrically coupled with a circuit of the instrument to which the cartridge 9700 is attached upon installation of the cartridge 9700 into the instrument. The resistance element 9732 is detectable by a control circuit of the instrument. The resistance element 9732 comprises a resistance value indicative of one or more properties of the replaceable staple cartridge 9700 such as, for example, cartridge color, type, size, length, staple height, staple diameter, etc.

Upon installation of the replaceable staple cartridge 9700 into an instrument, the control circuit, such as the control circuit 9004, for example, utilizes a lookup table of resistance values to identified the one or more properties, or characteristics, of the replaceable staple cartridge 9700. Such information may also be stored in the modular electronics package. The control circuit 9004 can compare the information gathered from the circuit 9730 and the information on the modular electronics package to identify the staple cartridge 9700 and verify the authenticity of the cartridge 9700, for example. In accordance with the present disclosure, where a modular electronics package is not installed and/or not required, the control circuit 9004 can identify the cartridge 9700 using only the circuit 9730. Additionally, the circuit 9730 can be screen printed onto the cartridge body 9710.

In accordance with the present disclosure, the modular electronics package 9740, when installed in the cartridge 9700, can be electrically coupled to the circuit 9730. Such an arrangement allows the modular electronics package 9740 to identify cartridge characteristics upon installation of the modular electronics package 9740 into the replaceable staple cartridge 9700. The modular electronics package 9740 may then be configured to make adjustments to firing stroke control feedback circuits, communicate this information to a surgical instrument and/or surgical robot, for example, etc.

In accordance with the present disclosure, sensor arrays may be provided on stapling end effectors to measure one or more parameters of tissue before, during, and/or after clamping, stapling, and cutting tissue. A control circuit can be provided to intelligently decode information gathered by these sensor arrays to more accurately define a state of the tissue during a surgical stapling procedure.

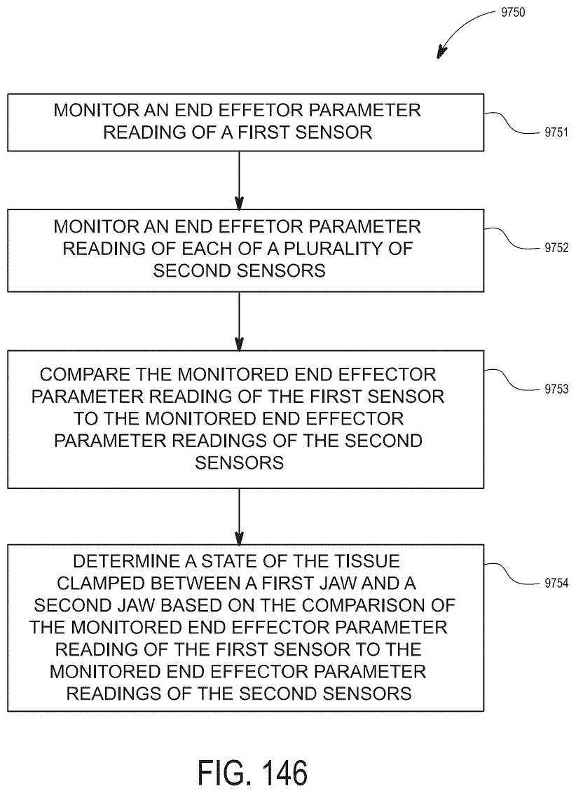

FIG. 146 depicts a logic flow diagram 9750 executable by a control circuit, such as the control circuit 9004, of a surgical system utilizing a sensor circuit in a stapling end effector. The steps of the control circuit can help accurately determine a state of the tissue within the stapling end effector. The control circuit is configured to monitor 9751 an end effector parameter reading of a first sensor positioned within the end effector. The control circuit is further configured to monitor 9752 an end effector parameter reading of each of a plurality of second sensors. The parameter reading may be any suitable parameter reading, such as, for example, tissue thickness, gap distance between the jaws, clamp pressure, gap distance between the tissue positioned between the jaws and one of the jaws etc. The parameter reading may be captured through any suitable sensor such as, for example, strain gauges, pressure gauges, optical sensors, capacitive sensors, distance sensors, force sensors, etc. The readings can be monitored before, during, and after tissue clamping, cutting, and stapling at a predetermined sampling rate.

The control circuit is further configured to compare 9753 the monitored end effector parameter reading of the first sensor to the monitored end effector parameter readings of the second sensors. Comparing this information allows the control circuit to analyze the readings of each of the sensors relative to each other and not in a vacuum where the end effector parameter readings of each sensor is viewed independently.

In accordance with the present disclosure, the first sensor may be distal to the plurality of second sensors, which may be distributed along a length of the stapling end effector. Positioning the first sensor distal to the plurality of the second sensors allows the control circuit to determine if the jaws of the stapling end effector have been overstuffed with tissue, for example. The control circuit can be further configured to determine 9754 a state of the tissue clamped between the first jaw and the second jaw of the stapling end effector based on the comparison of the monitored end effector parameter reading of the first sensor to the monitored end effector parameter readings of the second sensors.

In accordance with the present disclosure, the control circuit can be configured to determine if the jaws have been overstuffed or if the tissue clamped between the jaws of the stapling end effector is indeed thicker than expected, for example. If the jaws are overstuffed with tissue, the rotatable jaw to clamp the tissue may not be able to fully close, for example. Such a configuration may arise where a thick portion of tissue is stuck in the smaller gap between the jaws nearer a pivot location of the jaws. In such a configuration, a larger gap between tissue and the jaws may exist between the jaws distal to the second sensors (or nearer the distal end of the jaws, for example). In such a scenario, the plurality of second sensors may indicate that thick tissue is clamped between the jaws at the location of the second sensors. This can be determined by measuring little, to no, gap distance between the jaw on which the sensors are placed and the tissue itself, for example. Another method for determining this can include a significant pressure reading indicating the tissue is being squeezed substantially by the jaws at the location of the second sensors.

The control circuit is also configured to monitor the end effector parameter reading of the first sensor that is distal to the second sensors and compare the reading to the readings of the second sensors. In the scenario where the jaws are overstuffed (thick tissue present toward a proximal end of the jaws and thin, or no, tissue present toward the distal end), the reading of the first sensor may indicate that little, or no, tissue is present toward the distal end. This can be determined by a much greater than expected gap distance between the jaw on which the sensor is placed and the tissue itself, for example. Another method for determining this can include little, to no, pressure detected at the first sensor indicating the tissue nearer the first sensor is not substantially consuming the gap between the jaws of the stapling end effector.

In the scenario of overstuffing the jaws, a highly disproportionate difference may exist between the end effector parameter reading of the first sensor and the end effector parameter readings of the second sensors. This difference can be defined by a threshold percentage difference, for example. Once the control circuit identifies that a threshold percentage difference exists, action can be taken by the control circuit. In accordance with the present disclosure, the control circuit can be configured to alert a user that the jaws are overstuffed and/or prompt the user to unclamp, and re-clamp, tissue.

In accordance with the present disclosure, a highly disproportionate difference between the end effector parameter reading of the first sensor and the end effector parameter readings of the second sensors can indicate that the jaws are clamped onto a foreign object. In such a scenario, the control circuit may alert a user that the jaws are clamped onto a foreign object. Additionally, the control circuit can be configured to prompt a user to unclamp, and re-clamp, tissue. Highly disproportionate differences in strain values between the second sensors (an average strain across all of the second sensors) and the first sensor can indicate the presence of a foreign object.

In accordance with the present disclosure, a third sensor may be employed to measure the same, and/or a different end effector parameter. In such an instance, the control circuit can be configured to compare the highly disproportionate difference discussed above to the end effector parameter reading of the third sensor. In accordance with the present disclosure, the third sensor may comprise a Hall effect sensor. The third sensor can be used to validate, or verify, the state of tissue, or state of what is positioned between the jaws, determined when comparing the reading of the first sensor to the readings of the second sensors. A high Hall effect sensor reading can indicate an overstressed-tissue condition.

In an instance where the distal-most sensor indicates thick, or dense, tissue and the first sensor and/or second sensors indicate tissue of a nominal thickness, for example, the control circuit determines that calcified tissue and/or irregular tissue is positioned nearer the distal end of the jaws. The control circuit can then recommend not to fire with this determination of the state of the tissue.

In accordance with the present disclosure, the second sensors may comprise strain gauges positioned on the channel jaw, cartridge deck, and/or anvil jaw and the first sensor may comprise a Hall effect sensor. Additionally, one or more of the strain gauges may comprise half bridge, and/or full bridge, strain gauges. In such an instance, an electronics package module capable of receiving and decoding information gathered from the half bridge, and/or full bridge, strain gauges may be required. Any suitable strain gauge can be used. Strain gauges which measure stress and/or strain in multiple directions can allow a control circuit to determine the amount of twist experienced by the jaw to which they are attached such as, for example, anvil twist. Anvil twist is indicated by rotational twisting of the anvil relative to a longitudinal axis of the anvil.

In accordance with the present disclosure, the angle of the jaw can be measured with a first sensor, and a Hall effector sensor at the distal end can measure a gap distance between the jaws at the distal end. Additionally, a third sensor may be employed to measure tissue-conductivity in between the first sensor and the Hall effect sensor.

In accordance with the present disclosure, sensors may be selected so as to measure tissue location as well as tissue thickness. Alternatively, sensors may be selected so as to measure tissue composition. In such an instance, multiple complex sensors, which require an electronics control package with a processor to operate and use with a surgical system, such as the surgical system 9002, for example, can be utilized to measure various tissue parameters. Density, thickness, and/or electrical conductivity of the tissue are all examples of tissue parameters measurable by a complex sensor. In accordance with the present disclosure, the modular electronics package installed on the staple cartridge may comprise a multiplexer onboard the modular electronics package electrically couplable with the sensor circuits. The sensor circuits may utilize impedance spectroscopy to measure electrical properties of the tissue and/or whatever is positioned between the jaws. In accordance with the present disclosure, tissue perfusion may be measured using a sensor circuit. Additionally, multiple laser Doppler imaging sensors may be used to scan tissue.

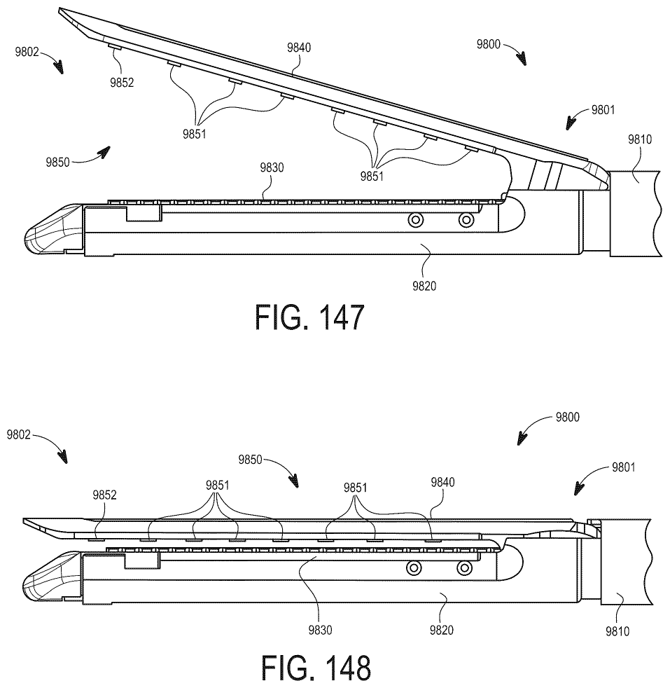

FIGS. 147-149 depict a surgical stapling end effector 9800. The surgical stapling end effector 9800 comprises a shaft 9810, a channel jaw 9820 configured to receive a replaceable staple cartridge 9830, and an anvil jaw 9840 movable relative to the channel jaw 9820. In accordance with the present disclosure, the channel jaw 9820 can be movable relative to the anvil jaw 9840 in addition to, or in lieu of, the anvil jaw 9840 being movable relative to the channel jaw 9820. The surgical stapling end effector 9800 comprises a sensor circuit 9850. The surgical stapling end effector 9800 can be employed with the logic flow diagram 9750 and a control circuit, such as the control circuit 9004.

The sensor circuit 9050 comprises a plurality of sensors 9851, 9852 mounted to the anvil jaw 9840. In accordance with the present disclosure, some or all of the sensors 9851, 9852 can be mounted to the channel jaw 9820 and/or the staple cartridge 9830. The plurality of sensors 9851, 9852 comprise a plurality of first sensors 9851 and a second sensor 9852 distal to the plurality of first sensors 9851. The sensors 9851, 9852 may comprise any suitable sensor to measure an end effector parameter. In accordance with the present disclosure, the sensors 9851 may comprise strain gauges to measure strain induced by clamping tissue between the jaws. Additionally, the sensor 9852 may comprise a Hall effect sensor configured to measure a gap distance between the jaws 9820, 9840 at a distal end 9802 of the surgical stapling end effector 9800. The surgical stapling end effector 9800 further comprises a proximal end 9801. FIG. 147 illustrates the jaws 9820, 9840 in an open position and FIG. 146 illustrates the jaws 9820, 9840 in a closed position.

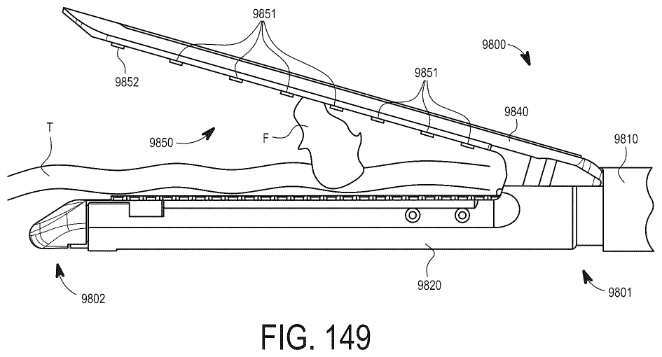

As can be seen in FIG. 149, tissue T and a foreign object F are positioned between the jaws 9820, 9840. The fact that a foreign object F is clamped between the jaws 9820 and not thick tissue can be determined by the control circuit comparing the outputs of the sensors 9851 and the output of the sensor 9852. For example, one or more of the sensors 9851 can indicate a disproportionate gap distance measurement, for example, compared to a gap distance measurement of the sensor 9852. This disproportionate difference can indicate that a foreign object is clamped between the jaws 9820, 9840. In accordance with the present disclosure, if the jaws 9820, 9840 are over stuffed with thick tissue positioned nearer the proximal end 9801, the comparison of the outputs of the sensors 9851 and sensor 9852 can indicate a disproportionate strain difference. This disproportionate strain difference can indicate that the jaws 9820, 9840 have been overstuffed with tissue. In accordance with the present disclosure, the sensors 9851 may comprise a plurality of sensor zones monitorable by the sensors 9851 to indicate the presence of calcified, or overly thick, tissue in each zone (proximal zone, intermediate zone, and distal zone, for example). In such an instance, the control circuit can determine which zones are overstuffed with tissue, for example. In accordance with the present disclosure, an average may be taken of the outputs of each sensor 9851, or each zone of sensors 9851, and the average can be compared to the output of the sensor 9852.

In accordance with the present disclosure, electrical traces can be positioned within one or more pathways, or channels, defined in and/or on the anvil jaw and/or the staple cartridge jaw and can be coupled with sensors along the length of the anvil jaw and/or staple cartridge jaw. Additionally, the pathways can be cut into the jaws with a laser. Alternatively, the electrical traces can be plated onto the jaws.

In accordance with the present disclosure, a wireless coil may be first used to check cartridge viability (authenticity, unfired, etc.) and, upon passing a first check through the wireless coil, the control circuit can be configured permit power and/or data transfer to the staple cartridge assembly through another coil and/or other electrical system (hardwired, electrical connector, etc.).

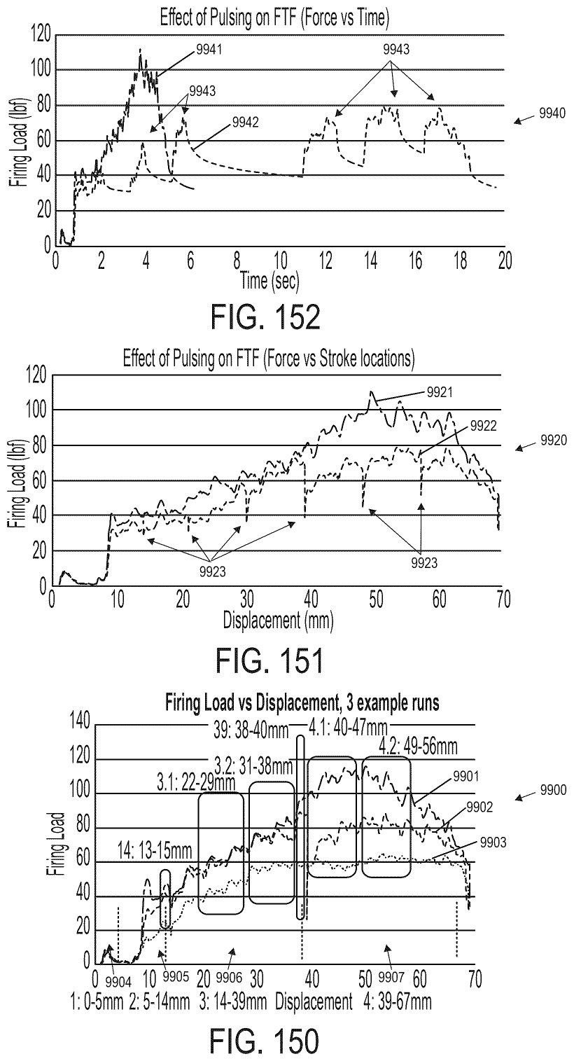

FIG. 150 is a graph 9900 of three firing strokes 9901, 9902, 9903 where an end effector contains multiple sensor zones 9904, 9905, 9906, 9907. Each sensor zone 9904, 9905, 9906, 9907 contains at least one sensor such as, for example, a strain gauge, configured to sense a parameter of the firing stroke (tissue thickness, tissue toughness, firing driver speed, tissue clamping pressure, etc.) within the corresponding sensor zone. Having sensors within discrete zones of the end effector can allow for more targeted measurements of tissue based on predictable behavior of the end effector within each zone. As a result, unpredictable sensor readings would indicate an abnormal condition such as, for example, over stuffing of the jaws.

For example, nearer the distal end of a stapling end effector, the cantilever beam effect of the jaw which is clamped from an open position to a closed position can be higher than the cantilever beam effector of the jaw nearer the proximal end of the stapling end effector. This cantilever beam effect can result in greater strain value measurements within these zones given the jaw's inability to resist deflection in this zone compared to more proximal zones where the movable jaw is much stiffer. A higher strain reading within this distal zone, for example, could be expected given the decreased stiffness of the jaw within this zone compared to others. This sensor reading can be compared to different sensor reading thresholds as compared to other zones given the difference in predictable behavior of the zone. Similarly, nearer the proximal end of the stapling end effector, the movable jaw may comprise a much greater stiffness and not be expected to experience the same amount of strain as more distal zones under a given tissue load. Thus, a predetermined tissue-overstuffing strain value indicative of tissue over-stuffing may be much lower in this zone as compared to a more distal zone. Given the greater stiffness of this portion of the movable jaw, the same amount increase, for example, of the measured strain value relative to the predetermined tissue-overstuffing strain value in both zones with different jaw stiffness, for example, may indicate a much more significant overstuffing scenario in the zone where the movable jaw is much stiffer. Placing sensors in discrete zones can allow a control circuit to determine which zone is experiencing overstuffing, for example.

FIGS. 151 and 152 illustrate the effects of pulsing a firing stroke relative to not pulsing a firing stroke of a stapling end effector. FIG. 151 is a graph 9920 of a first firing stroke 9921 and a second firing stroke 9922 of a stapling end effector plotting firing load (y axis) vs displacement (x axis). The difference between the strokes 9921, 9923 is that the second firing stroke 9922 includes a pulsed firing stroke where a firing motor is pulsed throughout the firing stroke to reduce firing load throughout the firing stroke. As can be seen in the graph 9920, the second firing stroke experiences, on average, less firing load over the duration of the firing stroke given the pulses 9923. Pulsing the firing motor in such a fashion can allow the tissue to relax, or soften, briefly so as to lighten the firing load on a cutting edge of a firing driver, for example. FIG. 152 is a graph 9940 of a first firing stroke 9941 and a second firing stroke 9942 where the second firing stroke 9942 is pulsed throughout the duration of the firing stroke 9942. As can be seen in the graph 9940, the firing load is maintained below a threshold throughout the duration of the second firing stroke 9942 at least because of the pulsing 9943 of the firing motor, for example.

Many of the surgical instrument systems described herein are motivated by an electric motor; however, the surgical instrument systems described herein can be motivated in any suitable manner. In accordance with the present disclosure, the surgical instrument systems described herein can be motivated by a manually-operated trigger, for example. In accordance with the present disclosure, the motors disclosed herein may comprise a portion or portions of a robotically controlled system. Moreover, any of the end effectors and/or tool assemblies disclosed herein can be utilized with a robotic surgical instrument system. U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535, for example, discloses several examples of a robotic surgical instrument system in greater detail.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one or more other embodiments without limitation. Also, where materials are disclosed for certain components, other materials may be used. According to various embodiments, a single component may be replaced by multiple components, and multiple components may be replaced by a single component, to perform a given function or functions. The foregoing description and following claims are intended to cover all such modification and variations.

While several configurations have been described, additional modifications are within the scope of the present disclosure, which is intended to cover any variations, uses, or adaptations of the disclosed configurations using its general principles.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. The instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

Any of the software components or functions described in this application, may be implemented as software code to be executed by a processor using any suitable computer language such as, for example, Python, Java, C++ or Perl using, for example, conventional or object-oriented techniques. The software code may be stored as a series of instructions, or commands on a computer readable medium, such as RAM, ROM, a magnetic medium such as a hard-drive or a floppy disk, or an optical medium such as a CD-ROM. Any such computer readable medium may reside on or within a single computational apparatus and may be present on or within different computational apparatuses within a system or network.

As used in any aspect herein, the term “control circuit” or “control system” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in one aspect. Thus, appearances of the phrases “in one aspect,” “in an aspect,” “in an exemplification,” and “in one exemplification” in various places throughout the specification are not necessarily all referring to the same aspect. The particular features, structures or characteristics may be combined in any suitable manner in various aspects.

It is worthy to note that any reference numbers included in the appended claims are used to reference exemplary embodiments/elements described in the present disclosure. Accordingly, any such reference numbers are not meant to limit the scope of the subject matter recited in the appended claims.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A method for sequential firings of staple cartridges, the sequential firings including a first firing that deploys first staples into a first tissue portion from a first staple cartridge, and a second firing that deploys second staples into a second tissue portion from a second staple cartridge, wherein the method comprises:
monitoring a parameter indicative of a tissue response associated with the first firing;
assessing the tissue response based on the parameter; and
adjusting an operational parameter associated with the second firing based on the tissue response during the first firing.

2. The method of claim 1, wherein the tissue response is a first tissue response assessed during a first segment of the first firing, and wherein the method further comprises implementing a first adjustment to an output of the motor based on the first tissue response during the first segment of the first firing.

3. The method of claim 2, further comprising implementing a second adjustment to the output of the motor during a second segment of the first firing, wherein the second segment is distal to the first segment, and wherein the second adjustment is based on an effectiveness of the first adjustment.

4. The method of claim 1, wherein assessing the tissue response based on the parameter comprises comparing a reading of a sensor to a predetermined threshold.

5. The method of claim 1, wherein the parameter is a force exerted against the drive shaft.

6. The method of claim 1, wherein adjusting the operational parameter comprises adjusting a predetermined threshold.

7. The method of claim 1, wherein adjusting the operational parameter is conditioned upon receiving an adjustment approval through a user interface.

8. The method of claim 1, wherein adjusting the operational parameter associated with the second firing comprises:
detecting an identifier of the second staple cartridge;
determining a staple cartridge type of the second staple cartridge based on the identifier; and
recommending a different staple cartridge type for the second firing based on the tissue response in the first firing.

9. The method of claim 1, wherein the different staple cartridge type comprises a different staple-size than the second staple cartridge.

10. The method of claim 1, wherein adjusting the operational parameter further comprises modifying the operational parameter in response to an override input through a user interface.

11. The method of claim 1, wherein monitoring the parameter comprises:
determining a first value of the parameter at a proximal portion of the first firing; and
determining a second value of the parameter at a distal portion of the first firing.

12. The method of claim 1, wherein the first firing comprises a proximal firing region and a distal firing region, wherein assessing the tissue response comprises determining a change in the tissue response between the proximal firing region and the distal firing region.

13. The method of claim 12, wherein adjusting the operational parameter associated with the second firing is based on the change in the tissue response between the proximal firing region and the distal firing region.

14. The method of claim 1, wherein adjusting the operational parameter associated with the second firing is further based on a temperature of a motor measured during the first firing.

15. The method of claim 1, wherein adjusting the operational parameter associated with the second firing is further based on a voltage condition of a battery during the first firing.

16. A method executable by a control circuit of a surgical system including an end effector and a motor powered by a power source, the method comprising:
setting a power source lower threshold;
transitioning the motor to an on state for a first period, the on state in which the motor drives a motion at the end effector to perform a tissue treatment event;
detecting a dropped voltage potential of the power source at the end of the first period;
conducting a first comparison between the dropped voltage potential and the power source lower threshold; and
transitioning the motor to an off state for a second period based on the first comparison, the off state in which the motor ceases to the motion at the end effector.

17. The method of claim 16, further comprising setting a power source recovery threshold, wherein the second period comprises a recovery period based on the dropped voltage potential reaching or dropping below the power source lower threshold, and wherein the recovery period corresponds to a time required for the voltage potential of the power source to recover to the power source recovery threshold.

18. The method of claim 1, wherein the second period is identical to the first period based on the dropped voltage potential being greater than the power source lower threshold.

19. The method of claim 16, further comprising:
detecting, at a first time, a first dropped voltage potential of the power source based on the motor transitioning to the on state;
detecting, at a second time subsequent to the first time, a second dropped voltage potential of the power source based on the motor transitioning to the on state; and
predicting a time that the voltage potential of the power source is expected to reach the power source lower threshold during a subsequent on state of the motor.

20. The method of claim 19, further comprising controlling the motor based on the prediction.
